(19) United States
(12) Patent Application Publication
(10) Pub. No.: US 2022/0287767 A1
(43) Pub. Date: Sep. 15, 2022

(54) Targeted Lung Denervation with Directionally-Adjustable Perfusion

(71) Applicant: Broncus Medical Inc., San Jose, CA (US)

(72) Inventors: Hong XU, Hangzhou (CN); Changjie CUI, Hangzhou (CN); Zhiyu LIU, Hangzhou (CN); Xiangxiang QIN, Hangzhou (CN); Thomas M. Keast, Sunnyvale, CA (US)

(73) Assignee: Broncus Medical Inc., San Jose, CA (US)

(21) Appl. No.: 17/658,947

(22) Filed: Apr. 12, 2022

Related U.S. Application Data
(63) Continuation-in-part of application No. PCT/CN2021/123705, filed on Oct. 14, 2021.
Application No. PCT/CN2021/123705 is a continuation-in-part of application No. PCT/CN2021/072952, filed on Jan. 20, 2021.
Application No. PCT/CN2021/072952 is a continuation-in-part of application No. PCT/CN2021/072953, filed on Jan. 20, 2021.
Application No. PCT/CN2021/072953 is a continuation-in-part of application No. PCT/CN2021/072954, filed on Jan. 20, 2021.
Application No. PCT/CN2021/072954 is a continuation-in-part of application No. PCT/CN2021/072955, filed on Jan. 20, 2021.
Application No. PCT/CN2021/072955 is a continuation-in-part of application No. PCT/CN2021/072956, filed on Jan. 20, 2021.
Application No. PCT/CN2021/072956 is a continuation-in-part of application No. PCT/CN2021/072957, filed on Jan. 20, 2021.
Application No. PCT/CN2021/072957 is a continuation-in-part of application No. PCT/CN2021/072959, filed on Jan. 20, 2021.

(30) Foreign Application Priority Data
Sep. 17, 2020 (CN) 202010980642.8
Dec. 31, 2020 (CN) 202011638301.9
Dec. 31, 2020 (CN) 202011638325.4
Dec. 31, 2020 (CN) 202011638331.X
Dec. 31, 2020 (CN) 202011640959.3
Dec. 31, 2020 (CN) 202011641170.X
Dec. 31, 2020 (CN) 202011641885.5
Dec. 31, 2020 (CN) 202011642360.3

(51) Int. Cl. A61B 18/14; A61B 18/12

(52) U.S. Cl. CPC A61B 18/1492; A61B 18/1206; A61B 2018/0022

(57) ABSTRACT

A lung denervation method comprises advancing a catheter along the airway; deploying an open loop in contact with the epithelium; simultaneously delivering radio frequency energy from a plurality of discrete spaced-apart locations along the loop to target regions according to a set of ablation parameters sufficient to heat and interrupt nerve functionality; and forming a liquid film between the loop and the epithelium for minimizing collateral damage. The method serves to destroy motor axons of the peripheral bronchial nerve, blocks parasympathetic transmission in the pulmonary and reduces acetylcholine release, reducing airway smooth muscle tension and mucus production. Related systems are described.

### BACKGROUND OF THE INVENTION

### 1. Field of the Invention

This invention relates to electrosurgery, and particularly, to radio frequency-based ablation systems for treating chronic bronchitis.

### 2. Description of the Related Art

Chronic obstructive pulmonary disease (chronic obstructive pulmonary disease) Obstructive Pulmonary Disease (COPD) is the most common disease of the respiratory system. In our country, according to the existing epidemiological survey evidence, the prevalence of COPD in adults over 40 years old is about 10%.

At present, COPD mainly relies on drug treatment, and anticholinergic drugs are used to specifically block M receptors, causing relaxation of airway smooth muscle, airway relaxation and mucus secretion, thereby reducing airway obstruction and relieving symptoms of COPD patients, while lung denervation Therapeutic ablation (Targeted Lung Denervation (TLD) pulmonary denervation therapy ablation is aimed at the parasympathetic nerve, blocking its innervation, thereby achieving a permanent anticholinergic effect. A feasibility clinical study of the method was completed in 2015, and further clinical trials are currently underway.

With the continuous improvement of the society's understanding of COPD and the continuous development of interventional technology, the treatment of COPD through airway interventional technology has been recognized by all walks of life. As one of the treatment methods, TLD is more thorough and efficient than drug treatment. and other advantages.

For example, a Chinese patent document with publication number CN111067617A discloses a radio frequency closure catheter, which mainly includes: a tube body, a handle device, a connecting cable and a connector sequentially connected from the distal end to the proximal end, wherein the tube body is connected from the distal end to the proximal end. The distal end is sequentially provided with a rubber head, a heating segment and a main body tube, and the surface of the heating segment is provided with an insulating outer sleeve with insulating and smoothing functions, and the interior of the heating segment is provided with an alloy wire for heating. A wound coil, the coil includes a proximal coil and a distal coil, the extension lines of the proximal coil and the distal coil respectively extend to the handle device through the inner cavity of the main body tube, and are connected through the connection A cable is connected to the connector, which is connected to an external device to provide radio frequency current.

However, in the prior art, the coordination relationship between the components of the radiofrequency ablation catheter is complex, which adversely affects production and assembly, and there is room for improvement in the actual treatment effect.

### SUMMARY OF THE INVENTION

Radiofrequency Ablation Catheter and System Thereof

In order to solve the technical problem, the invention discloses a radio frequency ablation catheter which comprises a pipe body, wherein a traction wire is inserted into the pipe body; the far end part of the pipe body is an annular section; the annular section can deform under the action of a traction wire; a deformation constraint sleeve is fixedly arranged in the pipe body in a penetrating mode; the traction wire penetrates through the deformation constraint sleeve in a penetrating mode; the deformation constraint sleeve is at least extended to the proximal end side of the annular section from the far end of the pipe body, the rigidity D1 of the proximal end of the deformation constraint sleeve is larger than the rigidity D2 located at the annular section, and the deformation constraint pipe is used for adjusting the deformation generated when the pipe body is under the action of the traction wire.

The elastic wire, the traction wire and the wire need to be arranged in the pipe body in a penetrating mode, wherein the elastic wire and the traction wire are located in the inner sleeve, and the wire is located in the radial gap of the inner sleeve and the outer sleeve. Mutual nesting of the inner sleeve and the outer sleeve realizes assembly of the elastic wire, the traction wire and the wire and is isolated from each other, so that unnecessary multiple cavities on the pipe body are avoided. Meanwhile, the inner and outer pipes and the outer sleeve can realize pre-assembling between components, so that the overall production efficiency and yield are improved.

The rigidity D1 is greater than the rigidity D2, and the technical effect brought directly is that the deformation constraint pipe is located at the proximal end side of the annular section and is not prone to deformation relative to the annular section. From the whole of the pipe body, under the action of the traction wire, the annular section is easier to deform compared with the proximal end of the annular section, so that the proximal end of the annular section provides an effect similar to the “base” for deformation of the annular section and is used for controlling the overall shape of the annular section in the space.

The following further provides several optional ways, but does not serve as an additional limitation on the above-mentioned general scheme, and is merely a further supplement or preferred. On the premise of no technical or logical contradiction, various alternatives can be combined independently for the above-mentioned general scheme, and can also be combined among a plurality of optional manners.

Optionally, a main cavity and a fluid cavity extending in parallel along the length direction of the pipe body are arranged in the pipe body, and an output hole communicated with the fluid cavity is formed in the side wall of the pipe body.

The fluid chamber is used for conveying the cooling medium to the output hole, so that the stability of the ablation process is ensured. The flow channel and the main cavity can independently ensure that the flow and the flow rate of the cooling medium are not influenced by components in the main cavity, meanwhile, the wire in the main cavity can also avoid contact between the cooling medium and the cooling medium, and the safety is improved.

Optionally, in the cross section of the annular section, the main cavity is eccentrically arranged relative to the central axis of the pipe body, and is close to the inner edge of the annular section

The annular shape of the distal end of the pipe body is a non-closed annular shape, and under the action of the traction wire, the annular radial dimension change can be realized (see FIGS. 1b and 1c), so that the size adaptation of the catheter to different lesions is achieved. In the embodiment, the traction wire is used for realizing the reduction of the annular radial size, and the elastic wire is used for keeping and recovering the annular radial size. Compared with the central axis of the pipe body, the main cavity is close to the annular inner edge, and it is difficult to understand that in the embodiment disclosed in FIG. 1b, the traction wire is closer to the annular inner edge than the elastic wire. When the traction wire is stressed to move, the traction wire realizes the force application of the elastic wire through the fixed position of the elastic wire, and the elastic wire is stressed to deform so as to realize the change of the radial dimension of the ring.

Optionally, on the cross section of the distal part, the fluid cavity channel is closer to the annular outer edge than the main cavity, and the output hole is located at the outer edge of the ring. The traction wire is closer to the annular inner edge than the elastic wire. When the traction wire exerts acting force on the elastic wire, the traction wire actually moves under the constraint of the inner sleeve pipe, and the acting force of the inner sleeve pipe can be generated. In the embodiment, the movement direction of the traction wire is limited through the cross-sectional shape of the inner sleeve, so that the actuation effect of the traction wire is improved. Meanwhile, the inner sleeve can avoid mutual interference between the traction wire and other components (such as wires) while restraining the traction wire, so that the overall stability of the guide pipe is improved. In one embodiment, the wire is located on one side of the inner sleeve in the direction of the central axis of the ring and abuts against the outer wall of the inner sleeve.

Optionally, the plane where the annular segment is located is intersected with the axial direction of the tube body and the intersection position is an inflection point of the tube body, and the proximal end side of the annular segment turns at the inflection point and extends to the proximal end; the deformation constraint tube is at least extended from the distal end of the tube body to the proximal end side of the annular segment, and the rigidity of the deformation constraint tube on the two sides of the inflection point is the same or different.

The plane where the annular section is located is intersected with the axial direction of the pipe body, so that a large contact area between the annular section and the target point can be realized; in a specific implementation mode, the shape can be formed in the pipe body pre-forming graph to form an inflection point, and the pipe body can be bent through the deformation constraint pipe or the elastic wire to form an inflection point. As shown in the figure, it should also be understood that the boundary between the hydrophobic segment and the key segment is also located near the inflection point, and the boundary between the support tube and the outer sleeve is also located near the inflection point.

Optionally, the deformation constraint pipe itself defines a penetrating channel, and the traction wire extends in the penetrating channel

The acting force of the traction wire can generate a composite effect, and the traction wire can synchronously generate axial compression of the deformation constraint pipe while driving the radial dimension change of the annular section (i.e., the axial bending of the deformation constraint pipe). Axial compression of the deformation constraint pipe at the position of the annular section can be used for realizing the change of the size of the annular section, but axial compression at the near end side of the annular section can influence the positioning effect of the annular section. Therefore, the deformation of the deformation constraint pipe at the near end side of the annular section needs to be limited.

Optionally, a main cavity and a fluid cavity extending in parallel along the length direction of the pipe body are arranged in the annular section, an inner sleeve and an outer sleeve which are nested with each other are arranged in the main cavity, the inner sleeve is arranged in the penetrating channel in a penetrating mode, and the traction wire is arranged in the inner sleeve in a penetrating mode; the deformation constraint pipe is arranged in the outer sleeve in a penetrating mode.

Through the separation of the inner sleeve and the outer sleeve, the main cavity effectively realizes multiple mutually nested channels and is located between the inner sleeve, the inner sleeve and the outer sleeve and between the outer sleeve and the pipe body. The separated main cavity is matched with the fluid cavity to realize independent setting of each pipeline. Each channel extends to the proximal end, and at the same time, a portion of the pipeline communicates with the outside at the distal end. In one embodiment, a main cavity and a fluid cavity extending in parallel along the length direction of the pipe body are arranged in the annular section, an output hole communicated with the fluid cavity is formed in the side wall of the outer edge of the annular section, and a wire hole communicated with the main cavity is formed in the side wall of the inner edge of the annular section.

Optionally, the deformation constraint pipe is a spiral spring and comprises a key section located at the proximal end side of the annular section and a spring section located at the annular section; at least a part of the key section is wrapped with a supporting pipe, and the supporting pipe is bonded and fixed with the key section.

According to the deformation constraint pipe in the embodiment, the rigidity of different positions is achieved through a spiral spring arranged differently. Compared with other arrangement schemes, the spiral spring has the advantages of low cost, good effect, flexible adjustment according to different requirements and the like. Meanwhile, the shape of the spiral spring is matched with the shape of the pipe body, so that the rigidity upper limit of the deformation constraint pipe can be improved on the premise of ensuring that the overall external size is small, and therefore different design requirements are met.

The invention further discloses a radio frequency ablation system The radio frequency ablation system comprises a radio frequency ablation catheter and an operation handle according to the technical scheme, an electrode used for releasing radio frequency energy is arranged on the annular segment, a wire penetrates through the radial gap of the inner sleeve and the outer sleeve, and the end part of the wire penetrates out of the outer sleeve and the pipe wall of the pipe body and is connected with the electrode.

The operating handle comprises: a handle body, wherein the proximal end of the pipe body is directly or indirectly fixed to the handle body; the connecting piece is slidably installed on the handle body, and a mounting hole is formed in the connecting piece; an avoiding channel is arranged on the connecting piece, and the wire penetrates through the connecting piece and extends to the outside of the handle body through the avoiding channel; a plug which is rotationally matched in the mounting hole, and a proximal end part of the traction wire is clamped and fixed in a radial gap of the plug and the mounting hole; a driving piece which is movably installed on the handle body and drives the connecting piece to slide so as to drive the traction wire.

The handle body provides support for each component for determining the relative position of the pipe body and the traction wire to achieve relative movement of the two. In the embodiment, the proximal end of the pipe body is indirectly fixed to the handle body through a weaving pipe. The handle body can provide a holding space for an operator at the same time, and can also be provided with a holding sheath for improving the holding hand feeling. The connector is constrained by the handle body to a motion path within the space. Therefore, the connecting piece is provided with the avoiding hole, so that the size of the connecting piece can be increased as much as possible without interfering with other parts of the connecting piece, so that the contact area of the connecting piece is increased, and the movement stability of the connecting piece is guaranteed. The plug realizes assembly of the traction wire and the connecting piece According to the design of the plug, the assembly is simple and easy to adjust, compared with the related technology, the plug can flexibly adjust the specific position of the traction wire combined with the connecting piece, and the length error of the traction wire can be released. Meanwhile, when the traction wire receives a large acting force, the rotary plug can play a role in preventing overload through rotation of the rotary plug, and the traction wire is prevented from being broken at a weak position.

Optionally, the connecting piece is provided with a traction mounting channel penetrating through the hole wall of the mounting hole, and the proximal end portion of the traction wire enters the radial gap through the traction mounting channel; the rotary plug is provided with a butt joint channel, and the rotary plug is rotated relative to the connecting piece by itself and is respectively provided with: a release state, wherein the traction installation channel and the butt joint channel are mutually aligned, and the traction wire enters the butt joint channel from the traction installation channel; a locking state, wherein the traction mounting channel and the butt joint channel are staggered mutually, and the traction wire enters the radial gap from the traction mounting channel and enters the butt joint channel after extending in the circumferential direction of the plug.

The traction mounting channel is used for allowing the traction wire to penetrate through, and after the traction wire penetrates through the radial gap, the rotary plug rotates to drive the traction wire to enter the interlayer between the plug and the mounting hole, so that clamping is achieved. In one embodiment, the plug is provided with an accommodating groove (not shown) for accommodating at least a portion of the traction wire. The receiving groove is formed in the circumferential direction of the rotary plug and is used for containing the traction wire in the rotation process of the rotary plug, so that the resistance of rotation can be reduced, and excessive stress is prevented from being caused to the traction wire; and the position of the traction wire can be limited, dislocation and other conditions can be avoided, and the stability is improved.

The radio frequency ablation catheter disclosed by the invention is compact in structure, the annular section can flexibly change according to requirements of different working conditions, and the radio frequency ablation system can realize smooth and controllable ablation process.

Specific beneficial technical effects will be further explained in the specific embodiments in combination with specific structures or steps.

Method and Apparatus for Controlling Perfusion of a Plurality of Channels of Syringe Pump, Syringe Pump and Storage Medium

Embodiments of the present invention aim to provide a method and an apparatus for controlling perfusion of a plurality of channels of a syringe pump, a syringe pump and a storage medium, which may not only reduce operation delay and errors caused by manual determination, but also improve the timeliness, the accuracy and the pertinence of perfusing the liquid in a process of performing an ablation task.

In one aspect, embodiments of the present invention provide a method for controlling perfusion of a plurality of channels of a syringe pump, which is applied to a computer terminal and used to control the syringe pump with a plurality of perfusion channels. The method includes: when an ablation task is triggered, controlling the syringe pump to open at least one perfusion channel so as to perform a perfusion operation through the opened perfusion channel at a preset initial flow rate; acquiring temperatures of a plurality of sites of an ablation object in real time by a plurality of temperature acquisition apparatuses; and controlling the syringe pump to open or close some or all of the perfusion channels and/or controlling the syringe pump to adjust flow rates of some or all of the perfusion channels according to real-time changes in the temperatures of the plurality of sites.

In one aspect, embodiments of the present invention further provide an apparatus for controlling perfusion of a plurality of channels of a syringe pump, which is configured to control the syringe pump with a plurality of perfusion channels. The apparatus includes: a control module, which is configured to when an ablation task is triggered, control the syringe pump to open at least one perfusion channel so as to perform a perfusion operation through the opened perfusion channel at a preset initial flow rate; a temperature acquisition module, which is configured to acquire temperatures of a plurality of sites of an ablation object in real time by a plurality of temperature acquisition apparatuses; and the control module is further configured to control the syringe pump to open or close some or all of the perfusion channels and/or control the syringe pump to adjust flow rates of some or all of the perfusion channels according to real-time changes in the temperatures of the plurality of sites.

In one aspect, embodiments of the present invention further provide an electronic apparatus, including a non-transitory memory and a processor, wherein the non-transitory memory stores an executable program code; the processor is electrically coupled with the non-transitory memory and a plurality of temperature acquisition apparatuses; and the processor calls the executable program code stored in the non-transitory memory to execute the method for controlling the perfusion of the plurality of channels of the syringe pump according to the foregoing embodiment.

In one aspect, embodiments of the present invention further provide a syringe pump, including a controller, a plurality of temperature acquisition apparatuses and a plurality of injection structures, wherein each of the injection structures includes a syringe, an extension tube, a push rod and a drive apparatus, wherein one end of the extension tube is connected with the syringe and the other end thereof is provided with at least one of the temperature acquisition apparatuses, and each of the injection structures forms a perfusion channel; and the controller is electrically coupled with the plurality of temperature acquisition apparatuses, electrically connected with the plurality of injection structures, and configured to execute steps in the method for controlling perfusion of the plurality of channels of the syringe pump according to the foregoing embodiment.

In one aspect, embodiments of the present invention further provide a non-transitory computer-readable storage medium on which a computer program is stored, wherein the computer program when run by a processor implements the method for controlling the perfusion of the plurality of channels of the syringe pump according to the foregoing embodiment.

In the embodiments provided in the present invention, when the ablation task is triggered, the syringe pump is controlled to open and pass through the at least one perfusion channel so as to perform the perfusion operation at the preset initial flow rate. Then, the syringe pump is controlled to open or close some or all of the perfusion channels and/or the flow rates of some or all of the perfusion channels are adjusted according to the temperatures, which are acquired by the plurality of temperature acquisition apparatuses in real time, of the plurality of sites of the ablation object. Accordingly, the perfusion of the plurality of channels of the syringe pump is intelligently adjusted dynamically based on the real-time changes in the temperatures of the plurality of sites of the ablation object in the process of performing the ablation task. Since the perfusion volume of the syringe pump is adjusted relatively purposefully and directionally as the temperatures of the different sites of the ablation object change, operation delays and errors caused by manual determination can be reduced. Meanwhile, the timeliness, the accuracy and the pertinence of perfusing the liquid in the process of performing the ablation task can be improved. Accordingly, the injury of the ablation operation to the ablation object is reduced, and the safety of the radio frequency ablation operation is improved.

Data Adjustment Method in Radio-Frequency Operation and Radio-Frequency Host

An embodiment of the present invention provides a data adjustment method in a radio-frequency operation and a radio-frequency host. During the radio-frequency operation, the radio-frequency output power or a preset range for physical characteristic data of a subject of the radio-frequency operation is adjusted to improve the safety and effectiveness of radio-frequency operation.

In an aspect, an embodiment of the present invention provides a data adjustment method in a radio-frequency operation, which includes: acquiring set power data corresponding to a radio-frequency operation, setting an output power of a radio-frequency signal according to the set power data, and outputting the radio-frequency signal to a subject of the radio-frequency operation; detecting physical characteristic data of the subject in real time, and determining whether the physical characteristic data exceeds a preset range; adjusting the radio-frequency output power if the physical characteristic data exceeds the preset range; and adjusting the preset range according to the physical characteristic data detected in real time in a preset period of time before a current moment if the physical characteristic data does not exceed the preset range.

In an aspect, an embodiment of the present invention also provides a radio-frequency host, which includes an acquisition module, configured to acquire set power data corresponding to a radio-frequency operation; a transmitting module, configured to set an output power of a radio-frequency signal according to the set power data, and output the radio-frequency signal to a subject of the radio-frequency operation; a detection module, configured to detect physical characteristic data of the subject in real time, and determine whether the physical characteristic data exceeds a preset range; and an adjustment module, configured to adjust the radio-frequency output power if the physical characteristic data exceeds the preset range, and adjust the preset range according to the physical characteristic data detected in real time in a preset period of time before a current moment if the physical characteristic data does not exceed the preset range.

In an aspect, an embodiment of the present invention also provides a radio-frequency host, which includes a storage and a processor, wherein the storage stores an executable program code; and the processor is coupled to the storage, and configured to call the executable program code stored in the storage, and implement the data adjustment method in a radio-frequency operation as described above.

As can be known from the above embodiments of the present invention, set power data corresponding to a radio-frequency operation is acquired, an output power of a radio-frequency signal is set according to the set power data, and the radio-frequency signal is outputted physical characteristic data of a subject of the radio-frequency operation is detected in real time during the radio-frequency operation, and whether the physical characteristic data exceeds a preset range is determined, wherein if the physical characteristic data exceeds the preset range, the radio-frequency output power is adjusted, to reduce the risk of the radio-frequency operation damaging the subject and improve the safety of the radio-frequency operation; and if the physical characteristic data does not exceed the preset range, the preset range of the physical characteristic data is adjusted, and the reasonableness of the preset range is automatically updated, to provide a more accurate data basis for subsequent radio-frequency operations, and improve the reasonableness and success rate of the radio-frequency operation.

Method for Protecting Radio Frequency Operation Abnormality, Radio Frequency Mainframe, and Radio Frequency Operation System

Embodiments of this application provide a method for protecting radio frequency operation abnormality, a radio frequency mainframe, and a radio frequency operation system, which can implement dual protection by stopping outputting radio frequency energy and cutting off a radio frequency energy path when a radio frequency operation is abnormal, thereby improving safety of radio frequency operations.

In one aspect, an embodiment of this application provides a method for protecting radio frequency operation abnormality, comprising: when detecting that a radio frequency mainframe continuously outputs radio frequency energy, detecting preset kinds of detection data of a radio frequency operation in real time; determining whether detected preset kinds of detection data meets a preset abnormal state; and if the preset abnormal state is met, controlling a radio frequency generating apparatus to stop outputting radio frequency energy and controlling an emergency stop apparatus to cut off a radio frequency energy output path of the radio frequency mainframe.

In one aspect, an embodiment of this application provides a radio frequency mainframe comprising a detecting apparatus, a radio frequency generating apparatus, and an emergency stop apparatus; wherein the detecting apparatus is configured to: when it is detected that the radio frequency mainframe continuously outputs radio frequency energy, detect preset kinds of detection data of a radio frequency operation in real time; the detecting apparatus is further configured to: determine whether detected preset kinds of detection data meets a preset abnormal state; and the detecting apparatus is further configured to: if the preset abnormal state is met, control the radio frequency generating apparatus to stop outputting radio frequency energy and control the emergency stop apparatus to cut off a radio frequency energy output path.

In one aspect, an embodiment of this application provides a radio frequency mainframe, comprising: a memory and a processor; wherein the memory stores executable program codes; the processor coupled with the memory calls the executable program codes stored in the memory to execute the aforesaid method for protecting radio frequency operation abnormality.

In one aspect, an embodiment of this application provides a radio frequency operation system, comprising: a radio frequency mainframe and an injection pump; wherein the radio frequency mainframe is configured to execute the aforesaid method for protecting radio frequency operation abnormality; and the injection pump is configured to inject liquid with a preset function to a radio frequency operated object under control of the radio frequency mainframe.

From the above embodiments of this application, it can be known that: when a radio frequency mainframe continuously outputs radio frequency energy, preset kinds of detection data of a radio frequency operation is detected in real time; it is determined whether detected data meets a preset abnormal state; and if yes, a radio frequency generating apparatus is controlled to stop outputting radio frequency energy and an emergency stop apparatus is controlled to cut off a radio frequency energy output path of the radio frequency mainframe. The above two manners of protection are performed at the same time to prevent any one manner from failing or malfunctioning and causing protection failure, a succeeding rate of protection is improved, and safety of radio frequency operations is improved.

Method and Apparatus for Dynamically Adjusting Radio Frequency Parameter and Radio Frequency Host

Embodiments of the present invention provide a method and an apparatus for dynamically adjusting a radio frequency parameter and a radio frequency host, which may realize that radio frequency data of a radio frequency object is dynamically adjusted by comparing the detected radio frequency data of an operation object with a preset radio frequency data standard range and a preset radio frequency data limit range, so as to improve the success rate and the safety of the radio frequency operation.

In one aspect, embodiments of the present invention provide a method for dynamically adjusting a radio frequency parameter, including: confirming an operation stage in which a radio frequency operation is and acquiring a radio frequency data standard range and a radio frequency data limit range corresponding to an operation object of the radio frequency operation and the operation stage, wherein the radio frequency data standard range is within the radio frequency data limit range; detecting radio frequency data of the operation object in real time, and comparing the radio frequency data of the operation object with the radio frequency data standard range and the radio frequency data limit range, respectively; if the radio frequency data detected in real time exceeds the radio frequency data standard range but does not exceed the radio frequency data limit range and lasts for a preset duration, controlling the radio frequency data to be within the radio frequency data standard range by controlling an injection volume of a syringe pump to the operation object; and if the radio frequency data detected in real time exceeds the radio frequency data limit range, stopping radio frequency energy from being output.

In one aspect, embodiments of the present invention further provide an apparatus for dynamically adjusting a radio frequency parameter, including:

an acquisition module, which is configured to confirm an operation stage in which a radio frequency operation is and acquire a radio frequency data standard range and a radio frequency data limit range corresponding to an operation object of the radio frequency operation and the operation stage, wherein the radio frequency data standard range is within the radio frequency data limit range; a detection module, which is configured to detect radio frequency data of the operation object in real time; a comparison module which is configured to compare the detected radio frequency data with the radio frequency data standard range and the radio frequency data limit range; and a control module, which is configured to if the radio frequency data detected in real time exceeds the radio frequency data standard range but does not exceed the radio frequency data limit range and lasts for a preset duration, control the radio frequency data to be within the radio frequency data standard range by controlling an injection volume of a syringe pump to the operation object, and if the radio frequency data detected in real time exceeds the radio frequency data limit range, stop radio frequency energy from being output.

In one aspect, embodiments of the present invention further provide a radio frequency host, including: a memory and a processor, wherein the memory stores an executable program code; and the processor coupled with the memory calls the executable program code stored in the memory to perform the method for dynamically adjusting the radio frequency parameter as described above.

It may be known from the above embodiments of the present invention that the radio frequency data standard range corresponding to the operation object of the radio frequency operation and the current operation stage is acquired, and the radio frequency data detected in real time is compared with the radio frequency data standard range and the radio frequency data limit range in real time, respectively. If the radio frequency data detected in real time exceeds the radio frequency data standard range but does not exceed the radio frequency data limit range and lasts for the preset duration, the radio frequency data is controlled to be within the radio frequency data standard range by controlling the injection volume of the syringe pump to the operation object. Accordingly, the radio frequency data is dynamically adjusted within the radio frequency data standard range, and the success rate of the radio frequency operation is improved. If the radio frequency data detected in real time exceeds the radio frequency data limit range, it is confirmed that problems occur in the radio frequency host of the current radio frequency operation or the operation object, and the radio frequency energy is stopped from being output. Therefore, the radio frequency host and the operation object are prevented from being damaged, and the safety of the radio frequency operation is improved.

Method and Apparatus for Safety Control of Radio Frequency Operation, and Radio Frequency Mainframe

Embodiments of this application provides a method and apparatus for safety control of radio frequency operation, and a radio frequency mainframe, which can improve safety and intelligence of radio frequency operations when connection between a radio frequency mainframe and a radio frequency operated object does not meet a standard.

An aspect of embodiments of this application provides a method for safety control of radio frequency operation, comprising: when connecting ends of a plurality of radio frequency circuits connects an operated object to a radio frequency mainframe, acquiring detected values of the plurality of radio frequency circuits; determining whether change amounts of the detected values reach a preset value range; if a quantity of target radio frequency circuits of which the change amounts of the detected values reach the preset value range is not less than a preset quantity, selecting the preset quantity of target radio frequency circuits from the target radio frequency circuits according to a preset selection rule as radio frequency input circuits, and inputting radio frequency energy into the radio frequency input circuits; if the quantity of target radio frequency circuits of which the change amounts of the detected values reach the preset value range is less than the preset quantity, not inputting radio frequency energy into any radio frequency circuit.

An aspect of embodiments of this application further provides an apparatus for safety control of radio frequency operation, comprising: an acquiring module configured to: when connecting ends of a plurality of radio frequency circuits connects an operated object to a radio frequency mainframe, acquire detected values of the plurality of radio frequency circuits; a determining module configured to determine whether change amounts of the detected values reach a preset value range; and a processing module configured to: if a quantity of target radio frequency circuits of which the change amounts of the detected values reach the preset value range is not less than a preset quantity, select the preset quantity of target radio frequency circuits from the target radio frequency circuits according to a preset selection rule as radio frequency input circuits, and input radio frequency energy into the radio frequency input circuits; wherein the processing module is further configured to: if the quantity of target radio frequency circuits of which the change amounts of the detected values reach the preset value range is less than the preset quantity, not input radio frequency energy into any radio frequency circuit.

An aspect of embodiments of this application further provides a radio frequency mainframe, comprising: a memory and a processor, wherein the memory stores executable program codes; the processor coupled with the memory calls the executable program codes stored in the memory to execute the aforesaid method for safety control of radio frequency operation.

From the above embodiments of this application, it can be known that: when an operated object is connected to a radio frequency mainframe through connecting ends of radio frequency circuits, according change amounts of detected values of the radio frequency circuits, it is determined whether a quantity of target radio frequency circuits of which the change amounts reach a preset value range reaches a preset quantity, that is, it is determined whether the connection between the connecting ends and the operated object meets a connection standard; if reaching, the preset quantity of target radio frequency circuits are selected from the target radio frequency circuits as radio frequency input circuits, and radio frequency signals are controlled to input; if not reaching, no radio frequency input is performed, so as to avoid subsequent radio frequency operations from being affected by connection that does not meet the standard. Accordingly, the above-described method for safety control of radio frequency operation can automatically determine whether connection between an operated object and radio frequency circuits meets a standard, and does not perform radio frequency energy input when radio frequency circuits meeting the standard do not meet requirement of radio frequency operation in quantity, thereby improving safety and intelligence of radio frequency operation.

Ablation Operation Prompting Method, Electronic Device and Computer-Readable Storage Medium

An object of the embodiments of the present invention is to provide an ablation operation prompting method, an electronic device, and a computer-readable storage medium, with which the status changes of an ablation site can be displayed in real time and intuitively during the implementation of the ablation operation, thereby improving the effectiveness and relevance of information prompts.

In an aspect, an embodiment of the present invention provides an ablation operation prompting method, applicable to a computer terminal. The method includes: acquiring an image of an ablation site and display the image on a screen, when an ablation task is triggered; acquiring position data of a currently-being-ablated target ablation point, and marking the target ablation point in the image according to the position data; acquiring the elapsed ablation time and the temperature of the target ablation point in real time, and determining the ablation status of the target ablation point according to the elapsed ablation time and the temperature; and generating a schematic real-time dynamic change diagram of the target ablation point according to the ablation status, and displaying the schematic diagram on the screen, to indicate the real-time ablation status change of the target ablation point.

In an aspect, an embodiment of the present invention further provides an ablation operation prompting device, which includes: an image display module, configured to acquire an image of an ablation site and display the image on a screen, when an ablation task is triggered; a marking module, configured to acquire position data of a currently-being-ablated target ablation point, and mark the target ablation point in the image according to the position data; an ablation status determination module, configured to acquire the elapsed ablation time and the temperature of the target ablation point in real time, and determine the ablation status of the target ablation point according to the elapsed ablation time and the temperature; and an ablation status prompting module, configured to generate a schematic real-time dynamic change diagram of the target ablation point according to the ablation status, and display the schematic diagram on the screen, to indicate the real-time ablation status change of the target ablation point.

In an aspect, an embodiment of the present invention further provides an electronic device, which includes a storage and a processor, wherein the storage stores an executable program code; and the processor is coupled to the storage, and configured to call the executable program code stored in the storage, and implement the ablation operation prompting method provided in the above embodiments.

In an aspect, an embodiment of the present invention further provides a non-transitory computer-readable storage medium, on which a computer program is stored, wherein when the computer program is executed by the processor, the ablation operation prompting method provided in the above embodiments is implemented.

According to various embodiments provided in the present invention, when an ablation task is triggered, an image of an ablation site is acquired and displayed on a preset prompting interaction interface where the image of the ablation site is marked with a currently-being-ablated target ablation point; according to the elapsed ablation time and the temperature of the target ablation point acquired in real time, a schematic real-time dynamic change diagram of the ablation status of the target ablation point is generated and displayed, so that the status changes of an ablation site can be displayed in real time and intuitively during the implementation of the ablation operation, thereby improving the effectiveness and relevance of information prompts.

Radio-Frequency Operation Prompting Method, Electronic Device, and Computer-Readable Storage Medium

An embodiment of the present invention provides a radio-frequency operation prompting method, an electronic device, and a computer-readable storage medium, with which visual prompting of the change of range of a to-be-operated area is realized, thereby improving the effectiveness of information prompts, and thus improving the success rate and effect of the radio-frequency operation.

In an aspect, an embodiment of the present invention provides a radio-frequency operation prompting method, applicable to a computer terminal. The method includes: acquiring physical characteristic data of an operating position in a subject of a radio-frequency operation in real time by multiple probes; obtaining a physical characteristic field of the subject of the radio-frequency operation according to the physical characteristic data acquired in real time; and obtaining the change of range of a to-be-operated area in a target operating area according to an initial range of the target operating area in the subject of the radio-frequency operation and the change of value of the physical characteristic data in the physical characteristic field, and displaying the change of range by a three-dimensional model.

In an aspect, an embodiment of the present invention further provides a radio-frequency operation prompting device, which includes: an acquisition module, configured to acquire physical characteristic data of an operating position in a subject of a radio-frequency operation in real time by multiple probes; a processing module, configured to obtain a physical characteristic field of the subject of the radio-frequency operation according to the physical characteristic data acquired in real time, and obtain the change of range of a to-be-operated area in a target operating area according to an initial range of the target operating area in the subject of the radio-frequency operation and the change of value of the physical characteristic data in the physical characteristic field, and a display module, configured to display the change of range by a three-dimensional model.

In an aspect, an embodiment of the present invention further provides an electronic device, which includes a storage and a processor, wherein the storage stores an executable program code; and the processor is coupled to the storage, and configured to call the executable program code stored in the storage, and implement the radio-frequency operation prompting method provided in the above embodiments.

In an aspect, an embodiment of the present invention further provides a non-transitory computer-readable storage medium, on which a computer program is stored, wherein when the computer program is executed by a processor, the radio-frequency operation prompting method provided in the above embodiments is implemented.

According to various embodiments provided in the present invention, multiple pieces of physical characteristic data of an operating position in a subject of a radio-frequency operation are acquired in real time by multiple probes, a physical characteristic field of the subject of the radio-frequency operation is obtained according to these pieces of data, then the change of range of a to-be-operated area in a target operating area is obtained according to an initial range of the target operating area in the subject of the radio-frequency operation and the change of value of the physical characteristic data in the physical characteristic field, and displayed by a three-dimensional model. As a result, the visual prompting of the change of range of the to-be-operated area is realized, the content of the prompt information is much rich, intuitive and vivid, and the accuracy and intelligence of determining the to-be-operated area is increased, thereby improving the effectiveness of information prompts, and thus improving the success rate and effect of the radio-frequency operation.

An embodiment of the present invention is a method for lung denervation along an airway in the lung comprising: advancing a catheter along the airway; deploying an open loop in contact with the epithelium of the airway; simultaneously delivering radio frequency energy from a plurality of discrete spaced-apart locations along the loop to target regions along the epithelium according to a set of ablation parameters sufficient to heat and interrupt the bronchial nerve functionality; and forming a liquid film between the loop and the epithelium for protecting damage to the epithelium.

In embodiments, the step of forming is performed by flowing a cooling agent from the discrete spaced-apart locations onto the epithelium.

In embodiments, the loop comprises an electrode at each discrete spaced-apart location for delivering radio frequency energy.

In embodiments, each electrode comprises an array of egress ports, through which the cooling agent is ejected.

In embodiments, non-targeted regions of the epithelium between the electrodes are protected by the cooling film.

In embodiments, the method further comprises retracting the loop, moving the catheter to a new location, deploying the open loop at the new location, and repeating the delivering and forming steps.

In embodiments, the step of moving comprises advancing and/or rotating.

In embodiments, the ablation parameters comprise a single electrode output energy of 1000-1500 J, and a power limit not to exceed 20 W, and in particular embodiments, the ablation parameters comprise a single electrode output energy of 1080 J˜1360 J and power limit of 12 W˜16 W.

In embodiments, the step of flowing is performed using iced saline.

In embodiments, the step of flowing comprises adjusting the flowrate of the cooling agent from high to low.

In embodiments, the method further comprises monitoring each location, and independently adjusting the flowrate of the cooling agent to each location based on the monitoring. In embodiments, the step of monitoring comprises monitoring temperature.

In embodiments, the method further comprises independently adjusting the flowrate of the cooling agent to each location such that the coolant is controllably directed to one or more desired areas of the airway and excludes one or more undesired areas. In embodiments, each area is monitored for the presence of the coolant, and the controlling is based on the monitoring. Optionally, the step of monitoring comprises monitoring temperature.

In embodiments, the method further comprises displaying ablation progress based on the monitoring.

In embodiments, an electrosurgical method of treating chronic bronchitis comprising: destroying motor axons of a peripheral bronchial nerve, blocking parasympathetic transmission in the pulmonary nerve and reducing acetylcholine release, thereby reducing mucus production, thereby improving airway obstruction; and simultaneously, during the destroying step, ejecting a cooling agent to a plurality of regions along the inner wall of the airway according to a plurality of customized flowrates based on temperature of each region.

In embodiments, the step of destroying is performed by applying radiofrequency energy to discrete circumferential locations.

In embodiments, the method further comprises displaying ablation progress based on monitoring the temperature of each region.

The description, objects and advantages of embodiments of the present invention will become apparent from the detailed description to follow, together with the accompanying drawings.

### BRIEF DESCRIPTION OF THE DRAWINGS

FIG. 1A is a schematic diagram of a radio frequency ablation catheter according to an embodiment;

FIG. 1B and FIG. 1C are schematic diagrams of the working process of an annular section;

FIG. 2A is a schematic diagram of the internal structure of a pipe body

FIG. 2B is an enlarged schematic view of A in FIG. 2A;

FIG. 2C is an enlarged schematic view of B in FIG. 2A;

FIG. 3A is a schematic view of an outer sleeve structure;

FIG. 3B is an enlarged schematic view of C in FIG. 3A;

FIG. 3C is an enlarged schematic view of D in FIG. 3A;

FIG. 3D is a structural schematic diagram of a deformation constraint tube;

FIG. 3E is a schematic diagram of an electrode structure;

FIG. 4A is a schematic diagram of an inner sleeve structure;

FIG. 4B is an enlarged schematic view of E in FIG. 4A;

FIG. 4C is an enlarged schematic view of F in FIG. 4A;

FIG. 5A is a schematic diagram of an output hole on a pipe body;

FIG. 5B is a cross-sectional view of the G-G′ of FIG. 5A;

FIG. 5C is a schematic view of an end face of a pipe body;

FIG. 5D is an enlarged schematic view of the output hole in FIG. 5B

FIG. 6A is a schematic diagram of a fitting portion of an annular section and a braided tube of a pipe body;

FIG. 6B is a partially enlarged schematic view of FIG. 6A;

FIG. 6C is a schematic view of an inner structure of a mating portion of an annular segment and a braided tube;

FIG. 6D is a partially enlarged schematic view of FIG. 6C;

FIG. 7A is an exploded view of an operating handle;

FIG. 7B is a schematic view of the internal structure of an operating handle;

FIG. 7C is a schematic view of the internal structure of an operating handle of another viewing angle;

FIG. 7D is a schematic view of an end cover structure;

FIG. 7E is a schematic view of the cooperation of an end cover and a driving member.

FIG. 8 is a diagram showing an application environment of a method for controlling perfusion of a plurality of channels of a syringe pump according to an embodiment of the present invention;

FIG. 9 is a schematic diagram showing an internal structure of a syringe pump according to an embodiment of the present invention;

FIG. 10 is a schematic diagram showing an internal structure of an injection structure in the syringe pump as shown in FIG. 9;

FIG. 11 is a flow chart of an implementation of a method for controlling perfusion of a plurality of channels of a syringe pump according to an embodiment of the present invention;

FIG. 12 is a flow chart of an implementation of a method for controlling perfusion of a plurality of channels of a syringe pump according to another embodiment of the present invention;

FIG. 13 is a flow chart of an implementation of a method for controlling perfusion of a plurality of channels of a syringe pump according to further embodiment of the present invention;

FIG. 14 is a flow chart of an implementation of a method for controlling yet perfusion of a plurality of channels of a syringe pump according to yet another embodiment of the present invention;

FIG. 15 is a schematic diagram of a layout of a plurality of perfusion channels and a plurality of temperature acquisition apparatuses in a method for controlling perfusion of a plurality of channels of a syringe pump according to an embodiment of the present invention;

FIG. 16 is a schematic diagram showing a structure of an apparatus for controlling perfusion of a plurality of channels of a syringe pump according to an embodiment of the present invention; and

FIG. 17 is a schematic diagram showing a hardware structure of an electronic apparatus according to an embodiment of the present invention.

FIG. 18 is a schematic diagram showing an application scenario of a data adjustment method in a radio-frequency operation provided in an embodiment of the present invention;

FIG. 19 is a schematic flow chart of a data adjustment method in a radio-frequency operation provided in an embodiment of the present invention;

FIG. 20 is a schematic flow chart of a data adjustment method in a radio-frequency operation provided in another embodiment of the present invention;

FIG. 21 is a schematic structural diagram of a radio-frequency host provided in an embodiment of the present invention; and

FIG. 22 is a schematic diagram showing a hardware structure in a radio-frequency host provided in an embodiment of the present invention.

FIG. 23 is a schematic diagram of an application scene of a method for protecting radio frequency operation abnormality provided by an embodiment of this application.

FIG. 24 is a schematic flow chart of a method for protecting radio frequency operation abnormality provided by an embodiment of this application.

FIG. 25 is a schematic flow chart of a method for protecting radio frequency operation abnormality provided by another embodiment of this application.

FIG. 26 is a schematic flow chart of a method for protecting radio frequency operation abnormality provided by another embodiment of this application.

FIG. 27 is a structural schematic diagram of a radio frequency mainframe provided by an embodiment of this application.

FIG. 28 is a structural schematic diagram of a radio frequency mainframe provided by another embodiment of this application.

FIG. 29 is a structural schematic diagram of a radio frequency mainframe provided by another embodiment of this application.

FIG. 30 is a structural schematic diagram of a radio frequency operation system provided by an embodiment of this application.

FIG. 31 is a schematic diagram showing an application scenario of a method for dynamically adjusting a radio frequency parameter according to an embodiment of the present invention;

FIG. 32 is a schematic flow chart of a method for dynamically adjusting a radio frequency parameter according to an embodiment of the present invention;

FIG. 33 is a schematic flow chart of a method for dynamically adjusting a radio frequency parameter according to another embodiment of the present invention;

FIG. 34 is a schematic diagram showing a structure of an apparatus for dynamically adjusting a radio frequency parameter according to an embodiment of the present invention; and

FIG. 35 is a schematic diagram showing a structure of a radio frequency host according to an embodiment of the present invention.

FIG. 36 is a schematic diagram of an application scene of a method for safety control of radio frequency operation provided by an embodiment of this application.

FIG. 37 is a schematic flow chart of a method for safety control of radio frequency operation provided by an embodiment of this application.

FIG. 38 is a schematic flow chart of a method for safety control of radio frequency operation provided by another embodiment of this application.

FIG. 39 is a structural schematic diagram of a radio frequency circuit in a method for safety control of radio frequency operation provided by an embodiment of this application.

FIG. 40 is a schematic flow chart of a method for safety control of radio frequency operation provided by another embodiment of this application.

FIG. 41 is a structural schematic diagram of an impedance detection circuit in a method for safety control of radio frequency operation provided by an embodiment of this application.

FIG. 42 is a structural schematic diagram of an apparatus for safety control of radio frequency operation provided by an embodiment of this application.

FIG. 43 is a structural schematic diagram of a radio frequency mainframe provided by an embodiment of this application.

FIG. 44 is a schematic diagram of an existing ablation operation prompting interface;

FIG. 45 shows an application environment of an ablation operation prompting method provided in an embodiment of the present invention;

FIG. 46 shows a flow chart of an ablation operation prompting method provided in an embodiment of the present invention;

FIG. 47 and FIG. 48 are schematic diagrams showing an image of an ablation site and the real-time dynamic change of the ablation status of a target ablation point in an ablation operation prompting method provided in an embodiment of the present invention;

FIG. 49 shows a flow chart of an ablation operation prompting method provided in another embodiment of the present invention;

FIG. 50 is a schematic diagram showing an operation track in an ablation operation prompting method provided in an embodiment of the present invention;

FIG. 51 is a schematic diagram showing a real-time temperature change curve and a real-time impedance change curve in an ablation operation prompting method provided in an embodiment of the present invention;

FIG. 52 is a schematic diagram showing the real-time impedance in an ablation operation prompting method provided in an embodiment of the present invention;

FIG. 53 is a schematic diagram showing the real-time impedance change in an ablation operation prompting method provided in an embodiment of the present invention;

FIG. 54 is a schematic diagram showing a picture of a screen on which all the schematic views are displayed in an ablation operation prompting method provided in an embodiment of the present invention;

FIG. 55 is a schematic structural diagram of an ablation operation prompting device provided in an embodiment of the present invention; and

FIG. 56 is a schematic diagram showing a hardware structure of an electronic device provided in an embodiment of the present invention.

FIG. 57 is a schematic diagram of an existing radio-frequency operation prompting interface;

FIG. 58 shows an application environment of a radio-frequency operation prompting method provided in an embodiment of the present invention;

FIG. 59 shows a flow chart of a radio-frequency operation prompting method provided in an embodiment of the present invention;

FIG. 60 is a schematic view of a tip of a radio-frequency operation catheter in a radio-frequency operation prompting method provided in an embodiment of the present invention;

FIG. 61 shows a flow chart of a radio-frequency operation prompting method provided in another embodiment of the present invention;

FIG. 62 is a schematic structural diagram of a radio-frequency operation prompting device provided in an embodiment of the present invention;

FIG. 63 is a schematic diagram showing a hardware structure of an electronic device provided in an embodiment of the present invention; and

FIGS. 64-67 illustrate a bronchoscopic TLD method in accordance with embodiments of the invention.

### DETAILED DESCRIPTION OF THE INVENTION

Before the present invention is described in detail, it is to be understood that this invention is not limited to particular variations set forth herein as various changes or modifications may be made to the invention described and equivalents may be substituted without departing from the spirit and scope of the invention. As will be apparent to those of skill in the art upon reading this disclosure, each of the individual embodiments described and illustrated herein has discrete components and features which may be readily separated from or combined with the features of any of the other several embodiments without departing from the scope or spirit of the present invention. In addition, many modifications may be made to adapt a particular situation, material, composition of matter, process, process act(s) or step(s) to the objective(s), spirit or scope of the present invention.

Methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

All existing subject matter mentioned herein (e.g., publications, patents, patent applications and hardware) is incorporated by reference herein in its entirety except insofar as the subject matter may conflict with that of the present invention (in which case what is present herein shall prevail).

The following are incorporated herein by reference in their entirety for all purposes:

PCT/CN2020/118645, filed Sep. 29, 2020, entitled “RADIO-FREQUENCY ABLATION CATHETER AND RADIO-FREQUENCY ABLATION SYSTEM”; PCT/CN2020/118646, filed Sep. 29, 2020, entitled “DETECTION MECHANISM, RADIO FREQUENCY ABLATION CATHETER AND RADIO FREQUENCY ABLATION SYSTEM”; PCT/CN2020/140380, filed Dec. 28, 2020, entitled “INJECTION PUMP PERFUSION CONTROL METHOD, DEVICE, SYSTEM AND COMPUTER READABLE STORAGE MEDIUM”; PCT/CN2021/072952, filed Jan. 20, 2021, entitled “PROTECTION METHOD FOR ABNORMAL RADIO FREQUENCY OPERATION, RADIO FREQUENCY HOST AND RADIO FREQUENCY OPERATING SYSTEM”; PCT/CN2021/072953, filed Jan. 20, 2021, entitled “INJECTION PUMP MULTI-PATH PERFUSION CONTROL METHOD, DEVICE, INJECTION PUMP AND STORAGE MEDIUM”; PCT/CN2021/072954, filed Jan. 20, 2021, entitled “METHOD, DEVICE AND RADIO FREQUENCY HOST FOR DYNAMICALLY ADJUSTING RADIO FREQUENCY PARAMETERS”; PCT/CN2021/072955, filed Jan. 20, 2021, entitled “METHOD FOR PROMPTING ABLATION OPERATION, ELECTRONIC DEVICE AND COMPUTER READABLE STORAGE MEDIUM”; PCT/CN2021/072956, filed Jan. 20, 2021, entitled “DATA ADJUSTMENT METHOD IN RADIO FREQUENCY OPERATION AND RADIO FREQUENCY HOST”; PCT/CN2021/072957, filed Jan. 20, 2021, entitled “RADIO FREQUENCY OPERATION SAFETY CONTROL METHOD, DEVICE AND RADIO FREQUENCY HOST”; PCT/CN2021/072959, filed Jan. 20, 2021, entitled “RADIO FREQUENCY OPERATION PROMPT METHOD, ELECTRONIC DEVICE AND COMPUTER READABLE STORAGE MEDIUM”; PCT/CN2021/076118, filed Feb. 8, 2021, entitled “RADIO-FREQUENCY ABLATION CATHETER AND RADIO-FREQUENCY ABLATION SYSTEM”; and PCT/CN2021/123705, filed Oct. 14, 2021, entitled “RADIOFREQUENCY ABLATION CATHETER AND SYSTEM THEREOF”.

Described herein are ablation methods and related systems.

Radiofrequency Ablation Catheter and System Thereof

The technical solutions in the embodiments of the present invention will be clearly and completely described below with reference to the accompanying drawings in the embodiments of the present invention. Obviously, the described embodiments are only a part of the embodiments of the present invention, but not all of the embodiments.

Based on the embodiments in the present invention, all other embodiments obtained by those of ordinary skill in the art without creative efforts shall fall within the protection scope of the present invention.

It should be noted that when a component is referred to as being “connected” to another component, it can be directly connected to the other component or an intervening component may also exist.

When a component is considered to be “set on” another component, it may be directly set on the other component or there may be a co-existing centered component.

Unless otherwise defined, all technical and scientific terms used herein have the same meaning as commonly understood by one of ordinary skill in the technical field to which this application belongs.

The terms used herein in the specification of the present invention are for the purpose of describing specific embodiments only, and are not intended to limit the present invention.

As used herein, the term “and/or” includes any and all combinations of one or more of the associated listed items.

In the prior art, in order to avoid the interference of various components in the tube body, elastic wires, traction wires and cables are often put through different cavities separately, and the isolation between the components is achieved through the side walls of the cavities; The shape of the segment (i.e. the distal part of the catheter) is mainly achieved by a shaped elastic filament with a relatively stable elastic modulus.

The inventor found that the opening of multiple cavities on the pipe body not only reduces the overall strength of the pipe body, but also greatly increases the production cost; more importantly, the complex multi-lumen pipeline puts forward higher requirements for assembly, increasing the At the same time, the process has a certain impact on the yield of the product. At the same time, the complex relationship of each component has a negative impact on production and assembly.

### Embodiment 1

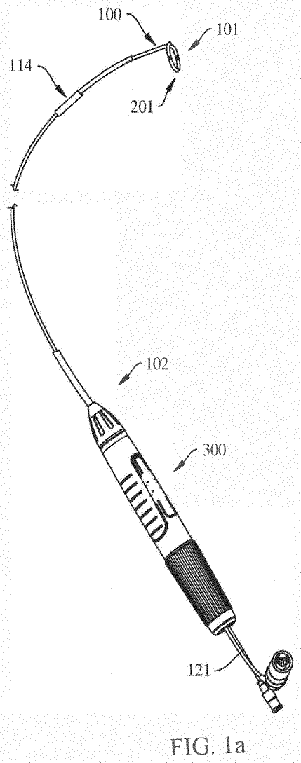

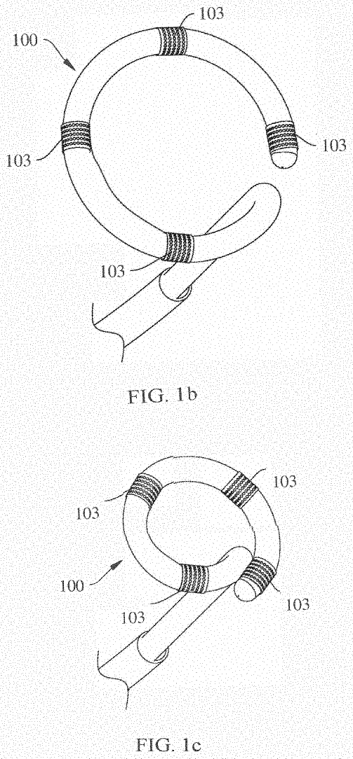

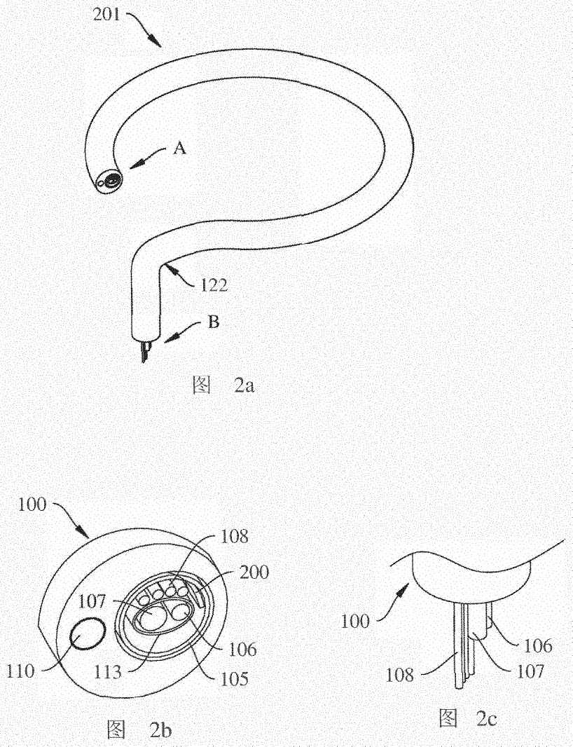

Referring to FIGS. 1a to 3c, the present invention discloses a radiofrequency ablation catheter, including a tube body 100, the tube body 100 has opposite distal ends 101 and proximal ends 102, and the outer wall of the distal end 101 of the tube body 100 is installed to explain The electrode 103 is capable of energy, the inner sleeve 104 and the outer sleeve 105 are arranged in the tube body 100, and the inner sleeve 104 is provided with an elastic wire 107 for shaping the distal end 101 of the tube body 100 and traction. The wire 106, the elastic wire 107 and the pulling wire 106 are arranged side by side and the two are fixed to each other at the position adjacent to the distal end 101 of the tube body 100;

A wire 108 is passed through the radial gap between the inner sleeve 104 and the outer sleeve 105, and the end of the wire 108 passes through the outer sleeve 105 and the tube wall of the tube body 100 and is connected to the electrode 103.

The elastic wire 107, the pulling wire 106 and the guide wire 108 need to be penetrated in the tube body 100, wherein the elastic wire 107 and the pulling wire 106 are located in the inner sleeve 104, and the guide wire 108 is located in the radial gap between the inner sleeve 104 and the outer sleeve 105.

The mutual nesting of the inner sleeve 104 and the outer sleeve 105 realizes the assembly and isolation of the elastic wire 107, the pulling wire 106 and the guide wire 108, thereby avoiding unnecessary multiple lumens on the tube body 100.

At the same time, the inner and outer tubes and the outer sleeve 105 can realize pre-assembly between various components, thereby improving the overall production efficiency and yield.

Regarding the matching relationship between the inner sleeve 104, the outer sleeve 105 and the tube body 100, referring to an embodiment, the tube body 100 is provided with a main cavity 109 and a fluid cavity 110 extending in parallel along the length direction of the tube body 100, The side wall of the tube body 100 is provided with an output hole 111 that communicates with the fluid channel 110;

The fluid channel 110 is used to deliver the cooling medium to the output hole 111, so as to ensure the stable progress of the ablation process.

The independence of the fluid channel 110 and the main channel 109 can ensure that the flow rate and flow rate of the cooling medium are not affected by the components in the main channel 109, and the wires 108 in the main channel 109 can also avoid contact with the cooling medium, improving safety.

In one embodiment, the main cavity 109 and the fluid cavity 110 are formed in a manner that the tube body 100 adopts a dual-lumen tube with an integrated structure, wherein one cavity is the fluid cavity 109 and the other cavity is the main cavity 110.

In another embodiment, the main channel 109 and the fluid channel 110 are formed in such a way that the tube body 100 adopts a double-layer tube structure nested inside and outside, wherein the inner tube is the fluid channel 109, and the diameters of the inner tube and the outer tube are The gap is the main channel 110.

In another embodiment, the main channel 109 and the fluid channel 110 are formed in a way that a part of the tube body adopts a double-lumen tube with an integrated structure, and a part of the tube body 100 adopts a double-layer tube structure nested inside and outside, wherein The inner tube interfaces with one of the dual lumen tubes to define the fluid channel 109, and the radial gap between the inner tube and the outer tube communicates with the other lumen of the dual lumen tube to define the main channel 110.

The ablation function of the distal end 101 is mainly realized by the cooperation of various components in the main lumen 109. In terms of specific form, referring to an embodiment, the elastic wire 107 is pre-shaped at the distal end of the tube body 100 into a ring shape and is positioned far away from the tube body 100. The end portion is correspondingly shaped, and on the cross section of the distal end portion of the tube body 100, the main lumen 109 is eccentrically arranged relative to the central axis of the tube body 100 and is close to the inner edge of the ring.

The ablation function of the distal end 101 is mainly realized by the cooperation of various components in the main lumen 109. In terms of specific form, referring to an embodiment, the distal end 101 of the tube body 100 is coiled into a ring shape, and the cross section at the distal end 101 Above, the main lumen 109 is arranged eccentrically relative to the central axis of the tube body 100 and is close to the inner edge of the ring.

The annular shape of the distal end 101 of the tube body 100 is actually a non-closed annular shape. Under the action of the pulling wire 106, the radial size of the annular shape can be changed (refer to FIG. 1b and FIG. 1c), so as to realize the size of the catheter for different lesions. adapt.

In this embodiment, the pulling wire 106 is used to reduce the radial size of the ring, and the elastic wire 107 is used to maintain and restore the radial size of the ring.

Compared with the central axis of the tube body 100, the main lumen 109 is close to the inner edge of the ring. It is not difficult to understand that in the embodiment disclosed in FIG. 4b, the traction wire 106 is closer to the inner edge of the ring than the elastic wire 107.

When the traction wire 106 is forced to move, the traction wire 106 exerts force on the elastic wire 107 through the fixed position with the elastic wire 107, and the elastic wire 107 is deformed by force to realize the change of the annular radial dimension.

Correspondingly, in the matching relationship between the fluid channel 110 and the main channel 109, in the embodiment disclosed with reference to FIG. Outer edge, the output hole 111 is located at the outer edge of the ring.

The fluid channel 110 being closer to the outer edge of the ring is not only convenient for distributing the main channel 109 and the fluid channel 110 on the tube body 100, but also facilitates the arrangement of the output hole 111.

During the ablation process, the ring generally contacts the adjacent tissue through its own outer edge or upper edge, and the electrode 103 thus realizes the transmission of radio frequency energy.

Therefore, the setting of the output hole 111 on the outer edge or the upper edge of the ring can more directly deliver the cooling medium to the ablation position, thereby ensuring the stable implementation of the ablation process.

The fluid channel 110 is also functionally capable of relieving stress from the annular outer edge.

Under the action of the pulling wire 106, the radial dimension of the ring changes. At this time, the inner edge of the ring is relatively compressed and the outer edge is relatively stretched. The fluid channel 110 and the main channel 109 opened in the tube body 100 are actually The stress-releasing space of the material of the tube body 100 is formed.

Regarding the details of the arrangement of the inner sleeve 104, in the embodiments disclosed with reference to FIGS. 2b and 4b, the cross section of the inner sleeve 104 is an ellipse, and the long axis direction of the ellipse is consistent with the radial direction of the ring.

When the pulling wire 106 exerts a force on the elastic wire 107, the pulling wire 106 actually moves under the restraint of the inner sleeve 104, and exerts a force on the inner sleeve 104.

In this embodiment, the restriction on the movement direction of the traction wire 106 is realized by the cross-sectional shape of the inner sleeve 104, so as to improve the actuation effect of the traction wire 106.

While restraining the pulling wire 106, the inner cannula 104 can also avoid mutual interference between the pulling wire 106 and other components (e.g., the guide wire 108), thereby improving the overall stability of the catheter.

Referring to an embodiment, the wire 108 is located on one side of the inner sleeve 104 in the direction of the central axis of the ring and abuts against the outer wall of the inner sleeve 104.

In addition to the functions of restraint and isolation, the inner sleeve 104 can also play other functions. Referring to an embodiment, the inner wall of the inner sleeve 104 is provided with a lubricating layer or the inner sleeve 104 is a lubricating material.

The realization of lubrication can reduce the movement resistance of the traction wire 106, improve the overall operating experience of the catheter, and also reduce wear and improve stability.

Regarding the selection of specific materials, refer to an embodiment, the inner sleeve 104 is a heat-shrinkable material, and the elastic wire 107 and the pulling wire 106 are tightened after heat-shrinking.

Specifically, the heat-shrinkable material may be a PTFE heat-shrinkable film or the like. In terms of length, the axial length of the inner sleeve 104 is the same as or slightly shorter than the axial length of the annular shape.

In this embodiment, the pre-assembly of the elastic wire 107 and the traction wire 106 can be realized by the heat shrinking of the inner sleeve 104, thereby facilitating subsequent assembly.

Regarding the setting of the outer sleeve 105, referring to an embodiment, a deformation restraining tube 200 is passed through the radial gap between the inner sleeve 104 and the outer sleeve 105, and the outer sleeve 105 is made of heat shrinkable material, and is bundled after heat shrinking. Tight deformation confines the tube 200.

A wire 108 is passed between the outer sleeve 105 and the inner sleeve 104, and the size of the gap between the two is ensured by the size of the deformation restraining tube 200.

On the one hand, the deformation restraint tube 200 is used to cooperate with the elastic wire 107 to maintain the shape of the tube body 100, and on the other hand, it is used to maintain the inner size of the main lumen 109 to prevent the main lumen 109 from being bent under the action of the pulling wire 106. or closed.

In order to realize this function, the deformation restraint tube 200 needs to support the inner wall of the main lumen 109.

Referring to an embodiment, the deformation restraining tube 200 is a coil spring and is wound around the outer circumference of the inner sleeve 104, and the wire 108 is passed between the coil spring and the outer wall of the inner sleeve 104.

The coil spring can be deformed in its own axial direction to release the change of the radial dimension of the ring, and at the same time, the coil spring can support the inner wall of the main lumen 109, thereby ensuring the stability of the catheter during the intervention process.

According to the above description, it is not difficult to understand that the fluid channel 110 is used to deliver the cooling medium to the output hole 111, so the arrangement of the output hole 111 needs to ensure the distribution effect of the cooling medium.

The present invention also discloses a radiofrequency ablation catheter, comprising a tube body 100 having opposite distal ends 101 and proximal ends 102, and a plurality of electrodes for energy release are installed on the outer wall of the distal end 101 of the tube body 100 103, a fluid channel 110 extending along the length direction of the tube body 100 is arranged in the tube body 100, and the side wall of the tube body 100 has a plurality of output holes 111 communicating with the fluid channel 110; the electrode 103 corresponds to the output holes 111 and the flow apertures of the output holes 111 corresponding to the electrodes 103 are equal or unequal.

The flow aperture of the output hole 111 refers to the flux area of the output hole 111 in the output direction of the fluid from the output hole 111.

A single electrode 103 may correspond to a plurality of output holes 111, so the flow pore size of the output holes 111 corresponding to a single electrode 103 refers to the total flow pore size.

The change in flow pore size can be achieved in a number of ways.

For example, the actual different pore diameters of the output holes 111 themselves change; for example, each electrode 103 corresponds to a different number of output holes 111; another example is the combination of the above two methods; further, the arrangement of the output holes 111 with different actual diameters can also be There are various options, for example, output holes 111 with smaller actual diameters are provided with several output holes 111 around the output holes 111 with larger actual diameters.

Equal or unequal flow apertures of the output holes 111 corresponding to the electrodes 103 have different technical advantages, and can be selected as needed.

Referring to an embodiment, the flow apertures corresponding to the electrodes 103 are equal.

When the pressure of the cooling medium in the fluid channel 110 is constant, the flow rate of the cooling medium obtained by each electrode 103 is different, so that the ablation process of each electrode 103 can be finely adjusted.

Referring to another embodiment, the flow apertures corresponding to the electrodes 103 are not equal.

Similar to the above, the setting method of this embodiment can balance the cooling medium flow rate corresponding to each electrode 103 when the pressure of the cooling medium in the fluid channel 110 is constant.

Regarding the details of the distribution of the electrodes 103 and the output holes 111, referring to an embodiment, the electrodes 103 are provided with a plurality of electrodes 103 and are arranged at intervals on the annular shape of the distal end 101 of the tube body 100. Equal or unequal output flows.

For the specific setting of equal or unequal output flow, please refer to the above related expressions about equal or unequal flow apertures, which will not be repeated here.

In one embodiment, from the proximal end 102 to the distal end 101, the flow apertures of the output holes 111 corresponding to the electrodes 103 tend to increase.

In this embodiment, the specific performance is that the number of output holes 111 corresponding to each electrode 103 changes.

Referring to the drawings, it can be seen that the number of output holes 111 is 1, 1, 2 and 3 in sequence from the proximal end 102 to the distal end 101.

The specific number can be adjusted as required, and can also be set as a single output hole 111 with a varying area corresponding to each electrode 103.

In specific products, the diffusion effect of the cooling medium can be further optimized.

Referring to an embodiment, the electrode 103 is provided with a wetting hole (not numbered).

The function of the infiltration hole is to uniformly disperse the cooling medium delivered by the output hole 111 between the electrode 103 and the target tissue, so as to improve the treatment process.

In addition to being arranged on the electrode 103, the wetting hole can also be implemented by a separate wetting cover. Specifically, the setting of the wetting hole can also be adjusted according to the difference of the cooling medium at different positions or the principle of the same arrangement with reference to the output hole 111.

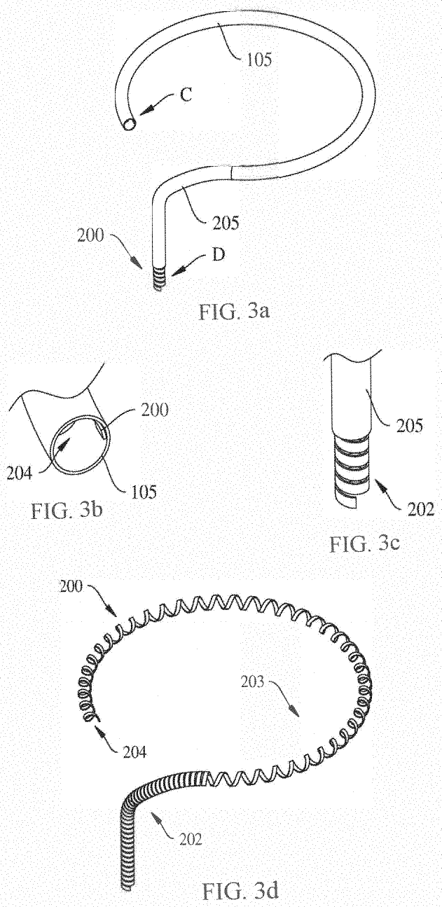

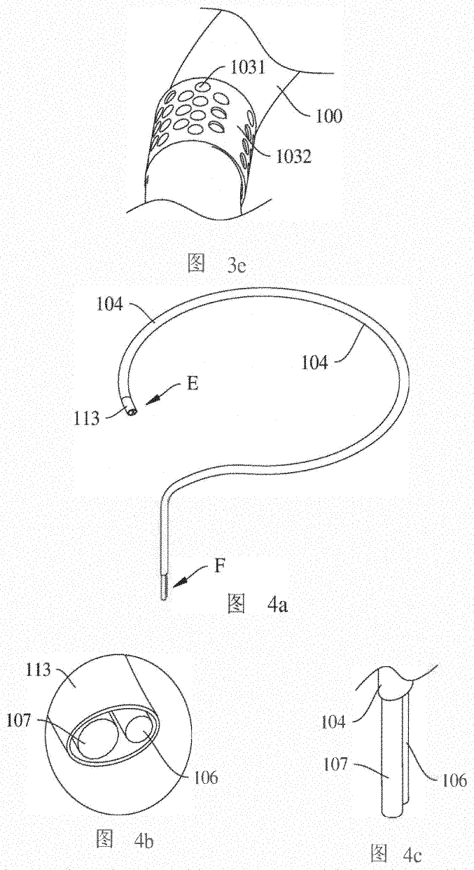

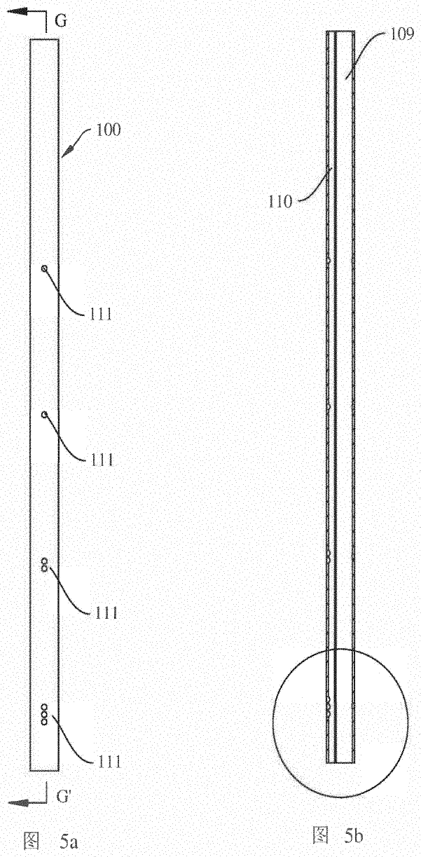

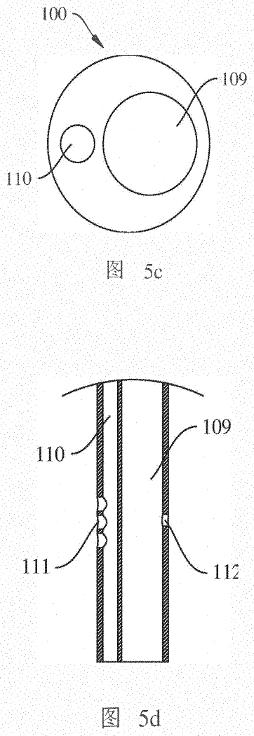

For example, in the embodiment disclosed with reference to FIG. 1b and FIG. 3c, there are multiple wetting holes to form a uniform heat exchange medium protective film outside the electrode 103.

There are multiple infiltration holes, which is more conducive to the balanced distribution of the heat exchange medium. The infiltration holes can be arranged in a certain way or regularly on the periphery of the equalization device, or they can be randomly distributed.

The heat exchange medium output from the heat exchange medium flow channel permeates and flows out to the outside of the equalization device through each infiltration hole, and then surrounds the electrode 103 to form a uniform heat exchange medium protective film. The specific distribution of the infiltration holes will also provide further preferred embodiments later.

The diameter of the wetting hole is 0.1-0.3 mm.

Appropriate pore size is more conducive to the distribution and formation of the heat exchange medium protective film. When the shape of the infiltration hole is non-circular, it can be converted with reference to the area of the circular hole to ensure the flow of heat exchange medium at the infiltration hole.

In one of the embodiments, the wetting holes are slit-shaped.

Compared with the general shape, the slit shape has an obvious length direction, for example, the length is more than 5 times the width, and the width of the slit can generally be set to about 0.1 mm.

The length direction of the slit extends along the axial or circumferential direction of the electrode 103, or forms a certain angle with the axial direction.

Regarding the processing method of the output hole 111, referring to an embodiment, a hollow pipe cutter (not shown) is used to puncture the side wall of the pipe body 100 on one side of the fluid channel 110, and after the puncture is in place, the pipe is passed through the inside of the pipe. The hollow pipe provided by the vacuum suction force will suck the residual material out of the fluid channel 110.

Compared with hot cutting, stamping, and other methods, the processing method of this embodiment has smooth ends of the output hole 111, which reduces the influence on the flow of the cooling medium in the fluid channel 110; meanwhile, the size and accuracy of the output hole 111 can be guaranteed, The cooling medium is adjusted for the output holes 111 to output the cooling medium satisfying the preset amount to each electrode 103, thereby ensuring the stable operation of the treatment process.

During the cutting process, because the pipe will exert a force on the pipe body 100 to cause deformation of the pipe body 100, the deformation may cause the pipe body 100 to be damaged or even the fluid channel 110 to be closed, resulting in unnecessary defects.

Referring to an embodiment, when the tube cutter cuts the side wall of the tube body 100, a lining is filled in the fluid channel 110 to support the fluid channel 110 and ensure the tube body 100, the fluid channel 110 and the main channel 109 Stability during processing of the tube body 100.

### Embodiment 2

Referring to FIGS. 1a to 5d, the present invention also discloses a radiofrequency ablation catheter, comprising a tube body 100, a traction wire 106 is passed through the tube body 100, and the distal end 101 of the tube body 100 is an annular segment 201. 201 can be deformed under the action of the pulling wire 106, and the tube body 100 is also provided with a deformation restraint tube 200. The deformation restraint tube 200 extends from the distal end 101 of the tube body 100 to at least the proximal end 102 side of the annular segment 201, and the deformation restraint tube 200 is located at the stiffness D1 on the proximal end 102 side of the annular segment 201 is greater than the stiffness D2 at the annular segment 201.

The stiffness D1 is greater than the stiffness D2, and the direct technical effect is that the deformation restraint tube 200 is not easily deformed at the proximal end 102 side of the annular segment 201 compared to the annular segment 201.

From the overall view of the tube body 100, under the action of the pulling wire 106, the annular segment 201 is more easily deformed than the proximal end 102 of the annular segment 201, so the proximal end 102 of the annular segment 201 provides the deformation of the annular segment 201 Similar to the role of the “base”, it is used to control the overall shape of the annular segment 201 in space.

From a principle point of view, the technical solution disclosed in the present invention provides different stiffnesses at different positions through the deformation constraining tube 200, so as to precisely control the deformation degree of each position during the deformation process of the distal end 101.

The most direct technical effect is to provide a basis for a stable and reliable treatment process and treatment effect, and to improve the operation experience.

Correspondingly, the stiffness of the deformation restraining tube 200, that is, the elastic modulus, can be further adjusted according to the usage requirements to meet different functional requirements.

For example, when the stiffness D1 of the deformation restraining tube 200 on the proximal end 102 side of the annular segment 201 is relatively large, the proximal end 102 of the annular segment 201 tends not to deform, reducing the displacement of the annular segment 201 compared to the tube body 100; for another example, When the stiffness D1 of the deformation restraining tube 200 on the proximal end 102 side of the annular segment 201 is relatively small, the proximal end 102 of the annular segment 201 tends to deform more, and can be slightly deformed with the deformation of the annular segment 201 under the action of the same pulling wire 106. deformation, so as to realize the displacement of the annular segment 201 relative to the pipe body 100.

Therefore, the segmented arrangement of the deformation restraining tube 200 in this embodiment can provide a structural basis for the realization of different functions.

In the realization form of the deformation restraint tube 200, in order to achieve different stiffness, specially processed materials can be used, such as memory alloys processed by different processes in sections, or elastic materials spliced in multiple sections.

Also refer to an embodiment, the deformation restraint tube 200 is a coil spring, and includes a dense spring segment 202 located at the proximal end 102 side of the annular segment 201 and a thin spring segment 203 located at the annular segment 201; the outer sleeve 105 at least wraps the thin spring. Section 203.

The deformation constraining tube 200 in this embodiment achieves stiffness at different positions by using coil springs with different densities.

Compared with other setting schemes, the coil spring has the advantages of low cost, good effect, and flexible adjustment according to different needs.

At the same time, the shape of the coil spring itself matches the shape of the tube body 100, which can increase the upper limit of the stiffness of the deformation restraining tube 200 on the premise of ensuring the overall small size, thereby meeting different design requirements.

In one embodiment, the plane where the annular segment 201 is located intersects with the axial direction of the tubular body 100 and the intersection is at the inflection point 122 of the tubular body 100, and the proximal side of the annular segment 201 turns at the inflection point 122 and extends toward the proximal end; deformation The restraint tube 200 extends from the distal end of the pipe body 100 to at least the proximal end side of the annular segment 201, wherein the rigidity of the deformation restraint tube 200 on both sides of the inflection point 122 is the same.

In a particular product, as shown in the figures, the compressive spring segment 202 extends to the distal side of the inflection point 122.

In another embodiment, the plane where the annular segment 201 is located intersects with the axial direction of the tubular body 100 and the intersection is at the inflection point 122 of the tubular body 100, and the proximal side of the annular segment 201 turns at the inflection point 122 and extends toward the proximal end; deformation The restraint tube 200 extends from the distal end of the pipe body 100 to at least the proximal end side of the annular segment 201, wherein the rigidity of the deformation restraint tube 200 on both sides of the inflection point 122 is different.

In a specific product, as shown in the drawings, the dense spring section 202 extends to the inflection point 122, and the other side of the inflection point 122 is the sparse spring section 203.

The boundary line between the dense spring section 202 and the thin spring section 203 may not be strictly located at the inflection point 122, but should be located near the inflection point 122.

It is not difficult to understand that the intersection of the plane where the annular segment 201 is located and the axial direction of the tube body 100 can achieve a larger contact area between the annular segment 201 and the target tissue, to form the inflection point 122, and the bending of the tube body 100 can also be achieved by deforming the restraining tube 200 or the elastic wire 107 to form the inflection point 122.

The solution shown in the figure should also be understood that the boundary line between the thinning spring section 203 and the dense spring section 202 is also located near the inflection point 122, and the boundary line between the support tube 205 and the outer sleeve 105 is also located near the inflection point 122.

At the same time, the self-shape of the helical elasticity can also be used for other functions.

Referring to an embodiment, the deformation restraining tube 200 itself defines a through channel 204 in which the pulling wire 106 extends.

The deformation restraining tube 200 itself also plays the function of protecting the inner pipeline and supporting the inner cavity of the tube body 100.

The force of the pulling wire 106 will produce a compound effect. While driving the radial dimension change of the annular segment 201 (i.e., the axial bending of the deformation constraining tube 200), the pulling wire 106 will also simultaneously generate the axial direction of the deformation constraining tube 200. compression.

The axial compression of the deformation restraining tube 200 at the position of the annular segment 201 can be used to realize the size change of the annular segment 201, but the axial compression at the proximal end 102 side of the annular segment 201 will affect the positioning effect of the annular segment 201.

It is therefore necessary to limit the deformation of the proximal end 102 side of the annular segment 201 to restrain the deformation of the tube 200.

Referring to an embodiment, at least a part of the seal spring segment 202 is covered with a support tube 205, and the support tube 205 is bonded and fixed to the seal spring segment 202.

The support tube 205 is sleeved on the seal spring segment 202, and the support tube 205 can display the axial bending of the seal spring segment 202 in addition to limiting the axial compression of the seal spring segment 202.

Therefore, it can be understood that the support tube 205 improves the overall stiffness of the packing spring section 202 afterward.

In addition to constraining the overall deformation of the packing spring segment 202, the support tube 205 can also prevent the displacement or dislocation between the adjacent elastic rings of the packing spring segment 202.

The displacement or dislocation between the adjacent elastic rings of the packing spring segment 202 can be achieved by reducing the spring pitch. Referring to the accompanying drawings, in this embodiment, the spring pitch of the packing spring segment 202 is set smaller.

In one embodiment, a transition section (not shown) is provided between the thinning spring section 203 and the dense spring section 202, and the spring pitch of the transition section starts from the thinning spring section 203 until the dense spring section 202 gradually shrinks. Adjacent spirals of 202 cancel each other out.

The abutment of the adjacent spirals of the sealing spring segment 202 can completely avoid the axial compression of the sealing spring segment 202, and the support sleeve can avoid the dislocation of the adjacent spirals of the sealing spring segment 202, thereby providing a stable working foundation for the working process of the annular segment 201.

Similarly, the spring thinning section 203 can also be provided with a sleeve. In a reference to an embodiment, the outer casing 105 is made of heat shrinkable material, and the outer circumference of the thinning spring section 203 and the support tube 205 is tightened after heat shrinking.

The spring thinning section 203 needs to achieve a larger degree of deformation, so it needs to be wrapped with a softer material.

The heat-shrinkable material can meet the above conditions and at the same time be easy to assemble, and can achieve a large tightening force through heat-shrinking.

The tightening force can reduce the displacement or dislocation between the adjacent elastic rings of the spring thinning section 203.

At the same time, the heat-shrinkable material can also avoid friction between the deformation restraint tube 200 and the tube body 100 during the change of the annular segment 201.

The heat shrinkable material can be a material such as PTFE heat shrinkable film.

In the overall assembly of the deformation restraining tube 200, referring to an embodiment, the annular segment 201 is provided with a main channel 109 and a fluid channel 110 extending in parallel along the length direction of the tube body 100, and the main channel 109 is provided with mutual The inner sleeve 104 and the outer sleeve 105 are nested, the inner sleeve 104 is inserted into the passage channel 204, the traction wire 106 is inserted into the inner sleeve 104; the deformation restraint tube 200 is inserted into the outer sleeve 105.

Through the separation of the inner and outer sleeves 105, the main lumen 109 actually realizes a plurality of mutually nested channels, which are respectively located in the inner sleeve 104, between the inner and outer sleeves 105, and between the outer sleeve 105 and the tube body 100.

The divided main channel 109 cooperates with the fluid channel 110 to realize the independent setting of each pipeline.

Each channel extends to the proximal end 102 side, and some pipelines communicate with the outside world at the distal end 101 side.

Referring to an embodiment, the annular segment 201 is provided with a main cavity 109 and a fluid cavity 110 extending in parallel along the length direction of the pipe body 100, and the outer edge sidewall of the annular segment 201 has an output communicating with the fluid cavity 110 The hole 111, the inner edge side wall of the annular segment 201 has a wire hole 112 that communicates with the main lumen 109.

The output hole 111 transports the cooling medium in the fluid channel 110 to between the electrode 103 and the tissue, so as to ensure the stable progress of the treatment process.

The wire hole 112 is used for passing the wire 108 so as to realize the power supply of the electrode 103.

The functions of the two are different, and their locations are relatively different.

During the treatment process, the outer edge of the annular segment 201 is in contact with the tissue, and the electrode 103 releases radio frequency energy, so a cooling medium is required to ensure the treatment process.

The wire 108 only needs to ensure stable connection with the electrode 103, so it does not need to occupy the outer edge space of the annular segment 201, and can be arranged on the side edge.

This arrangement also brings benefits to the arrangement of electrodes 103. In one embodiment, the electrode 103 is provided with a wetting hole 1031 for diffusing the cooling medium and a welding position 1032 for connecting the wire 108. On the premise that the lead hole 112 and the output hole 111 are dislocated, the welding position 1032 and the wetting hole 1031 can also be arranged separately.

The electrodes 103 do not actually come into contact with the deformation confinement tube 200, but at the corresponding positions of the two, referring to an embodiment, the annular segment 201 is provided with a plurality of electrodes 103 for releasing radio frequency energy. The distribution on segment 201 is as follows:

Each electrode 103 corresponds to the position of the spring thinning segment 203; or At least one of the electrodes 103 corresponds to the position of the sealing spring segment 202.

The corresponding relationship between the electrode 103 and the different positions of the deformation restraining tube 200 is actually the positional corresponding relationship between the electrode 103 and the part of the annular segment 201 with different degrees of deformation.

Therefore, in the case, the placement requirements of the electrodes 103 are inconsistent.

For example, when a certain case requires the position of the electrode 103 to be flexibly adjusted, the electrode 103 should be more inclined to be arranged on the thinning spring section 203, so as to achieve a larger change of position; for another example, when a certain case needs to be adjusted during the adjustment process, when a certain electrode 103 has a relatively stable position, individual electrodes 103 can be selected to be arranged on the sealing spring segment 202, so as to maintain relatively stable position during adjustment.

Regarding the relationship between the electrode 103 and the deformation confinement tube 200, the wire 108 connected to the electrode 103 needs to pass through the deformation confinement tube 200 and the wire hole 112 to achieve electrical connection.

Referring to an embodiment, the deformation restraining tube 200 defines a passage channel 204, the electrode 103 is connected to the lead 108, one end of the lead 108 extends from the passage channel 204 to the proximal end 102, and the other end is connected to the spring gap of the deformation restraining tube 200. Electrode 103 is connected.

The penetration channel 204 in the coil spring of the deformation restraining tube 200 can protect the wire 108 from the friction of the tube body 100. When the wire 108 is connected to the electrode 103 outside the tube body 100, it needs to cross the coil spring, and the spring gap is a suitable value. choice, especially in the spring thinning section 203.

The spring pitch of the spring thinning section 203 is relatively large, so the spring gap is relatively large, which facilitates the penetration of the wire 108.

If the electrode 103 corresponds to the sealing spring section 202, the wire 108 should be properly protected to prevent the wire 108 from being worn.

Regarding the overall assembly relationship between the deformation restraint tube and the tube body, referring to an embodiment, the radiofrequency ablation catheter includes a tube body 100, and the tube body 100 has opposite distal ends 101 and proximal ends 102. An electrode 103 for releasing energy is installed on the outer wall of the tube body 100, an inner sleeve 104 and an outer sleeve 105 nested with each other are arranged in the tube body 100, and the inner sleeve 104 is provided with a shape for the distal end 101 of the tube body 100. The elastic wire 107 and the pulling wire 106 are arranged side by side and the two are fixed to each other at the position adjacent to the distal end 101 of the tube body 100; There is a deformation restraint tube 200, the outer sleeve 105 is made of heat shrinkable material, and the deformation restraint pipe 200 is tightened after heat shrinkage;

A wire 108 is passed through the radial gap between the inner sleeve 104 and the outer sleeve 105, and the end of the wire 108 passes through the outer sleeve 105 and the tube wall of the tube body 100 and is connected to the electrode 103.

The elastic wire 107, the pulling wire 106 and the guide wire 108 need to be penetrated in the tube body 100, wherein the elastic wire 107 and the pulling wire 106 are located in the inner sleeve 104, and the guide wire 108 is located in the radial gap between the inner sleeve 104 and the outer sleeve 105.

The mutual nesting of the inner sleeve 104 and the outer sleeve 105 realizes the assembly and isolation of the elastic wire 107, the pulling wire 106 and the guide wire 108, thereby avoiding unnecessary multiple lumens on the tube body 100.

At the same time, the inner and outer tubes and the outer sleeve 105 can realize pre-assembly between various components, thereby improving the overall production efficiency and yield.

A wire 108 is passed between the outer sleeve 105 and the inner sleeve 104, and the size of the gap between the two is ensured by the size of the deformation restraining tube 200.

On the one hand, the deformation restraint tube 200 is used to cooperate with the elastic wire 107 to maintain the shape of the tube body 100, and on the other hand, it is used to maintain the inner size of the main lumen 109 to prevent the main lumen 109 from being bent under the action of the pulling wire 106. or closed.

In order to realize this function, the deformation restraint tube 200 needs to support the inner wall of the main lumen 109.

Referring to an embodiment, the deformation restraining tube 200 is a coil spring and is wound around the outer circumference of the inner sleeve 104, and the wire 108 is passed between the coil spring and the outer wall of the inner sleeve 104. The coil spring can be deformed in its own axial direction to release the change of the radial dimension of the ring, and at the same time, the coil spring can support the inner wall of the main lumen 109, thereby ensuring the stability of the catheter during the intervention process.

Regarding the matching relationship between the inner sleeve 104, the outer sleeve 105 and the tube body 100, referring to an embodiment, the tube body 100 is provided with a main cavity 109 and a fluid cavity 110 extending in parallel along the length direction of the tube body 100, The side wall of the tube body 100 is provided with an output hole 111 that communicates with the fluid channel 110;

The fluid channel 110 is used to deliver the cooling medium to the output hole 111, so as to ensure the stable progress of the ablation process.

The independence of the fluid channel 110 and the main channel 109 can ensure that the flow rate and flow rate of the cooling medium are not affected by the components in the main channel 109, and the wires 108 in the main channel 109 can also avoid contact with the cooling medium, improving safety.

The ablation function of the distal end 101 is mainly realized by the cooperation of various components in the main lumen 109. In terms of specific form, referring to an embodiment, the distal end 101 of the tube body 100 is coiled into a ring shape, and the cross section at the distal end 101 Above, the main lumen 109 is arranged eccentrically relative to the central axis of the tube body 100 and is close to the inner edge of the ring.

The annular shape of the distal end 101 of the tube body 100 is actually a non-closed annular shape. Under the action of the pulling wire 106, the radial size of the annular shape can be changed (refer to FIG. 1b and FIG. 1c), so as to realize the size of the catheter for different lesions. adapt.

In this embodiment, the pulling wire 106 is used to reduce the radial size of the ring, and the elastic wire 107 is used to maintain and restore the radial size of the ring.

Compared with the central axis of the tube body 100, the main lumen 109 is close to the inner edge of the ring. It is not difficult to understand that in the embodiment disclosed in FIG. 4b, the traction wire 106 is closer to the inner edge of the ring than the elastic wire 107.

When the pulling wire 106 is forced to move, the pulling wire 106 exerts force on the elastic wire 107 through the fixed position with the elastic wire 107, and the elastic force is deformed by the force to realize the change of the annular radial dimension.

Correspondingly, in the matching relationship between the fluid channel 110 and the main channel 109, in the embodiment disclosed with reference to FIGS. 5a-5d, Outer edge, the output hole 111 is located at the outer edge of the ring.

The fluid channel 110 being closer to the outer edge of the ring is not only convenient for distributing the main channel 109 and the fluid channel 110 on the tube body 100, but also facilitates the arrangement of the output hole 111. During the ablation process, the ring generally contacts the adjacent tissue through its outer edge, and the electrode 103 thus realizes the transmission of radio frequency energy. Therefore, the setting of the output hole 111 on the outer edge of the ring can more directly deliver the cooling medium to the ablation position, thereby ensuring the stable implementation of the ablation process. The fluid channel 110 also functionally relieves the stress of the annular outer edge. Under the action of the pulling wire 106, the radial dimension of the ring changes. At this time, the inner edge of the ring is relatively compressed and the outer edge is relatively stretched. The fluid channel 110 and the main channel 109 opened in the tube body 100 are actually The stress-releasing space of the material of the tube body 100 is formed.

Regarding the details of the arrangement of the inner sleeve 104, in the embodiments disclosed with reference to FIGS. 2b and 4b, the cross section of the inner sleeve 104 is an ellipse, and the long axis direction of the ellipse is consistent with the radial direction of the ring.

When the pulling wire 106 exerts a force on the elastic wire 107, the pulling wire 106 actually moves under the restraint of the inner sleeve 104, and exerts a force on the inner sleeve 104. In this embodiment, the restriction on the movement direction of the traction wire 106 is realized by the cross-sectional shape of the inner sleeve 104, so as to improve the actuation effect of the traction wire 106. While restraining the pulling wire 106, the inner cannula 104 can also avoid mutual interference between the pulling wire 106 and other components (e.g., the guide wire 108), thereby improving the overall stability of the catheter. Referring to an embodiment, the wire 108 is located on one side of the inner sleeve 104 in the direction of the central axis of the ring and abuts against the outer wall of the inner sleeve 104.

In addition to the functions of restraint and isolation, the inner sleeve 104 can also play other functions. Referring to an embodiment, the inner wall of the inner sleeve 104 is provided with a lubricating layer or the inner sleeve 104 is a lubricating material.

The realization of lubrication can reduce the movement resistance of the traction wire 106, improve the overall operating experience of the catheter, and also reduce wear and improve stability. Regarding the selection of specific materials, refer to an embodiment, the inner sleeve 104 is a heat-shrinkable material, and the elastic wire 107 and the pulling wire 106 are tightened after heat-shrinking. Specifically, the heat-shrinkable material may be a PTFE heat-shrinkable film or the like. In terms of length, the axial length of the inner sleeve 104 is the same as or slightly shorter than the axial length of the annular shape. In this embodiment, the pre-assembly of the elastic wire 107 and the traction wire 106 can be realized by the heat shrinking of the inner sleeve 104, thereby facilitating subsequent assembly.

According to the above description, it is not difficult to understand that the fluid channel 110 is used to deliver the cooling medium to the output hole 111, so the arrangement of the output hole 111 needs to ensure the distribution effect of the cooling medium.

For the setting and specific processing method of the output hole 111, reference may be made to the above related description, which will not be repeated here.

Combining Embodiment 1 and Embodiment 2, it is not difficult to understand that the present invention also discloses a radiofrequency ablation catheter, including a tube body 100. The tube body 100 has opposite distal ends 101 and proximal ends 102. An electrode 103 for releasing energy is installed on the outer wall of the part 101, an inner sleeve 104 and an outer sleeve 105 nested with each other are arranged in the tube body 100, and the inner sleeve 104 is pierced with a part of the distal end 101 of the tube body 100. The shaped elastic wire 107 and the pulling wire 106, the elastic wire 107 and the pulling wire 106 are arranged side by side and the two are fixed to each other at the position adjacent to the distal end 101 of the tube body 100;

A wire 108 is passed through the radial gap between the inner sleeve 104 and the outer sleeve 105, and the end of the wire 108 passes through the outer sleeve 105 and the tube wall of the tube body 100 and is connected to the electrode 103;

The distal end 101 of the tube body 100 is an annular segment 201, which can be deformed under the action of the traction wire 106. At least extending to the proximal end 102 side of the annular segment 201, the rigidity D1 of the deformation restraining tube 200 at the proximal end 102 side of the annular segment 201 is greater than the rigidity D2 at the annular segment 201.

In combination with the above, the elastic wire 107, the pulling wire 106 and the guide wire 108 need to be passed through the tube body 100, wherein the elastic wire 107 and the pulling wire 106 are located in the inner sleeve 104, and the guide wire 108 is located between the inner sleeve 104 and the outer sleeve 105. in the radial gap.

The mutual nesting of the inner sleeve 104 and the outer sleeve 105 realizes the assembly and isolation of the elastic wire 107, the pulling wire 106 and the guide wire 108, thereby avoiding unnecessary multiple lumens on the tube body 100.

At the same time, the inner and outer tubes and the outer sleeve 105 can realize pre-assembly between various components, thereby improving the overall production efficiency and yield.

The stiffness D1 is greater than the stiffness D2, and the direct technical effect is that the deformation restraint tube 200 is not easily deformed at the proximal end 102 side of the annular segment 201 compared to the annular segment 201.

From the overall view of the tube body 100, under the action of the pulling wire 106, the annular segment 201 is more easily deformed than the proximal end 102 of the annular segment 201, so the proximal end 102 of the annular segment 201 provides the deformation of the annular segment 201 Similar to the role of the “base”, it is used to control the overall shape of the annular segment 201 in space.

Therefore, the structure is compact, the annular segment can be flexibly changed according to the needs of different working conditions, and the radio frequency ablation system based on this can realize a smooth and controllable ablation process.

### Embodiment 3

1a, 7a to 7e, the present invention also discloses a radio frequency ablation system, including the radio frequency ablation catheter and the operating handle 300 in the above technical solution, and the tube body 100 of the radio frequency ablation catheter is provided with a traction for bending adjustment Wire 106, operating handle 300 includes:

The handle body 301, the proximal end of the tube body 100 is directly or indirectly fixed to the handle body 301;

The connector 302 is slidably mounted on the handle body 301, and the connector 302 is provided with a mounting hole 303; the connector 302 is provided with an escape channel 304 through which the fluid delivery tube 120 and the wire 108 pass through the connector 302 and extending to the outside of the handle body;

The cock 305 is rotatably fitted in the installation hole 303, and the proximal end portion of the traction wire 106 is clamped and fixed in the radial gap between the cock 305 and the installation hole 303;

The driving member 306 is movably mounted on the handle body 301, and drives the connecting member 302 to slide to drive the traction wire 106.

The handle body 301 provides support for each component, and is used to determine the relative position of the tube body 100 and the pulling wire 106 so as to realize the relative movement of the two.

In this embodiment, the proximal end of the tube body 100 is indirectly fixed to the handle body 301 through the braided tube 117.

At the same time, the handle body 301 can provide a holding space for the operator, and can also be provided with a holding sheath 307 to improve the holding feeling.

The connecting piece 302 restricts the movement path in the space through the handle body 301.

Therefore, setting the avoidance hole on the connector 302 can increase the volume of the connector 302 as much as possible without interfering with other components, thereby increasing the contact area with the handle body 301 and ensuring the stability of the movement of the connector 302.

The tap 305 enables the assembly of the pull wire 106 and the connector 302.

The design of the cock 305 makes the assembly simple and easy to adjust. Compared with the related art, the cock 305 can flexibly adjust the specific position where the pulling wire 106 is combined with the connecting piece 302, and can release the length error of the pulling wire 106.

At the same time, when the pulling wire 106 receives a large force, the cock 305 can play a role of preventing overload through its own rotation, so as to prevent the pulling wire 106 from breaking at a weak position.

Regarding the specific matching relationship of the pulling wire 106, referring to an embodiment, the connecting member 302 is provided with a pulling installation channel 308 penetrating the hole wall of the installation hole 303, and the proximal end portion of the pulling wire 106 enters the radial direction through the pulling installation channel 308 gap.

The pulling installation channel 308 is used for the pulling wire 106 to pass through. After the pulling wire 106 is inserted into the radial gap, the rotation of the cock 305 will drive the pulling wire 106 into the interlayer between the cock 305 and the installation hole 303, thereby realizing the clamping.

In one embodiment, the cock 305 is provided with a receiving groove (not shown) for receiving at least a part of the pulling wire 106.

The accommodating groove is opened along the circumference of the cock 305 and is used to accommodate the traction wire 106 during the rotation of the cock 305, which can reduce the resistance of rotation and avoid excessive stress on the traction wire 106; 106 position, to avoid the occurrence of dislocation and other situations, and improve the stability.

There are various ways for the cock 305 to drive the traction wire 106 to move together during its own rotation. For example, it can be realized by friction or by separately setting a clamping structure that protrudes from the cock 305. It can also be referred to in an embodiment. For the docking channel 309, the cock 305 rotates relative to the connecting piece 302 by itself to have:

In the released state (not shown), the pulling installation channel 308 and the docking channel 309 are aligned with each other, and the pulling wire 106 enters the docking channel 309 from the pulling installation channel 308;

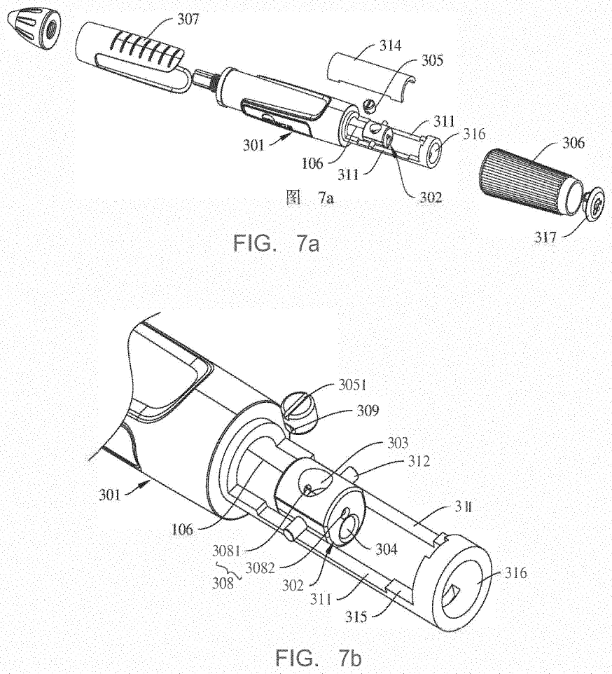

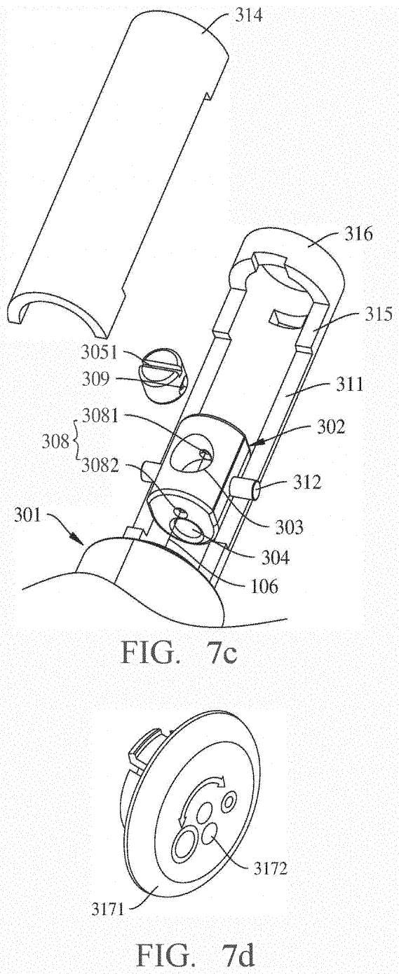

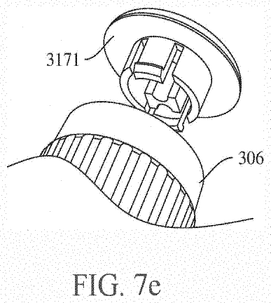

In the locked state (refer to FIG. 7b and FIG. 7c), the pulling installation channel 308 and the docking channel 309 are misaligned with each other, and the pulling wire 106 enters the radial gap through the pulling installation channel 308, and then extends along the circumferential direction of the cock 305 and then enters the docking channel 309.

The docking channel 309 can accommodate the traction wire 106 and drive the traction wire 106 to rotate together when the cock 305 rotates, thereby avoiding the disadvantage that the traction wire 106 is not strongly restrained in other structures.

The butt channel 309 allows the pulling wire 106 to be threaded back and forth, thereby increasing the clamping strength of the pulling wire 106 while the overall structure is unchanged.

Corresponding to the characteristics of reciprocating penetration, referring to an embodiment, the traction installation channel 308 includes a first channel 3081 at the distal end of the installation hole 303, and a second channel 3082 at the proximal end of the installation hole 303,

The cock 305 rotates relative to the connecting piece 302 by itself with:

In the released state, each pulling installation channel 308 and the docking channel 309 are aligned with each other and allow the pulling wire 106 to pass directly;

In the locked state, the docking channel 309 and each pulling installation channel 308 are mutually displaced, and the corresponding part of the pulling wire 106 is twisted, clamped and fixed in the radial gap.

The arrangement of the first channel 3081 and the second channel 3082 can not only be used to realize the reciprocating threading of the traction wire 106 in the docking channel 309, but in the one-way threading scheme, the setting of the first channel 3081 and the second channel 3082 The installation of the pulling wire 106 can be facilitated, allowing the pulling wire 106 to have an assembly margin at the proximal end side of the installation hole 303, and it is convenient to release the fitting error during the rotation of the cock 305.

When the cock 305 is in the locked state, there is an included angle between the pulling installation channel 308 and the docking channel 309.

The axial included angle between the traction installation channel 308 and the docking channel 309 is a fixed angle. When the cock 305 is in a locked state, the fixed angle is 30 degrees to 110 degrees, and the cock 305 is installed in the installation hole 303 by interference fit through the traction wire 106. Inside.

In terms of the driving form of the cock 305, a driving structure can be provided on the cock 305 to facilitate the application of the force. In an embodiment, the cock 305 is cylindrical and has a driving groove 3051 on the end surface.

The driving groove 3051 is formed by removing the material from the end face of the cock 305, which avoids the waste of separately provided materials.

The cylindrical overall structure facilitates the fitting and rotation of the cock 305 in the mounting hole 303.

The tube body 100 has a plurality of pipelines extending toward the proximal end, such as wires for delivering radio frequency signals, fluid pipelines for delivering cooling medium, etc. Therefore, the handle body 301 itself is also a channel for the aforementioned pipelines.

The connecting piece 302 needs to avoid interference with the above-mentioned pipeline during the sliding process.

Referring to an embodiment, the connecting member 302 is provided with an escape channel 304, and the corresponding components in the tube body 100 extending toward the proximal end of the operating handle 300 pass through the connecting member 302 through the escape channel 304.

Compared with the connector 302 avoiding the corresponding area of the pipeline as a whole, the connector 302 in this embodiment has the advantage of increasing the volume of the connector 302 as much as possible without interfering with other components.

The larger the contact area between the connecting piece 302 and the handle body 301 is, the stronger the sliding restraint ability that the handle body 301 can enhance, thereby ensuring the stability of the movement of the connecting piece 302; correspondingly, the larger the volume and mass of the connecting piece 302, the better the traction The more stable the driving effect of the wire 106 is, the better the operating feel can be obtained.

Regarding the arrangement details of the avoidance channel 304 and the installation hole 303, in reference to an embodiment, the installation hole 303 is a blind hole and does not intersect with the avoidance channel 304.

When the cock 305 in the mounting hole 303 clamps the traction wire 106, a large stress may be generated, and certain danger may be generated in an unexpected situation. The separated arrangement can avoid the above situation and improve the overall stability of the operating handle 300.

In terms of the matching details of the pulling installation channel 308 and the avoidance channel 304, referring to an embodiment, the connecting member 302 is provided with a pulling installation channel 308 penetrating the hole wall of the installation hole 303, and the proximal end portion of the pulling wire 106 passes through the pulling installation channel 308 enters the radial clearance; the avoidance channel 304 is arranged parallel to and below the traction mounting channel 308.

The lower part in this embodiment refers to FIG. 7b, the avoidance channel 304 is located below the traction installation channel 308, and the extending directions of the two are parallel to each other, which facilitates the passage of pipelines during the assembly process.

In addition to the sliding restraint of the connecting member 302 by the inner wall of the handle body 301, it can also be referred to an embodiment that the handle body 301 is a cylindrical structure, and the side wall is provided with a guide strip hole 311 extending in the axial direction, and the connecting member 302 slides Installed inside the cylindrical structure, the connecting member 302 is provided with a guide key 312 extending radially out of the guide strip hole 311, the driving member 306 is rotatably sleeved on the outer circumference of the handle body 301, and the inner wall of the driving member 306 is provided with The threaded structure of the guide key 312 is matched.

The guide bar hole 311 and the guide key 312 provide a sliding pair for limiting the movement of the connecting member 302.

At the same time, the threaded structure can accurately determine the position of the guide key 312 relative to the guide bar hole 311, thereby determining the relative position of the connecting piece 302 relative to the handle body 301 and realizing driving.

Regarding the specific setting of the handle body 301, referring to an embodiment, a part of the side wall of the cylindrical structure is a detachable cover 314, and the guide strip hole 311 is located at the seam 315 between the cover 314 and other parts of the cylindrical structure, the cock 305 is located on the side of the connector 302 facing the cover 314.

The detachable cover 314 actually provides an operation opening on the handle body 301 to facilitate the assembly of various components. Similarly, the cock 305 is located on the side of the connector 302 facing the cover 314.

Meanwhile, the guide strip hole 311 is arranged at the seam 315 to reduce the mechanically weak area on the handle body 301.

The driving force of the connecting member 302 comes from the rotation of the driving member 306, so the relative position of the driving member 306 relative to the handle body 301 needs to be determined.

Referring to an embodiment, a positioning ring 316 is formed by extending the material on the proximal side of the cylindrical structure relative to other parts of the cover body 314, and an end cover 317 is provided on the driving member 306 to cooperate with the positioning ring 316, and one end of the end cover 317 penetrates through. The driving member 306 is engaged with the positioning ring 316, and the other end is enlarged to form a positioning end 3171. The positioning end 3171 is used to determine the relative position of the driving member 306 and the handle body 301. The positioning end 3171 is provided with a pipeline hole 3172.

The positioning end 3171 of the end cover 317 can ensure the relative position of the driving member 306 and the handle body 301 during the rotation process, so as to realize the driving of the connecting member 302.

The handle body 301 is engaged with the end cover 317 through the positioning ring 316 formed of an integrated material, which can avoid the influence of the separated cover body 314 on the installation effect of the end cover 317, and the overall structure is compact and stable.

The present invention discloses a production process of a radiofrequency ablation catheter. The tube body 100 of the radiofrequency ablation catheter includes a relatively distal end 101 and a proximal end 102. The production process includes:

Passing the elastic wire 107 and the pulling wire 106 side by side in the inner sleeve 104, the distal ends 101 of both the elastic wire 107 and the pulling wire 106 are pre-fixed, and the inner sleeve 104 is heat-shrinked to obtain a pulling wire assembly;

An output hole 111 and a wire hole 112 are provided on the side wall of the tube body 100. The tube body 100 is provided with a fluid channel 110 and a main channel 109. The output hole 111 is communicated with the fluid channel 110, and the wire hole 112 is connected with the main channel. 109 Connected;

The electrode 103 is sleeved on the tube body 100, and the wire 108 connected to the electrode 103 enters the main lumen 109 through the wire hole 112 and extends to the proximal end 102;

After docking the support sleeve with the deformation restraint tube 200 with an inner cavity, it is inserted into the outer sleeve 105, and the outer sleeve 105 is heat-shrinked to obtain a shaping component, and then the shaping component is passed through the main cavity 109; The assembly passes through the inner cavity of the deformation restraining tube 200, and the distal end 101 of the pull wire assembly extends out of the deformation restraining tube 200 to the distal end 101 of the tube body 100 and is fixed.

The steps in this application may or may not be performed sequentially.

For example, the pulling wire assembly and the shaping assembly are pre-assembled to obtain corresponding parts, which facilitates the arrangement of the process and the deployment of the assembly process.

During the assembly process of the pulling wire assembly, the elastic wire 107 is used to maintain the shape of the tube body 100, and the pulling wire is used to drive the deformation of the elastic wire 107 to realize the deformation of the tube body 100.

Therefore, from a functional point of view, the pulling wire needs to move relative to the tube body 100 and the inner sleeve 104, while the elastic wire 107 can be selectively fixed or not fixed to the inner sleeve 104.

Referring to an embodiment, the elastic wire 107 is fixed to the inner sleeve 104 or the tube body 100.

Correspondingly, in another embodiment, the elastic wire 107 is provided separately from the inner sleeve 104 or the tube body 100.

The elastic wire 107 and the traction wire 106 can be fixed by direct welding, crimping and other operations, or can be connected and fixed by a third-party component.

Referring to an embodiment, the distal end 101 of the elastic wire 107 is pre-shaped into a ring shape, and the fixing of the elastic wire 107 and the distal end 101 of the pulling wire 106 to each other includes:

Covering the connecting cap 113 on the distal end 101 side of the elastic wire 107 and the pulling wire 106, and adjusting the pulling wire 106 to the inner side of the loop of the elastic wire 107;

A filler is added into the gap between the connecting cap 113, the elastic wire 107 and the pulling wire 106, and the connecting cap 113 is forced to tighten the elastic wire 107 and the pulling wire 106.

In this embodiment, the connection cap 113 functions as the third-party component mentioned above, and realizes the force connection between the traction wire 106 and the elastic wire 107.

The connecting cap 113 can be set in the form of one side open and the other side closed, or can be set in the form of a riveted tube with two ends open in the drawing.

The connection cap 113 can realize the clamping of the elastic wire 107 and the traction wire 106 through its own deformation, but in order to ensure the strength of the connection cap 113 after deformation, the deformation degree of the connection cap 113 is limited under the conventional material selection. The gripping force of the pull wire 106 is limited.

This problem is overcome by the filler in this example.

Under the condition that the driving force for the deformation of the connecting cap 113 is the same, the setting of the filler can increase the contact area and the holding force between the connecting cap 113, the elastic wire 107, and the pulling wire 106, thereby ensuring the connecting effect of the connecting cap 113.

Referring to an embodiment, the filler is a hot melt material.

The filler can change its own shape through hot melting, for example, from solid phase to liquid phase, so as to penetrate into the connection cap 113, elastic wire 107, and traction wire 106, and when the filler material changes back to the solid phase, it can fill in the gap between the above three.

Referring to an embodiment, the filler is solder.

Solder has the advantages of good fluidity in liquid state, good compatibility with elastic wire 107 and traction wire 106, high strength in solid state, low cost, easy to obtain and meet relevant requirements of industrial production.

The pull wire assembly also achieves the stability of the assembly itself through the heat shrinking of the inner sleeve 104.

The heat shrinking of the inner sleeve 104 can achieve the tightening of the elastic wire 107 and the pulling wire.

So as to determine the relative position of the two.

For example, the pulling wire 106 is located on one side of the elastic wire 107.

The inner sleeve 104 can also reduce the resistance of the pulling wire movement by its own material.

During the assembly process of the plastic component, the deformation restraint tube 200 can not only assist the elastic wire 107 to maintain the shape of the tube body 100, but also ensure the overall shape of the main lumen 109 and prevent the internal collapse of the tube body 100 during the deformation process.

Referring to an embodiment, after the shaping element is passed through the main cavity 109, the tube body 100 is further molded to fix the electrode 103.

In this embodiment, the electrode 103 is annular and is sleeved on the tube body 100.

Before assembling, the inner diameter of the electrode 103 is larger than the inner diameter of the tube body 100, which facilitates the installation of the electrode 103 and the connection of the wire 108.

The electrode 103 is molded by the tooling, the overall size or shape of the electrode 103 is changed, and the electrode 103 and the tube body 100 are fixed.

The plastic component is inserted into the main cavity 109 before molding, which can prevent the collapse of the main cavity 109 during the molding process to ensure the stability of the assembly.

Similar to the fact that the main lumen 109 may be forced to collapse during the molding process above, the fluid delivery tube 120 and the fluid channel 110 may also be forced to collapse during the molding process.

The difference is that the deformation confinement tube 200 needs to be installed in the main lumen 109 originally, so this problem can be overcome by passing the deformation confinement tube 200. However, in the fluid delivery tube 120 and the fluid channel 110, which are originally the delivery paths of the fluid, there are no components that can be pre-assembled to overcome the collapse problem. In order to overcome this problem, the fluid delivery tube 120 and the fluid delivery cavity can be filled with fluid to maintain their shape and prevent collapse during the molding process. Also refer to an embodiment, before molding, further comprising:

Passing the first lining member through the fluid delivery tube 120, and bonding the fluid delivery tube 120 on the proximal end 102 side of the fluid channel 110;

The second lining member is inserted into the fluid channel 110 from the distal end 101 side of the tubular body 100.

The first inner lining member and the second inner lining member have the same function, and may be the same or different in structure or size.

In use, the two enter the molding position from different directions.

In the actual entry process, the order of entry of the first lining piece and the second lining piece may be different, so it is necessary to pay attention to the interference of the two.

As described above, the deformation restraint tube 200 can restrain the tendency of the main lumen 109 to collapse.

However, the deformation restraining tube 200 itself is an elastic part, so during the molding process, the main cavity 109 may still be partially collapsed.

Referring to an embodiment, before the heat shrinkable outer sleeve 105, a third inner lining member is passed through the deformation restraining tube 200. In this embodiment, the third inner lining member restrains the collapse of the main lumen 109 by restraining the collapse of the deformation restraining tube 200.

In connection with the above-mentioned embodiments, the first inner lining member, the second inner lining member and the third inner lining member have the same functions, and are all used to restrain the tendency of the cavity or component to collapse.

In terms of structure, material and size, the three are consistent or inconsistent. Referring to an embodiment, the first inner lining member, the second inner lining member, and the third inner lining member are all nickel-titanium wires. The nickel-titanium material has excellent elasticity, which can avoid damage or hidden dangers caused by large stress while ensuring the internal dimensions of the cavity or component.

In the subsequent assembly process, referring to an embodiment, the production process further includes:

The elastic member 116 is passed through the elastic catheter 115, the distal end 101 of the elastic catheter 115 is connected to the deformation restraining tube 200, and the fluid delivery tube 120, the elastic wire 107 and the guide wire 108 are bonded and fixed on the distal end 101 side of the elastic catheter 115.

The above-mentioned tube body 100 focuses on the part of the distal end 101 of the catheter that can change its own size. During the process of the catheter entering the human body, the connection between the tube body 100 and the operating handle 300 and the penetration of each component are mainly realized through the elastic catheter 115.

Therefore, the elastic conduit 115 needs to be connected with the tube body 100 by force, and a passage for each component to pass through needs to be provided inside.

Referring to one embodiment, the fluid delivery tube 120, the pull wire 106 and the guide wire 108 extend toward the proximal end 102 through the elastic conduit 115.

In terms of performance, the elasticity of the elastic conduit 115 is mainly provided by the elastic member 116.

In terms of material, the material of the elastic conduit 115 may or may not be the same as that of the tube body 100.

In order to realize the fixation and assembly of the internal components of the elastic conduit 115, referring to an embodiment, the production process further includes: threading the wire 108, the fluid delivery tube 120 and the elastic conduit 115 in the braided tube 117, heat shrinking the braided tube 117, The guide wire 108, the fluid delivery tube 120, the elastic catheter 115 and the braided tube 117 are bonded and fixed, and the guide wire 108, the fluid delivery tube 120, and the elastic catheter 115 are bonded and fixed.

In order to realize the relative movement of the pulling wire 106 and the tube body 100, referring to an embodiment, the production process further includes:

Connect the braided tube 117 to the handle body 301, and thread the wire 108 and the fluid delivery tube 120 through the handle body 301;

The traction wire 106 is connected to the connecting piece 302, the connecting piece 302 is slidably installed with the handle body 301, and a driving piece 306 for driving the connecting piece 302 to slide relative to the handle body 301 is installed on the handle body 301.

Regarding the specific setting of the handle, referring to an embodiment, the connecting member 302 is provided with a traction installation channel 308 penetrating the hole wall of the installation hole 303, and the proximal end portion of the traction wire 106 enters the radial gap through the traction installation channel 308; avoid Channel 304 is disposed parallel to and below the tow mounting channel 308.

The lower part in this embodiment refers to FIG. 7b, the avoidance channel 304 is located below the traction installation channel 308, and the extending directions of the two are parallel to each other, which facilitates the passage of pipelines during the assembly process.

In addition to the sliding restraint of the connecting member 302 by the inner wall of the handle body 301, it can also be referred to an embodiment that the handle body 301 is a cylindrical structure, and the side wall is provided with a guide strip hole 311 extending in the axial direction, and the connecting member 302 slides Installed inside the cylindrical structure, the connecting member 302 is provided with a guide key 312 extending radially out of the guide strip hole 311, the driving member 306 is rotatably sleeved on the outer circumference of the handle body 301, and the inner wall of the driving member 306 is provided with The threaded structure of the guide key 312 is matched.

The guide bar hole 311 and the guide key 312 provide a sliding pair for limiting the movement of the connecting member 302.

At the same time, the threaded structure can accurately determine the position of the guide key 312 relative to the guide bar hole 311, thereby determining the relative position of the connecting piece 302 relative to the handle body 301 and realizing driving.

Regarding the specific setting of the handle body 301, referring to an embodiment, a part of the side wall of the cylindrical structure is a detachable cover 314, and the guide strip hole 311 is located at the seam 315 between the cover 314 and other parts of the cylindrical structure, the cock 305 is located on the side of the connector 302 facing the cover 314.

The detachable cover 314 actually provides an operation opening on the handle body 301 to facilitate the assembly of various components. Similarly, the cock 305 is located on the side of the connector 302 facing the cover 314.

Meanwhile, the guide strip hole 311 is arranged at the seam 315 to reduce the mechanically weak area on the handle body 301.

The driving force of the connecting member 302 comes from the rotation of the driving member 306, so the relative position of the driving member 306 relative to the handle body 301 needs to be determined.

Referring to an embodiment, a positioning ring 316 is formed by extending the material on the proximal side of the cylindrical structure relative to other parts of the cover body 314, and an end cover 317 is provided on the driving member 306 to cooperate with the positioning ring 316, and one end of the end cover 317 penetrates through. The driving member 306 is engaged with the positioning ring 316, and the other end is enlarged to form a positioning end 3171. The positioning end 3171 is used to determine the relative position of the driving member 306 and the handle body 301. The positioning end 3171 is provided with a pipeline hole 3172.

The present invention also discloses a radiofrequency ablation catheter, which is produced according to the production process of the radiofrequency ablation catheter in the above technical solution.

The production embodiment of the radiofrequency ablation catheter is exemplarily given in the above in conjunction with the specific operation steps and process parameters.

1. Elastic Wire 107 Stereotypes

The elastic wire 107 in this embodiment is made of nickel-titanium material, and in the actual product, it is expressed as a nickel-titanium wire, and the operations are as follows:

Step 1. Use cutting pliers to cut off the nickel-titanium wire;

Step 2. Wind the nickel-titanium wire on the core of the shaping die, and install the die sleeve;

Step 3. Put the mold into a high temperature furnace for shaping;

Step 4. Take out the mold and take off the shaped nickel-titanium wire, namely the elastic wire 107 above. The function of the elastic wire 107 is to shape the distal end of the tube body 100 into the annular segment 201.

2. Electrode 103 Welding

Step 1. Fix the electrode 103 on the electrode holder and place it in the field of view of the microscope, and adjust the microscope to ensure that the ring electrode 103 can be seen clearly;

Step 2. Gently scrape off the insulation layer of the distal end 101 of the wire 108 with a blade;

Step 3. Dip an appropriate amount of flux with solder wire and apply it to the welding position 1032 of the electrode 103 (the position where the wetting hole 1031 is not provided inside the electrode 103 is the welding position 1032);

Step 4. Cut off an appropriate amount of solder wire, and use a tool to weld the electrode 103 and the wire 108 together;

Step 5. Remove the electrode 103 from the fixture for self-checking;

3. Pull Wire 106 Welding

Step 1. Cut the self-winding number of the elastic wire 107 to a corresponding length of 1.25 turns, insert the pulling wire 106 and the distal end 101 of the shaped elastic wire 107 into the connecting cap 113, adjust the position of the pulling wire 106, and make the pulling wire 106 Inside the self-winding shape of the elastic wire 107;

Step 2. Clamp the elastic wire 107 and the pulling wire 106 on the proximal end 102 side of the connecting cap 113 with flat-nose pliers, cut off an appropriate amount of solder wire, that is, the filler material above, apply the flux to the distal end 101 of the connecting cap 113, and Welding is performed on the distal end 101 of the connecting cap 113;

Step 3. Check whether the proximal end 102 of the connecting cap 113 has solder flowing in, if not, soldering needs to be performed on the proximal end 102 of the connecting cap 113;

Step 4. Flatten the connection cap 113 with the ring-shaped compression pliers, and pull the traction wire 106 and the elastic wire 107 forcefully to check whether the connection is firm;

Step 5. Cut off an appropriate amount of PTFE heat-shrinkable film, namely the inner sleeve 104 above. The length of the inner sleeve 104 is the same as or slightly shorter than that of the tube body 100. Insert the traction wire 106 and the elastic wire 107 into the inner casing 104 to adjust the traction. The wire 106 is positioned so that the pull wire 106 is on the inside edge of the loop without kinks;

Step 6. Set the parameters of the hot air equipment to 400° C., clamp the connection cap 113 out of the hot air outlet with flat-nose pliers, and the corresponding position of the connection cap 113 avoids the hot air area, and heat shrink the inner sleeve 104 to tighten the traction wire 106 and the elastic silk 107;

Step 7. Insert the pulling wire 106 into the pulling outer sleeve 1061 until it is in contact with the inner sleeve 104 to obtain a pulling wire assembly.

4. Molded

4.1 Pipe Body 100 Punch

Step 1. Use a blade to cut the pipe body 100 with a length of 90-100 mm;

Step 2. Use the corresponding mold to punch holes on the punching machine.

4.2 Install Electrode 103

Step 1. Use tools to process the wire hole 112 on the corresponding side of the output hole 111 on the tube body 100. The wire hole 112 has low precision requirements, and common tools can be used to facilitate processing, such as tweezers, drill bits, drill needles, etc. Wall penetration between channel 109 and fluid channel 110;

Step 2. Intercept the length of the tube body 100: the proximal end 102 of the tube body 100 is about 20 mm away from the first output hole 111 (the corresponding vertical section in FIG. 2a is about 15 mm), and the distal end 101 of the tube body 100 is inclined. Cut to facilitate the installation of the electrode 103, and thin the proximal end 102 of the tube body 100 to facilitate taking over;

Step 3. Install the electrode 103 on the tube body 100, and intercept the length of the tube body 100: the distance between the distal end 101 of the tube body 100 and the nearest electrode 103 is less than or equal to 2 mm.

4.3 Heat Shrink Deformation Restraint Tube 200

Step 1. Intercept the length of the deformation restraint tube 200. The deformation restraint tube 200 is made of flat wire material with different densities. The flat wire material is 0.051 mm thick, 0.3 mm wide, 0.6 mm thick, and 40 mm long. mm to 70 mm, the length of the sealing spring segment 202 is 10 mm to 25 mm, the distance between the sealing spring segment 202 and the nearest electrode 103 is about 5-7 mm, and the proximal end 102 of the sealing spring segment 202 is about 1-2 mm beyond the proximal end 102 of the tube The proximal end 102 of the tube body 100 is flush, and the distal end 101 of the spring thinning section 203 exceeds the distal end 101 of the tube body 100 by at least 15 mm, and the passage 204 in the deformation restraint tube 200 is inserted into the third lining member, In this embodiment, the third inner lining member is a nickel-titanium wire with a diameter of 0.6 mm;

Step 2. Cut the support tube 205 about 25 mm, drip and apply 4011 glue on the seal spring section 202, and insert the support tube 205 into the seal spring section 202 for about 10 mm;

Step 3. Cut the outer sleeve 105, which is preferably a PTFE33# heat shrinkable tube in this embodiment, the length can cover the sparse spring section 203 and the exposed tight spring section 202, and the outer sleeve 105 is sleeved and heat-shrinked to obtain a plastic shape Assembly, the proximal end 102 of the outer sleeve 105 must be shrunk on the support tube 205, and the parameters of the hot air equipment are set to 400° C.

4.4 Electrode 103 Molding

Step 1. Insert the first inner lining member into the fluid delivery pipe 120. In this embodiment, the first inner lining member is preferably a nickel-titanium wire with a diameter of 0.35 mm;

Step 2. Insert the fluid delivery tube 120 into the proximal end 102 of the fluid channel 110 by about 8 mm, drip and apply 4011 glue at the fluid channel 110, and then penetrate the fluid delivery tube 120 into about 2 mm, and dry the 4011 glue;

Step 3. Insert the deformation restraint tube 200 into the main lumen 109 of the tube body 100 from the proximal end 102, and after the support tube 205 and the outer sleeve 105 are passed into the main lumen 109 at the junction, drip and apply 4011 glue on the support tube 205, Continue penetrating the deformation restraint tube 200 into the main lumen 109 until the proximal end 102 of the deformation restraint tube 200 is about 1 mm remaining compared to the proximal end 102 of the main lumen 109 or is flush with the proximal end 102 of the main lumen 109, Dry the surface glue;

Step 4. Insert the second lining member from the distal end 101 of the fluid channel 110. The second lining member is preferably a nickel-titanium wire with a diameter of 0.3 mm in this embodiment. A lining piece is pushed out, and the pipe body 100 is put into the molding machine for molding;

Step 5. After molding, take out the tube body 100, wipe it with 75% alcohol with a clean cloth, and pull out the second lining piece and the third lining piece;

Step 6. Intercept the length of the distal end 101 of the spring thinning section 203, and the interception standard is that the thin spring section 203 is exposed at the distal end 101 side of the tube body 100 for about 3 turns, and peel off the exposed part of the outer sleeve 105.

5. Braided Tube 117 Welding

5.1 Remote 101 Fixation

Step 1. Insert the pull wire assembly into the plastic assembly, place the connecting cap 113 in the deformation restraining tube 200, and apply a small amount of 4011 glue and UV glue between the connecting cap 113 and the deformation restraining tube 200 to fix it;

Step 2: Adjust the position of the fluid channel 110 to ensure that the fluid channel 110 is on the outer edge of the tube body 100, drip and apply a small amount of 4011 glue outside the connecting cap 113, and insert the connecting cap 113 into the main channel of the tube body 100 109, and apply UV glue dropwise to fix the distal end 101 side of the tube body 100, and glue a small amount and several times when applying glue;

Step 3: Pass the escort tube 114 through the distal end 101 of the tube body 100.

5.2 Elastic Conduit 115 Bonding

Step 1. Cut the elastic conduit 115 with a length of about 1200 mm, insert the elastic piece 116, and both ends of the elastic piece 116 are exposed to the elastic conduit 115 and glued with 4011 glue at the joint (the position of the first glue is left on the handle place);

Step 2. Cut the distal end 101 side of the elastic catheter 115 to expose the elastic member 116 by about 12 mm, cut the proximal end 102 side of the elastic catheter 115 to expose the elastic member 116 by about 15 mm, and insert the elastic catheter 115 into the traction wire 106 until the elastic member 116 offsets the deformation restraint tube 200 in the tube body 100;

Step 3. Use UV glue to stick the exposed elastic catheter 115, fluid delivery tube 120, elastic wire 107, and wire 108 firmly (a small amount of glue is applied, and all are covered, so as to avoid too much glue and cannot enter the braided tube 117).

5.3 Braided Tube 117 Welding

Step 1. Intercept the braided tube 117 against the elastic catheter 115, and the length satisfies the elastic catheter 115 to expose the braided tube 117;

Step 2, flaring the non-braided mesh segment of the braided tube 117 with a flaring tool;

Step 3. Straighten the guide wire 108, the fluid delivery tube 120, and the elastic conduit 115, and thread them into the braided tube 117;

Step 4. Insert the heat-shrinkable sleeve 118 and the glass sleeve 119 into the braided tube 117 for heat-shrinking, and set the parameters of the hot air equipment to 270° C.; wherein the heat-shrinkable sleeve 118 adopts a 14# FEP heat-shrinkable tube;

Step 5. Roughly scrape the proximal end 102 of the braided tube 117 with a blade, first use 4011 glue to glue the wire 108, the fluid delivery tube 120, the elastic conduit 115 and the braided tube 117 together, and then use UV glue;

Step 6. Glue the exposed wire 108, the fluid delivery tube 120, and the spring tube together with UV glue, and the bonding position of the glue point should not exceed the elastic conduit 115.

6. Handle Assembly

Step 1. Cut a heat shrinkable tube of about 70 mm, heat shrink it on the proximal end 102 of the braided tube 117 and wrap the UV glue point;

Step 2. Intercept 2 sections of 20-25 mm RE heat-shrinkable tubes, 1 section of 100 mm-long fluid rear-end delivery tube 121, and 1 section of 80 mm-long fluid rear-end delivery tube 121, and coaxially heat-shrink the 2 sections of blue heat-shrinkable tubes on the proximal end 102 of the 100 mm long fluid rear end delivery pipe 121, the hot air equipment parameter is set to 120° C.;

Step 3. Insert the 2-stage fluid back end delivery pipe 121 into the handle end cap, namely the end cap 317 above, and fix it with UV glue (there is a 100 mm long fluid back end delivery pipe 121 in the middle of the handle end cap. line hole 3172);

Step 4. Insert the locking cap and handle shell of the distal end 101 of the handle on the braided tube 117;

Step 5. Install the cock 305 into the mounting hole 303, peel off the exposed traction outer sleeve 1061, and pass the traction wire 106 into the first channel 3081, the docking channel 309, the second channel 3082, the fluid delivery tube 120 and the wires in sequence. 108 penetrates the avoidance channel 304, rotates the drive groove 3051 on the cock 305 with a tool, realizes the dislocation of the traction installation channel 308 and the docking channel 309, and realizes the installation of the traction wire 106; during the adjustment process, the cock 305 acts on the traction wire 106 Therefore, this step is also the adjustment of the position of the connecting piece 302, that is, use the tool to rotate the cock 305 to adjust the position of the connecting piece 302 (the connecting piece 302 cannot be located at the limit position of its own motion stroke before adjustment) and the bending stroke, after the adjustment, cut the proximal end 102 side of the traction wire 106 and fix the cock 305 with 4011 glue;

Step 6. Continue to install other handle components such as the holding sheath 307, and fix the handle end cap with 4011 glue;

Step 7. Seal the proximal end 102 of the fluid delivery tube 120 with UV glue, pull out the first liner, cut the fluid delivery tube 120 and install the luer connector;

Step 8. Connect the luer connector to the wire and fix it with the fluid rear end delivery tube 121 heat-shrinked with the RE heat-shrinkable tube.

Method and Apparatus for Controlling Perfusion of a Plurality of Channels of Syringe Pump, Syringe Pump and Storage Medium

In order to make objectives, technical solutions, and advantages of embodiments of the present invention clearer, the technical solutions in the embodiments of the present invention will be further described in detail below with reference to accompanying drawings in the embodiments of the present invention. Obviously, the described embodiments are only some, but not all of the embodiments of the present invention. Based on the embodiments of the present invention, all other embodiments obtained by those ordinarily skilled in the art without any creative labor shall fall within a protective scope of the present invention.

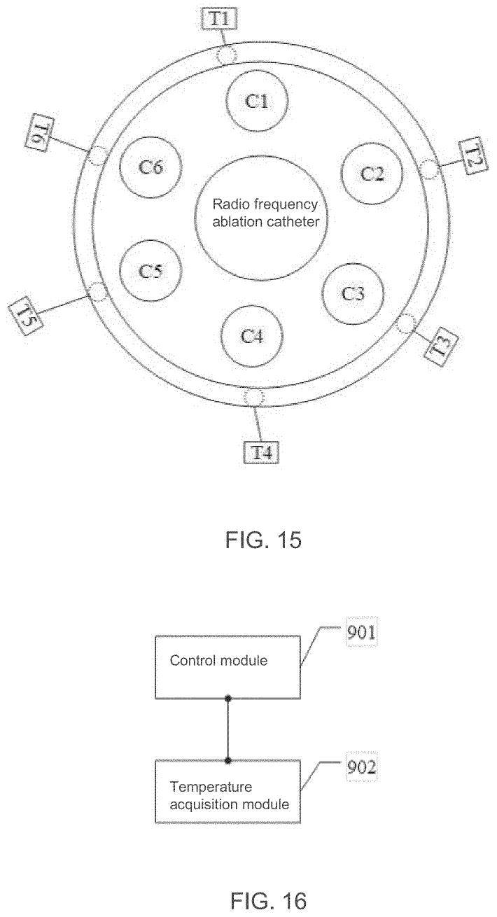

Reference is made to FIG. 15, which is a schematic diagram showing an application scenario of a method for controlling perfusion of a plurality of channels of a syringe pump according to an embodiment of the present invention. The method for controlling the perfusion of the plurality of channels of the syringe pump may be implemented by a radio frequency ablation control apparatus 10 or a syringe pump 20 in FIG. 15. Optionally, the method for controlling the perfusion of the plurality of channels of the syringe pump may be implemented by another computer device other than the radio frequency ablation control apparatus 10 or the syringe pump 20, such as a server, a desktop computer, a notebook computer, a laptop computer, a tablet computer, a personal computer and a smart phone.

As shown in FIG. 15, the radio frequency ablation system includes a radio frequency ablation control apparatus 10, a syringe pump 20 with a plurality of perfusion channels, a plurality of temperature acquisition apparatuses 30, a radio frequency ablation catheter 40 and a neutral electrode 50. The plurality of temperature acquisition apparatuses 30 may be arranged at a top end of the radio frequency ablation catheter 40, or may be arranged at a top end of an extension tube 232 of the syringe pump. The plurality of temperature acquisition apparatuses 30 are respectively configured to acquire temperatures of a plurality of different positions of the ablation site.

Particularly, by way of the radio frequency ablation control apparatus 10 as an execution body of the method for controlling the perfusion of the plurality of channels of the syringe pump according to the embodiment of the present invention, firstly, the top end of the radio frequency ablation catheter 40 for generating and outputting radio frequency energy and the top end of the extension tube 232 of the syringe pump 20 are inserted into the ablation object and reach the ablation site. Then, the neutral electrode 50 is brought into contact with the skin surface of the ablation object. The radio frequency current flows through the radio frequency ablation catheter 40, the tissue of the ablation object and the neutral electrode 50, thereby forming a loop. When the ablation task is triggered, the radio frequency ablation control apparatus 10 controls the radio frequency ablation catheter 40 to output radio frequency energy to the ablation site in a discharge fashion, so as to perform an ablation operation on the ablation site.

Meanwhile, the radio frequency ablation control apparatus 10 controls the syringe pump 20 to open at least one perfusion channel to perform the perfusion operation through the opened perfusion channel at a preset initial flow rate, so as to perfuse the ablation site with saline. Then, temperatures of a plurality of locations of the ablation site are acquired in real time by means of the plurality of temperature acquisition apparatuses 30, and the syringe pump 20 is controlled to open or close some or all of the perfusion channels and/or the syringe pump 20 is controlled to adjust flow rates of some or all of the perfusion channels according to real-time changes in the temperatures of the plurality of locations.

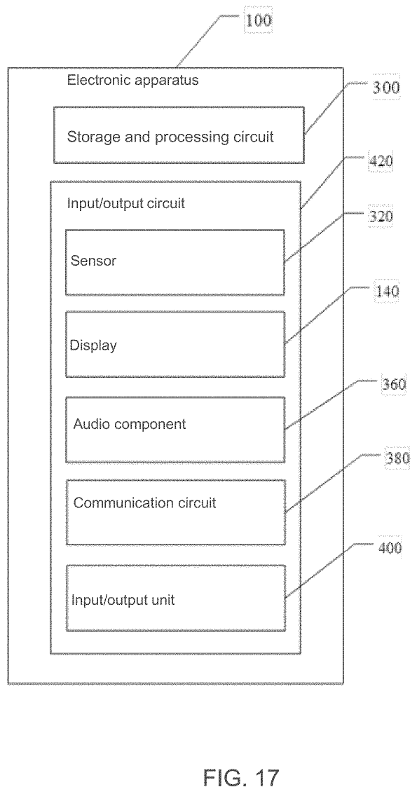

Reference is made FIG. 16 and FIG. 17, wherein FIG. 16 is a schematic diagram showing an internal structure of a syringe pump according to an embodiment of the application, and FIG. 17 is a schematic diagram showing an internal structure of an injection structure 23 in FIG. 16. For ease of understanding, FIG. 16 and FIG. 17 only show some of structures related to the embodiment. In practical applications, more or less structures than those shown in FIG. 16 and FIG. 17 exist. As shown in FIG. 16 and FIG. 17, the syringe pump 20 includes a controller 21, a plurality of temperature acquisition apparatuses 22 and a plurality of injection structures 23.

Each of the injection structures 23 includes a syringe 231, an extension tube 232, a push rod 233 and a drive apparatus 234. Each injection structure 23 forms a perfusion channel.

One end of each extension tube 232 is connected with a syringe and the other end thereof is provided with at least one temperature acquisition apparatus 22 (for ease of understanding, only one is shown in the figure). One end of the push rod 233 abuts against the syringe 231, and the push rod 233 is further connected with a drive apparatus 234 (such as a step motor).

Optionally, one end, which is provided with the temperature acquisition apparatus 22, of each extension tube 232 may be fixed around a top end 41 of the radio frequency ablation catheter 40 through a fixing structure. The fixing structure is for example a catheter. A plurality of through holes and a plurality of perforation holes penetrating a head end and a tail end of the catheter are provided in a sidewall of the catheter. Each extension tube 232 and the top end 41 of the radio frequency ablation catheter 40 respectively pass through the plurality of perforation holes. The temperature acquisition apparatus 22 disposed at one end of each extension tube 232 respectively penetrates out of the through hole in the sidewall of the catheter closest to itself after entering the perforation hole along with each extension tube 232. The plurality of temperature acquisition apparatuses 22 together form a claw-shaped structure for acquiring temperatures of different locations of the ablation site of the ablation object.

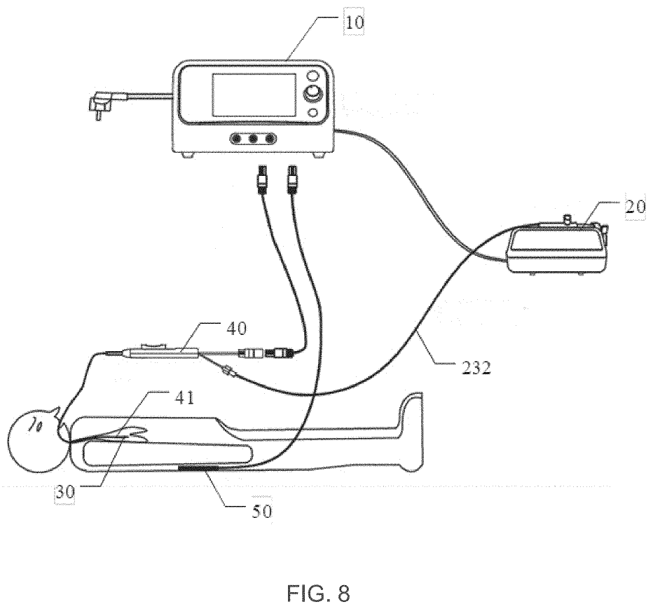

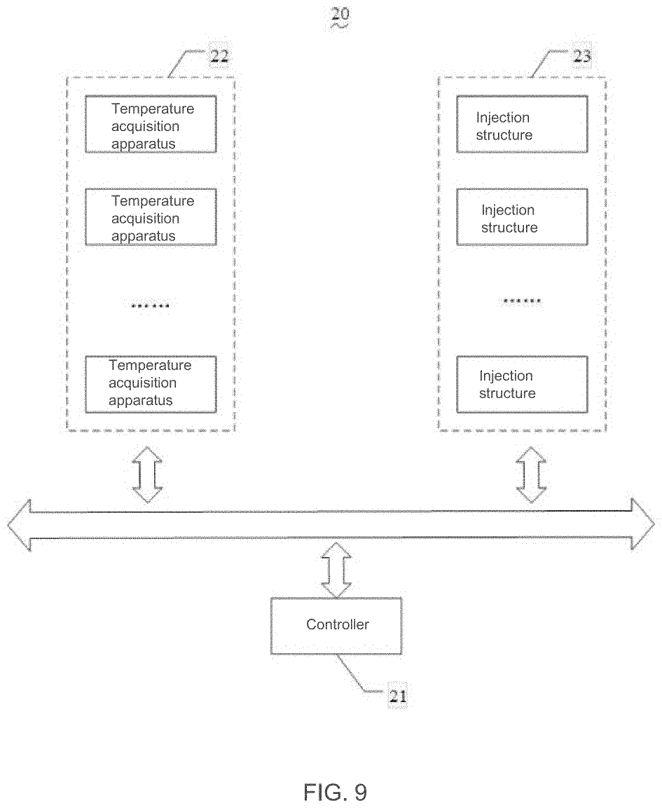

By way of an example of the 6 injection structures, as shown in FIG. 8, a dashed circle in the figure is the through hole in the sidewall of the catheter. The 6 injection structures surround the radio frequency ablation catheter to form 6 perfusion channels C1 to C6, and the 6 perfusion channels C1 to C6 correspond to 6 temperature acquisition apparatuses T1 to T6 respectively. The radio frequency ablation catheter may be a unipolar radio frequency ablation catheter or a multipolar radio frequency ablation catheter, which is not particularly limited in the present invention. When the radio frequency ablation catheter is the multipolar radiofrequency ablation catheter, at least one perfusion channel may be provided around each pole of the multipolar radiofrequency ablation catheter, or multiple perfusion channels may be shared by a plurality of poles.

The controller 21 opens or closes the corresponding perfusion channel by controlling whether the drive apparatus 234 drives the push rod 233 to move in a designated direction. For example, when the push rod 233 pushes a tail portion of a syringe 231 forwards, the perfusion channel is opened and the liquid in the syringe 231 may flow into the ablation object along the perfusion channel. When the push rod 233 stops pushing the tail portion of the syringe 231 forwards, the perfusion channel is closed and the liquid in the syringe 231 may not flow into the ablation object along the perfusion channel any more.

In addition, the controller 21 controls a movement speed of the push rod by using the drive apparatus 234 so as to control the flow rate of the liquid in the perfusion channel.

Optionally, a valve may be disposed on the extension pipe 232 of each injection structure, and the controller 21 opens or closes the corresponding perfusion channel by controlling on/off of the valve.

The controller 21 is electrically coupled with the plurality of temperature acquisition apparatuses 22 through a data line or a wireless network, and electrically connected with the plurality of injection structures 23, for performing steps of the method for controlling the perfusion of the plurality of channels of the syringe pump according to the embodiments shown in FIG. 11 to FIG. 14 below, for example,

when an ablation task is triggered, the syringe pump is controlled to open at least one perfusion channel to perform a perfusion operation through the opened perfusion channel at a preset initial flow rate;

temperatures of a plurality of sites of an ablation object are acquired in real time by a plurality of temperature acquisition apparatuses; and

the syringe pump is controlled to open or close some or all of the perfusion channels and/or the syringe pump is controlled to adjust flow rates of some or all of the perfusion channels according to real-time changes in the temperatures of the plurality of sites.

For a specific process for the controller 21 to implement its functions, reference may be made to relevant descriptions in the embodiments shown in FIG. 11 to FIG. 14 below, which will be omitted here.

It should be understood that the syringe pump 20 may further include other common structures such as a display screen and a power supply, which are not particularly limited in the present invention.

In the embodiments of the present invention, when the ablation task is triggered, the syringe pump is controlled to open and pass through the at least one perfusion channel so as to perform the perfusion operation at the preset initial flow rate. Then, the syringe pump is controlled to open or close some or all of the perfusion channels and/or the flow rates of some or all of the perfusion channels are adjusted according to the temperatures, which are acquired by the plurality of temperature acquisition apparatuses in real time, of the plurality of sites of the ablation object. Accordingly, the perfusion of the plurality of channels of the syringe pump is intelligently adjusted dynamically based on the real-time changes in the temperatures of the plurality of sites of the ablation object in the process of performing the ablation task. Since the perfusion volume of the syringe pump is adjusted relatively purposefully and directionally as the temperatures of the different sites of the ablation object change, operation delays and errors caused by manual determination can be reduced. Meanwhile, the timeliness, the accuracy and the pertinence of perfusing the liquid in the process of performing the ablation task can be improved. Accordingly, the injury of the ablation operation to the ablation object is reduced, and the safety of the radio frequency ablation operation is improved.

Reference is made to FIG. 11, which is a flow chart of an implementation of a method for controlling perfusion of a plurality of channels of a syringe pump according to an embodiment of the present invention. The method is used to control the syringe pump with a plurality of perfusion channels, such as the syringe pump 20 shown in FIG. 16 and FIG. 17. The method may be implemented particularly by the syringe pump 20 in FIG. 15, or may be implemented by the radio frequency ablation control apparatus 10 in FIG. 15, or may be implemented by another computer device electrically coupled to the syringe pump. As shown in FIG. 11, the method particularly includes the following steps.

In step S401, when the ablation task is triggered, the syringe pump is controlled to open at least one perfusion channel to perfuse the ablation object with a liquid through the opened perfusion channel at a preset initial flow rate.

Particularly, the ablation task may be triggered when for example a preset trigger time is reached, a trigger instruction sent by another computer device is received, or a notification event that a user performs an operation for triggering the ablation task is detected. The operation for triggering the ablation task is for example to press a physical or virtual button for triggering the ablation task.

Optionally, after each start of the syringe pump, a perfusion parameter is set to a preset initial value. The perfusion parameter may include but is not limited to an initial flow rate, total perfusion volume, perfusion time, the like.

When the ablation task is triggered, the radio frequency ablation catheter starts to perform an ablation operation on the ablation object to output radio frequency energy to the ablation object. Meanwhile, the syringe pump opens at least one perfusion channel pointed by the perfusion control instruction in response to the triggered perfusion control instruction, and performs the perfusion operation through the opened perfusion channel at the preset initial flow rate to perfuse the ablation object with a liquid so as to adjust the temperature of the ablation object, thereby avoiding burning external tissues of the ablation object due to too high temperature or preventing a situation that no ablation effect is achieved due to too low temperature.

The perfusion control command may be automatically triggered by the syringe pump when a preset event is detected, or may be sent to the syringe pump by the ablation control apparatus or another computer device electrically coupled to the syringe pump. The preset event includes that the user presses a preset physical or virtual button for triggering the perfusion control instruction or includes an event for triggering the ablation task.

In step S402, temperatures of a plurality of sites of the ablation object are acquired in real time by a plurality of temperature acquisition apparatuses.

Particularly, the temperatures of the plurality of different sites of the ablation object are acquired in real time by the plurality of temperature acquisition apparatuses so as to be used as reference data for dynamically adjusting the perfusion volume of the syringe pump.

Optionally, the temperatures of the plurality of sites of the ablation object may be acquired in real time in the following fashions:

acquiring temperature sample values of the plurality of sites of the ablation object in real time by the plurality of temperature acquisition apparatuses and filtering the acquired temperature sample values;

determining whether the filtered temperature sample values exceed a preset warning value range;

if the filtered temperature sample values exceed the preset warning value range, outputting alarm information to remind the user that the operation is abnormal and whether the user needs to stop the ablation operation; and

If the filtered temperature sample values do not exceed the preset warning value range, using the minimum value or average value of the filtered temperature sample values within a preset period (for example, within 10 seconds) as a temperature for controlling the perfusion of the syringe pump.

In step S403, the syringe pump is controlled to open or close some or all of the perfusion channels and/or the syringe pump is controlled to adjust flow rates of some or all of the perfusion channels according to real-time changes in the temperatures of the plurality of sites.

Particularly, the plurality of temperature acquisition apparatuses are configured in the radio frequency ablation system to acquire the temperatures of the plurality of different sites of the ablation object, respectively. The plurality of perfusion channels of the syringe pump are respectively configured to perfuse the plurality of different sites of the ablation object with a liquid. After the perfusion channel is opened, the liquid automatically flows to the corresponding site via the opened perfusion channel. One perfusion channel corresponds to at least one temperature acquisition apparatus.

According to the real-time acquired temperatures of the plurality of sites, real-time changes in the temperatures of the plurality of sites are analyzed. When a real-time change trend and a change amplitude of the plurality of temperatures meet a preset adjustment condition, the syringe pump is controlled to open or close some or all of the perfusion channels, and/or the syringe pump is controlled to adjust flow rates of some or all of the perfusion channels.

The preset adjustment condition means that for example the acquired temperature is greater than a preset maximum temperature, the acquired temperature is lower than a preset minimum temperature, and the like.

The syringe pump is controlled to open or close some or all of the perfusion channels and/or the syringe pump is controlled to adjust the flow rates of some or all of the perfusion channels, that is, the syringe pump is controlled to perform at least one of the following operations:

controlling the syringe pump to open some of the perfusion channels; controlling the syringe pump to close some of the perfusion channels; controlling the syringe pump to open all of the perfusion channels; controlling the syringe pump to close all of the perfusion channels; controlling the syringe pump to adjust the flow rates of some of the perfusion channels; and controlling the syringe pump to adjust flow rates of all of the perfusion channels.

The flow rate of the perfusion channel is the flow rate of the liquid in the perfusion channel. By controlling the flow rate of the liquid in the perfusion channel, the perfusion volume of the perfusion channel may be controlled, and further the temperature of the ablation object may be regulated.

Optionally, after opening the perfusion channel, the syringe pump may automatically perform the perfusion operation directly through the opened perfusion channel at the same time.

In the embodiments of the present invention, when the ablation task is triggered, the syringe pump is controlled to open and pass through the at least one perfusion channel so as to perform the perfusion operation at the preset initial flow rate. Then, the syringe pump is controlled to open or close some or all of the perfusion channels and/or the flow rates of some or all of the perfusion channels are adjusted according to the temperatures, which are acquired by the plurality of temperature acquisition apparatuses in real time, of the plurality of sites of the ablation object. Accordingly, the perfusion of the plurality of channels of the syringe pump is intelligently adjusted dynamically based on the real-time changes in the temperatures of the plurality of sites of the ablation object in the process of performing the ablation task. Since the perfusion volume of the syringe pump is adjusted relatively purposefully and directionally as the temperatures of the different sites of the ablation object change, operation delays and errors caused by manual determination can be reduced. Meanwhile, the timeliness, the accuracy and the pertinence of perfusing the liquid in the process of performing the ablation task can be improved. Accordingly, the injury of the ablation operation to the ablation object is reduced, and the safety of the radio frequency ablation operation is improved.

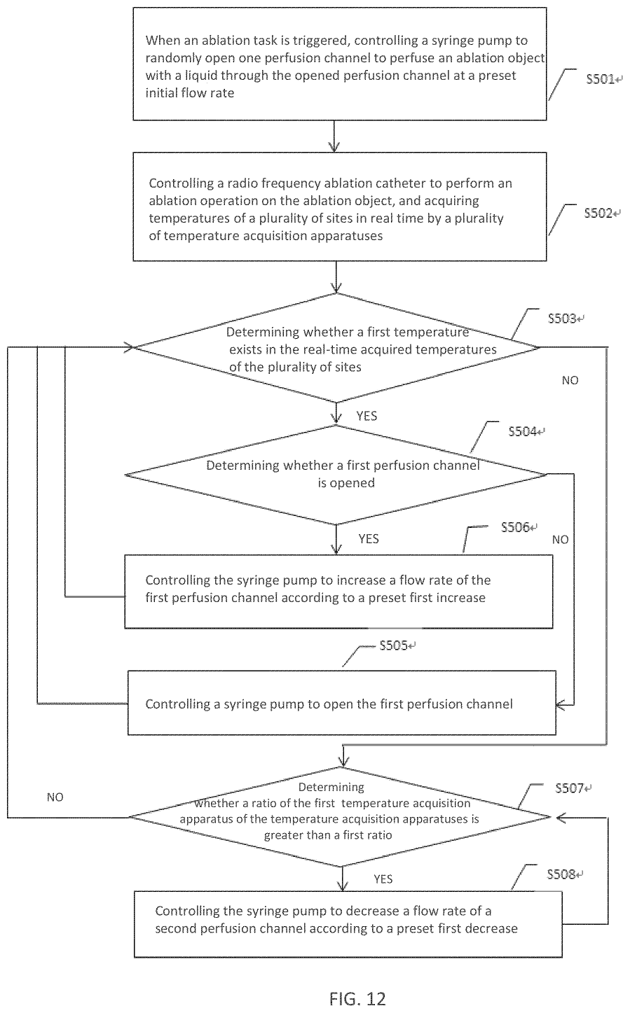

Reference is made to FIG. 12, which is a flow chart of an implementation of a method for controlling perfusion of a plurality of channels of a syringe pump according to another embodiment of the present invention. The method is used to control the syringe pump with a plurality of perfusion channels, such as the syringe pump 20 shown in FIG. 16 and FIG. 17. Particularly, the method may be implemented by the syringe pump 20 in FIG. 15, or may be implemented by the radio frequency ablation control apparatus 10 in FIG. 15, or may be implemented by another computer device electrically coupled to the syringe pump. For ease of description, the computer device is hereinafter collectively referred to as the control apparatus. As shown in FIG. 12, the method particularly includes the following steps.

In step S501, when an ablation task is triggered, the syringe pump is controlled to randomly open one perfusion channel to perfuse the ablation object with a liquid through the opened perfusion channel at a preset initial flow rate.

In step S502, a radio frequency ablation catheter is controlled to perform an ablation operation on the ablation object, and temperatures of a plurality of sites of the ablation object are acquired in real time by a plurality of temperature acquisition apparatuses.

When the ablation task is triggered, the control apparatus firstly controls the syringe pump to randomly open one perfusion channel, so as to perfuse the ablation object with the liquid through the opened perfusion channel at the initial flow rate. Then, the radio frequency ablation catheter is controlled to perform an ablation operation on the ablation object, and meanwhile, the temperatures of the plurality of the ablation object are acquired in real time by the plurality of temperature acquisition apparatuses.

In this way, before the radio frequency ablation catheter is controlled to perform the ablation operation, the syringe pump is firstly controlled to randomly open one perfusion channel so as to perfuse the ablation object with a small amount of liquid through the perfusion channel, which may prevent from affecting other subsequent regulation operations performed by utilizing impedance values due to overhigh initial impedances of special individual ablation objects. Moreover, perfusing the small amount of liquid will not cause other adverse effects on the ablation object.

For unfinished details of the step S501 and the step S502 in this embodiment, reference may be made to related descriptions of the step S401 and the step S402 in the embodiment shown in FIG. 11.

In step S503, it is determined whether a first temperature exists in the real-time acquired temperatures of the plurality of sites.

The first temperature is greater than a preset maximum temperature. Optionally, the preset maximum temperature may be preset in an execution body of the method for controlling the perfusion of the plurality of channels of the syringe pump according to the present invention on the basis of a user-defined operation.

If the first temperature exists in the real-time acquired temperatures of the plurality of sites, the method performs a step S504 of determining whether the first perfusion channel is opened.

Particularly, if the first temperature exists in the real-time acquired temperatures of the plurality of sites, indicating the temperatures of some sites of the ablation object exceed a limit, a risk of damage exists, and the perfusion volume needs to be increased to cool these sites, then it is determined that whether the first perfusion channel is opened. The first perfusion channel is configured to perfuse a first site with the liquid, and the first temperature is a temperature of the first site.

By way of an example, it is assumed that four temperature acquisition apparatuses T1 to T4 are respectively configured to acquire temperatures of four sites B1 to B4 of the ablation object, and respectively are in one-to-one correspondence to four perfusion channels C1 to C4 of the syringe pump. A preset maximum temperature is 90° C. (degrees Celsius). If the temperatures acquired by the four temperature acquisition apparatuses T1 to T4 are respectively 89.7° C., 91° C., 89.9° C. and 90.5° C., by this time, it may be known from the correspondence of the temperature acquisition apparatus, the ablation sites, the perfusion channels and the real-time acquired temperatures {(T1, B1, C1, 89.7° C.), (T2, B2, C2, 91° C.), (T3, B3, C3, 89.9° C.) and (T4, B4, C4, 90.5° C.)} and the preset maximum temperature of 90° C. that the first temperatures exceeding the limit are 91° C. and 90.5° C., the first sites that need to be cooled are B2 and B4, and the first perfusion channels that need to be regulated are C2 and C4.

Since when the ablation task is triggered, the control apparatus controls the syringe pump to randomly open one perfusion channel, it is necessary to determine whether the first perfusion channels C2 and C4 have been opened.

Identification information (such as serial numbers) of the plurality of perfusion channels of the syringe pump is stored in the control apparatus. Moreover, each time the control apparatus controls the syringe pump to open or close the perfusion channel, it generates a corresponding log in which the identification information, the flow rate, and the opening or closing time of the perfusion channel controlled to be opened or closed at this time are at least recorded. According to the log, the currently opened perfusion channel may be determined, such that it may be determined whether the first perfusion channels C2 and C4 have been opened.

If the first perfusion channel is not opened, the method performs a step S505 of controlling the syringe pump to open the first perfusion channel, and returns to perform the step S503.

If the first perfusion channel has been opened, the method performs a step S506 of controlling the syringe pump to increase the flow rate of the first perfusion channel according to a first preset increase, and returns to perform the step S503, until the flow rate of the first perfusion channel reaches a preset maximum flow rate.

Particularly, in one aspect, if the first perfusion channel is not opened, the method performs the step of controlling the syringe pump to open the first perfusion channel to perform the perfusion operation through all the opened first perfusion channels; and returns to perform the step of determining whether the first temperature exists in the real-time acquired temperatures of the plurality of sites. In another aspect, the method performs the step of if the first perfusion channel has been opened, controlling the syringe pump to increase the flow rate of the first perfusion channel according to the first preset increase, and returns to perform the step of determining whether the first temperature exists in the real-time acquired temperatures of the plurality of sites, and so forth, until the flow rates of all the first perfusion channels reach a preset maximum flow rate.

Following the example above, if the C2 in the first perfusion channels C2 and C4 has been opened but the C4 has not been opened, the syringe pump is controlled to open the C4 and the liquid is injected into a site B4 through the C4 at the initial flow rate. Meanwhile, the syringe pump is controlled to increase the flow rate of the C2 according to the first preset increase to increase the perfusion volume to a site B2, so as to achieve the purpose of rapidly cooling the sites B2 and B4.

If the first temperature does not exist in the real-time acquired temperatures of the plurality of sites, the method performs a step S507 of determining whether the ratio of the first temperature acquisition apparatus in the temperature acquisition apparatuses is greater than the first ratio.

Particularly, if the first temperature greater than a preset maximum temperature does not exist in the real-time acquired temperatures of the plurality of sites, indicating that the temperature of each site of the ablation object is within a safe range value, and the current ablation operation will cause no hurt to the ablation object, then it is determined whether the ratio of the first temperature acquisition apparatus in the temperature acquisition apparatuses is greater than the first ratio, so as to ensure that the ablation operation may achieve a desired ablation effect.

A temperature acquired by the first temperature acquisition apparatus lasts for a preset duration less than a preset minimum temperature, and the preset minimum temperature is the minimum temperature limit achieving the desired ablation effect. Optionally, the preset minimum temperature, the preset duration and the first ratio may be preset in an execution body of the method for controlling the perfusion of the plurality of channels of the syringe pump according to the present invention on the basis of a user-defined operation.

If the ratio of the first temperature acquisition apparatus in the temperature acquisition apparatuses is greater than the first ratio, the method performs a step S508 of controlling the syringe pump to reduce the flow rate of the second perfusion channel according to a first preset decrease, and returns to perform the step S507, until the flow rate of the second perfusion channel reaches a preset minimum flow rate.

If the ratio of the first temperature acquisition apparatus in the temperature acquisition apparatuses is not greater than the first ratio, the method returns to perform the step S503.

Particularly, in one aspect, if the ratio of the first temperature acquisition apparatus in the temperature acquisition apparatuses is greater than the first ratio, indicating that the overall temperature of the ablation object is low, the increase is slow, the current perfusion volume of the liquid is too large, and the desired ablation effect may not be achieved, then the method performs the step of controlling the syringe pump to reduce the flow rate of the second perfusion channel according to a first preset decrease; and returns to perform the step of determining whether the ratio of the first temperature acquisition apparatus in the temperature acquisition apparatuses is greater than the first ratio, and so forth, until the flow rate of the second perfusion channel reaches a preset minimum flow rate. The second perfusion channel is configured to perfuse the second site with the liquid, and the first temperature acquisition apparatus is configured to detect the temperature of the second site.

In another aspect, if the ratio of the first temperature acquisition apparatus in the temperature acquisition apparatuses is not greater than the first ratio, indicating that a rate of temperature rise of the ablation object is positive, and the current perfusion volume helps the temperature rise of the ablation object, then the method returns to perform the step of determining whether the first temperature exists in the real-time acquired temperatures of the plurality of sites.

In this way, according to the real-time change in the temperatures, the perfusion volume is gradually increased or decreased, which may improve the accuracy of controlling the perfusion, reduce the operation risk, and achieve a better ablation effect.

Following the example above, it is assumed that a preset minimum temperature is 65° C. (degrees Celsius), the first ratio is 49%, and the preset duration is 10 seconds. If the temperatures acquired by the four temperature acquisition apparatuses T1 to T4 are 64.2° C. (for 11 seconds)), 64.3° C. (for 9 seconds), 65.1° C. (for 6 seconds) and 64.2° C. (for 12 seconds), by this time, it may be known from the correspondence of the temperature acquisition apparatuses, the ablation sites, the perfusion channels and the real-time acquired temperatures {(T1, B1, C1, 64.2° C., 11 s), (T2, B2, C2, 64.3° C., 9s), (T3, B3, C3, 65.1° C., 6 s) and (T4, B4, C4, 64.2° C., 125)} and the preset minimum temperature of 65° C. that the first temperature acquisition apparatuses are T1 and T4, and the ratio of the first temperature acquisition apparatus in the temperature acquisition apparatuses is 2/4=50% (greater than the first ratio of 49%), and the second perfusion channels that need to be regulated are C1 and C4. Hence, the syringe pump is controlled to reduce the flow rates of the C1 and the C4 according to the first preset decrease so as to reduce the perfusion volume to the sites B1 and B4, so as to achieve the effect of increasing the temperature of the sites B1 and B4.

Optionally, in another embodiment of the present invention, respective preset maximum temperatures and respective preset minimum temperatures are set for the temperature acquisition apparatuses, and the first perfusion channel and the second perfusion channel are based on the preset maximum temperatures and the preset minimum temperatures corresponding to the temperature acquisition apparatuses. Then, according to the first perfusion channel and the second perfusion channel determined based on the preset maximum temperature corresponding to the temperature acquisition apparatuses, the method performs the steps S503 to S508.

Particularly, it is determined whether the first temperature exists in the real-time acquired temperatures of the plurality of sites, wherein the first temperature is greater than a preset maximum temperature corresponding to the temperature acquisition apparatus acquiring the first temperature.

In one aspect, if the first temperature exists in the real-time acquired temperatures of the plurality of sites, the method performs the step of determining whether the first perfusion channel is opened, wherein the first perfusion channel is configured to perfuse the first site with the liquid, and the first temperature is the temperature of the first site. If the first perfusion channel is not opened, the method performs the step of controlling the syringe pump to open the first perfusion channel. Meanwhile, the method returns to perform the step of determining whether the first temperature exists in the real-time acquired temperatures of the plurality of sites, wherein the first temperature is greater than the preset maximum temperature corresponding to the temperature acquisition apparatus acquiring the first temperature. If the first perfusion channel has been opened, the method performs the step of controlling the syringe pump to increase the flow rate of the first perfusion channel according to the first preset increase. Meanwhile, the method returns to perform the step of determining whether the first temperature exists in the real-time acquired temperatures of the plurality of sites, wherein the first temperature is greater than the preset maximum temperature corresponding to the temperature acquisition apparatus acquiring the first temperature, until the flow rate of the first perfusion channel reaches the preset maximum flow rate.

In another aspect, if the first temperature does not exist in the lure real-time acquired temperatures of the plurality of sites, the method performs the step of determining whether the ratio of the first temperature acquisition apparatus of the temperature acquisition apparatuses is greater than the first ratio, wherein the first temperature acquisition apparatus acquires the temperature for a preset duration less than the preset minimum temperature corresponding to the first temperature acquisition apparatus. If the ratio of the first temperature acquisition apparatus is greater than the first ratio, the method performs the step of controlling the syringe pump to decrease the flow rate of the second perfusion channel according to the first preset decrease. Meanwhile, the method returns to perform the step of determining whether the ratio of the first temperature acquisition apparatus of the temperature acquisition apparatuses is greater than the first ratio, until the flow rate of the second perfusion channel reaches the preset minimum flow rate, wherein the second perfusion channel is configured to perfuse a second site with the liquid, and the first temperature acquisition apparatus is configured to detect the temperature of the second site. If the ratio of the first temperature acquisition apparatus is not greater than the first ratio, then the method returns to perform the step of determining whether the first temperature exists in the real-time acquired temperatures of the plurality of sites, wherein the first temperature is greater than the preset maximum temperature corresponding to the temperature acquisition apparatus acquiring the first temperature.

Since the different sites of the ablation object may have a certain difference in surface shape, internal tissue structure, and tissue thickness, the temperature changes caused by the influence of radio frequency energy vary, and temperature limits required for qualitative change are different. In this way, corresponding maximum and minimum limits are preset for the plurality of temperature acquisition apparatuses configured to acquire the temperatures of the different sites of the ablation object, which may make the perfusion control more targeted. Accordingly, the accuracy of the perfusion operation may be improved, and further the ablation effect is improved.

In the embodiments of the present invention, when the ablation task is triggered, the syringe pump is controlled to open and pass through the at least one perfusion channel so as to perform the perfusion operation at the preset initial flow rate. Then, the syringe pump is controlled to open or close some or all of the perfusion channels and/or the flow rates of some or all of the perfusion channels are adjusted according to the temperatures, which are acquired by the plurality of temperature acquisition apparatuses in real time, of the plurality of sites of the ablation object. Accordingly, the perfusion of the plurality of channels of the syringe pump is intelligently adjusted dynamically based on the real-time changes in the temperatures of the plurality of sites of the ablation object in the process of performing the ablation task. Since the perfusion volume of the syringe pump is adjusted relatively purposefully and directionally as the temperatures of the different sites of the ablation object change, operation delays and errors caused by manual determination can be reduced. Meanwhile, the timeliness, the accuracy and the pertinence of perfusing the liquid in the process of performing the ablation task can be improved. Accordingly, the injury of the ablation operation to the ablation object is reduced, and the safety of the radio frequency ablation operation is improved.

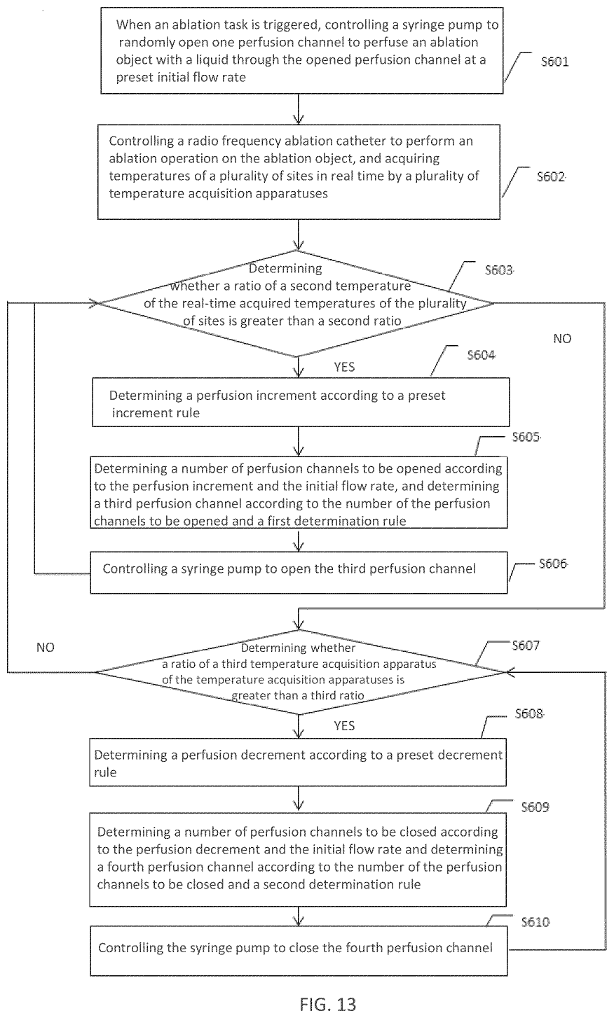

Reference is made to FIG. 13, which is a flow chart of an implementation of a method for controlling perfusion of a plurality of channels of a syringe pump according to another embodiment of the present invention. The method is used to control the syringe pump with a plurality of perfusion channels, such as the syringe pump 20 shown in FIG. 16 and FIG. 17. The method may be implemented by the syringe pump 20 in FIG. 15, or may be implemented by the radio frequency ablation control apparatus 10 in FIG. 15, or may be implemented by another computer device electrically coupled to the syringe pump. For ease of description, the computer device is hereinafter collectively referred to as a control apparatus. As shown in FIG. 13, the method particularly includes the following steps.

In step S601, when an ablation task is triggered, the syringe pump is controlled to randomly open one perfusion channel to perfuse the ablation object with a liquid through the opened perfusion channel at a preset initial flow rate.

In step S602, a radio frequency ablation catheter is controlled to perform an ablation operation on the ablation object, and temperatures of a plurality of sites of the ablation object are acquired in real time by a plurality of temperature acquisition apparatuses.

The step S601 and the step S602 are the same as the step S501 and the step S502 in the embodiment shown in FIG. 12. Particularly, reference may be made to related descriptions in the embodiment shown in FIG. 12, which will be omitted here.

In step S603, it is determined whether a ratio of the second temperature in the real-time acquired temperatures of the plurality of sites is greater than a second ratio.

The second temperature is greater than a preset maximum temperature. The second ratio may be preset in an execution body of the method for controlling the perfusion of the plurality of channels of the syringe pump according to the present invention on the basis of a user-defined operation.

If the ratio of the second temperature is greater than the second ratio, the method performs a step S604 of determining a perfusion increment according to a preset increment rule.

Particularly, if the ratio of the second temperature greater than the preset maximum temperature of the real-time acquired temperatures of the plurality of sites is greater than the second ratio, indicating that the overall temperature of the ablation object is relatively high, a risk of hurting these sites exists, and the perfusion volume needs to be increased to cool these sites, then the perfusion increment (that is, the perfusion volume that needs to be increased) is determined according to the preset increment rule.

The preset increment rule is to determine the perfusion increment according to the difference between the maximum temperature of the temperatures of the plurality of sites and the preset maximum temperature. The difference between the maximum temperature of the temperatures of the plurality of sites and the preset maximum temperature is in direct proportional to the perfusion increment, that is, the greater the difference is, the greater the perfusion increment required is.

Optionally, the perfusion increment and the perfusion decrement below may further be fixed values preset in the control apparatus according to the user-defined operation.

Particularly, the correspondence between a plurality of difference intervals and the preset perfusion increment may be preset in the control apparatus. Firstly, it is determined that which difference interval the maximum temperature of the temperatures of the plurality of sites falls into, and then the perfusion increment corresponding to the difference interval that the maximum temperature of the temperatures of the plurality of sites falls into is determined as the required perfusion increment according to the correspondence of the difference interval that the maximum temperature of the temperatures of the plurality of sites falls into and the preset correspondence.

In step S605, the number of perfusion channels to be opened is determined according to the perfusion increment and the initial flow rate, and the third perfusion channel is determined according to the number of the perfusion channels to be opened and a first determination rule.

It should be understood that the initial flow rate of each perfusion channel is the same. Every time the syringe pump controls the opening of the perfusion channel, it will perform the perfusion operation through the opened perfusion channel at the initial flow rate.

Particularly, the first determination rule is to determine the third perfusion channel successively according to a distance from the second temperature acquisition apparatus and the number of the perfusion channels to be opened, wherein a temperature acquired by the second temperature acquisition apparatus is maximum. Further, if a plurality of perfusion channels with the same distance from the second temperature acquisition apparatus exist, the required third perfusion channel is randomly determined from them.

According to the perfusion increment and the initial flow rate, a calculation formula for determining the number of the perfusion channels to be opened is for example described as follows: the value obtained by dividing the perfusion increment by the initial flow rate is rounded and then added to one.

For example, with reference to FIG. 15, it is assumed that 6 temperature acquisition apparatuses T1 to T6 correspond to 6 sites B1 to B6 of the ablation object, and the 6 perfusion channels C1 to C6 of the syringe pump are configured to perfuse the B1 to the B6 with the liquid, respectively. If the preset maximum temperature is 90° C., the second ratio is 50%, and the temperatures of the sites B1 to B6 acquired in real time from the T1 to the T6 are 90.2° C., 80.9° C., 90.1° C., 90.3° C., 80.8° C. and 90.3° C., respectively, then four second temperatures exist, which are 90.2° C., 90.1° C., 90.3° C. and 90.3° C., respectively. It may be seen that the ratio of the second temperature of all the temperatures acquired by the six temperature acquisition apparatuses T1 to T6 is 4/6≈67%, which is greater than the second ratio of 50%. Hence, according to the difference of 0.3° C. between the maximum temperature of 90.3° C. of the six temperatures and the preset maximum temperature of 90° C. and the correspondence between the plurality of preset difference intervals and the preset perfusion increment, the required perfusion increment is determined and is assumed to be 0.5 ml. Then, according to the initial flow rate of each perfusion channel (assumed to be 0.2 ml) and the determined perfusion increment, the number of the perfusion channels to be opened is determined to be [(0.5/0.2)]+1=3. Finally, the third perfusion channels to be opened are determined to be C5, C1 and C2, respectively successfully according to a distance from the second temperature acquisition apparatus T6 (acquiring the maximum temperature) and the number of the perfusion channels to be opened.

In step S606, the syringe pump is controlled to open the third perfusion channel, and the method returns to perform the step S603, until all the perfusion channels are opened.

Identification information of the plurality of perfusion channels of the syringe pump is stored in the control apparatus. The syringe pump is controlled to open the third perfusion channel according to the identification information. Meanwhile, the method returns to perform the step of determining whether the ratio of the second temperature of the real-time acquired temperatures of the plurality of sites is greater than the second ratio, until all the perfusion channels are opened.

Further, every time the control apparatus controls the syringe pump to open or close the perfusion channel, it generates a corresponding log in which the identification information, the flow rate, and the opening or closing time of the perfusion channel controlled to be opened or closed at this time is at least recorded. Before the syringe pump is controlled to open the third perfusion channel, it is determined whether the third perfusion channel has been opened according to the log. If the third perfusion channel has not been opened, the third perfusion channel is opened. If the third perfusion channel has been opened, the third perfusion channel is skipped, and a perfusion channel adjacent to the third perfusion channel is opened. Following the example above, if the C2 has been opened, the perfusion channel C3 adjacent to the C2 is opened. It should be understood that if the C3 has been opened, the perfusion channel C4 is opened sequentially, and so forth, until all the perfusion channels are opened, or the number of the opened third perfusion channels reaches the number of the perfusion channels to be opened.

Further, after all the perfusion channels are opened, if the ratio of the second temperature is still greater than the second ratio, indicating that the previous perfusion adjustment effect is poor, and the overall temperature of the ablation object is still too high, then the syringe pump is controlled to increase the flow rate of all the perfusion channels from the initial flow rate to a preset maximum flow rate at one time, so as to achieve a rapid cooling effect.

If the ratio of the second temperature is not greater than the second ratio, the method performs a step S607 of determining whether the ratio of the third temperature acquisition apparatus of the temperature acquisition apparatuses is greater than a third ratio.

Particularly, if the ratio of the second temperature greater than the preset maximum temperature of the real-time acquired temperatures of the plurality of sites is not greater than the second ratio, indicating that the overall temperature of the ablation object is within a safe value range, and the current ablation operation will cause no hurt to the ablation object, then it is determined whether the ratio of the third temperature acquisition apparatus of the temperature acquisition apparatuses is greater than the third ratio, so as to ensure that the ablation operation may achieve the desired ablation effect. The temperature acquired by the third temperature acquisition apparatus is less than a preset minimum temperature. The third ratio may be preset in an execution body of the method for controlling the perfusion of the plurality of channels of the syringe pump according to the present invention on the basis of a user-defined operation.

If the ratio of the third temperature acquisition apparatus is greater than the third ratio, the method performs a step S608 of determining a perfusion decrement according to a preset decrement rule.

If the ratio of the third temperature acquisition apparatus of all the temperature acquisition apparatuses is greater than the third ratio, indicating that the overall temperature of the ablation object is slightly low, the current perfusion volume of the liquid is too large, and the desired ablation effect may not be achieved, then the perfusion decrement is determined according to the preset decrement rule.

The preset decrement rule is to determine the perfusion decrement according to the difference between the minimum temperature of the temperatures of the plurality of sites and the preset minimum temperature. The difference between the minimum temperature and the preset minimum temperature is in direct proportional to the perfusion decrement, that is, the greater the difference is, the greater the perfusion decrement required is.

Particularly, the correspondence between the plurality of difference intervals and the preset perfusion decrement may be preset in the control apparatus. Firstly, it is determined that which difference interval the minimum temperature of the temperatures of the plurality of sites falls into, and then the perfusion decrement corresponding to the difference interval that the minimum temperature of the temperatures of the plurality of sites falls into is determined as the required perfusion decrement according to the difference interval that the minimum temperature of the temperatures of the plurality of sites falls into and the preset correspondence.

In step S609, the number of the perfusion channels to be closed is determined according to the perfusion decrement and the initial flow rate, and the fourth perfusion channel is determined according to the number of the perfusion channels to be closed and a second determination rule.

Particularly, according to the perfusion decrement and the initial flow rate, a calculation formula for determining the number of the perfusion channels to be closed is for example described as follows: the value obtained by dividing the perfusion decrement by the initial flow rate is rounded and then added to one.

The second determination rule is to determine the fourth perfusion channel according to a distance from the fourth temperature acquisition apparatus successfully according to the number of the perfusion channels to be closed, wherein the fourth temperature acquisition apparatus acquires the minimum temperature.

A method for determining the fourth perfusion channel is similar to that for determining the third perfusion channel. Particularly, reference may be made to related descriptions in the step S605, which will be omitted here.

In step S610, the syringe pump is controlled to close the fourth perfusion channel, and the method returns to the step S607, until all the perfusion channels are closed.

According to the identification information of each perfusion channel, the control apparatus controls the syringe pump to close the fourth perfusion channel, and the method returns to perform the step of determining whether the ratio of the third temperature acquisition apparatus of the temperature acquisition apparatuses is greater than the third ratio, until all the perfusion channels are closed.

Further, if the fourth perfusion channel to be closed has been closed, a perfusion channel adjacent to the fourth perfusion channel is closed. If the adjacent perfusion channel is closed, the next perfusion channel adjacent to the adjacent perfusion channel is sequentially closed, and so forth, until all the perfusion channels are closed, or the number of the closed fourth perfusion channels reaches the number of the perfusion channels to be closed.

If the ratio of the third temperature acquisition apparatus of all the temperature acquisition apparatuses is not greater than the third ratio, the method returns to the step S603 of determining whether the ratio of the second temperature of the real-time acquired temperatures of the plurality of sites is greater than the second ratio.

In this way, according to the real-time change in the temperatures, the perfusion volume is gradually increased or decreased, which may improve the accuracy of controlling the perfusion, reduce the operation risk, and achieve a better ablation effect.

For unfinished details in this embodiment, reference may be made to related contents in the embodiments shown in FIG. 11 and FIG. 12.

In the embodiments of the present invention, when the ablation task is triggered, the syringe pump is controlled to open and pass through the at least one perfusion channel so as to perform the perfusion operation at the preset initial flow rate. Then, the syringe pump is controlled to open or close some or all of the perfusion channels and/or the flow rates of some or all of the perfusion channels are adjusted according to the temperatures, which are acquired by the plurality of temperature acquisition apparatuses in real time, of the plurality of sites of the ablation object. Accordingly, the perfusion of the plurality of channels of the syringe pump is intelligently adjusted dynamically based on the real-time changes in the temperatures of the plurality of sites of the ablation object in the process of performing the ablation task. Since the perfusion volume of the syringe pump is adjusted relatively purposefully and directionally as the temperatures of the different sites of the ablation object change, operation delays and errors caused by manual determination can be reduced. Meanwhile, the timeliness, the accuracy and the pertinence of perfusing the liquid in the process of performing the ablation task can be improved. Accordingly, the injury of the ablation operation to the ablation object is reduced, and the safety of the radio frequency ablation operation is improved.

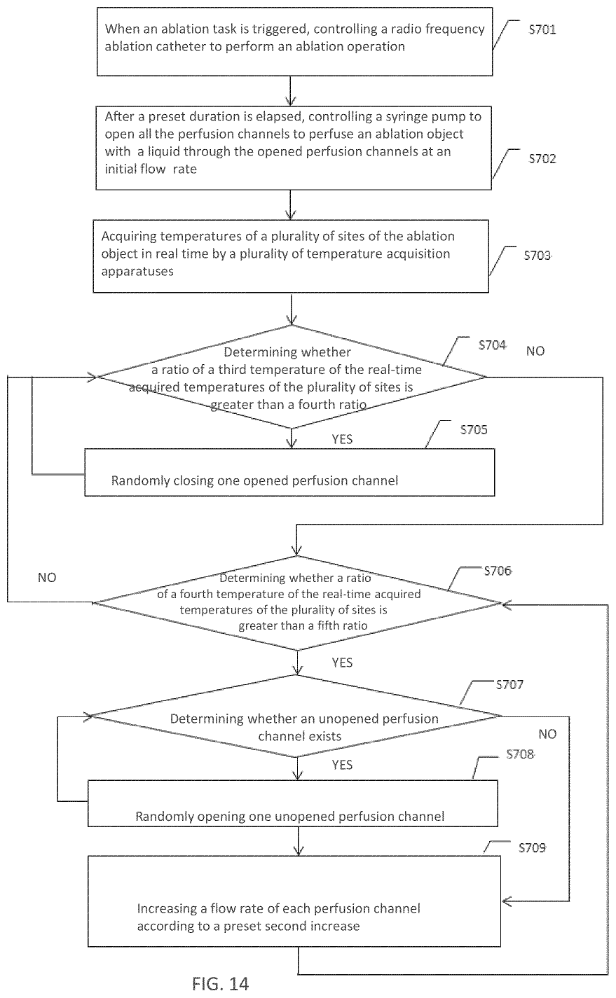

Reference is made to FIG. 14, which is a flowchart of an implementation of a method for controlling perfusion of a plurality of channels of a syringe pump according to another embodiment of the present invention. The method is used to control the syringe pump with a plurality of perfusion channels, such as the syringe pump 20 shown in FIG. 16 and FIG. 17. The method may be implemented by the syringe pump 20 in FIG. 15, or may be implemented by the radio frequency ablation control apparatus 10 in FIG. 15, or may be implemented by another computer device electrically coupled to the syringe pump. For ease of description, the computer device is hereinafter collectively referred to as a control apparatus. As shown in FIG. 14, the method particularly includes the following steps.

In step S701, when the ablation task is triggered, a radio frequency ablation catheter is controlled to perform an ablation operation.

Particularly, the ablation task may be triggered when for example a preset trigger time is reached, a trigger instruction sent by another control apparatus is received, or a notification event that a user performs an operation for triggering the ablation task is detected. The operation for triggering the ablation task is for example to press a physical or virtual button for triggering the ablation task.

Optionally, after each start of the syringe pump, a perfusion parameter is set to a preset initial value. The perfusion parameter may include but is not limited to an initial flow rate, total perfusion volume, perfusion time, and the like.

When the ablation task is triggered, the radio frequency ablation catheter is controlled to start performing an ablation operation on the ablation object, so as to output radio frequency energy to the ablation object.

In step S702, after a preset duration is elapsed, the syringe pump is controlled to open all the perfusion channels, so as to perfuse the ablation object with a liquid through the opened perfusion channel at the initial flow rate.

Particularly, the preset duration may be preset in an execution body of the method for controlling the perfusion of the plurality of channels of the syringe pump according to the present invention on the basis of a user-defined operation.

It should be understood that in order to achieve the desired ablation effect, the temperature of the ablation object needs to reach a certain degree. After the radio frequency ablation catheter is controlled to perform the ablation operation, the syringe pump is controlled to open all the perfusion channels to perform the perfusion operation when the temperature of the ablation object rises to a certain degree for a preset duration, which may prevent premature perfusion from influencing the rate of the temperature rise of the ablation object and ensure a better ablation effect.

In step S703, the temperatures of a plurality of sites of the ablation object are acquired in real time by a plurality of temperature acquisition apparatuses.

Particularly, the step S703, reference may be made to related descriptions of the step S402 in the embodiment shown in FIG. 11, which will be omitted here.

In step S704, it is determined whether a ratio of a third temperature of the real-time acquired temperatures of the plurality of sites is greater than a fourth ratio.

If the ratio of the third temperature is greater than the fourth ratio, the method performs a step S705 of randomly closing one opened perfusion channel, and returns to perform the step S704.

Particularly, the third temperature is less than a preset minimum temperature. If the ratio of the third temperature less than the preset minimum temperature of the real-time acquired temperatures of the plurality of sites is greater than a fourth ratio, indicating that the current perfusion volume is too large, and the overall temperature of the ablation object is unsatisfied, then one opened perfusion channel is randomly closed in order to decrease the perfusion volume. Meanwhile, the method returns to perform the step of determining whether the ratio of the third temperature of the real-time acquired temperatures of the plurality of sites is greater than the fourth ratio, until all the perfusion channels are closed.

If the ratio of the third temperature is not greater than the fourth ratio, the method performs a step S706 of determining whether the ratio of the fourth temperature of the real-time acquired temperatures of the plurality of sites is greater than a fifth ratio.

If the ratio of the fourth temperature is greater than the fifth ratio, the method performs a step S707 of determining whether an unopened perfusion channel exists.

If the unopened perfusion channel exists, the method performs a step S708 of randomly opening one unopened perfusion channel, and returns to perform the step S706, until all the perfusion channels are opened.

If no unopened perfusion channel exists, the method performs a step S709 of increasing the flow rate of each perfusion channel according to a preset second increase, and returns to perform the step S706, until the flow rate of each perfusion channel reaches a preset maximum flow rate.

If the ratio of the fourth temperature is not greater than the fifth ratio, the method returns to perform the step S704.

Particularly, if the ratio of the third temperature less than the preset minimum temperature of the real-time acquired temperatures of the plurality of sites is not greater than the fourth ratio, indicating that the overall temperature of the ablation object reaches the desired temperature, and the current perfusion volume is helpful for the temperature rise of the ablation object, then in order to prevent the ablation object from being hurt by excess temperature, it is determined whether the ratio of the fourth temperature of the real-time acquired temperatures of the plurality of sites is greater than the fifth ratio. The fourth temperature is greater than a preset maximum temperature. The fourth ratio and the fifth ratio may be preset in an execution body of the method for controlling the perfusion of the plurality of channels of the syringe pump according to the present invention on the basis of a user-defined operation.

In one aspect, if the ratio of the fourth temperature of the real-time acquired temperatures of the plurality of sites is greater than the fifth ratio, indicating that the overall temperature of the ablation object is too high, these sites may be hurt, and the perfusion volume needs to be increased to cool these sites, then it is determined whether an unopened perfusion channel exists. If the unopened perfusion channel exists, one unopened perfusion channel is randomly opened. Meanwhile, the method returns to perform the step of determining whether the ratio of the fourth temperature of the real-time acquired temperatures of the plurality of sites is greater than the fifth ratio, until all the perfusion channels are opened. If all the perfusion channels are opened, the flow rate of the perfusion channel is increased according to the second preset increase. Meanwhile, the method returns to perform the step of determining whether the ratio of the fourth temperature of the real-time acquired temperatures of the plurality of sites is greater than the fifth ratio, until the flow rate of the perfusion channel reaches the preset maximum flow rate. Furthermore, if the ratio of the fourth temperature of the real-time acquired temperatures of the plurality of sites after the flow rate of the perfusion channel reaches the preset maximum flow rate is still greater than the fifth ratio, alarm information is output.

In another aspect, if the ratio of the fourth temperature of the real-time acquired temperatures of the plurality of sites is not greater than the fifth ratio, the method returns to perform the step of determining whether the ratio of the third temperature of the real-time acquired temperatures of the plurality of sites is greater than the fourth ratio.

In this way, according to real-time change in the temperatures, the perfusion volume is gradually increased or decreased by increasing or decreasing the number of the perfusion channels, which may improve the accuracy of controlling the perfusion control, reduce the operation risk and achieve a better ablation effect.

For unfinished details in this embodiment, reference may further be made to related descriptions in the embodiments shown in FIG. 11 to FIG. 13.

In the embodiments provided in the present invention, when the ablation task is triggered, the radio frequency ablation catheter is firstly controlled to perform the ablation operation; and after the preset duration is elapsed, the syringe pump is controlled to open all the perfusion channels so as to perfuse the ablation object with the liquid through the opened perfusion channels at the initial flow rate. Then, the syringe pump is controlled to open or close some or all of the perfusion channels and/or the flow rates of some or all of the perfusion channels are adjusted according to the temperatures, which are acquired by the plurality of temperature acquisition apparatuses in real time, of the plurality of sites of the ablation object. Accordingly, the perfusion of the plurality of channels of the syringe pump is intelligently adjusted dynamically based on the real-time changes in the temperatures of the plurality of sites of the ablation object in the process of performing the ablation task. Since the perfusion volume of the syringe pump is adjusted relatively purposefully and directionally as the temperatures of the different sites of the ablation object change, operation delays and errors caused by manual determination can be reduced. Meanwhile, the timeliness, the accuracy and the pertinence of perfusing the liquid in the process of performing the ablation task can be improved. Accordingly, the injury of the ablation operation to the ablation object is reduced, and the safety of the radio frequency ablation operation is improved.

Reference is made to FIG. 16, which is a schematic diagram showing a structure of an apparatus for controlling perfusion of a plurality of channels of a syringe pump according to an embodiment of the present invention. For ease of description, only parts related to embodiments of the present invention are shown. The apparatus may be a syringe pump 20, a radio frequency ablation control apparatus 10 or other computer terminals shown in FIG. 15, or may be a virtual module running in the foregoing apparatus. The apparatus is configured to control the syringe pump with a plurality of perfusion channels, and particularly includes a control module 901 and a temperature acquisition module 902, wherein

the control module 901 is configured to when an ablation task is triggered, control the syringe pump to open at least one perfusion channel to perform a perfusion operation through the opened perfusion channel at a preset initial flow rate;

the temperature acquisition module 902 is configured to acquire temperatures of a plurality of sites of an ablation object in real time by a plurality of temperature acquisition apparatuses; and

the control module 901 is further configured to control the syringe pump to open or close some or all of the perfusion channels and/or control the syringe pump to adjust flow rates of some or all of the perfusion channels according to real-time changes in the temperatures of the plurality of sites.

Optionally, the control module 901 includes: a first control submodule, which is configured to when the ablation task is triggered, control the syringe pump to open one perfusion channel to perfuse the ablation object with a liquid through the opened perfusion channel at the preset initial flow rate; and

the first control submodule is further configured to control a radio frequency ablation catheter to perform the ablation operation on the ablation object.

Optionally, the control module 901 further includes:

a second control submodule, which is configured to:

determine whether a first temperature exists in the real-time acquired temperatures of the plurality of sites, wherein the first temperature is greater than a preset maximum temperature;

if the first temperature exists in the real-time acquired temperatures of the plurality of sites, determine whether the first perfusion channel is opened, wherein the first perfusion channel is configured to perfuse a first site with the liquid, and the first temperature is a temperature of the first site;

if the first perfusion channel is not opened, control the syringe pump to open the first perfusion channel and return to perform the step of determining whether the first temperature exists in the real-time acquired temperatures of the plurality of sites; and

if the first perfusion channel has been opened, control the syringe pump to increase a flow rate of the first perfusion channel according to a first preset increase and return to perform the step of determining whether the first temperature exists in the real-time acquired temperatures of the plurality of sites, until the flow rate of the first perfusion channel reaches the preset maximum flow rate.

Optionally, the second control submodule is further configured to:

after determining whether the first temperature exists in the temperatures of the plurality of sites, if the first temperature does not exist in the temperatures of the plurality of sites, determine whether a ratio of a first temperature acquisition apparatus of the temperature acquisition apparatuses is greater than a first ratio, wherein a preset temperature duration acquired by the first temperature acquisition apparatus is less than a preset minimum temperature;

if the ratio of the first temperature acquisition apparatus of the temperature acquisition apparatuses is greater than the first ratio, control the syringe pump to decrease a flow rate of a second perfusion channel at a first preset decrease and return to perform the step of determining whether the ratio of the first temperature acquisition apparatus of the temperature acquisition apparatuses is greater than the first ratio, until the flow rate of the second perfusion channel reaches a preset minimum flow rate, wherein the second perfusion channel is configured to perfuse a second site with the liquid, and the first temperature acquisition apparatus is configured to detect a temperature of the second site; and if the ratio of the first temperature acquisition apparatus of the temperature acquisition apparatuses is not greater than the first ratio, return to perform the step of determining whether the first temperature exists in the real-time acquired temperatures of the plurality of sites.

Optionally, the apparatus further includes: a setting module, which is configured to set respective preset maximum temperatures and respective preset minimum temperatures for the temperature acquisition apparatuses; and the second control submodule is further configured to determine the first perfusion channel and the second perfusion channel based on the respective preset maximum temperatures and the respective preset minimum temperatures.

Optionally, the control module 901 further includes: a third control submodule, which is configured to: determine whether a ratio of a second temperature in the real-time acquired temperatures of the plurality of sites is greater than a second ratio, wherein the second temperature is greater than a preset maximum temperature; if the ratio of the second temperature is greater than the second ratio, determine a perfusion increment according to a preset increment rule; determine a number of perfusion channels to be opened according to the perfusion increment and the initial flow rate and determine a third perfusion channel according to the number of the perfusion channels to be opened and a first determination rule; and control the syringe pump to open the third perfusion channel and return to perform the step of determining whether the ratio of the second temperature in the real-time acquired temperatures of the plurality of sites is greater than the second ratio, until all the perfusion channels are opened, wherein if the third perfusion channel has been opened, a perfusion channel adjacent to the third perfusion channel is opened.

Optionally, the third control submodule is further configured to after all the perfusion channels are opened, if the ratio of the second temperature is greater than the second ratio, control the syringe pump to increase the flow rates of all the perfusion channels to the preset maximum flow rate.

Optionally, the preset increment rule is to determine the perfusion increment according to a difference between the maximum temperature in the temperatures of the plurality of sites and the preset maximum temperature, wherein the difference between the maximum temperature and the preset maximum temperature is in direct proportional to the perfusion increment; and the first determination rule is to determine the third perfusion channel successively according to a distance from the second temperature acquisition apparatus and the number of the perfusion channels to be opened, wherein a temperature acquired by the second temperature acquisition apparatus is maximum.

Optionally, the third control submodule is further configured to: if the ratio of the second temperature is not greater than the second ratio, determine whether a ratio of a third temperature acquisition apparatus in the temperature acquisition apparatuses is greater than a third ratio, wherein a temperature acquired by the third acquisition apparatus is less than a preset minimum temperature; if the ratio of the third temperature acquisition apparatus is greater than the third ratio, determine a perfusion decrement according to a preset decrement rule; determine a number of perfusion channels to be closed according to the perfusion decrement and the initial flow rate and determine a fourth perfusion channel according to the number of the perfusion channels to be closed and a second determination rule; control the syringe pump to close the fourth perfusion channel and return to perform the step of determining whether the ratio of the third temperature acquisition apparatus of the temperature acquisition apparatuses is greater than the third ratio, until all the perfusion channels are closed, wherein if the fourth perfusion channel has been closed, a perfusion channel adjacent to the fourth perfusion channel is closed; and if the ratio of the third temperature acquisition apparatus is not greater than the third ratio, return to perform the step of determining whether the ratio of the second temperature in the real-time acquired temperatures of the plurality of sites is greater than the second ratio.

Optionally, the preset decrement rule is to determine the perfusion decrement according to a difference between the minimum temperature in the temperatures of the plurality of sites and the preset minimum temperature, wherein the difference between the minimum temperature and the preset minimum temperature is in direct proportional to the perfusion decrement; and the second determination rule is to determine the fourth perfusion channel successively according to a distance from the fourth temperature acquisition apparatus and the number of the perfusion channels to be closed, wherein a temperature acquired by the fourth temperature acquisition apparatus is minimum.

Optionally, the control module 901 further includes: a fourth control submodule, which is configured to when the ablation task is triggered, control a radio frequency ablation catheter to perform an ablation operation; and the fourth control submodule is further configured to after a preset duration is elapsed, control the syringe pump to open all the perfusion channels to perfuse the ablation object with the liquid through the opened perfusion channels at the initial flow rate.

Optionally, the fourth control submodule is further configured to: determine a ratio of a third temperature in the real-time acquired temperatures of the plurality of sites is greater than a fourth ratio, wherein the third temperature is less than a preset minimum temperature; if the ratio of the third temperature is greater than the fourth ratio, randomly close one opened perfusion channel and return to perform the step of determining whether the ratio of the third temperature in the real-time acquired temperatures of the plurality of sites is greater than the fourth ratio, until all the perfusion channels are closed; if the ratio of the third temperature is not greater than the fourth ratio, determine whether a ratio of a fourth temperature in the real-time acquired temperatures of the plurality of sites is greater than a fifth ratio, wherein the fourth temperature is greater than a preset maximum temperature; if the ratio of the fourth temperature is greater than the fifth ratio, determine whether an unopened perfusion channel exists; if the unopened perfusion channel exists, randomly open one unopened perfusion channel and return to perform the step of determining whether the ratio of the fourth temperature in the real-time acquired temperatures of the plurality of sites is greater than the fifth ratio, until all the perfusion channels are opened; if no unopened perfusion channel exists, increase the flow rate of the perfusion channel according to a preset second increase and return to perform the step of determining whether the ratio of the fourth temperature in the real-time acquired temperatures of the plurality of sites is greater than the fifth ratio, until the flow rate of the perfusion channel reaches the preset maximum flow rate; and if the ratio of the fourth temperature is not greater than the fifth ratio, return to the step of determining whether the ratio of the third temperature in the real-time acquired temperatures of the plurality of sites is greater than the fourth ratio.

Specific processes of implementing respective functions by the modules may refer to relevant contents in the embodiments as shown in FIG. 11 to FIG. 16, which will be omitted here.

In the embodiments provided in the present invention, when the ablation task is triggered, the syringe pump is controlled to open and pass through the at least one perfusion channel so as to perform the perfusion operation at the preset initial flow rate. Then, the syringe pump is controlled to open or close some or all of the perfusion channels and/or the flow rates of some or all of the perfusion channels are adjusted according to the temperatures, which are acquired by the plurality of temperature acquisition apparatuses in real time, of the plurality of sites of the ablation object. Accordingly, the perfusion of the plurality of channels of the syringe pump is intelligently adjusted dynamically based on the real-time changes in the temperatures of the plurality of sites of the ablation object in the process of performing the ablation task. Since the perfusion volume of the syringe pump is adjusted relatively purposefully and directionally as the temperatures of the different sites of the ablation object change, operation delays and errors caused by manual determination can be reduced. Meanwhile, the timeliness, the accuracy and the pertinence of perfusing the liquid in the process of performing the ablation task can be improved. Accordingly, the injury of the ablation operation to the ablation object is reduced, and the safety of the radio frequency ablation operation is improved.

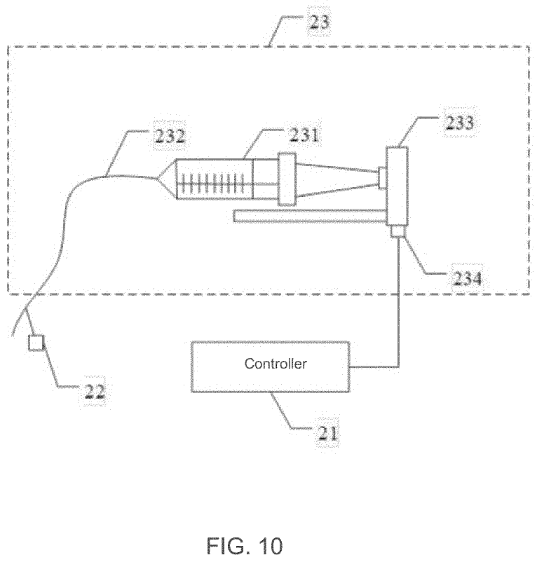

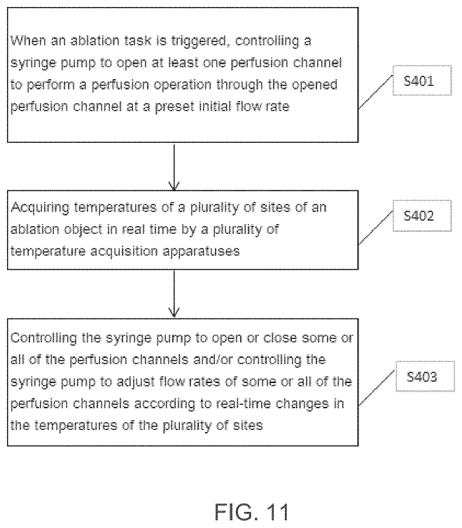

Reference is made to FIG. 10, which is a schematic diagram showing a hardware structure of an electronic apparatus according to an embodiment of the present invention.

Exemplarily, the electronic apparatus may be any one of various types of computer system devices that are non-removable or removable or portable and perform wireless or wired communication. Particularly, the electronic apparatus may be a desktop computer, a server, a mobile phone or a smart phone (for example, an iPhone™-based phone, an Android™-based phone), a portable game device (for example, Nintendo DS™, PlayStation Portable™, Gameboy Advance™, iPhone™), a laptop computer, a PDA, a portable Internet device, a music player and a data storage device, and other handheld devices. The electronic apparatus may further be other wearable devices (for example, such as electronic glasses, electronic clothes, electronic bracelets, electronic necklaces, smart watches or head-mounted devices (HMD)). In some instances, the electronic apparatus may perform multiple functions (for example, playing music, displaying video, storing pictures, and receiving and transmitting phone calls).

As shown in FIG. 17, the electronic apparatus 100 may include a control circuit, wherein the control circuit may include a storage and processing circuit 300. The storage and processing circuit 300 may include a memory, such as a hard disk drive memory, a non-transitory or non-volatile memory (such as a flash memory or other electronically programmable restricted deletion memory configured to form a solid-state drive), and a volatile memory (for example, a static or dynamic random access memory and the like), and the like, which are not limited in the embodiment of the present invention. The processing circuit in the storage and processing circuit 300 may be configured to control the operation of the electronic apparatus 100. The processing circuit may be implemented based on one or more microprocessors, microcontrollers, digital signal processors, baseband processors, power management units, audio codec chips, application specific integrated circuits, display driver integrated circuits, and the like. The processor may be electrically coupled with the plurality of temperature acquisition apparatuses (such as minitype temperature sensors).

The storage and processing circuit 300 may be configured to run software in the electronic apparatus 100, such as an Internet browsing application, a Voice over Internet Protocol (VOIP) telephone calling application, an email application, a media player application, an operating system function, and the like. The software may be configured to perform some control operations, for example, image capture based on a camera, ambient light measurement based on an ambient light sensor, proximity sensor measurement based on a proximity sensor, an information display function realized based on a status indicator such as a status indicator lamp of a LED, touch event detection based on a touch sensor, a function associated with displaying information on a plurality of (for example, layered) displays, an operation associated with performing a wireless communication function, an operation associated with acquiring and generating an audio signal, a control operation associated with acquiring and processing of button press event data, and other functions in the electronic apparatus 100, which are not limited in the embodiment of the present invention.

Further, the memory stores an executable program code, and a processor coupled with the memory calls the executable program code stored in the memory to perform the method for controlling the perfusion of the plurality of channels of the syringe pump described in the embodiments shown in FIG. 11 to FIG. 14 above.

The executable program code includes various modules in the apparatus for controlling the perfusion of the plurality of channels of the syringe pump described in the embodiment shown in FIG. 16, for example, a control module 901 and a temperature acquisition module 902. Respective functions of the control module 901 and the temperature acquisition module 902 may particularly refer to relevant descriptions in the embodiment as shown in FIG. 16, which will be omitted here.

The electronic apparatus 100 may further include an input/output circuit 420. The input/output circuit 420 may be configured to enable the electronic apparatus 100 to input and output data, that is, to allow the electronic apparatus 100 to receive data from an external device and further allow the electronic apparatus 100 to output data from the electronic apparatus 100 to the external device. The input/output circuit 420 may further include a sensor 320. The sensor 320 may include an ambient light sensor, a light-based or capacitive proximity sensor, and a touch sensor (for example, a light-based touch sensor and/or a capacitive touch sensor, wherein the touch sensor may be a part of a touch display screen, or may be independently used as a touch sensor), an acceleration sensor, other sensors and the like.

The input/output circuit 420 may further include one or more displays, for example, a display 140. The display 140 may include one or a combination of more than one of a liquid crystal display, an organic light emitting diode display, an electronic ink display, a plasma display, and a display using other display technologies. The display 140 may include a touch sensor array (i.e., the display 140 may be a touch display screen). The touch sensor may be a capacitive touch sensor formed by an array of transparent touch sensor electrodes (for example, indium tin oxide (ITO) electrodes), or a touch sensor formed by using other touch technologies, such as sonic touch, pressure-sensitive touch, resistance touch and optical touch, which are not restricted by the embodiment of the present invention.

The electronic apparatus 100 may further include an audio component 360. The audio component 360 may be configured to provide audio input and output functions for the electronic apparatus 100. The audio component 360 in the electronic apparatus 100 may include a speaker, a microphone, a buzzer, a tone generator, and other components for generating and detecting sounds.

The communication circuit 380 may be configured to provide the electronic apparatus 100 with the ability to communicate with an external device. The communication circuit 380 may include an analog and digital input/output interface circuit, and a wireless communication circuit based on a radio frequency signal and/or an optical signal. The wireless communication circuit in the communication circuit 380 may include a radio frequency transceiver circuit, a power amplifier circuit, a low noise amplifier, a switch, a filter and an antenna. For example, the wireless communication circuit in the communication circuit 380 may include a circuit for supporting near field communication (NFC) by transmitting and receiving a near-field coupled electromagnetic signal. For example, the communication circuit 380 may include a near-field communication antenna and a near-field communication transceiver. The communication circuit 380 may further include a cellular phone transceiver and an antenna, a wireless local area network transceiver circuit and an antenna, and the like.

The electronic apparatus 100 may further include a battery, a power management circuit and other input/output units 400. The input/output unit 400 may include a button, a joystick, a click wheel, a scroll wheel, a touch pad, a keypad, a keyboard, a camera, a light emitting diode, and other status indicators.

The user may input a command through the input/output circuit 420 to control the operation of the electronic apparatus 100, and may use the output data of the input/output circuit 420 to realize the reception of status information and other outputs from the electronic apparatus 100.

Further, embodiments of the present invention further provide a computer-readable storage medium. The computer-readable storage medium may be provided in the electronic apparatus in each of the foregoing embodiments, and the computer-readable storage medium may be a memory in the storage and processing circuit 300 in the embodiment as shown in FIG. 17. A computer program is stored on the computer-readable storage medium, and when being executed by the processor, implements the method for controlling the perfusion of the plurality of channels of the syringe pump according to the foregoing embodiments as shown in FIG. 11 to FIG. 14. Further, the computer readable storage medium may further be a U disk, a mobile hard disk, a read-only memory (ROM), a RAM, a magnetic disk or an optical disk, and other various media that may store the program code.

In the several embodiments provided in the present invention, it should be understood that the disclosed apparatus and method may be implemented in other ways. For example, the apparatus embodiments described above are merely illustrative. For example, the division of the modules is only a logical function division, or other divisions in practical implementations, for example, multiple modules or components may be combined or may be integrated into another system, or some features may be ignored or not performed. In addition, the displayed or discussed mutual coupling or direct coupling or communication connection may be indirect coupling or communication connection through some interfaces, apparatuses or modules, and may be in electrical, mechanical or other forms.

The modules described as separate components may or may not be physically separated, and the components displayed as modules may or may not be physical modules, that is, they may be located in one place, or they may be distributed onto a plurality of network modules. Some or all of the modules may be selected according to actual needs to achieve the objectives of the solutions of the embodiments.

In addition, the functional modules in the various embodiments of the present invention may be integrated into one processing module, or each module may exist alone physically, or two or more modules may be integrated into one module. The above-mentioned integrated modules may be implemented in the form of hardware or a software functional module.

If the integrated module is implemented in the form of the software function module and sold or used as an independent product, it may be stored in a computer-readable storage medium. Based on such an understanding, the technical solution of the present invention substantively, or a part thereof making a contribution to the prior art, or all or part of the technical solution may be embodied in the form of a software product stored in a readable storage medium, and the readable storage medium includes several instructions to enable a computer device (which may be a personal computer, a server, or a network device) to perform all or part of the steps of the methods according to the various embodiments of the present invention. The above-mentioned readable storage medium includes a U disk, a mobile hard disk, a ROM, a RAM, a magnetic disk or an optical disk, and other media that may store the program code.

It should be noted that for simplicity of description, the foregoing method embodiments are all expressed as a series of action combinations, but because according to the present invention, some steps may be performed in other sequences or simultaneously, those skilled in the art should appreciate that the present invention is not limited by the described sequence of actions. Secondly, those skilled in the art should further appreciate that the embodiments described in the specification are all preferred embodiments, and the involved actions and modules are not necessarily all required by the present invention.

In the above-mentioned embodiments, descriptions of the embodiments have particular emphasis respectively. For parts that are not described in detail in a certain embodiment, reference may be made to related descriptions of other embodiments.

The above is a description of the method and the apparatus for controlling the perfusion of the plurality of sites of the syringe pump, the syringe pump and the computer-readable storage medium according to the present invention. For those skilled in the art, according to the ideas of the embodiments of the present invention, changes may be made to specific implementations and application scopes. In summary, the content of this specification should not be construed as a limitation on the present invention.

Data Adjustment Method in Radio-Frequency Operation and Radio-Frequency Host

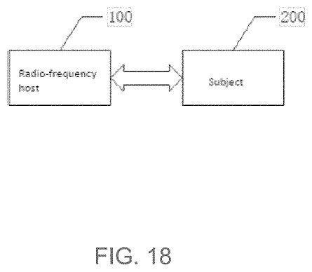

FIG. 18 is a schematic diagram showing an application scenario of a data adjustment method in a radio-frequency operation provided in an embodiment of the present invention. The data adjustment method in a radio-frequency operation includes, during the radio-frequency operation, outputting a radio-frequency signal at a set power, detecting physical characteristic data of a subject of the radio-frequency operation in real time, and determining whether to adjust the radio-frequency output power or the physical characteristic data according to the change of the physical characteristic data. As a result, the data of the radio-frequency operation tends to be more reasonable, to improve the success rate and safety of the radio-frequency operation.

Particularly, an implementation body of the data adjustment method is a radio-frequency host that may be specifically a radio-frequency ablation instrument or other devices. As shown in FIG. 18, a radio-frequency host 100 is connected to a subject 200, and then a radio-frequency operation is started, in which the radio-frequency host 100 transmits a radio-frequency signal to the subject 200 by a radio-frequency generator. In the radio-frequency operation, as the nature of the subject 200 changes, physical characteristic data also changes. The subject 200 can be any object that needs the radio-frequency operation. For example, when the radio-frequency host 100 is a radio-frequency ablation instrument, the subject 200 can be an organism that needs to ablate abnormal tissues in the body.

The radio-frequency host 100 has an input interface that can be externally connected to a movable storage such as U disk, or externally connected to an input device such as keyboard and mouse, to read data from the removable storage or acquire data inputted by a user from the input device. The radio-frequency host 100 may also be connected to a server over a network, to obtain, from the server, big data from all radio-frequency hosts connected to the server, wherein the big data includes various historical data related to the radio-frequency operation.

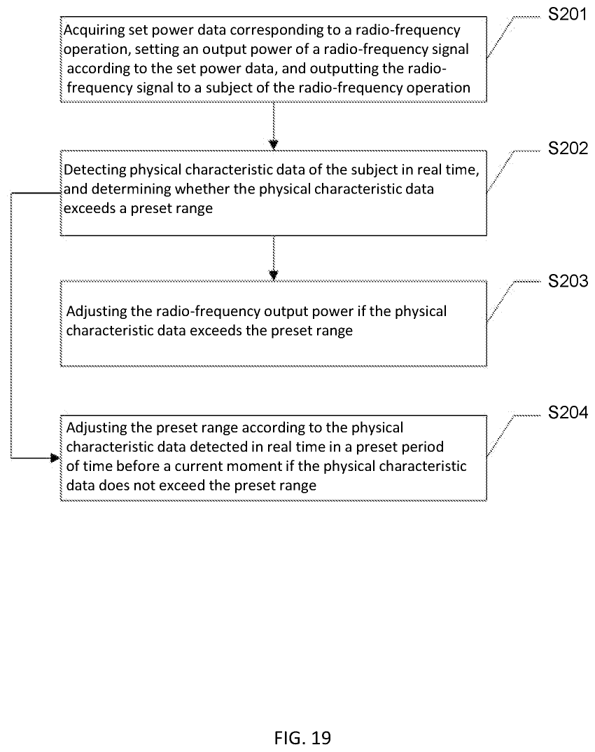

FIG. 19 is a schematic flow chart of a data adjustment method in a radio-frequency operation provided in an embodiment of the present invention. The method is applicable to the radio-frequency host as shown in FIG. 18. As shown in FIG. 19, the method includes specifically:

Step S201: acquiring set power data corresponding to a radio-frequency operation, setting an output power of a radio-frequency signal according to the set power data, and outputting the radio-frequency signal to a subject of the radio-frequency operation.

Particularly, the set power data can be obtained by obtaining, from a server, historical radio-frequency operation data of all radio-frequency hosts in a network, or obtained from set data entered by a user into the radio-frequency host.

Step S202: detecting physical characteristic data of the subject in real time, and determining whether the physical characteristic data exceeds a preset range, wherein the physical characteristic data includes the temperature and impedance of the subject.

In the radio-frequency operation, the radio-frequency signal outputted on the subject has radio-frequency energy, and a site receiving the radio-frequency operation has changed physical characteristic data under the action of the radio-frequency energy,

The preset range is a numerical interval defined by a lowest value and a highest value, and the method of obtaining the lowest value and the highest value is the same as the method of obtaining the set power data in Step S201. That is, the lowest value and the highest value can be obtained by obtaining, from a server, historical radio-frequency operation data of all radio-frequency hosts in a network, or obtained from set data entered by a user into the radio-frequency host.

Step S203: adjusting the radio-frequency output power if the physical characteristic data exceeds the preset range.

If the physical characteristic data is higher than the highest value of the preset range or lower than the lowest value of the preset range, it is determined to exceed the preset range. Then, the radio-frequency output power is adjusted, to reduce or increase the physical characteristic data.

In this embodiment, the calculation of the actual power detected in real time requires the measurement of the corresponding voltage and current, and then the real-time power is calculated according to a product of the voltage and current.

Step S204: adjusting the preset range according to the physical characteristic data detected in real time in a preset period of time before a current moment if the physical characteristic data does not exceed the preset range.

If the physical characteristic data does not exceed the preset range, the preset range is adjusted according to the physical characteristic data detected in real time in a preset period of time before a current moment. The adjusted physical characteristic data can be used as historical radio-frequency operation data, and set a data basis for a preset range of the physical characteristic data of a next radio-frequency operation, thus making the data be of great referential value, and improving the accuracy of the radio-frequency operation.

In the embodiments of the present invention, set power data corresponding to a radio-frequency operation is acquired, an output power of a radio-frequency signal is set according to the set power data, and the radio-frequency signal is outputted physical characteristic data of a subject of the radio-frequency operation is detected in real time during the radio-frequency operation, whether the physical characteristic data exceeds a preset range is determined, wherein if the physical characteristic data exceeds the preset range, the radio-frequency output power is adjusted, to reduce the risk of the radio-frequency operation damaging the subject and improve the safety of the radio-frequency operation; and if the physical characteristic data does not exceed the preset range, the preset range of the physical characteristic data is adjusted, and the reasonableness of the preset range is automatically updated, to provide a more accurate data basis for subsequent radio-frequency operations, and improve the reasonableness and success rate of the radio-frequency operation.

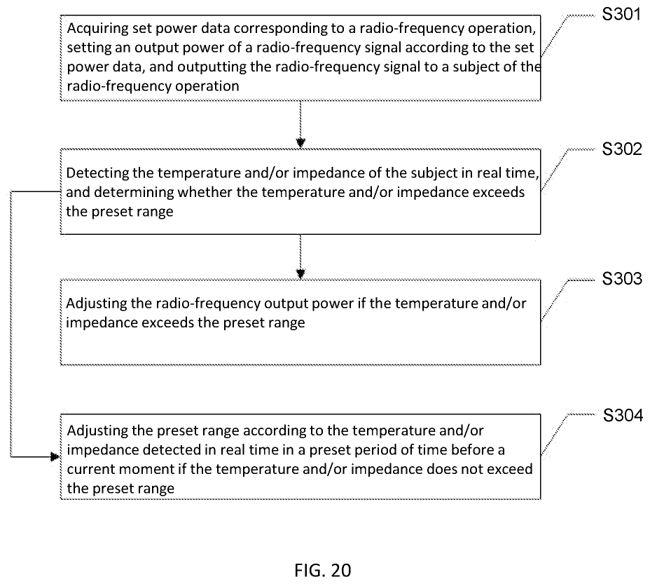

FIG. 20 is a schematic flow chart of a data adjustment method in a radio-frequency operation provided in another embodiment of the present invention. The method is applicable to the radio-frequency host as shown in FIG. 18. As shown in FIG. 20, the method includes specifically:

Step S301: acquiring set power data corresponding to a radio-frequency operation, setting an output power of a radio-frequency signal according to the set power data, and outputting the radio-frequency signal to a subject of the radio-frequency operation.

Particularly, the set power data can be obtained through the following two methods.

In a first method, historical radio-frequency operation data corresponding to the task and subject of the radio-frequency operation is obtained from a server. Then the historical radio-frequency operation data is classified according to the task of the radio-frequency operation and the nature of the subject. For example, the historical radio-frequency operation data where task No. 1 is performed and the subject is A is classified into one category, the historical radio-frequency operation data where task No. 2 is performed and the subject is A is classified into one category, the historical radio-frequency operation data where task No. 1 is performed and the subject is B is classified into one category, and so on. Because of the same task, and the same nature of the subject, the corresponding relationship between each category of historical radio-frequency operation data and the radio-frequency operation time is also the same.

Therefore, when the radio-frequency operation is performed, radio-frequency power data is queried from the corresponding historical radio-frequency operation data according to the task and subject of the current radio-frequency operation, the queried radio-frequency power data is used as the set power data, the output power of the radio-frequency signal in various periods of time of the radio-frequency operation is set according to the corresponding relationship between the set power data and the radio-frequency operation time, and the radio-frequency signal having the output power is outputted to the subject. Particularly, the output power of the radio-frequency signal in the historical radio-frequency operation data is determined as the set power data, wherein the set power data is specifically a change trend curve representing the corresponding relationship between the radio-frequency operation time and the output power. From the change trend curve, the output power in an operation time corresponding to the current stage of the current radio-frequency operation is acquired, and the acquired output power is set as the output power of the radio-frequency signal.

In a second method, the set power data can be obtained from the set data entered by a user into the radio-frequency host. Particularly, the set power data is acquired from the set data in the removable storage connected to the radio-frequency host, or the set power data is acquired from the set data inputted via an input device of the radio-frequency host. The set power data is a numerical interval including a maximum value of the set power and a minimum value of the set power, and

a median value of the numerical interval is set as the output power of the radio-frequency signal. The radio-frequency signal having the output power is outputted to the subject.

Step S302: detecting the temperature and/or impedance of the subject in real time, and determining whether the temperature and/or impedance exceeds the preset range.

Step S303: adjusting the radio-frequency output power if the temperature and/or impedance exceeds the preset range.

Particularly, the radio-frequency output power can be adjusted as follows. If the temperature or impedance of the subject detected in real time is greater than the maximum value of the preset range, the output power of the radio-frequency signal is reduced to a preset first target power; and

if both the temperature and the impedance of the subject detected in real time are less than the minimum value of the preset range, the output power of the radio-frequency signal is increased to a preset second target power.

Due to the high temperature generated by the radio-frequency energy, the impedance of the site of the subject receiving the radio-frequency operation is caused to increase. Accordingly, the temperature and/or impedance of the subject detected is detected in real time. If they exceed the preset range, and generally are greater than the maximum value of the preset range, the output power of the radio-frequency signal is reduced to the preset first target power, If the temperature and/or impedance of the subject detected still exceeds the preset range, the output power of the radio-frequency signal is further reduced to a next target power lower than the first target power. The target power for each reduction is preset in the radio-frequency host.

If a radio-frequency probe provided on the radio-frequency host is a multi-electrode radio-frequency probe, the radio-frequency output power may also be adjusted as follows. If the temperature or impedance of the subject detected in real time is greater than the maximum value of the preset range, the total power needed to be set is determined according to the minimum impedance of each electrode of the multi-electrode radio-frequency probe, and the real-time total power of the radio-frequency probe of the radio-frequency host is detected. The power adjustment is calculated by the default proportional integral differential (PID) algorithm according to the total power needed to be set and the real-time total power, and the target power is calculated according to the power adjustment and the current output power of the radio-frequency signal. Then, the radio-frequency output power is reduced to the target power.

Particularly, the impedances of multiple electrodes of the multi-electrode radio-frequency probe are detected, and an individual electrode with the smallest impedance is determined. According to the impedance of the individual electrode, the preset power of the individual electrode, and the impedances of other electrodes of the multi-electrode radio-frequency probe than the individual electrode, the powers of other electrodes are calculated, and the sum of the power of each electrode is taken as the total power needed to be set.

The power calculation formula is P=U²/R. Since each electrode of the multi-electrode radio-frequency probe is connected to the same voltage output, and each electrode has the same voltage at the site of the radio-frequency operation. The power of each electrode depends on the impedance R, and the power P increases with the decrease of R. The power of each individual electrode is delimited by the set total power, and can be equal to, but cannot exceed the set total power. The set total power is the sum of the power of each electrode.

Particularly, the current total power needed to be set is calculated according to the impedance of the electrode;

According to the formula P=U²/R, it can be deduced that the relationship between the power Pₗᵢₘ of the electrode with the smallest impedance and the power Pₙ of other individual electrodes is:

$$\frac{{P}_{lim}}{{P}_{n}} = \frac{{U}^{2} / {R}_{lim}}{{U}^{2} / {R}_{n}} = \frac{{R}_{n}}{{R}_{lim}} , that is ,$$

$${P}_{n} = \frac{{P}_{lim} {R}_{lim}}{{R}_{n}} .$$

Pₗᵢₘ is the power of the known electrode with the smallest impedance, and according to Rₗᵢₘ and the impedances Rₙ of other individual electrodes, Pₙ corresponding to each individual electrode can be obtained. The total power P needed to be set is calculated by the formula

$$P = {\sum }_{i = 1}^{n} {P}_{i} .$$

According to the currently measured real-time total power and the total power P needed to be set, the total power increment ΔP can be obtained according to the PID algorithm. The PID algorithm is accomplished by

$$Formula 1$$

$$u ( k ) = {K}_{P} \{ e r r ( k ) + \frac{T}{{T}_{I}} {\sum }_{j = 0}^{k} e r r ( j ) + \frac{{T}_{D}}{T} [ e r r ( k ) - e r r ( k - 1 ) ] \} ; or$$

$$Formula 2$$

$$u ( k ) = {K}_{P} e r r ( k ) + {K}_{I} {\sum }_{j = 0}^{k} err ( j ) + {K}_{D} [ err ( k ) - err ( k - 1 ) ] ;$$

wherein

$${K}_{P} , {K}_{I} = {K}_{P} \frac{T}{{T}_{I}} , and {K}_{D} = {K}_{P} \frac{{T}_{D}}{T}$$

are respectively the proportional coefficient, integral coefficient and differential coefficient of the PID algorithm, T is the sampling time, T_{I} is the integration time (also referred to as the integral coefficient), T_{D} is the differential time (also referred to as the differential coefficient), err(k) is the difference between the total power needed to be set and the real-time total power, and u(k) is the output.

By using the incremental PID algorithm ΔP=u(k)−u(k−1), it can be obtained from Formula 2 above:

ΔP=K_{P}[err(k)−err(k−1)]+K_{I}err(k)+K_{D}[err(k)˜2err(k−1)+err(k−2)]

The output adjustment is calculated according to ΔP, and the adjustment has a one-to-one mapping relationship with ΔP, because the power adjustment is achieved by controlling a voltage signal from a power board, the output voltage corresponds to an input digital signal of a digital-to-analog converter, and the adjustment actually corresponds to this digital signal. The mapping relationship enables a corresponding relationship between the output and ΔP, for example, the output of 1 means that the corresponding power increment ΔP is 0.1 w. In this way, the control of ΔP is achieved according to the mapping relationship.

The current power is increased by a value of ΔP to obtain the target power. When ΔP is a negative value, the increment ΔP means to reduce the radio-frequency output power, to lower the temperature. Otherwise, when ΔP is a positive value, it means to increase the radio-frequency output power, to increase the temperature.

The radio-frequency output power is adjusted to the target power and then outputted.

If the temperature or impedance of the subject detected in real time is less than the minimum value of the preset range, the method to adjust the power is as described above.

Step S304: adjusting the preset range according to the temperature and/or impedance detected in real time in a preset period of time before a current moment if the temperature and/or impedance does not exceed the preset range.

Particularly, according to the various temperatures and/or impedances detected in real time in the preset period of time, and a default selection algorithm, a target value is selected from various temperatures and/or impedances in the preset time to update the end values of the preset range, wherein the end values include a minimum and a maximum value.

More specifically, the preset period of time is 10 sec. Taking temperature as an example, a minimum value among various temperatures in 10 seconds before the current moment is selected as the minimum value of the preset range, and a maximum value among various temperatures is selected as the maximum value of the preset range; or a median value of various temperatures in 10 seconds before the current moment is calculated, and the to-be-updated end values corresponding to the median value is calculated according to the median value with reference to the difference between the median value and the end values of the preset range before updating, wherein calculated end values are the end values of the updated preset range.

In the embodiments of the present invention, set power data corresponding to a radio-frequency operation is acquired, an output power of a radio-frequency signal is set according to the set power data, and the radio-frequency signal is outputted, the temperature and impedance of a subject of the radio-frequency operation is detected in real time during the radio-frequency operation, and whether the temperature and/or impedance exceeds a preset range is determined, wherein if the temperature or impedance is greater than the maximum value of the preset range, the output power of the radio-frequency signal is reduced, to reduce the risk of the radio-frequency operation damaging the subject and improve the safety of the radio-frequency operation; if the temperature and impedance are both lower than the minimum value of the preset range, the output power of the radio-frequency signal is increased, to improve the effect of the radio-frequency operation; and further, if the temperature and/or impedance does not exceed the preset range, the reasonableness of the preset range is automatically updated, to provide a more accurate data basis for subsequent radio-frequency operations, and improve the reasonableness and success rate of the radio-frequency operation.

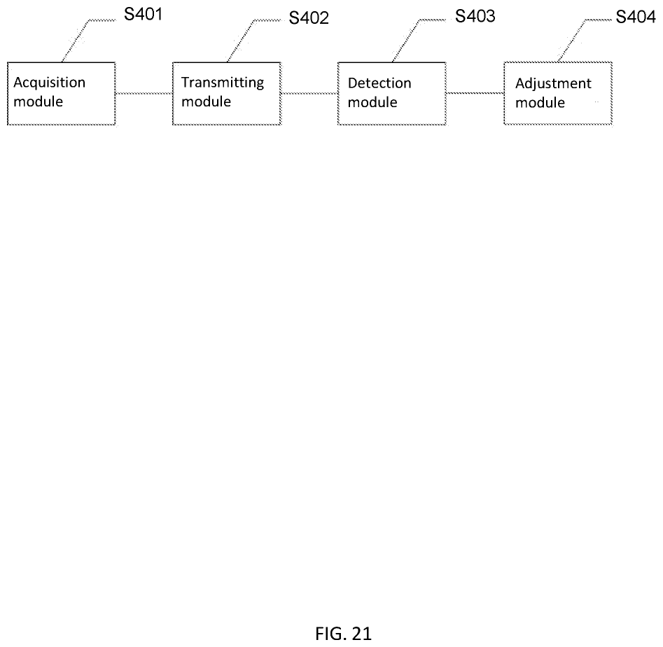

FIG. 21 is a schematic structural diagram of a radio-frequency host provided in an embodiment of the present invention. For ease of description, only the parts relevant to the embodiments of the present invention are shown. The radio-frequency host is a radio-frequency host for implementing the data adjustment method in a radio-frequency operation described in the above embodiments. The radio-frequency host includes:

an acquisition module 401, configured to acquire set power data corresponding to a radio-frequency operation;

a transmitting module 402, configured to set an output power of a radio-frequency signal according to the set power data, and output the radio-frequency signal to a subject of the radio-frequency operation;

a detection module 403, configured to detect physical characteristic data of the subject in real time, and determine whether the physical characteristic data exceeds a preset range; and

an adjustment module 404, configured to adjust the radio-frequency output power if the physical characteristic data exceeds the preset range,

and adjust the preset range according to the physical characteristic data detected in real time in a preset period of time before a current moment if the physical characteristic data does not exceed the preset range.

The various modules in the radio-frequency host serve to implement the following functions. Set power data corresponding to a radio-frequency operation is acquired, an output power of a radio-frequency signal is set according to the set power data, and the radio-frequency signal is outputted, physical characteristic data of a subject of the radio-frequency operation is detected in real time during the radio-frequency operation, and whether the physical characteristic data exceeds a preset range is determined, wherein if the physical characteristic data exceeds the preset range, the radio-frequency output power is adjusted, to reduce the risk of the radio-frequency operation damaging the subject and improve the safety of the radio-frequency operation; if the physical characteristic data does not exceed the preset range, the preset range of the physical characteristic data is adjusted, and the reasonableness of the preset range is automatically updated, to provide a more accurate data basis for subsequent radio-frequency operations, and improve the reasonableness and success rate of the radio-frequency operation.

Further, the detection module 403 is further configured to detect the temperature and/or impedance of the subject in real time.

Further, the adjustment module 404 is further configured to reduce the radio-frequency output power to a preset first target power, if the temperature or impedance of the subject detected in real time is greater than the maximum value of the preset range; and increase the radio-frequency output power to a preset second target power if the temperature and impedance of the subject detected in real time are both less than the minimum value of the preset range.

If a radio-frequency probe provided on the radio-frequency host is a multi-electrode radio-frequency probe, the detection module 403 is further configured to determine the total power needed to be set according to the minimum impedance of each electrode of the multi-electrode radio-frequency probe, if the temperature or impedance of the subject detected in real time is greater than the maximum value of the preset range, and detect the real-time total power of the radio-frequency probe of the radio-frequency host; and the adjustment module 404 is further configured to calculate a power adjustment by default PID algorithm according to the total power needed to be set and the real-time total power, calculate a target power according to the power adjustment and the current output power of the radio-frequency signal, and reduce the radio-frequency output power to the target power.

The adjustment module 403 is also configured to select a target value from various temperatures and/or impedances to update end values of the preset range according to the various temperatures and/or impedances detected in real time in the preset period of time and a default selection algorithm.

The acquisition module 401 is further configured to acquire historical radio-frequency operation data corresponding to the task and subject of the radio-frequency operation; and determine the output power of the radio-frequency signal in the historical radio-frequency operation data as the set power data, wherein the set power data is a change trend curve representing the corresponding relationship between the radio-frequency operation time and the output power.

The transmitting module 402 is further configured to acquire the output power in an operation time corresponding to the current stage of the current radio-frequency operation, and set the acquired output power as the output power of the radio-frequency signal.

The acquisition module 401 is further configured to acquire the set power data from an externally connected removable storage, or acquire the set power data inputted from an input device, wherein the set power data is a numerical interval including a maximum value of the set power and a minimum value of the set power.

The transmitting module 402 is further configured to set a median value of the numerical interval as the output power of the radio-frequency signal.

In the embodiments of the present invention, set power data corresponding to a radio-frequency operation is acquired, an output power of a radio-frequency signal is set according to the set power data, and the radio-frequency signal is outputted, the temperature and impedance of a subject of the radio-frequency operation is detected in real time during the radio-frequency operation, and whether the temperature and/or impedance exceeds a preset range is determined, wherein if the temperature or impedance is greater than the maximum value of the preset range, the radio-frequency output power is reduced, to reduce the risk of the radio-frequency operation damaging the subject and improve the safety of the radio-frequency operation; if the temperature and impedance are both lower than the minimum value of the preset range, the output power of the radio-frequency signal is increased, to improve the effect of the radio-frequency operation; and further, if the temperature and/or impedance does not exceed the preset range, the reasonableness of the preset range is automatically updated, to provide a more accurate data basis for subsequent radio-frequency operations, and improve the reasonableness and success rate of the radio-frequency operation.

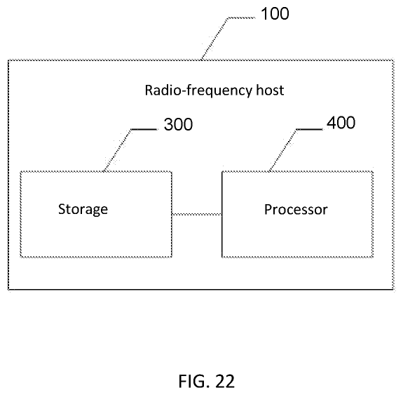

Further, as shown in FIG. 22, an embodiment of the present invention also provides a radio-frequency host, which includes a storage 300 and a processor 400, wherein the processor 400 may be a central processor in the radio-frequency host provided in the above embodiments. The storage 300 is, for example, hard drive storage, a non-volatile storage (such as flash memory or other storages that are used to form solid-state drives and are electronically programmable to confine the deletion, etc.), and a volatile storage (such as static or dynamic random access storage), which is not limited in the embodiments of the present invention.

The storage 300 stores an executable program code; and the processor 400 is 300 coupled to the storage 300, and configured to call the executable program code stored in the storage, and implement the data adjustment method in a radio-frequency operation as described above.

Further, an embodiment of the present invention further provides a computer-readable storage medium. which can be provided in the radio-frequency host in each of the above embodiments, and may be the storage 300 in the embodiment shown in FIG. 22. A computer program is stored in the computer-readable storage medium, and when the program is executed by a processor, the data adjustment method in a radio-frequency operation according to the embodiments shown in FIG. 19 and FIG. 20 is implemented. Further, the computer-readable storage medium may also be a U disk, a removable hard disk, a read-only storage (ROM, Read-Only Memory), RAM, a magnetic disk or an optical disk and other media that can store program codes.

In the above embodiments, emphasis has been placed on the description of various embodiments. Parts of an embodiment that are not described in detail may be found in the description of other embodiments.

The data adjustment method in a radio-frequency operation and the radio-frequency host provided in the present invention have been described above. Changes can be made to the specific implementation and the scope of the present invention by those skilled in the art according to the idea of the embodiments of the present invention. Therefore, the disclosure of this specification should not be construed as a limitation of the present invention.

Method for Protecting Radio Frequency Operation Abnormality, Radio Frequency Mainframe, and Radio Frequency Operation System

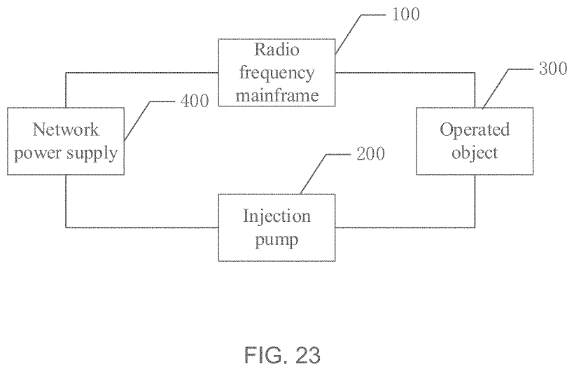

Referring to FIG. 23, which is a schematic diagram of an application scene of a method for protecting radio frequency operation abnormality provided by an embodiment of this application. The method for protecting radio frequency operation abnormality can be configured to: when a radio frequency mainframe continuously outputs energy, if it is detected that a radio frequency operation appears an abnormal state, protect the radio frequency mainframe and a radio frequency operated object from being damaged through multiple manners, thereby improve safety of radio frequency operation.

Specifically, as shown in FIG. 23, a radio frequency mainframe 100, an injection pump 200, and an operated object 300 are interconnected to form a radio frequency operating system, which is powered by a network power supply 400. The network power supply 400 is a common AC220V voltage, and a power supply system of the radio frequency mainframe 100 itself performs necessary processing and splitting for the network power supply 400 and then outputs power to other apparatuses or modules of the radio frequency mainframe 100. Among them, the radio frequency mainframe 100 may specifically be a radio frequency ablation apparatus or other equipment, which outputs radio frequency energy to the operating object 300. During radio frequency operations, according to requirements of the operations, the injection pump 200 injects liquid into the operating object 300 for cooling, impedance reduction, etc.

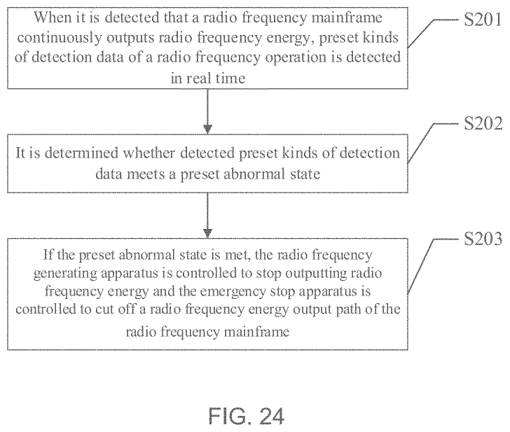

Referring to FIG. 24, which is a schematic flow chart of a method for protecting radio frequency operation abnormality provided by an embodiment of this application. The method can be applied in the radio frequency mainframe as shown in FIG. 23. As shown in FIG. 24, the method specifically comprises the follows.

Step S201, when it is detected that a radio frequency mainframe continuously outputs radio frequency energy, preset kinds of detection data of a radio frequency operation is detected in real time.

An executing main body of this embodiment is a radio frequency mainframe, the radio frequency mainframe includes at least a detecting apparatus, a controlling apparatus, a radio frequency generating apparatus, and an emergency stop apparatus, the controlling apparatus is connected to the detecting apparatus, the radio frequency generating apparatus, and the emergency stop apparatus respectively, and the detecting apparatus is further connected to the radio frequency generating apparatus.

The detecting apparatus is used to detect various data of a radio frequency operation process, including data of radio frequency energy output, an impedance and a temperature of an operated object, voltages and currents of circuits, etc. In addition, the detecting apparatus is further provided therein with a processor that is independent of the controlling apparatus of the radio frequency mainframe, including a single-chip microcomputer, an MCU (Microcontroller Unit), a CPU (Central Processing Unit), etc., which can realize functions of data collection, data distribution, data analysis, and so on.

The radio frequency apparatus is used to emit radio frequency energy acting on an operated object.

The emergency stop apparatus is used to stop a radio frequency operation quickly when an emergency state set in a system occurs.

The controlling apparatus is a processor of the radio frequency mainframe, which is used to acquire data, analyzed data, and control starting and stopping of functions of all apparatuses or modules in the radio frequency mainframe according to analysis result.

Specifically, the radio frequency energy data includes output power and output time of radio frequency energy, the detecting apparatus detects data of radio frequency energy emitted by the radio frequency generating apparatus; when the output power of radio frequency energy reaches preset working power and the output time reaches a preset output time length, it is determined that the radio frequency main frame continuously outputs radio frequency energy to the operated object, and a stable radio frequency operation stage is entered.

Step S202, it is determined whether detected preset kinds of detection data meets a preset abnormal state.

The preset abnormal state includes an abnormality determination standard for the preset kinds of detection data.

Furthermore, information of the abnormal state is also set in the controlling apparatus.

Step S203, if the preset abnormal state is met, the radio frequency generating apparatus is controlled to stop outputting radio frequency energy and the emergency stop apparatus is controlled to cut off a radio frequency energy output path of the radio frequency mainframe.

When the detection data of the detecting apparatus meets the preset abnormal state, dual protection including the following two manners is performed for a radio frequency operation appearing abnormality.

One manner is controlling the radio frequency generating apparatus to stop outputting radio frequency energy, and the other is to control the emergency stop apparatus to cut off a radio frequency energy output path of the radio frequency mainframe. It can be prevented that unexpected failure of any one manner causes radio frequency energy to output continuously and brings damage to the operated object and the radio frequency mainframe.

In this embodiment of this application, when a radio frequency mainframe continuously outputs radio frequency energy, preset kinds of detection data of a radio frequency operation is detected in real time; it is determined whether detected data meets a preset abnormal state; and if yes, a radio frequency generating apparatus is controlled to stop outputting radio frequency energy and an emergency stop apparatus is controlled to cut off a radio frequency energy output path of the radio frequency mainframe. The above two manners of protection are performed at the same time to prevent any one manner from failing or malfunctioning and causing protection failure, a succeeding rate of protection is improved, and safety of radio frequency operations is improved.

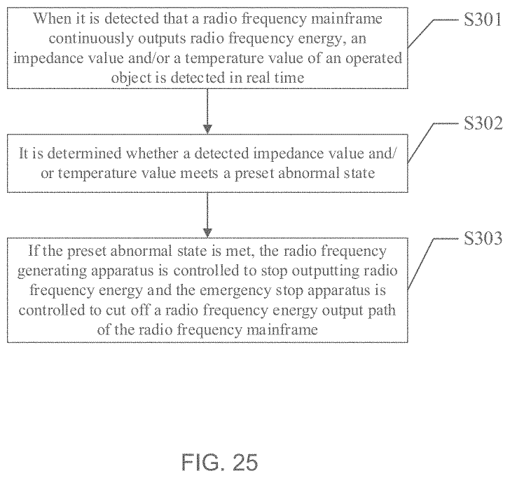

Referring to FIG. 25, which is an implementation flow chart of a method for protecting radio frequency operation abnormality provided by another embodiment of this application. The method can be applied in the radio frequency mainframe as shown in FIG. 23. As shown in FIG. 25, the method specifically comprises the follows.

Step S301, when it is detected that a radio frequency mainframe continuously outputs radio frequency energy, an impedance value and/or a temperature value of an operated object is detected in real time.

A too high impedance value or temperature value of the operating object may cause irreversible damage to the operated object, and is an important protection direction. During a radio frequency operation process, an impedance value or a temperature value of an operating object is detected in real time, or the impedance value and the temperature value are detected at the same time.

Step 302, it is determined whether a detected impedance value and/or temperature value meets a preset abnormal state.

Specifically, it is determined whether a detected impedance value of an operated object is higher than a first preset impedance threshold, and whether a duration time length of being higher than the first preset impedance threshold is larger than a preset time length, wherein the preset time length is preferably 3 seconds; and/or whether a temperature value of an operated object is higher than a first preset temperature threshold, and whether a duration time length of being higher than the first preset temperature threshold is larger than the preset time length.

Both the first preset impedance threshold and the first preset temperature threshold are upper limit values, and specifically relate to content of the present radio frequency operation and a type of the operated object, which are not specifically limited.

If the impedance value of the operated object is higher than the first preset impedance threshold, and the duration time length of being higher than the first preset impedance threshold is larger than the preset time length, or if the temperature value of the operated object is higher than the first preset temperature threshold, and the duration time length of being higher than the first temperature impedance threshold is larger than the preset time length, it is determined that the preset abnormal state is met. That is, when any one value of the impedance value and the temperature value of the operated object is higher than an upper limit value, it can be determined that the current radio frequency operation appears the preset abnormal state.

Step S303, if the preset abnormal state is met, the radio frequency generating apparatus is controlled to stop outputting radio frequency energy and the emergency stop apparatus is controlled to cut off a radio frequency energy output path of the radio frequency mainframe.

Specifically, on one hand, detected detection data of the preset kind is sent to the controlling apparatus, the controlling apparatus can analyze that the preset abnormal state occurs according to the detection data, send stop information for stopping generating and outputting radio frequency energy to the radio frequency generating apparatus, so as to control the radio frequency generating apparatus to stop outputting radio frequency energy.

Alternatively, after determining that the preset abnormal state occurs, the detecting apparatus directly sends abnormal state prompt information to the controlling apparatus; the controlling apparatus sends stop information for stopping generating and outputting radio frequency energy to the radio frequency generating apparatus according to the prompt information.

On the other hand, the detecting apparatus controls the emergency stop apparatus to cut off a radio frequency energy output path of the radio frequency mainframe; specifically, cutting information is send to the emergency stop apparatus connected to the detecting apparatus, and the emergency stop apparatus cuts off a radio frequency energy output path between the radio frequency mainframe and the operated object.

As described above, by the detecting apparatus and the controlling apparatus, which are two different apparatuses, the radio frequency generating apparatus and the emergency stop apparatus are respectively controlled to simultaneously stop delivering radio frequency energy to the operated object, so that failure risk of completing control by the same one apparatus is avoided, a succeeding rate of stopping delivering is improved, and safety of protecting the operated object and the radio frequency mainframe is further improved.

Other technical details of the above steps refer to description of the embodiment shown in aforementioned FIG. 24, and are not repeated here.

In this embodiment of this application, when a radio frequency mainframe continuously outputs radio frequency energy, an impedance value and/or a temperature value of a radio frequency operated object is detected in real time; if any one of the impedance value and the temperature value is higher than a preset upper limit value, a radio frequency generating apparatus is controlled to stop outputting radio frequency energy and an emergency stop apparatus is controlled to cut off a radio frequency energy output path of the radio frequency mainframe. The above two manners of protection are performed at the same time, a succeeding rate of protection is improved, and safety of radio frequency operations is improved.

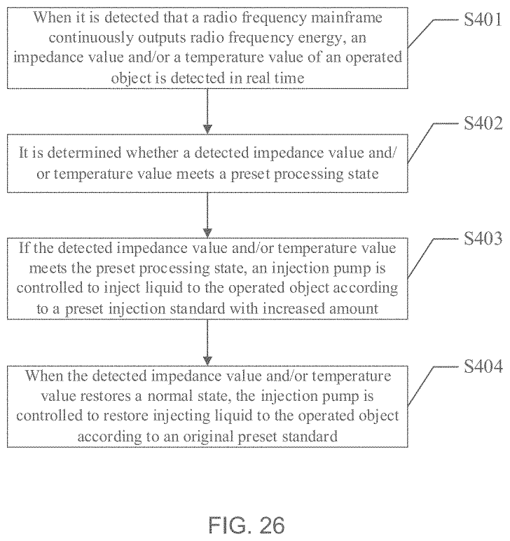

Referring to FIG. 26, which is an implementation flow chart of a method for protecting radio frequency operation abnormality provided by another embodiment of this application. The method can be applied in the radio frequency mainframe as shown in FIG. 23. As shown in FIG. 26, the method specifically comprises the follows.

Step 401, when it is detected that a radio frequency mainframe continuously outputs radio frequency energy, an impedance value and/or a temperature value of an operated object is detected in real time.

Step S402, it is determined whether a detected impedance value and/or temperature value meets a preset processing state.

It is detected in real time whether the impedance value of the operated object is higher than a second preset impedance threshold, or it is detected in real time whether an increasing ratio of the impedance value of the operated object is higher than a first preset ratio; if being higher than the second preset impedance threshold or higher than the first preset ratio, it is determined that the preset processing state is met. That is, although no preset abnormal state occurs, when the impedance value of the operated object is higher than a normal value or its increasing ratio is larger than a normal ratio, it is represented that the operated object appears abnormality, but an extent requiring stopping radio frequency energy output is not reached, thus it is determined that the preset processing state is met.

It is detected in real time whether the temperature value of the operated object is higher than a second preset temperature threshold, or it is detected in real time whether an increasing ratio of the temperature value of the operated object is higher than a second preset ratio; if being higher than the second preset temperature threshold or higher than the second preset ratio, it is determined that the preset processing state is met. That is, although no preset abnormal state occurs, when the temperature value of the operated object is higher than a normal value or its increasing ratio is larger than a normal ratio, it is represented that the operated object appears abnormality, but an extent requiring stopping radio frequency energy output is not reached, thus it is determined that the preset processing state is met.

Step S403, if the detected impedance value and/or temperature value meets the preset processing state, an injection pump is controlled to inject liquid to the operated object according to a preset injection standard with increased amount.

When the impedance value and/or the temperature value of the operated object appears abnormal increase, but does not reach the extent requiring stopping radio frequency energy output, an injection pump is controlled to inject liquid used to decrease the impedance value and/or the temperature value to the operated object according to a first preset injection standard with increased amount. Increasing injection amount can decrease the impedance value and the temperature value of the operated object.

Step S404, when the detected impedance value and/or temperature value restores a normal state, the injection pump is controlled to restore injecting liquid to the operated object according to an original preset standard.

When it is detected that the impedance value of the operated object is lower than a third preset impedance threshold, and/or the temperature value of the operated object is lower than a third preset temperature threshold, it is determined that the impedance value and/or the temperature value of the operated object has restored to a normal value. Thus, liquid is injected to the operated object according to the original preset injection standard with decreased amount, and injection amount according to the impedance value and/or the temperature value in a normal state is restored.

In this embodiment of this application, when it is detected that a radio frequency mainframe continuously outputs radio frequency energy, an impedance value and/or a temperature value of an operated object is detected in real time. If the impedance value and/or the temperature value increases to meet a preset processing state, an injection pump is controlled to increase injection mount to decrease the impedance value and/or the temperature value; if the impedance value and/or the temperature value restores to a normal value, the injection pump is controlled to restore to an original injection amount to keep the impedance value and/or the temperature value of the operated object being at the normal value. By the above-mentioned dynamic adjustment for abnormality of the impedance value and/or the temperature value that does not reaching the extent of stopping radio frequency energy output, it is possible to perform protection for the radio frequency object and the radio frequency mainframe in advance, and improve safety of radio frequency operations.

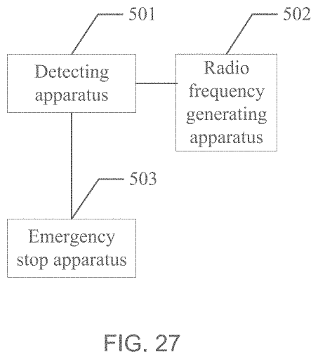

Referring to FIG. 27, which is a structural schematic diagram of a radio frequency mainframe provided by an embodiment of this application. In order to facilitate illustration, only parts relating to embodiments of this application are shown. The radio frequency mainframe is the radio frequency mainframe shown in above FIG. 23-FIG. 26, which comprises a detecting apparatus 501, a radio frequency generating apparatus 502, and an emergency stop apparatus 503.

Among them, the detecting apparatus 501 is configured to: when it is detected that the radio frequency mainframe continuously outputs radio frequency energy, detect preset kinds of detection data of a radio frequency operation in real time; the detecting apparatus 501 is further configured to: determine whether detected preset kinds of detection data meets a preset abnormal state; and the detecting apparatus 501 is further configured to: if the preset abnormal state is met, control the radio frequency generating apparatus 502 to stop outputting radio frequency energy and control the emergency stop apparatus 503 to cut off a radio frequency energy output path.

In this embodiment of this application, when a radio frequency mainframe continuously outputs radio frequency energy, preset kinds of detection data of a radio frequency operation is detected in real time; it is determined whether detected data meets a preset abnormal state; and if yes, a radio frequency generating apparatus is controlled to stop outputting radio frequency energy and an emergency stop apparatus is controlled to cut off a radio frequency energy output path of the radio frequency mainframe. The above two manners of protection are performed at the same time to prevent any one manner from failing or malfunctioning and causing protection failure, a succeeding rate of protection is improved, and safety of radio frequency operations is improved.

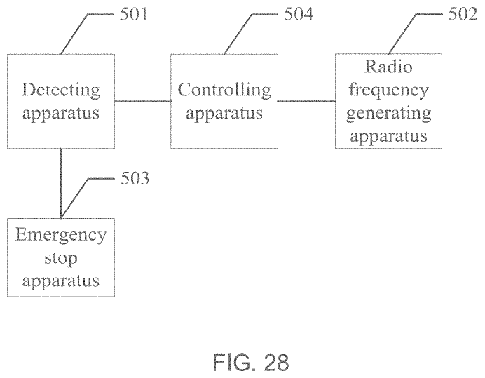

Referring to FIG. 28, which is a structural schematic diagram of a radio frequency mainframe provided by another embodiment of this application. The radio frequency mainframe is the radio frequency mainframe shown in above FIG. 23-FIG. 27, which differs from the embodiment shown in above FIG. 27 as follows.

Furthermore, the radio frequency mainframe further comprises a controlling apparatus 504.

The detecting apparatus 501 is further configured to transmit detection data meeting the preset abnormal state to the controlling apparatus 504 and thereby trigger the controlling apparatus 504 to control the radio frequency generating apparatus 502 to stop outputting radio frequency energy. The controlling apparatus 504 is configured to transmit information for stopping outputting radio frequency energy to the radio frequency generating apparatus 502 according to received detection data.

The controlling apparatus 504 can be further configured to analyze that the preset abnormal state occurs according to the detection data, and send stop information for stopping generating and outputting radio frequency energy to the radio frequency generating apparatus 502.

In another aspect, the detecting apparatus 501 is further configured to: after determining that the preset abnormal state occurs, directly send abnormal state prompt information to the controlling apparatus 504; the controlling apparatus 504 is further configured to send stop information for stopping generating and outputting radio frequency energy to the radio frequency generating apparatus according to the prompt information.

The detecting apparatus 501 is further configured to send cutting information to the emergency stop apparatus 503, and thereby cut off a radio frequency energy output path between the radio frequency mainframe and the operated object.

Furthermore, the detecting apparatus 501 is further configured to detect an impedance value and/or a temperature value of the operated object of the radio frequency operation in real time.

The detecting apparatus 501 is further configured to determine whether the impedance value of the operated object is higher than a first preset impedance threshold, and whether a duration time length of being higher than the first preset impedance threshold is larger than a preset time length, and/or whether the temperature value of the operated object is higher than a first preset temperature threshold, and whether a duration time length of being higher than the first preset temperature threshold is larger than the preset time length.

The detecting apparatus 501 or the controlling apparatus 504 is further configured to: if the impedance value of the operated object is higher than the first preset impedance threshold, and the duration time length of being higher than the first preset impedance threshold is larger than the preset time length, or if the temperature value of the operated object is higher than the first preset temperature threshold, and the duration time length of being higher than the first temperature impedance threshold is larger than the preset time length, determine that the preset abnormal state is met.

The detecting apparatus 501 is further configured to: detect in real time whether the impedance value of the operated object is higher than a second preset impedance threshold, or detected in real time whether an increasing ratio of the impedance value of the operated object is higher than a first preset ratio; if being higher than the second preset impedance threshold or higher than the first preset ratio, send the aforesaid detection information or prompt information to the controlling apparatus 504.

The controlling apparatus 504 is further configured to: according to the detection information or prompt information, send controlling instruction to an injection pump to control injection pump to inject liquid used to decrease the impedance value to the operated object according to a first preset injection standard with increased amount.

The detecting apparatus 501 is further configured to: detect in real time whether the temperature value of the operated object is higher than a second preset temperature threshold, or it is detected in real time whether an increasing ratio of the temperature value of the operated object is higher than a second preset ratio; if being higher than the second preset temperature threshold or higher than the second preset ratio, transmit the aforesaid detection information or prompt information to the controlling apparatus 504 for control.

The controlling apparatus 504 is further configured to: according to the detection information or prompt information, transmit controlling instruction to the injection pump and thereby make the injection pump inject liquid used to decrease the temperature to the operated object according to a first preset injection standard with increased amount.

In this embodiment of this application, when a radio frequency mainframe continuously outputs radio frequency energy, an impedance value and/or a temperature value of a radio frequency operated object is detected in real time; if any one of the impedance value and the temperature value is higher than a preset upper limit value, a radio frequency generating apparatus is controlled to stop outputting radio frequency energy and an emergency stop apparatus is controlled to cut off a radio frequency energy output path of the radio frequency mainframe. The above two manners of protection are performed at the same time, a succeeding rate of protection is improved, and safety of radio frequency operations is improved.

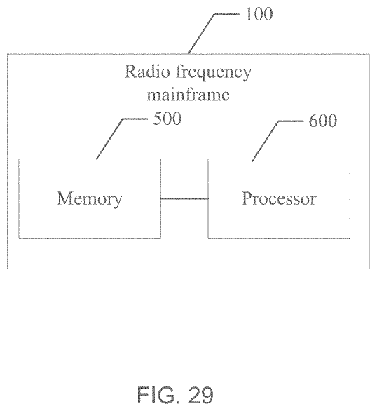

As shown in FIG. 29, an embodiment of this application further provides a radio frequency mainframe, which comprises a memory 500 and a processor 600, the processor 600 can be the detecting apparatus in the aforementioned embodiments, and can also be the controlling apparatus. The memory 500 is, for example, a hard disk drive memory, a non-volatile memory (such as a flash memory or other electronically programmable and deletion-restricted memory used to form a solid state drive, etc.), a volatile memory (such as a static or dynamic random access memory, etc.), and so on, The embodiments of this application are not limited.

The memory 500 stores executable program codes. A processor 600 coupled with the memory 500 calls the executable program codes stored in the memory to execute the above-described method for protecting radio frequency operation abnormality.

Further, an embodiment of this application further provides a computer-readable storage medium, the computer-readable storage medium can be set in the radio frequency mainframes in the above embodiments, and the computer-readable storage medium can be the memory 500 in the embodiment in the above embodiment shown in FIG. 29. The computer-readable storage medium stores a computer program, and the program, when being executed by a processor, implements the method for protecting radio frequency operation abnormality described in the embodiments shown in above FIG. 24, FIG. 25, and FIG. 26. Further, the computer-readable storage medium can also be a U-disk, a mobile hard disk, a read-only memory (ROM), a RAM, a magnetic disk or a CD-ROM, and other media that can store program codes.

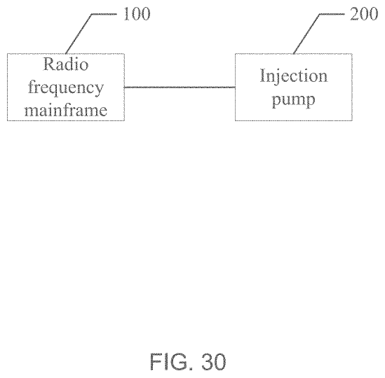

Further, referring to FIG. 30, an embodiment of this application further provides a radio frequency operation system, which comprises a radio frequency mainframe 100 and an injection pump 200.

The radio frequency mainframe 100 is configured to implement the method for protecting radio frequency operation abnormality described as FIG. 24, FIG. 25, and FIG. 26. The injection pump 200 is configured to inject liquid with a preset function to a radio frequency operated object under control of the radio frequency mainframe.

Other technical details refer to description of above-described embodiments.

Method and Apparatus for Dynamically Adjusting Radio Frequency Parameter and Radio Frequency Host

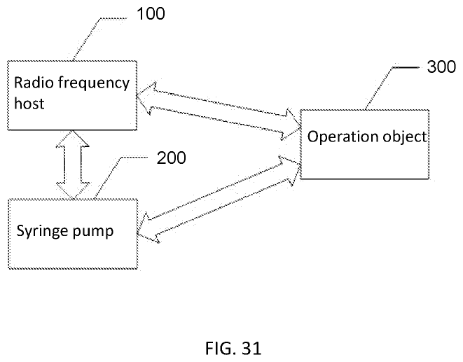

FIG. 31 is a schematic diagram showing an application scenario of a method for dynamically adjusting a radio frequency parameter according to an embodiment of the present invention. The method for dynamically adjusting the radio frequency parameter may be used to compare radio frequency data with a standard range and a limit range corresponding to a current operation stage by detecting the radio frequency data during a radio frequency operation, and confirm whether a problem occurs in the radio frequency operation, thereby interfering the radio frequency operation having the problem, such that the radio frequency operation may continue to be performed smoothly, the serious problem may be interrupted in time, and the safety of the radio frequency operation is improved.

Particularly, an execution main body of the method is a radio frequency host, and the radio frequency host may particularly be a device such as a radio frequency ablation instrument. As shown in FIG. 31, the radio frequency host 100 is connected with a syringe pump 200, and the radio frequency host 100 and the syringe pump 200 are connected with the operation object 300 as well. When the radio frequency operation starts, the radio frequency host 100 sends radio frequency energy to the operation object 300 by a radio frequency generation apparatus. The radio frequency host 100 controls the injection pump 200 to inject the operation object 300 with a cooling liquid. The radio frequency host 100 has radio frequency data standard ranges and radio frequency data limit ranges of various stages of an initial stage, a middle stage and a final stage of performing the radio frequency operation on the operation object 300. During the radio frequency operation, when characters of the operation object 300 change, the radio frequency data acting on the operation object will change therewith.

Further, the radio frequency data standard range and the radio frequency data limit range may be a numerical range, including the maximum value and the minimum value. If the real-time radio frequency data of the operation object 300 is greater than the maximum value or less than the minimum value of the standard range, the radio frequency data is enabled to be within the standard range by controlling an injection volume of the syringe pump. The injection volume may be controlled by controlling an injection flow rate. If the real-time radio frequency data of the operation object 300 is greater than the maximum value or less than the minimum value of the limit range, it is confirmed that a problem occurs in the radio frequency host 100 or the operation object 300, and the radio frequency operation has to be stopped. The radio frequency data standard range and the radio frequency data limit range of the radio frequency data may further be a radio frequency data change rate, that is, a radio frequency data slope. Within a preset detection duration, the real-time radio frequency data forms an excessive slope, which exceeds a preset slope, the purpose of adjusting the radio frequency data is achieved by adjusting the injection volume of the syringe pump, or the radio frequency operation is stopped to eliminate failures. Accordingly, the safety of the radio frequency operation is improved.

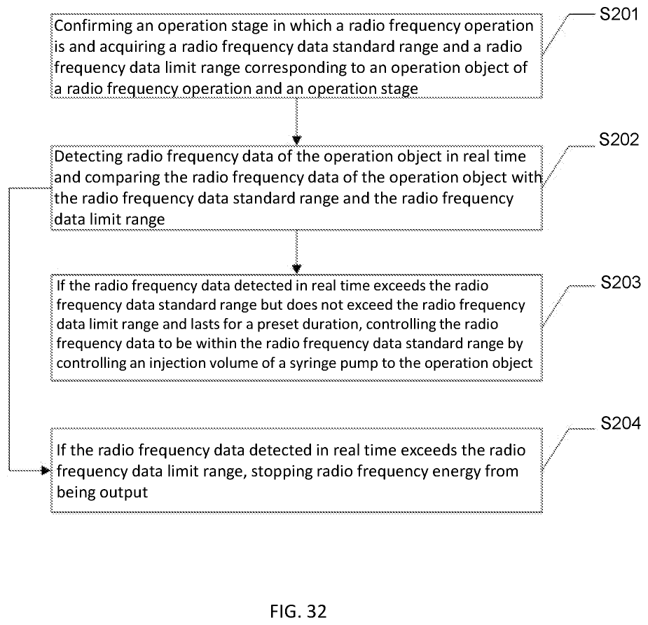

Reference is made to FIG. 32, which is a schematic flow chart of a method for dynamically adjusting a radio frequency parameter according to an embodiment of the present invention. The method may be applied to a radio frequency host as shown in FIG. 31. As shown in FIG. 32, the method particularly includes the following steps.

In step S201, a current operation stage in which a radio frequency operation is PAD confirmed, and a radio frequency data standard range and a radio frequency data limit range corresponding to an operation object of the radio frequency operation and the operation stage are acquired.

The radio frequency data standard range is within the radio frequency data limit range, that is, the minimum value of the radio frequency data standard range is greater than the minimum value of the radio frequency data limit range, and the maximum value of the radio frequency data standard range is greater than the maximum value of the radio frequency data limit range.

Particularly, for different types of operation objects or individual differences of the same type of operation objects, the radio frequency data standard range will be different. For different radio frequency operation stages of the same operation object, the radio frequency data standard range and the radio frequency data limit range are different. The operation object may be any object performing the radio frequency operation. For example, during radio frequency ablation, the operation object may be an abnormal tissue of a biological body, and the abnormal tissue is eliminated or reduced by means of ablation.

The radio frequency host has information on the radio frequency data standard ranges and the radio frequency data limit ranges of a specific operation object at different operation stages inside, for being read by a detection apparatus of the radio frequency host, and based on the radio frequency data of the operation object of the current radio frequency operation and the operation stage detected in real time, and being compared with the radio frequency data standard range and the radio frequency data limit range, respectively.

In step S202, the radio frequency data of the operation object is detected in real time, and compared with the radio frequency data standard range and the radio frequency data limit range, respectively.

The radio frequency operation will generate the radio frequency data when acting on the operation object, wherein the radio frequency data may particularly include an impedance value, a temperature value, a current value and a voltage value. The radio frequency host detects the above radio frequency data of the operation object in real time, and these radio frequency data feeds back whether the current radio frequency operation is normal.

In step S203, if the radio frequency data detected in real time exceeds the radio frequency data standard range but does not exceed the radio frequency data limit range and lasts for a preset duration, the radio frequency data is controlled to be within the radio frequency data standard range by controlling the injection volume of the injection pump to the operation object.

When it is detected that the radio frequency data of the test object in the current operation stage exceeds the radio frequency data standard range and lasts for the preset duration, the radio frequency data is adjusted to reach the normal standard range by controlling the injection volume of the syringe pump, the instability of the radio frequency data due to accidental factors is further eliminated and the intelligence of detection is improved.

In step S204, if the radio frequency data detected in real time exceeds the radio frequency data limit range, the radio frequency energy is stopped from being output.

If the radio frequency data detected in real time exceeds the radio frequency data standard range, indicating that a serious problem occurs in the radio frequency operation, in order to protect the safety of the radio frequency host and the operation object, the radio frequency energy is immediately stopped from being output. Particularly, a detection module may send the radio frequency data to a processor of the radio frequency host, and the processor sends a stop signal to a radio frequency signal generation apparatus of the radio frequency host, such that the radio frequency signal generation apparatus is stopped from outputting the radio frequency signal.

In the embodiment of the present invention, the radio frequency data standard range corresponding to the operation object of the radio frequency operation and the current operation stage is acquired, and the radio frequency data detected in real time is compared with the radio frequency data standard range and the radio frequency data limit range, respectively. If the radio frequency detected in real time exceeds the radio frequency data standard range but does not exceed the radio frequency data limit range and lasts for the preset duration, the radio frequency data is controlled to be within the radio frequency data standard range by controlling the injection volume of the syringe pump to the operation object. Accordingly, the radio frequency data is dynamically adjusted within the radio frequency data standard range, and the success rate of the radio frequency operation is improved. If the radio frequency data detected in real time exceeds the radio frequency data limit range, it is confirmed that a problem exists in the radio frequency host of the current radio frequency operation or the operation object, and the radio frequency energy is stopped from being output. Therefore, the radio frequency host and the operation object are prevented from being damaged, and the safety of the radio frequency operation is improved.

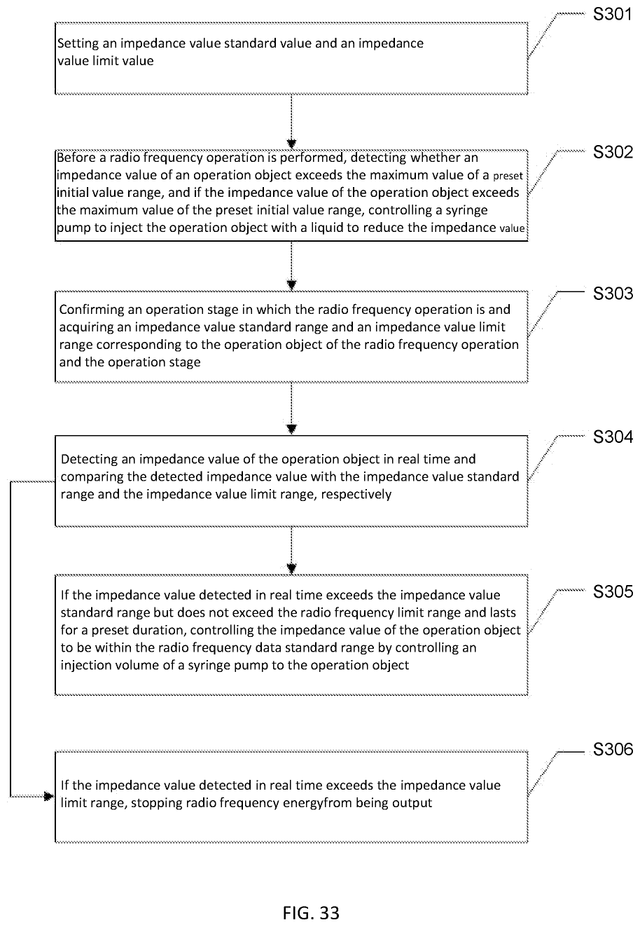

Reference is made to FIG. 33, which is a flow chart of an implementation of a method for dynamically adjusting a radio frequency parameter according to another embodiment of the present invention. The method may be applied to a radio frequency host as shown in FIG. 31. As shown in FIG. 33, the method particularly includes the following steps.

In step S301, an impedance value standard range and an impedance value limit range are set.

In response to a setting operation of a user, an input interface of the lowest value, the highest value and a change rate of the impedance value is displayed, wherein the setting operation may be a user input, or may be called from a memory of the radio frequency host or a database of a server connected with the radio frequency host according to an instruction from the user.

A first lowest value, a first highest value and a first change rate of the impedance value set by the user are acquired, the first lowest value and the first highest value input by the user are used as the lowest value and the highest value of a standard numerical interval, and the first change rate input by the user is used as the standard slope.

A second lowest value, a second highest value and a second change rate of the impedance value set by the user are acquired, the second lowest value and the second highest value input by the user are used as the lowest value and the highest value of a limit numerical interval, and the second change rate set by the user is used as the standard slope.

Among them, the first lowest value is higher than the second lowest value, the first highest value is lower than the second lowest value, the first change rate is lower than the second change rate, and the low change rate indicates that the value changes a little per unit time.

Particularly, the impedance value is set corresponding to a model of the radio frequency host, a task of the radio frequency operation and a nature of the operation object, and an operation position of the radio frequency operation on the operation object. Such a correspondence relationship is known and may be input by the user or stored in related devices such as the radio frequency host or the server in advance.

In step S302, before the radio frequency operation is performed, it is detected whether the impedance value of the operation object exceeds the highest value of a preset initial value range, and if the impedance value of the operation object exceeds the highest value of the preset initial value range, the syringe pump is controlled to inject the operation object with a liquid to reduce the impedance value.

If the detected impedance value of the operation object is higher than the highest value of the preset initial value range, the syringe pump is controlled to inject the operation object with the liquid to reduce the impedance value of the operation object, until the impedance value meets the preset initial value range. That is, before the radio frequency operation starts, the initial impedance value of the operation object is set to be within the normal initial value range, so as to reduce the influence on the detection and the determination based on the impedance value during the radio frequency operation due to the deviation of the initial impedance value from the normal range after the radio frequency operation starts.

The initial value range corresponds to the nature of the operation object and a specific position of the radio frequency operation on the operation object, and is a general range obtained based on actual measurement values of a plurality of operation objects. For example, when the radio frequency operation is radio frequency ablation, the operation object is a human body and a specific location is a lung tissue, and the initial value range is 250Ω to 350Ω (ohm).

In step S303, a current operation stage in which the radio frequency operation is PAD confirmed, and an impedance value standard range and an impedance value limit range corresponding to the operation object of the radio frequency operation and the operation stage are acquired.

The impedance value standard range may be a standard numerical interval of the impedance value, and the standard numerical interval is a numerical interval including the lowest value and the highest value. During the radio frequency operation, the impedance value standard range is preferably 150Ω to 500Ω, or a standard slope set according to the nature of the operation object, that is, a standard change rate of the impedance value without being adjusted is confirmed according to the nature of the operation object under a premise that the impedance value of the operation object is not less than 150Ω and is not greater than 500Ω during the radio frequency operation. No adjustment on a real-time change rate of the impedance value below the standard change rate is required for the operation object. The standard change rate is the standard slope.

The impedance value limit range may be a limit numerical interval of the impedance value, and the limit numerical interval is a numerical interval including the lowest value and the highest value. During the radio frequency operation, the impedance value limit range is preferably 50Ω to 600Ω, or is a limit slope set according to the nature of the operation object, that is, a limit change rate of a safe impedance value is confirmed according to the nature of the operation object under a premise that the impedance value of the operation object is not less than 50Ω and is not greater than 600Ω during the radio frequency operation. A real-time change rate of the impedance value below the standard change rate is safe for the operation object. The standard change rate is the standard slope.

Specific values of standard numerical interval, the standard slope, the limit numerical interval and the limit slope of the impedance value are related to the operation object and the operation stage in which the radio frequency operation is performed, and will be not particularly limited.

In step S304, the impedance value of the operation object is detected in real time, and the detected impedance value is compared with the impedance value standard range and the impedance value limit range, respectively.

The impedance value of the operation object may be directly detected by an impedance detection circuit, or a current value of the operation object may be detected by a current detection circuit, and a voltage value of the operation object may be detected by a voltage detection circuit. The impedance value of the operation object is calculated according to a calculation formula of the current value, the voltage value and the impedance value.

The impedance value detected in real time is compared with the standard numerical interval of the impedance value corresponding to the current operation stage of the operation object and/or the standard slope of the impedance value in real time, and compared with the limit numerical interval of the impedance value corresponding to the current operation stage of the operation object and/or the limit slope of the impedance value.

In step S305, if the impedance value detected in real time exceeds the impedance value standard range but does not exceed the radio frequency data limit range and lasts for the preset duration, the impedance value of the operation object is controlled to be within the radio frequency data standard range by controlling the injection volume of the injection pump to the operation object.

Particularly, if the impedance value of the operation object detected in real time is lower than the lowest value of the standard numerical interval and/or a decrease rate of the impedance value of the operation object is greater than the first standard slope and lasts for the preset duration, the injection pump is controlled to reduce the amount of the liquid injected into the operation object according to a preset first injection volume.

Any one or both of two cases where the impedance value of the operation object is lower than the lowest value of the standard numerical interval and lasts for the preset duration and the decrease rate of the impedance value of the operation object is greater than the first standard slope and lasts for the preset duration indicates or indicate that the impedance value of the operation object is too low or decreases too quickly and it is necessary to increase the impedance value. Therefore, the injection volume of the injection pump to the operation object is reduced, and the injection volume may be controlled by controlling an injection flow rate of the liquid. Within a fixed duration, the greater the flow rate is, the greater the injection volume is.

If the impedance value of the operation object detected in real time is higher than the highest value of the standard numerical interval and/or the increase rate of the impedance value of the operation object is greater than the second standard slope and lasts for the preset duration, the injection pump is controlled to increase the amount of the liquid injected into the operation object according to a preset second injection volume.

Any one or both of two cases where the impedance value of the operation object is higher than the highest value of the standard numerical interval and lasts for the preset duration and the increase rate of the impedance value of the operation object is greater than the second standard slope and lasts for the preset duration indicates or indicate that the impedance value of the operation object is too high or increases too rapidly and it is necessary to reduce the impedance value. Therefore, the injection volume of the injection pump to the operation object is increased.

Further, the impedance value may change due to accidental factors. In order to prevent the instability of a working process of the syringe pump due to frequent adjustment on the impedance value from influencing the effect of the radio frequency operation, the accidental factors may be eliminated after the preset duration is elapsed and the step of dynamically adjusting the impedance value of the operation object is started.

If the impedance value of the operation object may not be controlled to be within the radio frequency data standard range by controlling the injection volume of the injection pump to the operation object after a preset adjustment duration is elapsed, the radio frequency operation is stopped.

In step S306, if the impedance value detected in real time exceeds the impedance value limit range, the radio frequency energy is stopped from being output.

Particularly, the limit slope of the impedance value includes a first limit slope used to indicate a decrease rate of the impedance value and a second limit slope used to indicate the increase rate of the impedance value. The limit numerical interval, the first limit slope and the second limit slope are limit abnormal values in nature, that is, no matter what operation stage in which the impedance value of the operation object detected in real time is, the radio frequency energy has to be stopped immediately from being output to the operation object provided that at least one of the following first conditions is met, the first conditions may include: the impedance value is higher than the maximum value of the limit numerical interval, the impedance value is lower than the minimum value of the limit numerical interval, the decrease rate of the impedance value is greater than the first limit slope, and the increase rate of the impedance value is greater than the second limit slope. Among them, the decrease rate of the impedance value is a ratio of a decrement of the impedance value of the operation object per unit duration to the unit duration; and the increase rate of the impedance value is a ratio of an increment of the impedance value of the operation object per unit duration to the unit duration.

Further, while the radio frequency energy is stopped from being output to the operation object, a text indicator is displayed and an audible and visual alarm is given. Particularly, when the impedance value of the operation object detected in real time is lower than the lowest value of the limit numerical interval or the decrease rate of the impedance value of the operation object detected in real time is greater than the first limit slope, a first text indicator is displayed and the audible and visual alarm is given.

When the impedance value of the operation object detected in real time is higher than the highest value of the limit numerical interval or the increase rate of the impedance value of the operation object detected in real time is greater than the first limit slope, a second text indicator is displayed and the audible and visual alarm is given.

The first text indicator for example displays a text “LLL” on a display screen of the radio frequency host, and the second text indicator for example displays a text “HHH” on the display screen of the radio frequency host. The audible and visual alarm includes both an audible alarm and a visual alarm.

Further, when it is detected that the impedance value of the operation object exceeds the above non-limit abnormal value, the text indicator may be displayed and the audible and visual alarm may be given. Particularly, the text indicator and the audible and visual alarm may be different from those when the impedance value of the operation object exceeds the limit abnormal value in terms of contents and forms. The audible and visual alarm is distinguished from the above audible and visual alarm in the terms of forms.

Further, after it is detected that the impedance value of the operation object exceeds the above-mentioned non-limit abnormal value and lasts for the preset duration, the radio frequency energy is stopped from being output to the operation object, and the text indicator is displayed and the audible and visual alarm is given. At this time, the text indicator and the audible and visual alarm are the same as those when the impedance value of the operation object exceeds the limit abnormal value in the terms of contents and forms.

Further, if the detected impedance value of the operation object exceeds the radio frequency data standard range, a correspondence relationship between the impedance value of the operation object and the time of the radio frequency operation is displayed on a display interface in the form of a line with a first color; and if the detected impedance value of the operation object does not exceed the radio frequency data standard range, a correspondence relationship between the impedance value of the operation object and the time of the radio frequency operation is displayed on the display interface in the form of a line with a second color.

The reflectivity of the first color is higher than that of the second color, the greater the reflectivity is, the more the light dazzles, and the higher a reminding degree to human eyes is. For example, a red color reflects 67% of light, a yellow color reflects 65% of light, a green color reflects 47% of light, and a cyan color reflects 36% of light. The first color may be the red or yellow color, and the second color may be the green or cyan color.

For technical details of the above steps, reference is made to the description of the embodiment as shown in FIG. 32, which will be omitted here.

In the embodiment of the present invention, before the radio frequency operation is performed, the impedance value of the operation object is reduced to be within the normal initial value range in a fashion of injecting the liquid by the syringe pump, so as to improve the accuracy of detecting the impedance value during the subsequent radio frequency operation and improve the success rate of the radio frequency operation. The standard numerical interval of the impedance value and the standard change slope of the impedance value corresponding to the operation object of the radio frequency operation and the current operation stage are acquired. The impedance value of the operation object detected in real time is compared with the standard numerical interval and/or the standard slope and with the limit numerical interval and/or the limit slope in real time. The radio frequency data is controlled to be within the radio frequency data standard range by controlling the injection volume of the syringe pump to the operation object. Accordingly, the radio frequency data is dynamically adjusted within the radio frequency data standard range, and the success rate of the radio frequency operation is improved. If the impedance value of the operation object detected in real time exceeds the limit numerical interval and/or the change rate of the impedance value is greater than the limit slope, it is confirmed that a problem occurs in the radio frequency host of the current radio frequency operation or the operation object, the radio frequency energy is stopped from being output. As a result, the radio frequency host and the operation object are prevented from being damaged, and the safety of the radio frequency operation is improved. The text indicator is displayed and the audible and visual alarm is given, which further remind a radio frequency operator of paying attention to the safety of the radio frequency operation.

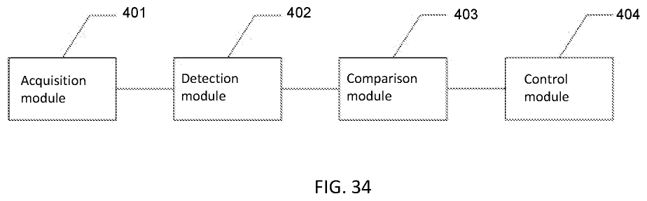

Reference is made to FIG. 34, which is a schematic diagram showing a structure of an apparatus for dynamically adjusting a radio frequency parameter according to an embodiment of the present invention. In order to facilitate the illustration, parts related to the embodiment of the present invention are only shown. The apparatus may be disposed in the above radio frequency host. The apparatus includes: an acquisition module 401, which is configured to confirm an operation stage in which a radio frequency operation is and acquire a radio frequency data standard range and a radio frequency data limit range corresponding to an operation object of the radio frequency operation and the operation stage, wherein the radio frequency data standard range is within the radio frequency data limit range; a detection module 402, which is configured to detect radio frequency data of the operation object in real time; a comparison module 403, which is configured to compare the detected radio frequency data with the radio frequency data standard range and the radio frequency data limit range; and a control module 404, which is configured to if the radio frequency data detected in real time exceeds the radio frequency data standard range but does not exceed the radio frequency data limit range and lasts for a preset duration, control the radio frequency data to be within the radio frequency data standard range by controlling an injection volume of the syringe pump to the operation object, and if the radio frequency data detected in real time exceeds the radio frequency data limit range, stop radio frequency energy from being output.

Further, the acquisition module 401 is further configured to acquire a standard numerical interval of an impedance value of the operation object, a standard change slope of the impedance value of the operation object, a limit numerical interval of the impedance value of the operation object, and a limit slope of the impedance value change of the operation object in the operation stage.

The acquisition module 401 is further configured to display an input interface of the lowest value, the highest value and the change rate of the impedance value in response to a setting operation of a user, acquire a first lowest value, a first highest value, a first change rate, a second lowest value, a second highest value and a second change rate use the first lowest value and the first highest value as the lowest value and the highest value of the standard numerical interval and use the first change rate input by the user as the standard slope.

The second lowest value and the second highest value are taken as the lowest value and the highest value of the limit numerical interval, and the second change rate input by the user is taken as the limit slope.

Further, the limit slope includes a first limit slope used to indicate a decrease rate of the impedance value and a second limit slope used to indicate an increase rate of the impedance value. The control module 404 is further configured to when the impedance value of the operation object detected in real time meets at least one of preset conditions, stop from outputting radio frequency energy to the operation object, wherein the preset conditions include: the impedance value of the operation object detected in real time exceeds the limit numerical interval, the decrease rate of the impedance value of the operation object detected in real time is greater than the first limit slope, and the increase rate of the impedance value of the operation object detected in real time is greater than the second limit slope.

Further, the apparatus further includes a pre-warning module (not shown in the drawing), wherein the pre-warning module is configured to when the impedance value of the operation object detected in real time is lower than the lowest value of the limit numerical interval or the decrease rate of the impedance value of the operation object detected in real time is greater than the first limit slope, display a first text indicator and give an audible and visual alarm; and when the impedance value of the operation object detected in real time is higher than the highest value of the limit numerical interval or the increase rate of the impedance value of the operation object detected in real time is greater than the second limit slope, display a second text indicator and give the audible and visual alarm.

Further, the standard slope includes a first standard slope used to indicate a decrease rate of the impedance value and a second standard slope used to indicate an increase rate of the impedance value. The control module 404 is further configured to if the impedance value of the operation object detected in real time is lower than the lowest value of the standard numerical interval and/or a decrease rate of the impedance value of the operation object is greater than the first standard slope and lasts for the preset duration, control the injection pump to reduce the amount of the liquid injected into the operation object according to a preset first injection volume; and if the impedance value of the operation object detected in real time is higher than the highest value of the standard numerical interval and/or an increase rate of the impedance value of the operation object is greater than the second standard slope and lasts for the preset duration, control the injection pump to increase the amount of the liquid injected into the operation object according to a preset second injection volume.

Further, the detection module 402 is further configured to before the radio frequency operation is performed, detect whether the impedance value of the operation object exceeds the highest value of a preset initial value range.

The control module 404 is further configured to if the impedance value of the operation object exceeds the highest value of the preset initial value range, control the syringe pump to inject the operation object with the liquid to reduce the impedance value, until the impedance value meets a preset initial value range.

Further, the apparatus further includes a display module (not shown in the drawing), wherein the display module is configured to if the detected impedance value of the operation object exceeds the radio frequency data standard range, display a correspondence relationship between the impedance value of the operation object and the time of the radio frequency operation on a display interface in the form of a line with a first color; if the detected impedance value of the operation object does not exceed the radio frequency data standard range, display a correspondence relationship between the impedance value of the operation object and the time of the radio frequency operation on the display interface in the form of a line with a second color; and wherein the reflectivity of the first color is higher than that of the second color.

In the embodiment of the present invention, before the radio frequency operation is performed, the impedance value of the operation object is reduced to be within the normal initial value range in a fashion of injecting the liquid by the syringe pump, so as to improve the accuracy of detecting the impedance value during the subsequent radio frequency operation and improve the success rate of the radio frequency operation. The standard numerical interval of the impedance value and the standard change slope of the impedance value corresponding to the operation object of the radio frequency operation and the current operation stage are acquired. The impedance value of the operation object detected in real time is compared with the standard numerical interval and/or the standard slope and with the limit numerical interval and/or the limit slope in real time. The radio frequency data is controlled to be within the radio frequency data standard range by controlling the injection volume of the syringe pump to the operation object. Accordingly, the radio frequency data is dynamically adjusted within the radio frequency data standard range, and the success rate of the radio frequency operation is improved. If the impedance value of the operation object detected in real time exceeds the limit numerical interval and/or the change rate of the impedance value is greater than the limit slope, it is confirmed that a problem occurs in the radio frequency host of the current radio frequency operation or the operation object, and the radio frequency energy is stopped from being output. As a result, the radio frequency host and the operation object are prevented from being damaged, and the safety of the radio frequency operation is improved. The text indicator is displayed and the audible and visual alarm is given, which further remind a radio frequency operator of paying attention to the safety of the radio frequency operation.

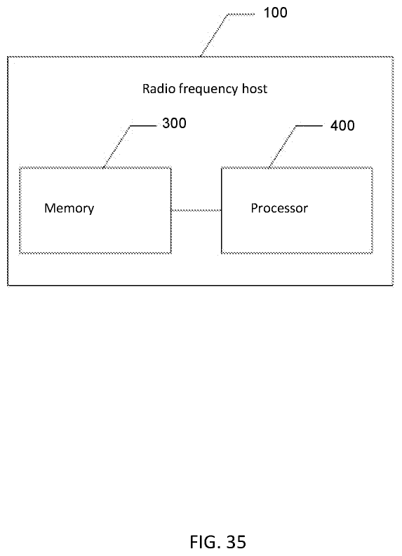

As shown in FIG. 35, embodiments of the present invention further provide a radio frequency host, including a memory 300 and a processor 400. The processor 400 may be a control module 404 in the apparatus for dynamically adjusting the radio frequency parameter in the foregoing embodiment. The memory 300 may be for example a hard disk drive memory, a non-volatile memory (such as a flash memory or another electronically programmable and restricted delete memory used to form a solid-state drive and the like), a volatile memory (such as a static or dynamic random access memory and the like) and the like, which will not be limited in the embodiment of the present invention.

The memory 300 stores an executable program code; and the processor 400 coupled with the memory 300 calls the executable program code stored in the memory to execute the method for dynamically adjusting the radio frequency parameter as described above.

Further, embodiments of the present invention further provide a computer-readable storage medium. The computer-readable storage medium may be provided in the radio frequency host in each of the foregoing embodiments, and the computer-readable storage medium may be a memory 300 in the embodiment as shown in FIG. 35. A computer program is stored on the computer-readable storage medium, and when being executed by the processor, implements the method for dynamically adjusting the radio frequency parameter according to the embodiments as shown in FIG. 32 and FIG. 33. Further, the computer-readable storable medium may further be a U disk, a mobile hard disk, a read-only memory (ROM), a RAM, a magnetic disk or an optical disk, and other various media that may store the program code.

It should be noted that for simplicity of description, the foregoing method embodiments are all expressed as a series of action combinations, but because according to the present invention, some steps may be performed in other sequences or simultaneously, those skilled in the art should appreciate that the present invention is not limited by the described sequence of actions. Secondly, those skilled in the art should further appreciate that the embodiments described in the specification are all preferred embodiments, and the involved actions and modules are not necessarily all required by the present invention.

In the above-mentioned embodiments, descriptions of the embodiments have particular emphasis respectively. For parts that are not described in detail in a certain embodiment, reference may be made to related descriptions of other embodiments.

The above is a description of the method and the apparatus for dynamically adjusting the radio frequency parameter and the radio frequency host according to the present invention. For those skilled in the art, changes may be made to specific implementations and application scopes according to the ideas of the embodiments of the present invention. In summary, the content of this specification should not be construed as a limitation on the present invention.

Method and Apparatus for Safety Control of Radio Frequency Operation, and Radio Frequency Mainframe

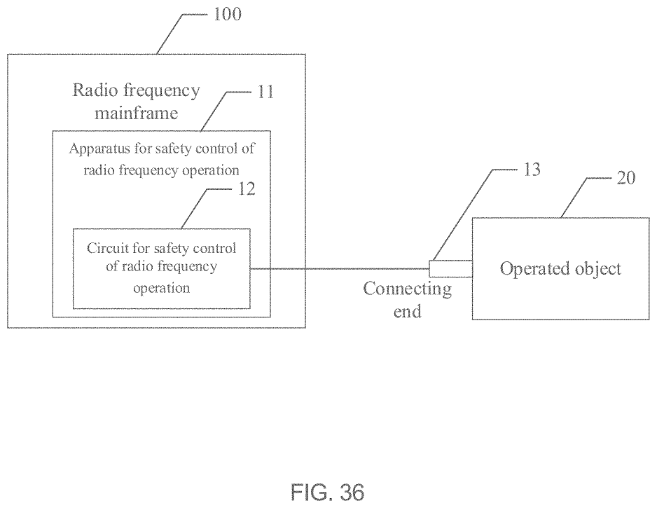

Referring to FIG. 36, which is a schematic diagram of an application scene of a method for safety control of radio frequency operation provided by an embodiment of this application; the method for safety control of radio frequency operation can be used to control safety problems relating to connection between a radio frequency mainframe and a radio frequency operated object through the radio frequency mainframe, and thereby improve safety and intelligence of radio frequency operations.

Specifically, the radio frequency mainframe may be a device such as a radio frequency ablation apparatus, and the radio frequency operated object may be any object that needs a radio frequency operation. For example, when the radio frequency mainframe is a radio frequency ablation apparatus, the radio frequency operated object may be an animal body that needs to ablate mutated tissues in the body. As shown in FIG. 36, a radio frequency mainframe 100 is connected with an operation object 200. The radio frequency mainframe 100 is provided therein with a radio frequency operation safety control device 11 and a radio frequency circuit 12. The radio frequency circuit 12 is used to detect whether a connection between the radio frequency mainframe 100 and the operated object 200 meets a standard, and the radio frequency circuit 12 has a connection terminal 13 (specifically, it may be a neutral electrode), the radio frequency mainframe 100 is connected with the operated object 200 through the connection terminal 13. The radio frequency mainframe 100 controls whether to output a radio frequency signal and the output power of the output radio frequency signal through the radio frequency operation safety control device 11.

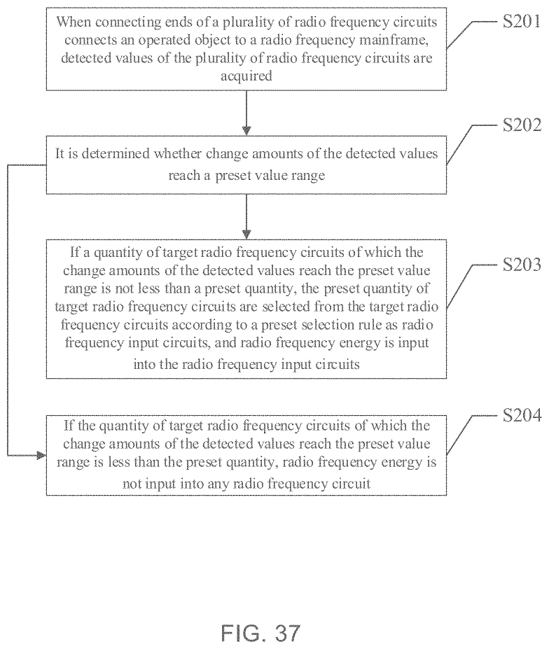

Referring to FIG. 37, which is a schematic flow chart of a method for safety control of radio frequency operation provided by an embodiment of this application. The method can be applied to the radio frequency mainframe shown in FIG. 36, as shown in FIG. 37, the method specifically comprises the follows.

Step S201, when connecting ends of a plurality of radio frequency circuits connects an operated object to a radio frequency mainframe, detected values of the plurality of radio frequency circuits are acquired.

Specifically, an execution subject of this embodiment is the radio frequency mainframe, and the radio frequency mainframe can detect whether a connecting end has been connected to the operation object. The connecting end is used to connect an operated object to the radio frequency mainframe, and whether tightness of the connection meets a connection standard is a prerequisite of whether the radio frequency mainframe can complete a radio frequency operation smoothly and safely.

The connecting end can be a neutral electrode; the operated object is an object or target for the radio frequency mainframe to perform a radio frequency operation.

The radio frequency circuit includes a detection circuit, which is used to detect whether a connection between a connecting end and an operated object meets a connection standard; when the connecting end is a neutral electrode, the detection circuit specifically detects whether an attachment degree between the neutral electrode and the operated object meets an attachment standard.

The radio frequency circuit further includes a radio frequency radio module; under control of the radio frequency mainframe, the radio frequency module inputs a radio frequency signal into the radio frequency circuit to execute a radio frequency operation for the operated object.

Step S202, it is determined whether change amounts of the detected values reach a preset value range.

Whether a change amount of a detected value reaches a preset value range is a determination standard for the radio frequency mainframe to determine whether a connection between a connecting end and an operated object meets a connection standard.

Specifically, the detected value can be an impedance value, and can also be a change value of a voltage value of a primary coil of a transformer.

If a change amount of the detected values reaches a preset value range, the radio frequency mainframe determines that the connection between the connecting end and the operated object meets the connection standard; if the change amount of the detected values does not reach the preset value range, the radio frequency mainframe determines that the connection between the connecting end and the operated object does not meet the connection standard.

Step S203, if a quantity of target radio frequency circuits of which the change amounts of the detected values reach the preset value range is not less than a preset quantity, the preset quantity of target radio frequency circuits are selected from the target radio frequency circuits according to a preset selection rule as radio frequency input circuits, and radio frequency energy is input into the radio frequency input circuits.

For example, if the preset quantity is 2, that is, target radio frequency circuits of which connections between connecting ends and the operated object meet the connection standard reach 2, 2 of the target radio frequency circuits are selected from these target radio frequency circuits according to a preset selection rule as radio frequency input circuits, and radio frequency energy is input thereto to be provided to the operated object.

It should be noted that each radio frequency circuit and its connecting end have their own preset numbers. According to these numbers, the radio frequency mainframe can know which operation area of the radio frequency operation the connecting terminal of the radio frequency circuit is connected to, and determine how many radio frequency circuits each operation area needs to connect, that is, how many connecting ends are connected, according to nature of the current radio frequency operation.

The selection rule is: according to an operation area and the number of connecting ends required by the current radio frequency operation, to select a preset quantity of target radio frequency circuits from the target radio frequency circuits meeting the connection standard as the radio frequency input circuits.

Distribution of the target radio frequency circuits should be capable of meeting selection requirement, and both the operation area and the quantity of the distribution are met.

Step S204, if the quantity of target radio frequency circuits of which the change amounts of the detected values reach the preset value range is less than the preset quantity, radio frequency energy is not input into any radio frequency circuit.

If the quantity of target radio frequency circuits of which the change amounts of the detected values reach the preset value range is less than the preset quantity, it is unable to meet requirement of the present radio frequency operation. Therefore, no radio frequency energy is output, that is, radio frequency energy is not input into any radio frequency circuit. It is also possible to simultaneously trigger an alarm module to issue an alarm, including flashing of an alarm light and tweeting of alarm sound.

In this embodiment of this application, when an operated object is connected to a radio frequency mainframe through connecting ends of radio frequency circuits, according change amounts of detected values of the radio frequency circuits, it is determined whether a quantity of target radio frequency circuits of which the change amounts reach a preset value range reaches a preset quantity, that is, it is determined whether the connection between the connecting ends and the operated object meets a connection standard; if reaching, the preset quantity of target radio frequency circuits are selected from the target radio frequency circuits as radio frequency input circuits, and radio frequency signals are controlled to input; if not reaching, no radio frequency input is performed, so as to avoid subsequent radio frequency operations from being affected by connection that does not meet the standard. Accordingly, the above-described method for safety control of radio frequency operation can automatically determine whether connection between an operated object and radio frequency circuits meets a standard, and does not perform radio frequency energy input when radio frequency circuits meeting the standard do not meet requirement of radio frequency operation in quantity, thereby improving safety and intelligence of radio frequency operation.

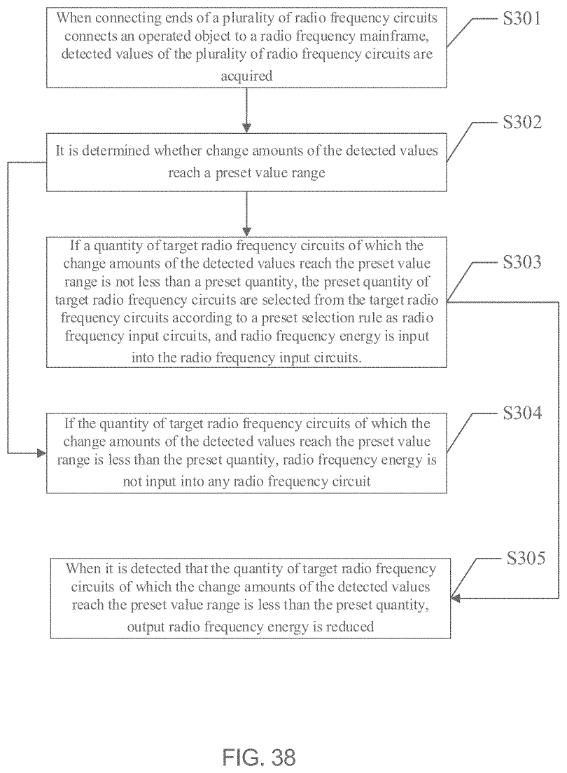

Referring to FIG. 38, which is an implementation flow chart of a method for safety control of radio frequency operation provided by another embodiment of this application. The method can be applied to the radio frequency mainframe shown in FIG. 36, as shown in FIG. 38, the method specifically comprises the follows.

Step S301, when connecting ends of a plurality of radio frequency circuits connects an operated object to a radio frequency mainframe, detected values of the plurality of radio frequency circuits are acquired.

Step S302, it is determined whether change amounts of the detected values reach a preset value range.

Specifically, a change amount of a detected value is a change amount of a voltage of a primary coil of a transformer in a radio frequency circuit.

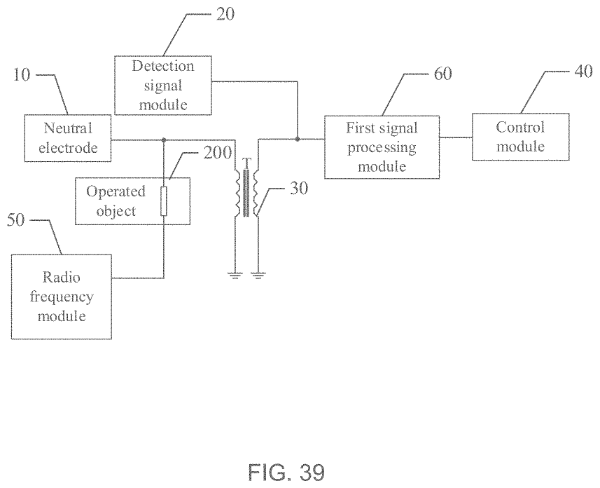

Referring to FIG. 39, FIG. 39 is a structural schematic diagram of a radio frequency circuit. Each radio frequency circuit has a connecting end, that is, a neutral electrode 10. In this embodiment, it is possible connect a plurality of radio frequency circuits to an operated object at the same time. Furthermore, each radio frequency further includes a detection signal module 20, a transformer 30, a control module 40, and a radio frequency module 50; wherein the detection signal module 20 and the neutral electrode module 10 are respectively connected to a primary coil and a secondary coil of the transformer 30, and the neutral electrode 10 forms a loop with the secondary coil of the transformer 30 when being attached to the operated object; the detection signal module 20 sends a detecting signal; the control module 40 can specifically be processor such as a CPU, which can be a CPU in the radio frequency mainframe and can also be an individual CPU of the radio frequency circuit, can control the detection signal module 20 to input the detecting signal into the circuit, and can also determine whether a connection between the neutral electrode 10 and the radio frequency mainframe meets a connection standard according to a lowering value of a voltage of the primary coil of the transformer 30, and control the radio frequency module 50 to output radio frequency energy to the operated object 200. The radio frequency circuit further includes a first signal processing module 60, the first signal processing module 60 includes a filter, a resonators, an amplifier, etc., so as to perform filtering, amplification, and so on for the detection signal, and the specific circuit structure is not particularly limited.

Specifically, when neutral electrodes of a plurality of radio frequency circuits are attached to the operated object to connect the operated object to the radio frequency mainframe, the control module controls the detection signal module to send a detecting signal to the transformer, and acquires voltage values of primary coils of transformers of a plurality of radio frequency circuits. When a voltage value lowers to the preset value range, it is determined that an attachment degree between a neutral electrode 10 and the operated object 200 meets a preset standard. The preset value range is preferably 1.6-2.0V (volt).

A specific implementation manner in a circuit can be that: it is possible to set high electric level signals and low electric level signals according to the preset value range, for example, a voltage signal of 2.3-2.8V is set to be a high electric level signal, and a voltage signal of 0.6-1.0V is set to be a low electric level signal. Thus, when the control module detects that a detecting signal changes from a high electric level signal to a low electric level signal, it can be determined that an attachment degree between a neutral electrode 10 and the operated object 200 meets the preset standard; if a change amount of a voltage value does not reach the preset value range, the signal electric level does not generate an obvious change, the control module 40 determines that an attachment degree between a neutral electrode 10 and the operated object 200 does not meet the preset standard.

Step S303, if a quantity of target radio frequency circuits of which the change amounts of the detected values reach the preset value range is not less than a preset quantity, the preset quantity of target radio frequency circuits are selected from the target radio frequency circuits according to a preset selection rule as radio frequency input circuits, and radio frequency energy is input into the radio frequency input circuits.

Among them, the selecting the preset quantity of target radio frequency circuits from the target radio frequency circuits according to a preset selection rule as radio frequency input circuits specifically comprises: acquiring preset numbers of the target radio frequency circuits; determining connecting areas corresponding to the target radio frequency circuits according to the numbers; and correspondingly selecting the radio frequency input circuits in the target radio frequency circuits according to operation areas of the current radio frequency operation and a quantity of circuits required by each operation area.

Step S304, if the quantity of target radio frequency circuits of which the change amounts of the detected values reach the preset value range is less than the preset quantity, radio frequency energy is not input into any radio frequency circuit.

Technical details of the above steps refer to the description of the embodiment shown in above FIG. 37, and are not repeated here.

Step S305, when it is detected that the quantity of target radio frequency circuits of which the change amounts of the detected values reach the preset value range is less than the preset quantity, output radio frequency energy is reduced.

After radio frequency energy is input, if it is detected that the quantity of target radio frequency circuits of which the change amounts of the detected values reach the preset value range is less than the preset quantity, it means that there is a problem in the connection between a connecting end currently inputting radio frequency energy and the operated object, and a connection standard is not met. If the quantity of radio frequency circuits that do not meet the connection standard does not reach a preset quantity, that is, requirement of the radio frequency operation is not met, output radio frequency energy is reduced to avoid causing damage to the radio frequency mainframe or the radio frequency operated object. At the same time, an alarm can be performed to prompt operating users to check the connection situation between the connection ends and the operated object. The alarm can be an audible and visual alarm or text display on a display screen of the radio frequency mainframe.

At this time, a method of detecting whether the change amounts of the detected values reaches the preset value range can be implemented by detecting a lowering amount of a voltage of a primary coil of a transformer, as in the above step S302, and can also be implement by detecting an output impedance value of a radio frequency input circuit, which are specifically as follows.

Specifically, reducing output radio frequency energy refers to lowering power of an output radio frequency signal. In this situation, radio frequency energy is reduced to be a value that is not equal to 0, and radio frequency energy with a safe strength is still output. Alternatively, the connection between the radio frequency input circuit and the operated object is cut off; in this situation, radio frequency energy is directly reduced to 0.

In this embodiment of the this application, when an operated object is connect to a radio frequency mainframe through connecting ends of radio frequency circuits, before radio frequency energy is input, according change amounts of detected values of the radio frequency circuits, it is determined whether a quantity of target radio frequency circuits of which the change amounts reach a preset value range reaches a preset quantity, that is, it is determined whether the connection between the connecting ends and the operated object meets a connection standard; if reaching, the preset quantity of target radio frequency circuits are selected from the target radio frequency circuits as radio frequency input circuits, and radio frequency signals are controlled to input; if not reaching, no radio frequency input is performed, so as to avoid subsequent radio frequency operations from being affected by connection that does not meet the standard. Accordingly, the above-described method for safety control of radio frequency operation can automatically determine whether connection between an operated object and radio frequency circuits meets a standard, and does not perform radio frequency energy input when radio frequency circuits meeting the standard do not meet requirement of radio frequency operation in quantity, thereby improving safety and intelligence of radio frequency operation. Furthermore, after radio frequency energy is input, it is further possible to detect the change amounts of the detected values continuously and in real time; if it is detected that the quantity of target radio frequency circuits of which the change amounts of the detected values reach the preset value range is less than the preset quantity, output radio frequency energy is reduced, so as to reduce risk of damage to the radio frequency mainframe and the operated object, and further improve safety and intelligence of radio frequency operations.

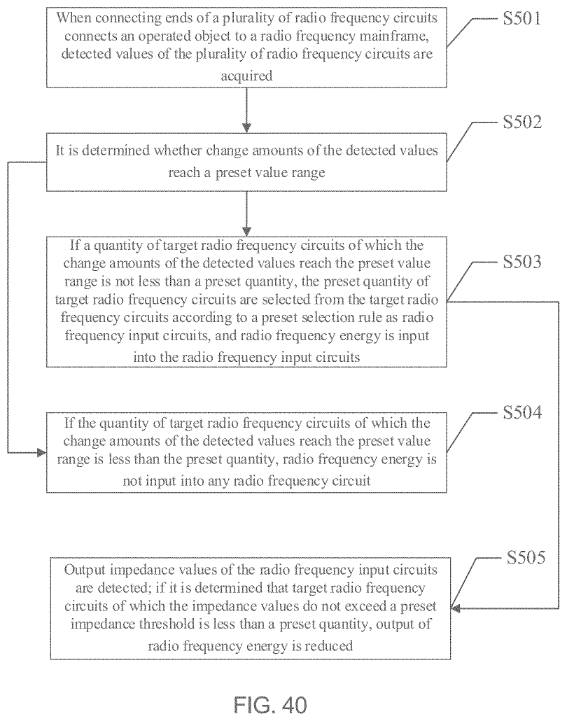

Referring to FIG. 40, which is an implementation flow chart of a method for safety control of radio frequency operation provided by another embodiment of this application. The method can be applied to the radio frequency mainframe shown in FIG. 36, as shown in FIG. 40, the method specifically comprises the follows.

Step S501, when connecting ends of a plurality of radio frequency circuits connects an operated object to a radio frequency mainframe, detected values of the plurality of radio frequency circuits are acquired.

Step S502, it is determined whether change amounts of the detected values reach a preset value range.

Step S503, if a quantity of target radio frequency circuits of which the change amounts of the detected values reach the preset value range is not less than a preset quantity, the preset quantity of target radio frequency circuits are selected from the target radio frequency circuits according to a preset selection rule as radio frequency input circuits, and radio frequency energy is input into the radio frequency input circuits.

Step S504, if the quantity of target radio frequency circuits of which the change amounts of the detected values reach the preset value range is less than the preset quantity, radio frequency energy is not input into any radio frequency circuit.

Step S505, output impedance values of the radio frequency input circuits are detected; if it is determined that target radio frequency circuits of which the impedance values do not exceed a preset impedance threshold is less than a preset quantity, output of radio frequency energy is reduced.

An output impedance value of each impedance detection circuit is detected, it is determined whether the impedance values exceed a preset impedance threshold, and a quantity of target radio frequency circuits of which the impedance values do not exceed the preset impedance threshold is counted. If the target radio frequency circuits of which the impedance values do not exceed the preset impedance threshold is less than the preset quantity, a frequency of an output radio frequency signal is lowered or a connection between a radio frequency input circuit and the operated object is cut off.

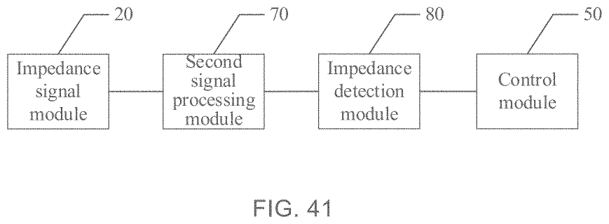

Referring to FIG. 41, FIG. 41 is a structural schematic diagram of an impedance detection circuit. Each impedance detection circuit is connected to an output end of a radio frequency input circuit to which a radio frequency signal is input, and each impedance detection circuit includes an impedance detection signal module 60, a second signal processing module 70, an impedance detection module 80, and the control module 40. The second signal processing module 70 includes a filter, a resonator, an amplifier, etc., so as to perform filtering, amplification, and so on for an impedance detection signal, and the specific circuit structure is not particularly limited. The impedance detection module 80 can include an impedance detection circuit configured to directly detect impedance values, or include a current detection circuit and a voltage detection circuit for indirectly calculating impedance values through detected currents and voltages, the specific circuit structure is not particularly limited.

In this embodiment of the this application, when an operated object is connect to a radio frequency mainframe through connecting ends of radio frequency circuits, before radio frequency energy is input, according change amounts of detected values of the radio frequency circuits, it is determined whether a quantity of target radio frequency circuits of which the change amounts reach a preset value range reaches a preset quantity, that is, it is determined whether the connection between the connecting ends and the operated object meets a connection standard; if reaching, the preset quantity of target radio frequency circuits are selected from the target radio frequency circuits as radio frequency input circuits, and radio frequency signals are controlled to input; if not reaching, no radio frequency input is performed, so as to avoid subsequent radio frequency operations from being affected by connection that does not meet the standard. Accordingly, the above-described method for safety control of radio frequency operation can automatically determine whether connection between an operated object and radio frequency circuits meets a standard, and does not perform radio frequency energy input when radio frequency circuits meeting the standard do not meet requirement of radio frequency operation in quantity, thereby improving safety and intelligence of radio frequency operation. Furthermore, after radio frequency energy is input, it is further possible to detect impedance values of the radio frequency input circuit in real time; if it is detected that the quantity of target radio frequency circuits of which the impedance values exceed a preset impedance threshold and reaches a preset value range is less than a preset quantity, output radio frequency energy is reduced, so as to reduce risk of damage to the radio frequency mainframe and the operated object, and further improve safety and intelligence of radio frequency operations.

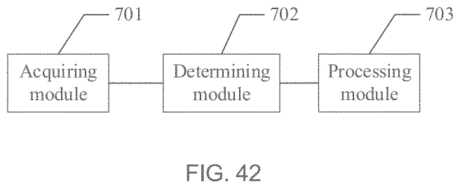

Referring to FIG. 42, which is a structural schematic diagram of an apparatus for safety control of radio frequency operation provided by an embodiment of this application. In order to facilitate description, only parts relating to embodiments of this application are shown. The apparatus can be arranged in the above-described radio frequency main frame, and the apparatus includes: an acquiring module 701 configured to: when connecting ends of a plurality of radio frequency circuits connects an operated object to a radio frequency mainframe, acquire detected values of the plurality of radio frequency circuits; a determining module 702 configured to determine whether change amounts of the detected values reach a preset value range; and a processing module 703 configured to: if a quantity of target radio frequency circuits of which the change amounts of the detected values reach the preset value range is not less than a preset quantity, select the preset quantity of target radio frequency circuits from the target radio frequency circuits according to a preset selection rule as radio frequency input circuits, and input radio frequency energy into the radio frequency input circuits; wherein the processing module 703 is further configured to: if the quantity of target radio frequency circuits of which the change amounts of the detected values reach the preset value range is less than the preset quantity, not input radio frequency energy into any radio frequency circuit.

Furthermore, the processing module 703 is further configured to: when it is detected that the quantity of target radio frequency circuits of which the change amounts of the detected values reach the preset value range is less than the preset quantity, reduce output radio frequency energy, specifically for lowering power of an output radio frequency signal; or cut off a connection between a radio frequency input circuit and an operated object.

The processing module 703 is further configured to: acquire preset numbers of the target radio frequency circuits; determine connecting areas corresponding to the target radio frequency circuits according to the numbers; and correspondingly select the radio frequency input circuits in the target radio frequency circuits according to operation areas of the current radio frequency operation and a quantity of circuits required by each operation area.

Furthermore, the connecting end is a neutral electrode, each radio frequency circuit has a neutral electrode, and each radio frequency circuit includes a detection signal module, a transformer, and a control module.

Thus, the processing module 703 is further configured to: when neutral electrodes of a plurality of radio frequency circuits are attached to the operated object to connect the operated object to the radio frequency mainframe, control the control module to control the detection signal module to send a detecting signal to the transformer, and acquire voltage values of primary coils of transformers of a plurality of radio frequency circuits.

Furthermore, the determining module 702 is further configured to determine whether the voltage values of primary coils of transformers of radio frequency circuits are lowered to reach a preset value range.

The processing module 703 is further configured to: detect an output impedance value of each radio frequency input circuit, determine whether the impedance values exceed a preset impedance threshold, and count a quantity of target radio frequency circuits of which the impedance values do not exceed the preset impedance threshold; and if the target radio frequency circuits of which the impedance values do not exceed the preset impedance threshold is less than the preset quantity, lower a frequency of an output radio frequency signal or cut off a connection between a radio frequency input circuit and the operated object.

Specifically, the processing module 703 detects the output impedance values by controlling impedance detection circuits. Among them, each impedance detection circuit is connected to an output end of a radio frequency input circuit, and each impedance detection circuit includes an impedance detection signal module, an impedance detection module, and the control module. The processing module 703, through the control module, control the impedance detection signal module to output an impedance detection signal, and the impedance detection module detects an output impedance value of each radio frequency input circuit.

In this embodiment of the this application, when an operated object is connect to a radio frequency mainframe through connecting ends of radio frequency circuits, before radio frequency energy is input, according change amounts of detected values of the radio frequency circuits, it is determined whether a quantity of target radio frequency circuits of which the change amounts reach a preset value range reaches a preset quantity, that is, it is determined whether the connection between the connecting ends and the operated object meets a connection standard; if reaching, the preset quantity of target radio frequency circuits are selected from the target radio frequency circuits as radio frequency input circuits, and radio frequency signals are controlled to input; if not reaching, no radio frequency input is performed, so as to avoid subsequent radio frequency operations from being affected by connection that does not meet the standard. Accordingly, the above-described method for safety control of radio frequency operation can automatically determine whether connection between an operated object and radio frequency circuits meets a standard, and does not perform radio frequency energy input when radio frequency circuits meeting the standard do not meet requirement of radio frequency operation in quantity, thereby improving safety and intelligence of radio frequency operation. Furthermore, after radio frequency energy is input, it is further possible to detect the change amounts of the detected values or the detected values in real time; if it is detected that the change amounts of the detected values reach a preset value range, or the quantity of target radio frequency circuits of which the impedance values exceed a preset impedance threshold and reach a preset value range is less than a preset quantity, output radio frequency energy is reduced, so as to reduce risk of damage to the radio frequency mainframe and the operated object, and further improve safety and intelligence of radio frequency operations.

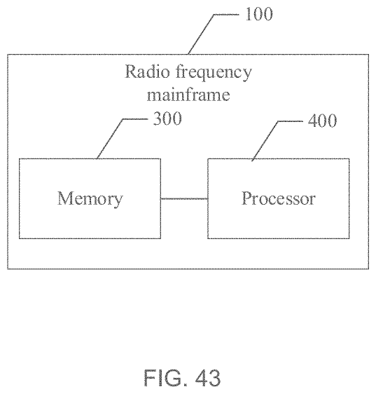

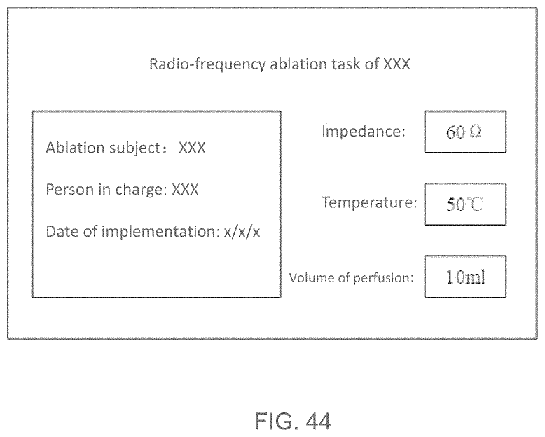

As shown in FIG. 43, an embodiment of this application further provides a radio frequency mainframe, which includes a memory 300 and a processor 400, the processor 400 can be an apparatus for safety control of radio frequency operation in the above embodiment, and can also be the processing module 703 in the apparatus for safety control of radio frequency operation. The memory 300 can be, for example, a hard disk drive memory, a non-volatile memory (such as a flash memory or other electronically programmable restricted deletion memory used to form solid-state drives, etc.), volatile memory (such as a static or dynamic random access memory, etc.), and so on, embodiments of this application do not limit here.

The memory 300 stores executable program codes; the processor 400 coupled with the memory 300 calls the executable program codes stored in the memory to execute the above-described method for safety control of radio frequency operation.

Further, an embodiment of this application further provides a computer-readable storage medium, the computer-readable storage medium can be set in the radio frequency mainframes in the above embodiments, and the computer-readable storage medium can be the memory 300 in the above embodiment shown in FIG. 43. The computer-readable storage medium stores a computer program, and the program, when being executed by a processor, implements the method for safety control of radio frequency operation described in the embodiments shown in above FIG. 37, FIG. 38, and FIG. 40. Further, the computer-readable storage medium can also be a U-disk, a mobile hard disk, a read-only memory (ROM), a RAM, a magnetic disk or a CD-ROM, and other media that can store program codes.

It should be noted that regarding the foregoing method embodiments, for simplicity of description, they are all expressed as a series of action combinations, but those skilled in the art should know that the present invention is not limited by the described sequence of actions. Because according to the present invention, certain steps can be performed in other orders or simultaneously. Secondly, those skilled in the art should also know that the embodiments described in the specification are all preferred embodiments, and the involved actions and modules are not necessarily all required by the present invention.

In the above-mentioned embodiments, the description of each embodiment has its own emphasis. For parts that are not described in detail in a certain embodiment, reference may be made to related descriptions of other embodiments.

Ablation Operation Prompting Method, Electronic Device and Computer-Readable Storage Medium

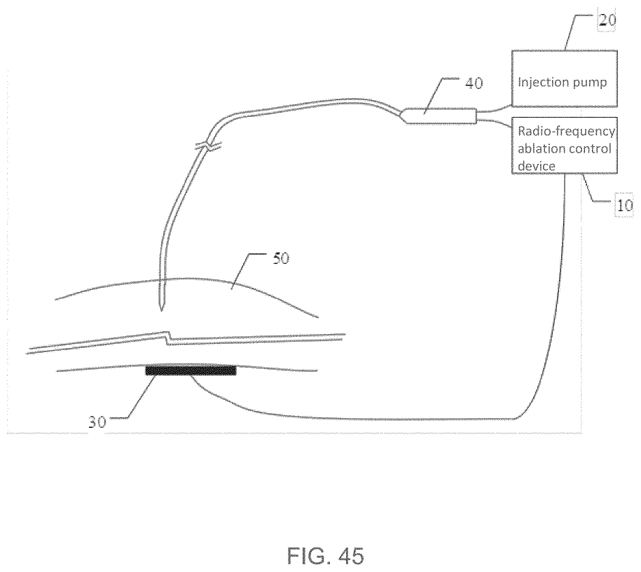

FIG. 45 is a schematic view showing an application scenario of an ablation operation prompting method provided in an embodiment of the present invention. The ablation operation prompting method can be implemented by a radio-frequency ablation control device 10 shown in FIG. 45, or implemented by other computer equipment that has established a data connection with the radio-frequency ablation control device 10.

As shown in FIG. 45, the radio-frequency ablation control device 10 is connected to a syringe pump 20, a neutral electrode 30 and a radio-frequency ablation catheter 40. The radio-frequency ablation control device 10 is provided with a built-in display screen (not shown).

Specifically, before an ablation task is implemented, an energy emitting end of the radio-frequency ablation catheter 40 for generating and outputting radio-frequency energy and an extension tube (not shown) of the syringe pump 20 are inserted into the body of an ablation subject 50 (such as an emphysema patient), reaching an ablation site. Then, the neutral electrode 30 is brought into contact with the skin surface of the ablation subject 50. A radio-frequency current flows through the radio-frequency ablation catheter 40, the tissue of the ablation subject and the neutral electrode 30, to form a circuit.

When an ablation task is triggered, the radio-frequency ablation catheter 40 is controlled by the radio-frequency ablation control device 10 to outputting radio-frequency energy to the ablation site by discharging to implement an ablation operation on the ablation site. Meanwhile the syringe pump 20 performs a perfusion operation on the ablation subject through the extension tube, wherein physiological saline is infused into the ablation site, to adjust the impedance and temperature of the ablation site.

At the same time, the radio-frequency ablation control device 10 acquires a locally pre-stored image of the ablation site and displays the image on the display screen.

Moreover, the radio-frequency ablation control device 10 also acquires position data of a currently-being-ablated target ablation point by for example, a camera (not shown) installed near the energy emitting end of the radio-frequency ablation catheter, and marks the target ablation point in the image according to the position data.

Further, the radio-frequency ablation control device 10 also acquires the elapsed ablation time and the temperature of the target ablation point in real time by a built-in timer and a temperature sensor (not shown) installed near the energy emitting end, determines the ablation status of the target ablation point according to the elapsed ablation time and the temperature, and then generate a schematic real-time dynamic change diagram of the target ablation point according to the ablation status, and display the schematic diagram on the screen, to indicate to a user the real-time ablation status change of the target ablation point.

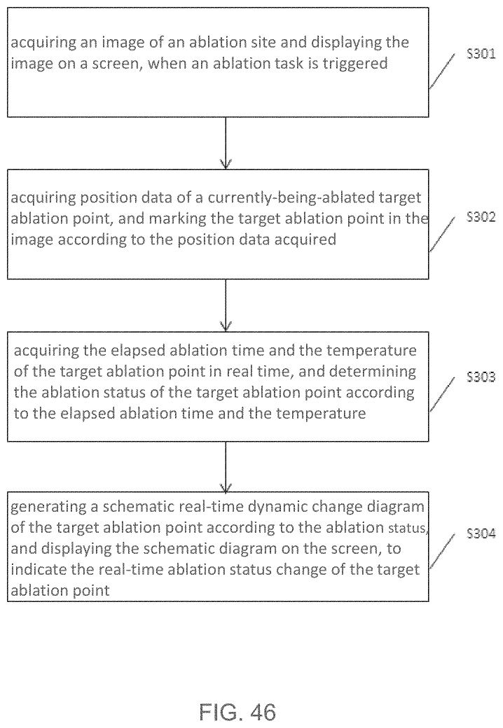

FIG. 46 shows a flow chart of an ablation operation prompting method provided in an embodiment of the present invention. The method can be implemented by a radio-frequency ablation control device 10 shown in FIG. 45, or implemented by other computer terminals connected thereto. For ease of description, in the following embodiments, the radio-frequency ablation control device 10 is used as an implementation body. As shown in FIG. 46, the method includes specifically:

Step S301: acquiring an image of an ablation site and displaying the image on a screen, when an ablation task is triggered.

Specifically, before Step S301 is implemented, besides the radio-frequency ablation control device, a main control device of an image acquisition system can also acquire a holographic image of an ablation site in each ablation subject intended to receive an ablation operation by an ultrasound medical imaging device or an endoscopic system in advance, and store the acquired holographic image in an image database. When an ablation task is triggered, identification information of a target ablation subject corresponding to the ablation task is acquired, and then the image of the ablation site in the target ablation subject is acquired according to the identification information by querying the image database, and displayed on a screen.

The screen may be a display screen built in the radio-frequency ablation control device, or an external display screen that has established a data connection with the radio-frequency ablation control device. The holographic image can be an original image taken, or a two-dimensional or three-dimensional static or dynamic image converted from the original image by calling PROE, AUTOCAD, Adobe Photoshop, Python Matplotlib, or other image processing programs.

It can be understood that in addition to PROE, AUTOCAD, Adobe Photoshop, and Python Matplotlib, there are still many other applications currently used for image processing. The program used can be determined according to actual needs, and is not particularly limited in the present invention.

Optionally, the preset prompting interaction interface can be used as a carrier of the image and various schematic diagrams involved in the various embodiments of the present invention, to facilitate the layout design and the management and positioning of each schematic diagram. For example, the image is displayed in a first preset area of the preset prompting interaction interface.

The preset prompting interaction interface is a graphical user interface (GUI). It can be understood that an application of the prompting interaction interface is preset in the radio-frequency ablation control device. Before the ablation operation is implemented, the application is automatically called, to display the prompting interaction interface on the screen. The preset prompting interaction interface includes multiple areas, respectively used to display different prompt information. The processing logic of the application can automatically divide areas, based on the number of information that needs to be displayed.

Specifically, the acquired image of the ablation site is stored in a first storage location corresponding to a first preset area of the prompting interaction interface. The application automatically refreshes the prompting interaction interface according to a preset period, and add the image to the first preset area for display, if the image is read from the first storage location when implement a refresh operation. Alternatively the acquired image of the ablation site is displayed in the first preset area of the preset prompting interaction interface in the form of overlapped images.

Step S302: acquiring position data of a currently-being-ablated target ablation point, and marking the target ablation point in the image according to the position data.

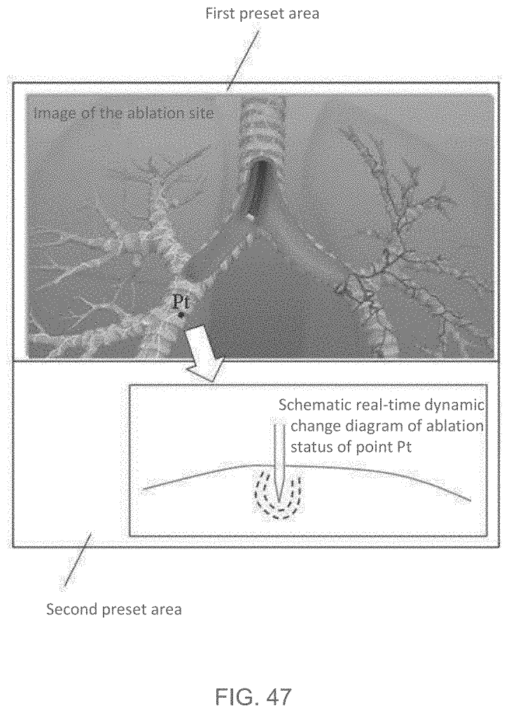

Specifically, position coordinate of the currently-being-ablated target ablation point in the image of the ablation site is acquired according to any one of the following three preset positioning methods. Then, the target ablation point is marked in the image of the ablation site according to the position data and a preset marking logic, wherein the marking logic is a general designation of the preset various operations required for marking as shown in FIG. 47, according to the position coordinate, a circular icon Pt is drawn at a corresponding position in the image and used as a mark.

The first preset positioning method is to obtain the position data of the target ablation point through an endoscope. Specifically, a picture of a current ablation operation captured by an endoscope is acquired and compared with the image, to obtain the position data of the target ablation point in the image.

It can be understood that a camera is provided at the tip of the endoscope; and when an ablation operation is implemented, the camera is inserted together with an ablation catheter into the body of an ablation subject, approaching the ablation site, to capture a picture of the ablation site in real time. The camera sends the captured pictures back to the radio-frequency ablation control device while the pictures are captured. The feature points in an area in the ablation site where the ablation operation is being performed in the picture returned by the endoscope are extracted and matched with the feature points in the image displayed in Step S301; and position data corresponding to a feature point with the highest matching degree is determined as the position data of the target ablation point. The position data is a position coordinate, and the coordinate system of the position coordinate is a two-dimensional or three-dimensional coordinate system established by using a centroid of the ablation site in the displayed image or an end point of the image as the origin.

The second preset positioning method is to obtain the position data of the target ablation point through an ultrasound medical imaging device. Specifically, an ultrasound image of the target ablation point is obtained by using an ultrasound medical imaging device; and according to the ultrasound image, the position data of the target ablation point in the image is obtained.

The working principle of the ultrasound medical imaging device is to irradiate the human body with ultrasonic waves, and obtain a visible image of the nature and structure of human tissues, by receiving and processing echoes carrying feature information of nature or structures of human tissues, such as a section shape of the ablation site. At present, many kinds of ultrasound medical imaging devices are available, and the ultrasound medical imaging device used is not particularly limited in the present invention. image recognition of the ultrasound image is performed, to determine the position data of the target ablation point in the ultrasound image. Then, the position data of the target ablation point in the displayed image is determined according to the corresponding relationship between each ablation site in the ultrasound image and each ablation site in the image displayed in Step S301.

The third preset positioning method is to obtain the position data of the target ablation point by electromagnetic navigation technology. Specifically, the actual position data of the target ablation point is obtained by for example electromagnetic navigation bronchoscopy (ENB), and then the position data of the target ablation point in the displayed image is determined according to the corresponding relationship between the real ablation site and each ablation site in the image displayed in Step S301.

Step S303: acquiring the elapsed ablation time and the temperature of the target ablation point in real time, and determining the ablation status of the target ablation point according to the elapsed ablation time and the temperature.

Specifically, in an ablation task, at least one ablation site needs to be ablated, each ablation site includes multiple ablation points, and for each ablation point, a corresponding ablation operation needs to be performed. A timer is preset in the radio-frequency ablation control device; and whenever an ablation operation of an ablation point is started, the timer records the elapsed ablation time of the ablation point. When the position of the ablation operation changes, the current timing is ended, and a new round of timing is restarted.

Moreover, during the timing, the temperature near the target ablation point is also obtained by a temperature sensor in real time. Then, according to the elapsed implementation time of the ablation operation (that is, the elapsed ablation time) and the temperature, the ablation status of the target ablation point, for example, the shape and size of the tissue ablated, is determined.

For the same ablation point, timing separately according to the temperature can be performed, that is, the durations at different temperatures are statistically counted. The shape and size of the tissue ablated can be determined according to the length, width and height of the ablated area starting from the target ablation point (hereinafter collectively referred to as the ablated length, width and height of the target ablation point). The ablated length, width and height of the target ablation point are calculated according to Formulas 1-3 below:

(time of a temperature continuously reaching a preset temperature/preset unit of ablation time)*preset ablation length=ablated length; Formula 1:

(time of a temperature continuously reaching a preset temperature/preset unit of ablation time)*preset ablation width=ablated width; Formula 2:

(time of a temperature continuously reaching the preset temperature/preset unit of ablation time)*preset ablation height=ablated height. Formula 3:

The preset temperature and the preset unit ablation time are respectively the critical temperature and the unit critical time allowing the ablation site to undergo qualitative changes (i.e., achieve the expected ablation effect). The preset ablation length, preset ablation height and preset ablation width are the length, width and height of the ablated area increased with the elapse of a preset unit of ablation time after the ablation site reaches the preset temperature. The preset temperature, preset unit of ablation time, and preset ablation length, width and height can be set according to the user's custom operation.

It is experimentally confirmed that after the temperature of the ablation site reaches the critical temperature, the increase in the length, width, and height of the ablated area is generally constant in every time interval. Therefore, according to the above Formulas 1 to 3, the ablated length, width and height of the target ablation point can be obtained. Then, according to the obtained ablated length, width and height and the coordinate of the target ablation point, the shape, size, and boundary coordinates of the ablated area around the target ablation point are obtained.

Step S304: generating a schematic real-time dynamic change diagram of the target ablation point according to the ablation status, and displaying the schematic diagram on the screen, to indicate the real-time ablation status change of the target ablation point.

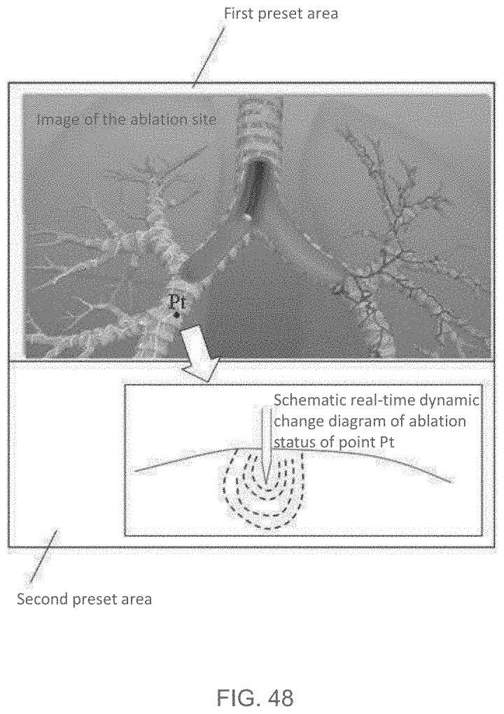

Specifically, by calling the above image processing program, and according to the coordinate of the target ablation point, the shape and size of the ablated area around the target ablation point, and the boundary coordinates of the ablated area, a schematic real-time dynamic change diagram of the ablation status of the target ablation point as shown in FIG. 47 and FIG. 48 is generated. The schematic real-time dynamic change diagram contains visualizations of the process of continuously expanding the boundary of the ablated area. Each circle of dotted lines in the schematic real-time dynamic change diagrams of the ablation status of the target ablation point as shown in FIG. 47 and FIG. 48 indicates the boundary of each expansion of the ablated area. Further, the boundary of the outermost circle can also be displayed in a flashing manner, to make the schematic diagram more indicative.

Optionally, the schematic real-time dynamic change diagram is displayed in a second preset area of the preset prompting interaction interface. The second preset area may be set within an area embraced by the first preset area. Alternatively, it may also be set near the first preset area.

Further, the positional relationship between the first preset area and the second preset area may also be determined according to the number of contents that needs to be displayed in the prompting interaction interface. For example, when there are more contents to be displayed, the generated schematic real-time dynamic change diagram is displayed by overlapping in the vicinity of the target ablation point in the image displayed in the first preset area, to save the space occupied. When there are less contents to be displayed, the schematic real-time dynamic change diagram is displayed in an area next to the first preset area, thereby improving the flexibility of information display.

In the embodiments of this application, when an ablation task is triggered, an image of an ablation site is acquired and displayed on a preset prompting interaction interface where the image of the ablation site is marked with a currently-being-ablated target ablation point; according to the elapsed ablation time and the temperature of the target ablation point acquired in real time, a schematic real-time dynamic change diagram of the ablation status of the target ablation point is generated and displayed, so that the status changes of an ablation site can be displayed in real time and intuitively during the implementation of the ablation operation, thereby improving the effectiveness and relevance of information prompts.

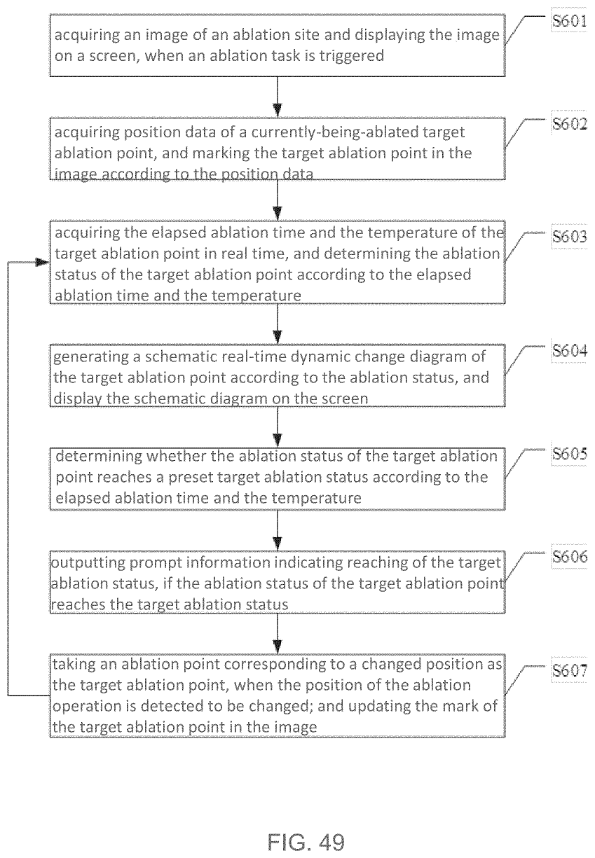

FIG. 49 shows a flow chart of an ablation operation prompting method provided in another embodiment of the present invention. The method can be implemented by a radio-frequency ablation control device 10 shown in FIG. 45, or implemented by other computer terminals connected thereto. For ease of description, in the following embodiments, the radio-frequency ablation control device 10 is used as an implementation body. As shown in FIG. 49, the method includes specifically:

Step S601: acquiring an image of an ablation site and displaying the image on a screen, when an ablation task is triggered.

Step S602: acquiring position data of a currently-being-ablated target ablation point, and marking the target ablation point in the image according to the position data.

Step S603: acquiring the elapsed ablation time and the temperature of the target ablation point in real time, and determining the ablation status of the target ablation point according to the elapsed ablation time and the temperature.

Step S604: generating a schematic real-time dynamic change diagram of the target ablation point according to the ablation status, and display the schematic diagram on the screen, to indicate the real-time ablation status change of the target ablation point.

Steps S601 to S604 are the same as Steps S301 to S304 in the embodiment shown in FIG. 46, and details may be made reference to the relevant description in the embodiment shown in FIG. 46, and will not be repeated here.

Step S605: determining whether the ablation status of the target ablation point reaches a preset target ablation status according to the elapsed ablation time and the temperature.

Step S606: outputting prompt information indicating reaching of the target ablation status, if the ablation status of the target ablation point reaches the target ablation status.

Specifically, according to the elapsed ablation time and the temperature of the target ablation point, the ablated length, width and height of the target ablation point are determined, and whether the ablated length, width and height of the target ablation point reach corresponding preset standard values respectively are determined. If they reach the corresponding preset standard values respectively, the ablation status of the target ablation point is determined to reach the preset target ablation status, and prompt information indicating reaching of the target ablation status is outputted. The prompt information indicating reaching of the target ablation status can be output in at least one form of a prompt text, a prompt graphic, a prompt sound, and a prompt light, etc.

Step S607: taking an ablation point corresponding to a changed position as the target ablation point, when the position of the ablation operation is detected to be changed; updating the mark of the target ablation point in the image and returning to Step S603, until the ablation operation is ended.

Specifically, the method described in Step S602 can be used, wherein the position data of the ablation operation is acquired in real time, and whether the position of the ablation operation has changed is detected according to the position data. If the position of the ablation operation is detected to be changed, it means that the ablation point has changed. Therefore, an ablation point corresponding to a changed position is used as a new target ablation point, and a changed position coordinate is used as the position data of the new target ablation point. Then according to the position data, the mark of the target ablation point is updated in the image displayed on the screen. For example, the mark of the previous target ablation point is hidden or deleted, and the mark of the new target ablation point is added to the image at the same time. The method of adding the mark of the new target ablation point is the same as that for the previous target ablation point and will not be repeated here.

Moreover, the process is returned to Step S603: acquiring the elapsed ablation time and the temperature of the target ablation point in real time, and determining the ablation status of the target ablation point according to the elapsed ablation time and the temperature, until the ablation operation is ended. The ablation operation can be automatically ended by the radio-frequency ablation control device when an abnormal event or other preset events are detected, or ended according to the user's operation.

Optionally, in another embodiment of the present invention, after Step S601 of acquiring an image of an ablation site and displaying the image on a screen when an ablation task is triggered. the method further includes: acquiring a first drawing parameter of a target operation track of the ablation operation, and drawing the target operation track at a corresponding position of the image according to the first drawing parameter; acquiring the position change data of the ablation point in real time, determining a second drawing parameter of the real-time operation track of the ablation operation according to the position change data, and drawing the real-time operation track at a corresponding position of the image according to the second drawing parameter; and analyzing in real time whether the amplitude of the real-time operation track deviating from the target operation track is greater than a preset amplitude, and outputting prompt information for track deviation warning when the real-time operation track deviates from the target operation track by an amplitude greater than the preset amplitude.

Specifically, the first drawing parameter of the target operation track of each ablation operation to be performed is stored in the radio-frequency ablation control device locally, or ENBW in a database server that has established a data connection with the radio-frequency ablation control device. The radio-frequency ablation control device can query the first drawing parameter of the target operation track of the corresponding ablation operation locally or from the database server according to the identification information of the triggered ablation task.

It can be understood that the ablation site is composed of multiple ablation points, the ablation operation needs to be performed on each ablation point one by one. The target operation track is a preset preferred route to be taken when the ablation operation is performed for each ablation point, and used to assist the user in the ablation operation, to achieve a better ablation effect.

Optionally, the first drawing parameter can be generated according to a route parameter input by the user, or calculated according to the preset ablation target and the shape and size of the ablation site.

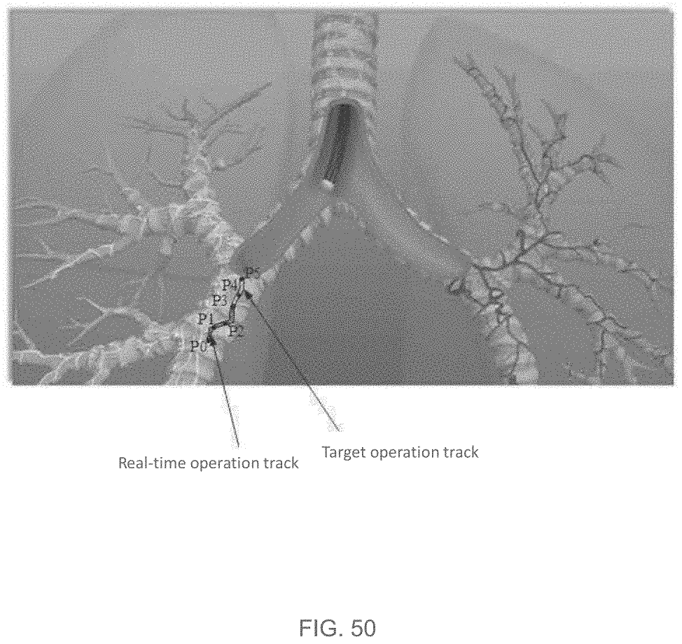

The first drawing parameter may specifically include, but is not limited to, position coordinates and drawing order of various ablation points (such as points P0 to P5 in FIG. 50) in the target operation track, wherein P0 is the starting point, and P5 is the end point), and the line characteristics of the target operation track. The line characteristics of the target operation track can include, but are not limited to, for example: line type (such as: dashed line, or solid line), shadow, thickness, and color, etc.

Further, the method described in Step S302 can be used, wherein the position data of the ablation operation is acquired in real time. Then, the position change data of the ablation operation is obtained according to the position data obtained in real time, wherein the position change data includes: the initial position coordinate of the ablation operation and the position coordinates after each position change. Whenever a position change of the ablation operation is detected, the changed position is marked as an ablation point and the position coordinate of the ablation point is recorded. Then, the second drawing parameter is determined according to the position coordinate of the marked ablation point and the preset drawing logic, and the real-time operation track is drawn according to the determined second drawing parameter (as shown in FIG. 50), to present a display effect of the real-time operation track dynamically extending over time.

The second drawing parameter can include, but is not limited to: position coordinates and drawing order of various ablation points (i.e. various marked ablation points) in the real-time operation track, and the line characteristics of the real-time operation track. The line characteristics of the real-time operation track can include, but are not limited to, for example: line type (such as: dashed line, or solid line), shadow, thickness, and color, etc.

The coordinate system of the above-mentioned position coordinates is a two-dimensional or three-dimensional coordinate system established by using a centroid of the ablation site in the displayed image or an end point of the image as the origin.

Optionally, different colors and/or different types of lines can be used respectively in drawing the target operation track and the real-time operation track, to highlight the difference between target operation track and the real-time operation track, thereby further improving the effectiveness of information prompts.

Further, when the real-time operation track is drawn, the position coordinate of each ablation point in the drawn real-time operation track is compared with the position coordinate of each corresponding ablation point in the target operation track (corresponding to the ablation point in the real-time operation track in the drawing order), to obtain the coordinate variation therebetween. Then, the number of ablation points with a coordinate variation that is greater than a preset variation is counted. If the number is greater than a preset number, the amplitude of the real-time operation track deviating from the target operation track is determined to be greater than a preset amplitude, and prompt information for track deviation warning is outputted. Optionally, the prompt information can be in the form of a text and/or a graphic, and displayed in the first preset area of the prompting interaction interface.

Therefore, the target operation track and the real-time operation track are drawn, and the amplitude of deviation therebetween is analyzed. When the amplitude of deviation between the two is greater than the preset amplitude, the prompt information is displayed, so as to avoid the adverse effect of the user's improper operation on the ablation effect, improve the universality and intelligence of information prompts, and further improve the effectiveness of information prompts.

Optionally, in another embodiment of the present invention, the method further includes: acquiring the temperature of the ablation site in real time when the ablation task is triggered; drawing a real-time temperature change curve according to the temperature acquired in real time and displaying it on the screen; and analyzing in real time whether the temperature exceeds a preset warning value, and outputting prompt information for temperature warning in a display area of the temperature change curve when the temperature exceeds the warning value.

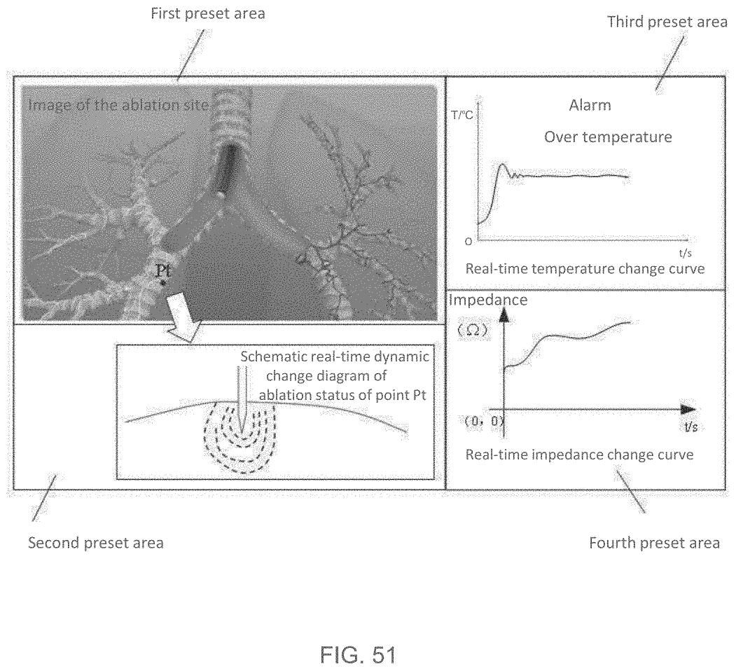

Specifically, the temperature of the ablation site can be obtained in real time by a temperature sensor set near the ablation site, and then a real-time temperature change curve as shown in FIG. 51 is drawn by calling a curve drawing function (such as: PLOT function) according to the temperature acquired in real time. The horizontal axis of the real-time temperature change curve is a time axis, and used to indicate the time at which each temperature value is obtained (t/s,). The vertical axis of the real-time temperature change curve represents the temperature (T/° C.). Optionally, the real-time temperature change curve can be drawn in a third preset area of the prompting interaction interface.

It can be understood that in addition to the PLOT function, many more functions are available for curve drawing, and the function used can be selected according to actual needs in practical applications and is not particularly limited in the present invention.

Further, when the real-time temperature change curve is drawn, whether the obtained temperature exceeds a preset warning value is analyzed in real time, and outputting prompt information for temperature warning in a display area (for example, the third preset area) of the temperature change curve when the temperature exceeds the warning value. The prompt information for temperature warning can be displayed in the form of a text and/or a graphic, as shown FIG. 51.

Therefore, by drawing the real-time temperature change curve, and when the acquired temperature exceeds the warning value, warning information is outputted. This promotes the user to understand the temperature change of the ablation site in time and achieve the effect of temperature warning, thus further improving the universality and effectiveness of information prompts, and improving the safety of the ablation operation. In addition, by outputting the prompt information for temperature warning in the display area of the temperature change curve, the prompt information is made more directional.

Optionally, in another embodiment of the present invention, the method further includes: acquiring the impedance of the ablation site in real time when the ablation task is triggered; and drawing a real-time impedance change curve on a screen according to the impedance obtained in real time.

Specifically, by setting an impedance sensor installed at a tip of the radio-frequency ablation catheter, the impedance of the ablation site is acquired in real time; and a real-time impedance change curve as shown in FIG. 51 is drawn on the screen, by calling the above curve drawing function, according to the acquired impedance. The horizontal axis of the real-time impedance change curve represents time (t/s). The vertical axis of the real-time impedance change curve represents the impedance (a). Optionally, the real-time impedance change curve can be drawn in a fourth preset area of the prompting interaction interface.

Therefore, by drawing the real-time impedance change curve, the user is promoted to understand the impedance changes of the ablation site in time, to adjust the ablation operation in time, thus further improving the universality and effectiveness of information prompts, and improving the safety of the ablation operation.

Optionally, in another embodiment of the present invention, the method further includes: acquiring the impedance of the ablation site in real time, when the ablation task is triggered; determining a target interval corresponding to the impedance according to the impedance acquired in real time and the impedance ranges respectively corresponding to multiple preset ablation impedance prompting intervals; and drawing a schematic real-time impedance diagram on the screen according to the impedance, the impedance range and the target interval. The schematic real-time impedance diagram includes: description information of the multiple ablation impedance prompting intervals, description information of a preset reference impedance, and description information of the corresponding relationship between the impedance and the target interval. The multiple ablation impedance prompting intervals include: a non-ablatable impedance interval, an ablatable impedance interval, and an optimal ablatable impedance interval.

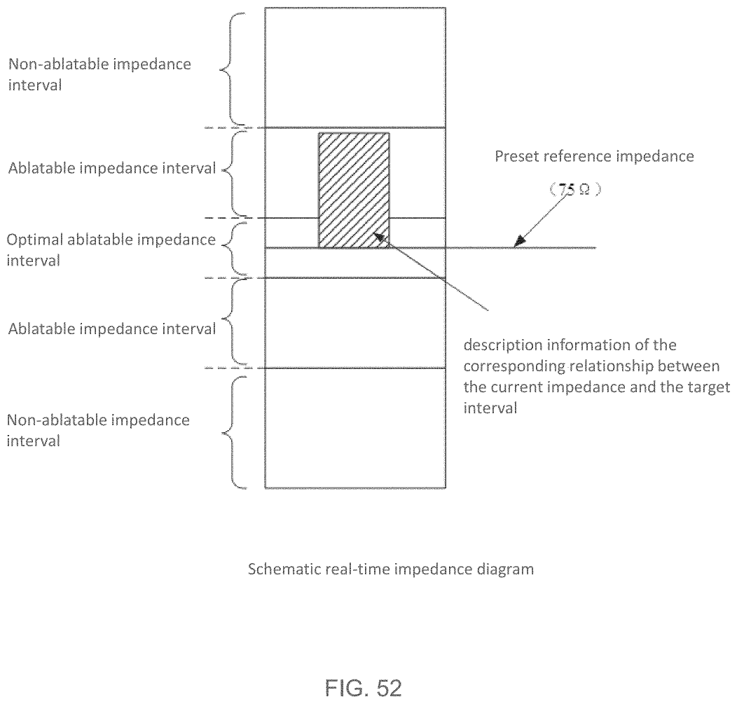

Further, as shown in FIG. 52, the description information of the multiple ablation impedance prompting intervals includes: multiple vertically laminated columns (6 columns in FIG. 52, and the number is not limited to 6 in actual applications) and the corresponding description texts and indicating graphics (such as the dashed line and curly brackets in FIG. 52) of the multiple ablation impedance prompting intervals; and the description information of the preset reference impedance includes: the description text and the indicating graphic (for example, arrow) of the preset reference impedance.

The multiple columns respectively correspond, from top to bottom, to an upper range of the non-ablatable impedance interval, an upper range of the ablatable impedance interval, an upper range of the optimal ablatable impedance interval, a lower range of the optimal ablatable impedance interval, a lower range of the ablatable impedance interval, and a lower range of the non-ablatable impedance interval.

The upper and lower limits of the non-ablatable impedance interval, ablatable impedance interval, and the optimal ablatable impedance interval can be preset in the radio-frequency ablation control device according to the user's custom operation. Optionally, the preset reference impedance may be a median value of the optimal ablation impedance interval or an average value of the upper limit and the lower limit. In this case, if the preset reference impedance is set according to the user's custom operation, the upper and lower limits of the non-ablatable impedance interval, the ablatable impedance interval, and optimal ablation impedance interval can be determined according to the preset reference impedance and the fluctuation range of each impedance interval preset by the user.

Further, the number of columns, the number and range of the ablation impedance prompting interval, and the correspondence between the column and each ablation impedance prompting interval may also be determined according to the number of electrodes on the radio-frequency ablation catheter, or set according to the user's custom operation.

Further, taking the column corresponding to the optimal ablatable impedance interval as a center, the height increases as the distance of the other columns in the multiple columns from the column increases. Therefore, by setting different sizes of columns to identify different ablation impedance intervals, the user is allowed to get to know the ablation impedance interval corresponding to the current impedance clearly, thereby further improving the eye-catching and intuitiveness of the prompt information, and improving the effectiveness of information prompts.

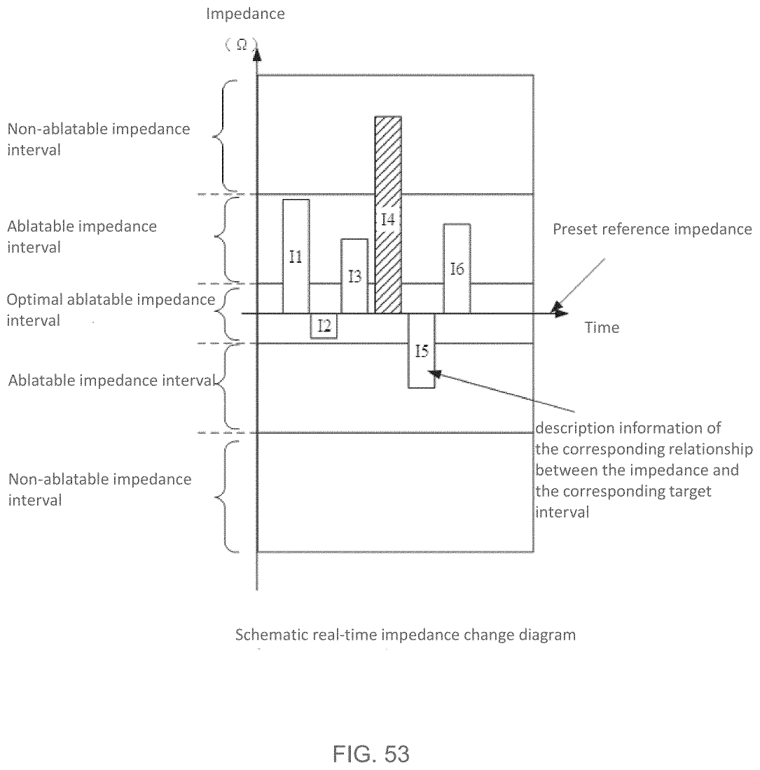

Optionally, in another embodiment of the present invention, the method further includes: acquiring the impedance of the ablation site in real time when the ablation task is triggered; determining a target interval corresponding to the impedance according to the impedance acquired in real time and the impedance ranges respectively corresponding to multiple preset ablation impedance prompting intervals; and drawing a schematic real-time impedance change diagram on the screen (as shown in FIG. 53) according to the impedance, the impedance range and the target interval. The schematic real-time impedance change diagram includes: two-dimensional coordinate axes, description information of the multiple ablation impedance prompting intervals, and description information of the corresponding relationship between each impedance and respective target interval. The multiple ablation impedance prompting intervals include: a non-ablatable impedance interval, an ablatable impedance interval, and an optimal ablatable impedance interval.

The horizontal axis of the two-dimensional coordinate axes is a time axis, indicating the acquisition time of each impedance. Moreover, the horizontal axis is also used to indicate the preset reference impedance. The vertical axis indicates, from bottom to top in a positive direction, an upper range of the optimal ablatable impedance interval, an upper range of the ablatable impedance interval, and an upper range of the non-ablatable impedance interval, and indicates, from top to bottom in a negative direction, a lower range of the optimal ablatable impedance interval, a lower range of the ablatable impedance interval, and a lower range of the non-ablatable impedance interval.

Optionally, the schematic real-time impedance change diagram is drawn on the preset prompting interaction interface.

Therefore, by drawing the schematic real-time impedance change diagram, the user is promoted to get to know the impedance changes of the ablation site in real time, and the user is reminded to adjust the ablation operation in time by an impedance warning, thus further improving the universality and effectiveness of information prompts.

Optionally, in another embodiment of the present invention, the description information of the corresponding relationship between the impedance and the target interval includes: graphics of different colors with preset shapes, wherein the different colors respectively correspond to different ablation impedance prompting intervals. For example, the red color corresponds to the non-ablatable impedance interval, the yellow color corresponds to the ablatable impedance interval, and the green color corresponds to the optimal ablatable impedance interval. Therefore, by using different colors, different ablation impedance intervals can be effectively distinguished, thereby further improving the effectiveness of information prompts.

Optionally, the height of the graphic is determined according to the difference between the impedance and the upper limit or lower limit of the corresponding target interval.

Preferably as shown in FIG. 53, the graphic has a bar shape, and as the impedance approaches the limit of the corresponding target interval, the length of the bar increases. The schematic real-time impedance change diagram can not only indicate the user with the real-time impedance change, but also indicate the user whether there is a safety risk in the current ablation operation. For example, the target interval corresponding to the impedance 12 in FIG. 53 is an optimal ablatable impedance interval, and indicating that the ablation operation at this time can achieve the best ablation effect; and the target interval corresponding to the impedance 14 is a non-ablatable impedance interval, indicating that the impedance of the ablation site is too high or too low, and there is a safety risk in the current ablation operation.

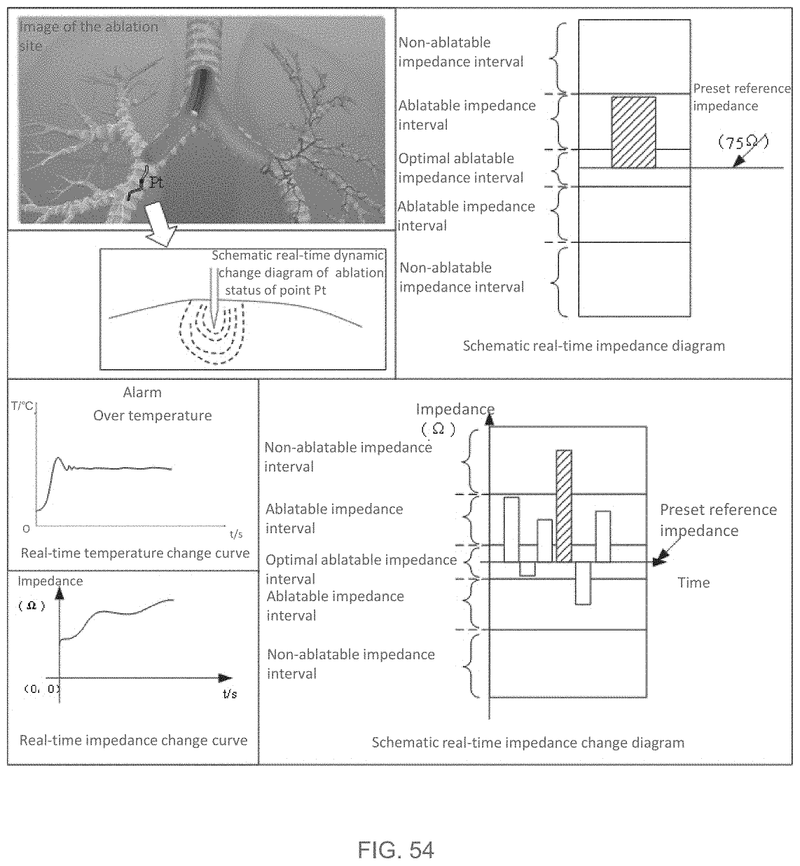

It should be noted that the schematic real-time impedance diagram, the schematic real-time impedance change diagram, the real-time impedance change curve, and other schematic diagrams do not conflict with each other. In practical use, one or more schematic diagrams illustrating a selective operation can be drawn and displayed according to the selective operation of the user. For example, as shown in FIG. 54, all the schematic diagrams are displayed on the preset prompting interaction interface. FIG. 47, FIG. 48, and FIGS. E50 to E54 are merely exemplary. In practical use, other more or less information, for example, specific temperature, impedance, system time, described information of the ablation subject, described information of the person in charge of the ablation operation, can be included in a different arrangement according to the practical needs or user's choice.

Further, the radio-frequency ablation catheter includes a single-electrode radio-frequency ablation catheter and a multi-electrode radio-frequency ablation catheter, wherein the single-electrode radio-frequency ablation catheter is correspondingly provided with a single impedance sensor, and the multi-electrode radio-frequency ablation catheter is correspondingly provided with multiple impedance sensors. The target interval can be determined according to the type of radio-frequency ablation catheter. Specifically, the determining a target interval corresponding to the impedance, according to the impedance acquired in real time and the impedance ranges respectively corresponding to multiple preset ablation impedance prompting intervals, includes: when the radio-frequency ablation catheter is a single-electrode radio-frequency ablation catheter, determining a target interval corresponding to the single impedance in real time according to the single impedance acquired in real time and the impedance ranges respectively corresponding to multiple ablation impedance prompting intervals; and when the radio-frequency ablation catheter is a multi-electrode radio-frequency ablation catheter, analyzing whether the multiple impedances obtained in real time correspond to the same ablation impedance prompting interval according to the impedance ranges respectively corresponding to multiple ablation impedance prompting intervals, wherein

if the multiple impedances correspond to the same ablation impedance prompting interval, the corresponding ablation impedance prompting interval is used as the target interval, and if the multiple impedances correspond to multiple ablation impedance prompting intervals, an ablation impedance prompting interval that covers the most impedances is used as the target interval.

Specifically, before implementing an ablation task, the radio-frequency ablation control device acquires model information of the connected radio-frequency ablation catheter, determines the type of the radio-frequency ablation catheter according to the acquired model information. Then, the target interval is determined according to the type determined. Taking a six-electrode radio-frequency ablation catheter as an example, the six-electrode radio-frequency ablation catheter is assumed to be correspondingly provided with κ impedance sensors, if the 6 impedances obtained by the 6 impedance sensors all fall in the optimal ablatable impedance interval, the corresponding target interval is determined to be the optimal ablatable impedance interval, and if 4 of the 6 impedances fall in the ablatable impedance interval and 2 impedances fall in the optimal ablatable impedance interval, the corresponding target interval is determined to be the ablatable impedance interval.

Optionally, in another embodiment of the present invention, after Step S601, the method further includes: when the prompt information outputted on the screen or the drawn schematic diagram has target information containing preset keywords, performing a screencapture operation, and saving the captured picture; and displaying all the pictures saved on the screen according to the priority of the target information, after the ablation task is ended.

Specifically, whether the prompt information outputted on the screen or the drawn schematic diagram has target information containing preset keywords is analyzed in real time, wherein the preset keywords have alert meaning, and may include, but is not limited to, for example: alarm, reminder, attention, and warning. If there is target information that contains the preset keywords, a screencapture operation is performed, and the captured picture is stored in the radio-frequency ablation control device locally or at a preset location of a cloud server. Then, after the ablation task is ended, all the pictures saved are displayed on the screen according to the timing sequence upon capture or the priority of the target information, to indicate to the user the abnormal conditions during the implementation of the entire ablation operation, thereby further improving the scope of application of information prompts, and improve the universality and intelligence of information prompts. A higher priority of the target information indicates a higher severity or importance of the corresponding prompt information.

Optionally, when the preset prompting interaction interface is used as a carrier, whether the prompt information outputted on the prompting interaction interface or the drawn schematic diagram has target information containing preset keywords is analyzed in real time.

It should be noted that for ease of description, the steps in each embodiment of the present invention are numbered in sequence; however, the numbering sequence does not constitute a restriction on the order of implementation. Some steps can be implemented at the same time, for example, Step S602 and Step S603 can be implemented at the same time, Step S604 and step S605 can also be implemented at the same time, and the generation and display of other schematic diagrams involved may also be implemented at the same time.

In the embodiments of this application, when an ablation task is triggered, an image of an ablation site is acquired and displayed on a preset prompting interaction interface where the image of the ablation site is marked with a currently-being-ablated target ablation point; according to the elapsed ablation time and the temperature of the target ablation point acquired in real time, a schematic image showing the real-time dynamic change of the ablation status of the target ablation point is generated and displayed, so that the status changes of an ablation site can be displayed in real time and intuitively during the implementation of the ablation operation, thereby improving the effectiveness and relevance of information prompts.

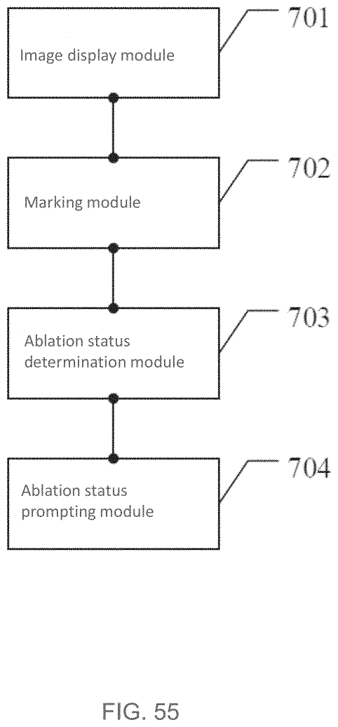

FIG. 55 is a schematic structural diagram of an ablation operation prompting device provided in an embodiment of the present invention. For ease of description, only the parts relevant to the embodiments of the application are shown. The device may be a computer terminal, or a software module configured on the computer terminal. As shown in FIG. 55, the device includes an image display module 701, a marking module 702, an ablation status determination module 703 and an ablation status prompting module 704.

The image display module 701 is configured to acquire an image of an ablation site and display the image on a screen, when an ablation task is triggered.

The marking module 702 is configured to acquire position data of a currently-being-ablated target ablation point, and mark the target ablation point in the image according to the position data.

The ablation status determination module 703 is configured to acquire the elapsed ablation time and the temperature of the target ablation point in real time, and determine the ablation status of the target ablation point according to the elapsed ablation time and the temperature.

The ablation status prompting module 704 is configured to generate a schematic diagram showing the real-time dynamic change of the target ablation point according to the ablation status, and display the schematic diagram on the screen, to indicate the real-time ablation status change of the target ablation point.

Optionally, the device further includes: a target operation track drawing module, configured to acquire a first drawing parameter of a target operation track of the ablation operation, and draw the target operation track at a corresponding position of the image according to the first drawing parameter; a real-time operation track drawing module, configured to acquire the position change data of the ablation point in real time, determine a second drawing parameter of a real-time operation track of the ablation operation according to the position change data, and draw the real-time operation track at a corresponding position of the image according to the second drawing parameter; and a track warning module, configured to analyze in real time whether the amplitude of the real-time operation track deviating from the target operation track is greater than a preset amplitude, and outputting prompt information for track deviation warning when the real-time operation track deviates from the target operation track by an amplitude greater than the preset amplitude.

Optionally, the device further includes: a temperature warning module, configured to acquire the temperature of the ablation site in real time, when the ablation task is triggered; draw a real-time temperature change curve on the screen according to the temperature acquired in real time; and analyze in real time whether the temperature exceeds a preset warning value, and outputting prompt information for temperature warning in a display area of the temperature change curve when the temperature exceeds the warning value.

Optionally, the device further includes: an impedance acquisition module, configured to acquire the impedance of the ablation site in real time, when the ablation task is triggered; and a real-time impedance change curve drawing module, configured to draw a real-time impedance change curve on the screen according to the impedance obtained in real time.

Optionally, the device further includes: a target interval determination module, configured to determine a target interval corresponding to the impedance, according to the impedance acquired in real time and the impedance ranges respectively corresponding to multiple preset ablation impedance prompting intervals; and a schematic real-time impedance diagram drawing module, configured to draw a schematic real-time impedance diagram on the screen according to the impedance, the impedance range and the target interval. The schematic real-time impedance diagram includes: description information of the multiple ablation impedance prompting intervals, description information of a preset reference impedance, and description information of the corresponding relationship between the impedance and the target interval. The multiple ablation impedance prompting intervals include: a non-ablatable impedance interval, an ablatable impedance interval, and an optimal ablatable impedance interval.

Optionally, the description information of the multiple ablation impedance prompting intervals includes: multiple vertically laminated columns and the corresponding description texts and indicating graphics of the multiple ablation impedance prompting intervals. The description information of the preset reference impedance includes: the description text and the indicating graphic of the preset reference impedance.

The multiple columns respectively correspond, from top to bottom, to an upper range of the non-ablatable impedance interval, an upper range of the ablatable impedance interval, an upper range of the optimal ablatable impedance interval, a lower range of the optimal ablatable impedance interval, a lower range of the ablatable impedance interval, and a lower range of the non-ablatable impedance interval.

Taking the column corresponding to the optimal ablatable impedance interval as a center, the height increases as the distance of the other columns in the multiple columns from the column increases.

Optionally, the device further includes: a schematic real-time impedance change diagram drawing module, configured to draw a schematic real-time impedance change diagram on the screen according to the impedance, the impedance range and the target interval. The schematic real-time impedance change diagram includes: two-dimensional coordinate axes, description information of the multiple ablation impedance prompting intervals, and description information of the corresponding relationship between each impedance and respective target interval. The multiple ablation impedance prompting intervals include: a non-ablatable impedance interval, an ablatable impedance interval, and an optimal ablatable impedance interval.

The horizontal axis of the two-dimensional coordinate axes is a time axis, indicating the acquisition time of each impedance and also the preset reference impedance. The vertical axis of the two-dimensional coordinate axes indicates, from bottom to top in a positive direction, an upper range of the optimal ablatable impedance interval, an upper range of the ablatable impedance interval, and an upper range of the non-ablatable impedance interval, and indicates, from top to bottom in a negative direction, a lower range of the optimal ablatable impedance interval, a lower range of the ablatable impedance interval, and a lower range of the non-ablatable impedance interval.

Optionally, the description information of the corresponding relationship includes: graphics of different colors with preset shapes, wherein the different colors respectively correspond to different ablation impedance prompting intervals.

The height of the graphic is determined according to the difference between the impedance and the upper limit or lower limit of the corresponding target interval.

Optionally, the target interval determination module is specifically configured to determine a target interval corresponding to the single impedance in real time according to the single impedance acquired in real time and the impedance ranges respectively corresponding to multiple ablation impedance prompting intervals when the radio-frequency ablation catheter is a single-electrode radio-frequency ablation catheter; and analyze whether the multiple impedances obtained in real time correspond to the same ablation impedance prompting interval according to the impedance ranges respectively corresponding to multiple ablation impedance prompting intervals, when the radio-frequency ablation catheter is a multi-electrode radio-frequency ablation catheter, determine the corresponding ablation impedance prompting interval as the target interval, if the multiple impedances correspond to the same ablation impedance prompting interval, and an ablation impedance prompting interval that covers the most impedances is used as the target interval.

Optionally, the marking module 702 includes: a first positioning module, configured to acquire a picture of a current ablation operation captured by an endoscope and compare the captured picture with the image, to obtain the position data of the target ablation point in the image.

Optionally, the marking module 702 further includes: a second positioning module, configured to obtain an ultrasound image of the target ablation point; and acquire the position data of the target ablation point in the image according to the ultrasound image.

Optionally, the marking module 702 further includes: a third positioning module, configured to acquire the position data of the target ablation point, by electromagnetic navigation technology.

Optionally, the device further includes: a screencapture module, configured to perform a screencapture operation when the prompt information outputted on the screen or the drawn schematic diagram has target information containing preset keywords, and save the captured picture; and a display module, configured to display all the pictures saved on the screen according to the priority of the target information, after the ablation task is ended.

Optionally, the device further includes: an ablation status analyzing module, configured to determine whether the ablation status of the target ablation point reaches a preset target ablation status according to the elapsed ablation time and the temperature, and output prompt information indicating reaching of the target ablation status if the ablation status of the target ablation point reaches the target ablation status.

The marking module 702 is further configured to take an ablation point corresponding to a changed position as the target ablation point, when the position of the ablation operation is detected to be changed; update the mark in the image and return to implement the step of acquiring the elapsed ablation time and the temperature of the target ablation point in real time, and determining the ablation status of the target ablation point according to the elapsed ablation time and the temperature, until the ablation operation is ended.

The specific process for the above modules to implement their respective functions can be made reference to the relevant description in the embodiments shown in FIG. 46 to FIG. 54, and will not be repeated here.

In the embodiments of this application, when an ablation task is triggered, an image of an ablation site is acquired and displayed on a preset prompting interaction interface where the image of the ablation site is marked with a currently-being-ablated target ablation point; according to the elapsed ablation time and the temperature of the target ablation point acquired in real time, a schematic image showing the real-time dynamic change of the ablation status of the target ablation point is generated and displayed, so that the status changes of an ablation site can be displayed in real time and intuitively during the implementation of the ablation operation, thereby improving the effectiveness and relevance of information prompts.

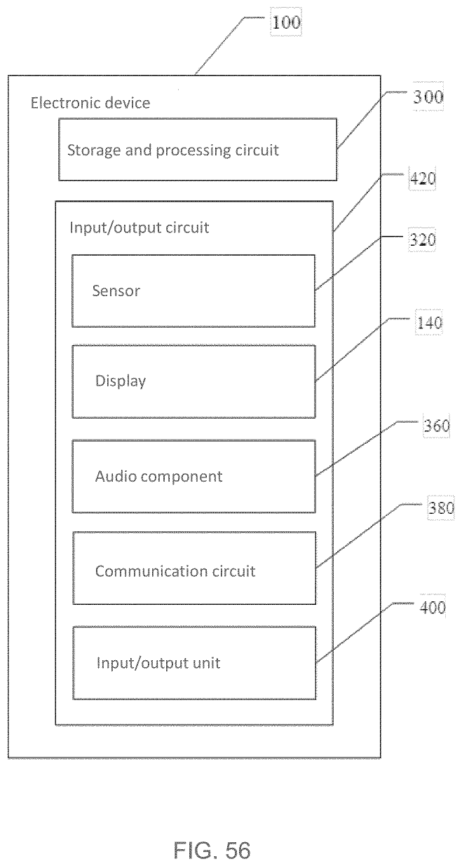

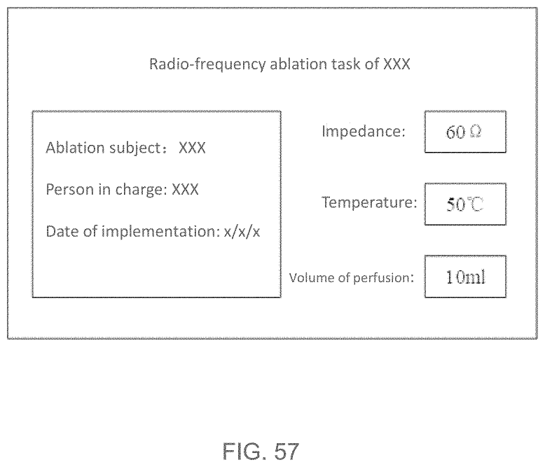

FIG. 56 is a schematic diagram showing a hardware structure of an electronic device provided in an embodiment of the present invention.

Exemplarily, the electronic device may be any of various types of computer devices that are non-movable or movable or portable and capable of wireless or wired communication. Specifically, the electronic device can be a desktop computer, a server, a mobile phone or smart phone (e.g., a phone based on iPhone™, and Android™), a portable game device (e.g. Nintendo DS™, PlayStation Portable™, Gameboy Advance™, iPhone™), a laptop computer, PDA, a portable Internet device, a portable medical device, a smart camera, a music player, a data storage device, and other handheld devices such as watches, earphones, pendants, and headphones, etc. The electronic device may also be other wearable devices (for example, electronic glasses, electronic clothes, electronic bracelets, electronic necklaces and other head-mounted devices (HMD)).

In some cases, the electronic device can implement a variety of functions, for example: playing music, displaying videos, storing pictures and receiving and sending phone calls.

As shown in FIG. 56, the electronic device 100 includes a control circuit, and the control circuit includes a storage and processing circuit 300. The storage and processing circuit 300 includes a storage, for example, hard drive storage, non-volatile storage (such as flash memory or other storages that are used to form solid-state drives and are electronically programmable to confine the deletion, etc.), and volatile storage (such as static or dynamic random access storage) which is not limited in the embodiments of the present invention. The processing circuit in the storage and processing circuit 300 can be used to control the operation of the electronic device 100. The processing circuit can be implemented based on one or more microprocessors, microcontrollers, digital signal processors, baseband processors, power management units, audio codec chips, application specific integrated circuits, and display driver integrated circuits.

The storage and processing circuit 300 can be used to run the software in the electronic device 100, such as Internet browsing applications, Voice over Internet Protocol (VOIP) phone call applications, Email applications, media player applications, and functions of operating system. The software can be used to perform some control operations, for example, camera-based image acquisition, ambient light measurement based on an ambient light sensor, proximity sensor measurement based on a proximity sensor, information display function implemented by a status indicator based on a status indicator lamp such as light emitting diode, touch event detection based on a touch sensor, functions associated with displaying information on multiple (e.g. layered) displays, operations associated with performing wireless communication functions, operations associated with the collection and generation of audio signals, control operations associated with the collection and processing of button press event data, and other functions in electronic device 100, which are not limited in the embodiments of the present invention.

Further, the storage stores an executable program code. The processor coupled to the storage calls the executable program code stored in the storage, and implement the ablation operation prompting method described in the embodiments shown in FIGS. 46 to 54.

The executable program code includes various modules in the ablation operation prompting device described in the embodiment shown in FIG. 55, for example, the image display module 701, the marking module 702, the ablation status determination module 703, and the ablation status prompting module 704. The specific process for the above modules to implement their functions can be made reference to the relevant description in the embodiments shown in FIG. 55, and will not be repeated here.

The electronic device 100 may also include an input/output circuit 420. The input/output circuit 420 can be used to enable the electronic device 100 to implement the input and output of data, that is, the electronic device 100 is allowed to receive data from an external device and the electronic device 100 is also allowed to output data from the electronic device 100 to the external device. The input/output circuit 420 may further include a sensor 320. The sensor 320 may include one or a combination of an ambient light sensor, a proximity sensor based on light and capacitance, a touch sensor (e.g., light-based touch sensor and/or capacitive touch sensor, wherein, wherein the touch sensor may be part of the touch screen, or it can also be used independently as a touch sensor structure), an accelerometer, and other sensors, etc.

The input/output circuit 420 may also include one or more displays, for example, display 140. The display 140 may include a liquid crystal display, an organic light emitting diode display, an electronic ink display, a plasma display, and a display that uses other display technologies. The display 140 may include a touch sensor array (i.e., the display 140 may be a touch display screen). The touch sensor can be a capacitive touch sensor formed by an array of transparent touch sensor electrodes (such as indium tin oxide (ITO) electrodes), or a touch sensor formed using other touch technologies, for example, sonic touch, pressure sensitive touch, resistive touch, and optical touch, which is not limited in the embodiments of the present invention.

The electronic device 100 can also include an audio component 360. The audio component 360 can be used to provide audio input and output functions for the electronic device 100. The audio component 360 in the electronic device 100 includes a speaker, a microphone, a buzzer, and a tone generator and other components used to generate and detect sound.

A communication circuit 380 can be used to provide the electronic device 100 with an ability to communicate with an external device. The communication circuit 380 may include an analog and digital input/output interface circuit, and a wireless communication circuit based on radio-frequency signals and/or optical signals. The wireless communication circuit in the communication circuit 380 may include a radio-frequency transceiver circuit, a power amplifier circuit, a low-noise amplifier, a switch, a filter, and an antenna. For example, the wireless communication circuit in the communication circuit 380 may include a circuit for supporting near field communication (NFC) by transmitting and receiving near-field coupled electromagnetic signals. For example, the communication circuit 380 may include a near-field communication antenna and a near-field communication transceiver. The communication circuit 380 may also include a cellular phone transceiver and antenna, and a wireless LAN transceiver circuit and antenna, etc.

The electronic device 100 may also further include a battery, a power management circuit and other input/output units 400. The input/output unit 400 may include a button, a joystick, a click wheel, a scroll wheel, a touchpad, a keypad, a keyboard, a camera, a light-emitting diode and other status indicators, etc.

The user can control the operation of the electronic device 100 by inputting a command through the input/output circuit 420, and the output data from the input/output circuit 420 enables the receiving of status information and other outputs from the electronic device 100.

Further, an embodiment of the present invention further provides a non-transitory computer-readable storage medium. The non-transitory computer-readable storage medium can be configured in the server in each of the above embodiments, and a computer program is stored in the non-transitory computer-readable storage medium. When the program is executed by the processor, the ablation operation prompting method described in the embodiments shown in FIG. 46 to FIG. 54 is implemented.

In the embodiments described above, emphasis has been placed on the description of various embodiments. Parts of an embodiment that are not described or illustrated in detail may be found in the description of other embodiments.

Those of ordinary skill in the art will recognize that the exemplary modules/units and algorithm steps described in connection with the embodiments disclosed herein may be implemented by electronic hardware, or a combination of computer software and electronic hardware. Whether such functions are implemented by hardware or software depends on the particular application and design constraints of the technical solutions. Skilled artisans may implement the described functions in varying ways for each particular application. but such implementation is not intended to exceed the scope of the present invention.

In the embodiments provided in the present invention, it can be understood that the disclosed device/terminal and method may be implemented in other ways. For example, the device/terminal embodiments described above are merely illustrative. For example, a division of the modules or elements is merely division of logical functions, and there may be additional divisions in actual implementation. For example, multiple units or components may be combined or integrated into another system, or some features may be omitted or not performed. Alternatively, the couplings or direct couplings or communicative connections shown or discussed with respect to one another may be indirect couplings or communicative connections via some interfaces, devices or units may be electrical, mechanical or otherwise.

The units described as separate components may or may not be physically separate, and the components shown as units may or may not be physical units, i.e. may be located in one place, or may be distributed over a plurality of network elements. Some or all of the units may be selected to achieve the objectives of the solution of the present embodiment according to practical requirements.

In addition, the functional units in the various embodiments of the present invention may be integrated into one processing unit, may be physically separate from each other or may be integrated in one unit by two or more units. The integrated units described above can be implemented either in the form of hardware, or software functional units.

The integrated unit, if implemented in the form of a software functional unit and sold or used as a stand-alone product, may be stored in a computer readable storage medium. Based on such an understanding, all or part of the processes of the methods in the above-described embodiments implemented in the present invention, may also be implemented by a computer program instructing related hardware. The computer program may be stored in a computer-readable storage medium, and performs the steps of the various method embodiments described above when executed by the processor. The computer program includes a computer program code, which may be in the form of source code, object code, or executable file, or in some intermediate form. The computer readable medium may include: any entity or device capable of carrying the computer program code, recording media, U disks, removable hard disks, magnetic disks, optical disks, computer memory, read-only memory (ROM), random access memory (RAM), electric carrier wave signals, telecommunication signals and software distribution media. It should be noted that the computer readable medium may contain content that may be appropriately augmented or subtracted as required by legislation and patent practice within judicial jurisdictions, e.g., the computer-readable medium does not include electrical carrier wave signals and telecommunications signals in accordance with legislation and patent practices in some jurisdictions.

The above-described embodiments are merely illustrative of, and not intended to limit the technical solutions of the present invention. Although the present invention has been described in detail with reference to the foregoing embodiments, it should be understood by those of ordinary skill in the art that the technical solutions of the above-mentioned embodiments can still be modified, or some of the technical features thereof can be equivalently substituted; and such modifications and substitutions do not cause the nature of the corresponding technical solution to depart from the spirit and scope of the embodiments of the present disclosure and are intended to be included within the scope of this application.

Radio-Frequency Operation Prompting Method, Electronic Device, and Computer-Readable Storage Medium

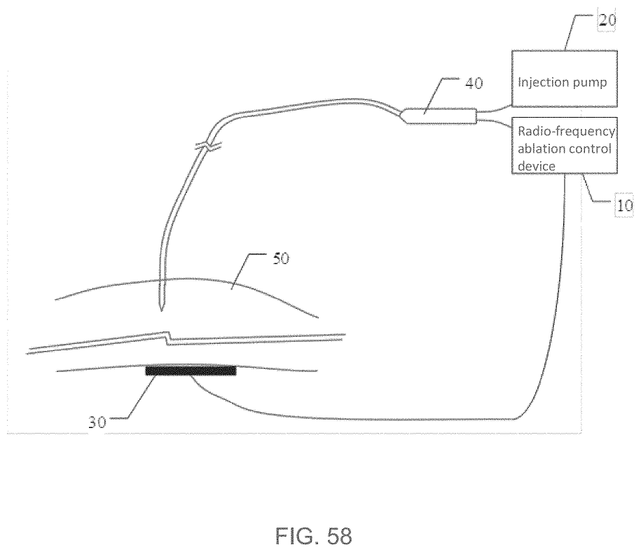

FIG. 58 is a schematic view showing an application scenario of a radio-frequency operation prompting method provided in an embodiment of the present invention. The radio-frequency operation prompting method can be implemented by a radio-frequency host 10 shown in FIG. 58, or implemented by other computer equipment that has established a data connection with the radio-frequency host 10.

As shown in FIG. 58, the radio-frequency host 10 is connected to a syringe pump 20, a neutral electrode 30 and a radio-frequency operation catheter 40. The radio-frequency host 10 is provided with a built-in display screen (not shown).

Specifically, before an operation task is implemented, an energy emitting end of the radio-frequency operation catheter 40 for generating and outputting radio-frequency energy and an extension tube (not shown) of the syringe pump 20 are inserted into the body of a subject 50 (such as an abnormal tissue mass). Then, the neutral electrode 30 is brought into contact with the skin surface of the subject 50. A radio-frequency current flows through the radio-frequency operation catheter 40, the subject 50 and the neutral electrode 30, to form a circuit.

When the operation task is triggered, the radio-frequency operation catheter 40 is controlled by the radio-frequency host 10 to output radio-frequency energy to an operation site by discharging, so as to implement a radio-frequency operation on the operation site. Moreover, the syringe pump 20 performs a perfusion operation on the subject through the extension tube, wherein physiological saline is infused into the operation site, to adjust the impedance and temperature of the operation site.

Moreover, the radio-frequency host 10 acquires physical characteristic data of an operating position in the subject of the radio-frequency operation in real time by multiple probes (not shown) provided at a tip of the radio-frequency operation catheter 40, obtains a physical characteristic field of the subject of the radio-frequency operation according to the physical characteristic data acquired in real time; and then obtains the change of range of a to-be-operated area in a target operating area according to an initial range of the target operating area in the subject of the radio-frequency operation and the change of value of the physical characteristic data in the physical characteristic field, and displays the change of range by a three-dimensional model.

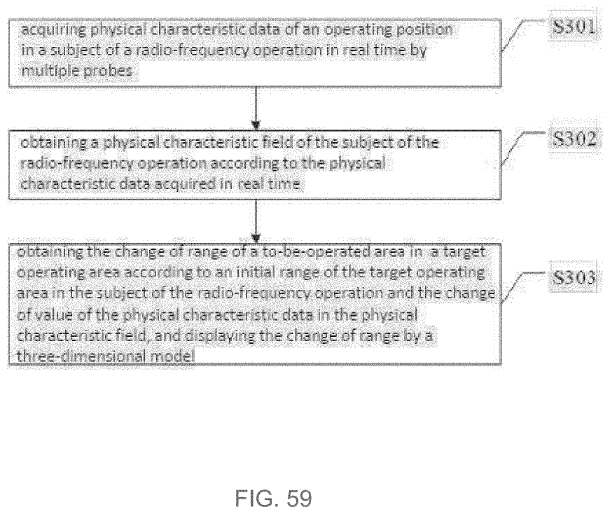

FIG. 59 shows a flow chart of a radio-frequency operation prompting method provided in an embodiment of the present invention. The method can be implemented by the radio-frequency host 10 shown in FIG. 58, or implemented by other computer terminals connected thereto. For ease of description, in the following embodiments, the radio-frequency host 10 is used as an implementation body. As shown in FIG. 59, the method includes specifically:

Step S301: acquiring physical characteristic data of an operating position in a subject of a radio-frequency operation in real time by multiple probes.

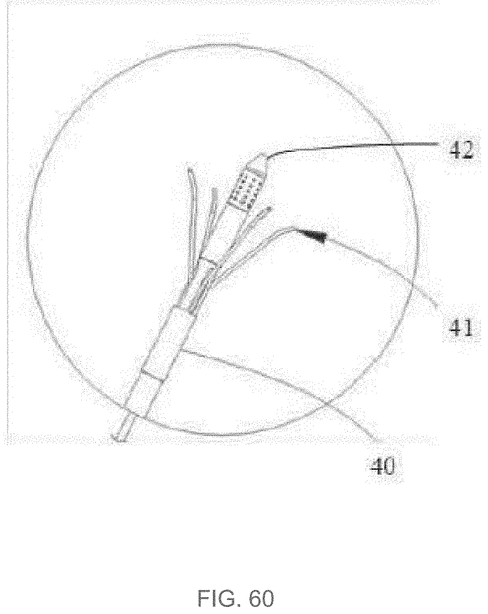

As shown in FIG. 60, multiple probes 41 are arranged around a central electrode 42 for outputting radio-frequency energy provided at a tip of the radio-frequency operation catheter 40, and located on different planes, to form a claw-shaped structure collectively. Each probe is provided with a physical characteristic data acquiring device, configured to acquire physical characteristic data of a pierced or touched position.

Particularly, when the radio-frequency operation catheter is controlled by the radio-frequency host to perform a radio-frequency operation, the probe comes into contact with an operating site of the subject of the radio-frequency operation along with the central electrode, to detect the physical characteristic data of different positions in the operating site in real time. The physical characteristic data can be specifically temperature or impedance, or both temperature and impedance.

The subject of the radio-frequency operation refers to any subject or object that can receive radio-frequency operation such as radio-frequency ablation. For example, when the radio-frequency operation is radio-frequency ablation, the subject of the radio-frequency operation may be a biological tissue, and the operating position may be an abnormal tissue in the biological tissue.

Step S302: obtaining a physical characteristic field of the subject of the radio-frequency operation according to the physical characteristic data acquired in real time.

Specifically, the physical characteristic data includes temperature data and impedance data, and correspondingly, the physical characteristic field can include a temperature field and a resistance field.

The temperature field is a set of temperatures at various points in the subject of the radio-frequency operation, reflecting the spatial and temporal distribution of temperature; and can generally be expressed as a function of spatial coordinates and time of an object. That is, t=f(x, y, z, t), wherein x, y, and z are three rectangular coordinates in space, and r is the time coordinate. In the prior art, many specific algorithms for temperature field are available, which are not particularly limited in the present invention.

Similar to the temperature field, the impedance field is a set of impedances at various points in the subject of the radio-frequency operation, and is a function of time and spatial coordinates, reflecting the spatial and temporal distribution of impedance.

Step S303: obtaining the change of range of a to-be-operated area in a target operating area according to an initial range of the target operating area in the subject of the radio-frequency operation and the change of value of the physical characteristic data in the physical characteristic field, and displaying the change of range by a three-dimensional model.

The target operating area is an implementation area of the present radio-frequency operation in the subject of the radio-frequency operation, and the initial range of the target operating area can be obtained by X-ray scanning and other transmission scanning techniques.

The to-be-operated area is an area where the present radio-frequency operation has not been performed or the effect after the present radio-frequency operation has not reached the standard, and an area where the radio-frequency operation still needs to be performed.

The value of the physical characteristic data of each point in the physical characteristic field changes at any time as the radio-frequency operation progresses, and the value of the physical characteristic data represents the stage of the radio-frequency operation. Particularly, when the value of the physical characteristic data reaches a preset threshold, it indicates the end of the radio-frequency operation (i.e., achieving the desired effect). When the value of the physical characteristic data is less than the preset threshold, it indicates that the radio-frequency operation is not ended (i.e., not achieving the desired effect) or does not start. The area where the radio-frequency operation is not ended or does not start is the to-be-operated area. That is, according to the value of the physical characteristic data in the physical characteristic field, the range of the to-be-operated area can be determined. As the measured value of each point in the physical characteristic field changes, the range of the to-be-operated area changes accordingly, showing such a trend that as the past time of radio-frequency operation increases, the range of the to-be-operated area becomes smaller and smaller.

By default 3D model display software, the change of range of the to-be-operated area in the target operating area is displayed on a display interface of the radio-frequency host, to intuitively present it to a radio-frequency operation personnel to get to know the status of the radio-frequency operation.

In the embodiments of this application, multiple pieces of physical characteristic data of an operating position in a subject of a radio-frequency operation are acquired in real time by multiple probes, a physical characteristic field of the subject of the radio-frequency operation is obtained according to these pieces of data, then the change of range of a to-be-operated area in a target operating area is obtained according to an initial range of the target operating area in the subject of the radio-frequency operation and the change of value of the physical characteristic data in the physical characteristic field, and the range of change is displayed by a three-dimensional model. As a result, the visual prompting of the change of range of the to-be-operated area is realized, the content of the prompt information is much rich, intuitive and vivid, and the accuracy and intelligence of determining the to-be-operated area is increased, thereby improving the effectiveness of information prompts, and thus improving the success rate and effect of the radio-frequency operation.

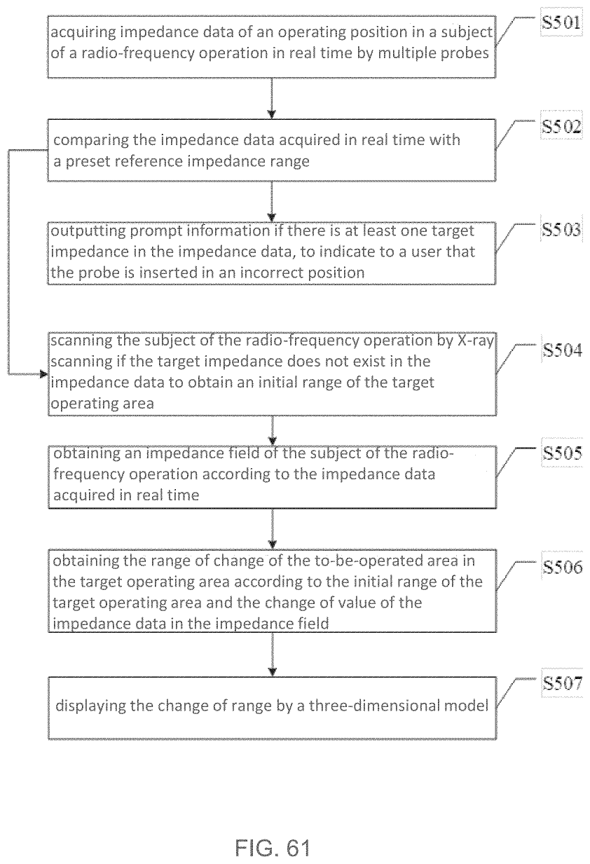

FIG. 61 shows a flow chart of a radio-frequency operation prompting method provided in another embodiment of the present invention. The method can be implemented by the radio-frequency host 10 shown in FIG. 58, or implemented by other computer terminals connected thereto. For ease of description, in the following embodiments, the radio-frequency host 10 is used as an implementation body. As shown in FIG. 61, the method includes specifically:

Step S501: acquiring impedance data of an operating position in a subject of a radio-frequency operation in real time by multiple probes.

Step S502: comparing the impedance data acquired in real time with a preset reference impedance range.

Step S503: outputting prompt information if there is at least one target impedance in the impedance data, to indicate to a user that the probe is inserted in an incorrect position.

As shown in FIG. 60, multiple probes 41 are arranged around a central electrode 42 provided at a tip of the radio-frequency operation catheter 40, and located on different planes, to form a claw-shaped structure collectively. Each probe is provided with an impedance acquiring device, configured to acquire the impedance data of a pierced or touched position.

It can be understood that the impedance of normal biological tissues and the impedance of abnormal biological tissues are different, and a reference impedance database is configured in the radio-frequency host 10. The reference impedance database is configured to store the reference impedance range corresponding to each of different types of abnormal biological tissues (e.g., tumors, inflammations, and cancers). By querying the reference impedance database, the reference impedance range corresponding to the type of abnormality in the subject of the current radio-frequency operation can be obtained. Optionally, the reference impedance database can also be configured in the cloud.

The value of the target impedance is not within the reference impedance range. The impedance data acquired by the multiple probes is compared with the reference impedance range queried, if there is at least one target impedance in the acquired impedance data, it means that the probe does not completely cover the site where the radio-frequency operation needs to be performed, and the expected result may not be achieved if the radio-frequency operation is performed at the current position. Consequently, preset prompt information is output on the display screen, to indicate to the user that the probe is inserted in an incorrect position.

Further, the prompt information also includes position information of a probe that obtains the target impedance, so that the user can determine a displacement direction of the probe according to the position information, making the information prompt more intelligent.

Further, after the prompt information is outputted, the process is returned to Step S501 every preset period of time until the target impedance does not exist in the impedance data; or in response to a control command triggered by the user by pressing a preset physical or virtual button, Step S501 is performed again.

Therefore, by using the reference impedance range, prompting is made when the probe is inserted in an incorrect position, to realize the navigation of the probe positioning operation. This can make the information prompt more intelligent, and increase the speed of probe positioning, thus shortening the overall time of radio-frequency operation, and improving the operation efficiency.

Step S504: scanning the subject of the radio-frequency operation by X-ray scanning if the target impedance does not exist in the impedance data to obtain an initial range of the target operating area.

Specifically, if the target impedance does not exist in the acquired impedance data, that is, all the acquired impedances fall into the reference impedance range, indicating that the probe completely covers the site where the radio-frequency operation needs to be performed, a three-dimensional image of a target site is obtained by scanning the target site of the subject of the radio-frequency operation by X-ray scanning. Then the three-dimensional image is recognized, to obtain the position coordinates of each probe in the three-dimensional image, Then, the range of coverage of the probe is determined according to the obtained position coordinates, and the range of coverage is determined as the initial range of the target operating area. The X-ray scanning includes, for example, Computed Tomography (CT).

Step S505: obtaining an impedance field of the subject of the radio-frequency operation according to the impedance data acquired in real time.

Specifically, the impedance field is a set of impedances at various points in the subject of the radio-frequency operation, and is a function of time and spatial coordinates, reflecting the spatial and temporal distribution of impedance.

Step S506: obtaining the change of range of the to-be-operated area in the target operating area according to the initial range of the target operating area and the change of value of the impedance data in the impedance field.

The to-be-operated area is an area where the present radio-frequency operation has not been performed or the effect after the present radio-frequency operation has not reached the standard, and an area where the radio-frequency operation still needs to be performed. The value of the impedance data of each point in the impedance field changes at any time as the radio-frequency operation progresses, and the value of the impedance data represents the stage of the radio-frequency operation. For example, when the value of the impedance data reaches a preset threshold, it indicates that the radio-frequency operation achieves the desired effect. When the value of the impedance data is less than the preset threshold, it indicates that the radio-frequency operation does not achieve the desired effect or does not start. The area where the radio-frequency operation does not achieve the desired effect or does not start is the to-be-operated area. That is, according to the value of the impedance data in the impedance field, the range of the to-be-operated area can be determined. As the measured value of each point in the impedance field changes, the range of the to-be-operated area changes accordingly, showing such a trend that as the past time of radio-frequency operation increases, the range of the to-be-operated area becomes smaller and smaller.

Specifically, the value of the impedance data of each point in the impedance field is compared with a preset threshold (i.e., preset impedance threshold), and the boundary of the to-be-operated area is determined according to the points where the value of the impedance data is greater than the preset threshold.

It can be understood that the points where the value of the impedance data is greater than the preset threshold are fitted together by using a default fitting algorithm, to obtain the range of the area in the target operating area that has achieved the expected effect. The initial range of the target operating area is compared with the range of the area that has achieved the expected effect, to obtain the range and boundary of the to-be-operated area. The preset fitting algorithm includes, but is not limited to, for example, the least square method or the Matlab curve fitting algorithm, which is not particularly limited in the present invention.

Further, when the value of the impedance data of the operating position is greater than the preset threshold, the range of a radiation area of the operating position is determined according to the value of the impedance data of the operating position, a detection angle of the probe corresponding to the operating position and a preset radiation distance; and the boundary of the to-be-operated area is determined according to the range of the radiation area.

It can be understood that since the radio-frequency energy outputted by the central electrode of the radio-frequency operation catheter is radiated into the biological tissue along a specific direction, the impedance change has a radiation range.

The detection angle of the probe, that is, the angle at which the probe used to detect the impedance of a certain operating position is pierced into or brought into contact with the operating position. According to the detection angle, the direction of radiation can be determined. According to the value of the impedance data of the operating position, the direction of the radiation, and the preset radiation distance, the range of the radiation area of the operating position, that is, the depth of the boundary of the range of the area that has achieved the desired effect, can be determined.

Step S507: displaying the change of range by a three-dimensional model.

Specifically, according to the three-dimensional image of the target operating area obtained by X-ray scanning and the change of range of the to-be-operated area in the target operating area, and by using algorithms such as Marching Cubes based on surface rendering, or Ray-casting, Shear-warp, Frequency Domain, and Splatting based on volume rendering, a three-dimensional model of change of range is established for the to-be-operated area, and displayed on a preset display interface.

In the Marching Cubes algorithm, a series of two-dimensional slice data is deemed as a three-dimensional data field, and then a three-dimensional surface mesh modeling a three-dimensional model is established by extracting the isosurface of the three-dimensional data. In this way, the three-dimensional model is established. The algorithm based on volume rendering is to directly convert the discrete data in a three-dimensional space into a final three-dimensional image, without generating intermediate geometric primitives. The central idea is to define an opacity for each voxel, take the transmission, emission and reflection of each voxel to light into account.

Optionally, in another embodiment of the present invention, the multiple probes are configured to acquire temperature data of an operating position in a subject of a radio-frequency operation in real time, and the method includes the following steps:

Step S701: acquiring temperature data of an operating position in a subject of a radio-frequency operation in real time by multiple probes.

Step S702: obtaining an initial range of a target operating area by scanning the subject of the radio-frequency operation by X-ray scanning.

Step S703: obtaining a temperature field of the subject of the radio-frequency operation according to the temperature data acquired in real time.

Step S704: obtaining the range of change of a to-be-operated area in the target operating area according to the initial range of the target operating area and the change of value of the temperature data in the temperature field.

Step S705: displaying the change of range by a three-dimensional model.

The Steps S701 to S705 are similar to Step S501 and Steps S504 to S507, and related description can be made reference to Step S501 and Steps S504 to S507, and will not be repeated here.

Optionally, in another embodiment of the present invention, the multiple probes are configured to acquire temperature data and impedance data of an operating position in a subject of a radio-frequency operation in real time, and the method includes the following steps:

Step S801: acquiring temperature data and impedance data of an operating position in a subject of a radio-frequency operation in real time by multiple probes.

Step S802: comparing the impedance data acquired in real time with a preset reference impedance range.

Step S803: outputting prompt information if there is at least one target impedance in the impedance data, to indicate to a user that the probe is inserted in an incorrect position.

Step S804: obtaining the initial range of the target operating area by scanning the subject of the radio-frequency operation by X-ray scanning, if the target impedance does not exist in the impedance data.

Step S805: obtaining an impedance field and a temperature field of the subject of the radio-frequency operation according to the impedance data and the temperature data acquired in real time.

Step S806: obtaining the range of change of a to-be-operated area in the target operating area according to the initial range of the target operating area, the change of value of the impedance data in the impedance field and the change of value of the temperature data in the temperature field.

Step S807: displaying the change of range by a three-dimensional model.

The Steps S801 to S805 and Step S807 are similar to Steps S501 to S505 and Step S507, and related description can be made reference to Step S501 and Steps S505 to S507, and will not be repeated here.

Unlikely, Step S805 specifically includes: comparing the value of the temperature data of each point in the temperature field with a preset temperature threshold, and determining a first boundary of the to-be-operated area according to points where the value of the temperature data is greater than the preset temperature threshold; and after a preset period of time, comparing the value of the impedance data at each point in the impedance field with a preset impedance threshold, and calibrating the first boundary of the to-be-operated area according to points where the value of the impedance data is greater than the preset impedance threshold, to obtain a second boundary, wherein the second boundary is determined as the boundary of the to-be-operated area.

The step of calibrating the first boundary of the to-be-operated area according to points where the value of the impedance data is greater than the preset impedance threshold to obtain a second boundary is that at the same point, if the value of the impedance data is greater than the preset impedance threshold, but the value of the temperature data is not greater than the preset temperature threshold, the impedance data prevails, and the location corresponding to this point is determined as the location that achieves the desired effect.

Similarly, the first boundary is determined by using the temperature field, and then the first boundary is calibrated by using the impedance field, to achieve a complementary effect, and make the final boundary determined more accurate.

Optionally, the method also includes: determining the unit change of the to-be-operated area periodically according to the change of range of the to-be-operated area; and determining the remaining time before the end of the current radio-frequency operation according to the unit change and the current volume of the to-be-operated area, and outputting the remaining time as prompt information on a display interface.

Specifically, an interval is determined according to a preset time, and the change of range of the to-be-operated area is divided by the past implementation time of the radio-frequency operation every preset period of time, to obtain the unit change of the to-be-operated area, for example: the volume of the to-be-operated area reduced per second. Then, the current volume of the to-be-operated area is divided by the unit change, to obtain the remaining time before the end of the current radio-frequency operation.

The initial volume of the target operating area minus the unit changes of the to-be-operated area is the current volume of the to-be-operated area. The initial volume of the target operating area can be determined based on the three-dimensional image of the target operating area obtained by X-ray scanning in Step S504.

Therefore, by prompting the remaining time before the end of the current radio-frequency operation, the user is allowed to get to know the process of radio-frequency operation, thus further improving the intelligence of information prompts.

In the embodiments of this application, multiple pieces of physical characteristic data of an operating position in a subject of a radio-frequency operation are acquired in real time by multiple probes, a physical characteristic field of the subject of the radio-frequency operation is obtained according to these pieces of data, then the change of range of a to-be-operated area in a target operating area is obtained according to an initial range of the target operating area in the subject of the radio-frequency operation and the change of value of the physical characteristic data in the physical characteristic field, and the range of change is displayed by a three-dimensional model. As a result, the visual prompting of the change of range of the to-be-operated area is realized, the content of the prompt information is much rich, intuitive and vivid, and the accuracy and intelligence of determining the to-be-operated area is increased, thereby improving the effectiveness of information prompts, and thus improving the success rate and effect of the radio-frequency operation.

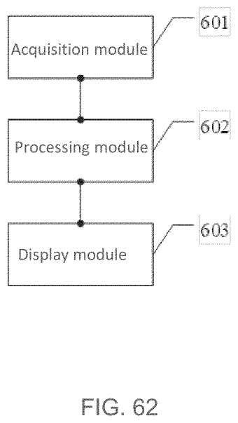

FIG. 62 is a schematic structural diagram of a radio-frequency operation prompting device provided in an embodiment of the present invention. For ease of description, only the parts relevant to the embodiments of the application are shown. The device may be a computer terminal, or a software module configured on the computer terminal. As shown in FIG. 62, the device includes an acquisition module 601, a processing module 602, and a display module 603.

The acquisition module 601 is configured to acquire physical characteristic data of an operating position in a subject of a radio-frequency operation in real time by multiple probes.

The processing module 602 is configured to obtain a physical characteristic field of the subject of the radio-frequency operation according to the physical characteristic data acquired in real time, and obtain the change of range of a to-be-operated area in a target operating area according to an initial range of the target operating area in the subject of the radio-frequency operation and the change of value of the physical characteristic data in the physical characteristic field.

The display module 603 is configured to display the change of range by a three-dimensional model.

Further, the processing module 602 is further configured to compare the value of the physical characteristic data of each point in the physical characteristic field with a preset threshold; and determine a boundary of the range of the to-be-operated area according to points where the value of the physical characteristic data is greater than the preset threshold.

The processing module 602 is further configured to determine the range of a radiation area of the operating position according to the value of the physical characteristic data of the operating position, a detection angle of the probe corresponding to the operating position and a preset radiation distance, when the value of the physical characteristic data of the operating position is greater than the preset threshold. The value of the physical characteristic data in the radiation area is greater than the preset threshold; and the boundary of the to-be-operated area is determined according to the range of the radiation area.

Further, the physical characteristic data includes temperature data and/or impedance data, and the physical characteristic field includes a temperature field and/or a resistance field.

Further, when the physical characteristic data includes temperature data and impedance data, and the physical characteristic field includes a temperature field and a resistance field, the processing module 602 is further configured to compare the value of the temperature data of each point in the temperature field with a preset temperature threshold, and determine a first boundary of the to-be-operated area according to points where the value of the temperature data is greater than the preset temperature threshold; and compare the value of the impedance data at each point in the impedance field with a preset impedance threshold after a preset period of time, and calibrate the first boundary of the to-be-operated area according to points where the value of the impedance data is greater than the preset impedance threshold to obtain a second boundary wherein the second boundary is determined as the boundary of the to-be-operated area.

Further, the processing module 602 is further configured to determine the unit change of the to-be-operated area periodically according to the change of range of the to-be-operated area; and determine the remaining time before the end of the current radio-frequency operation according to the unit change and the current volume of the to-be-operated area.

The display module 603 is configured to output the remaining time as prompt information on a display interface.

Further, when the physical characteristic data includes impedance data, the processing module 602 is further configured to compare the impedance data acquired in real time with a preset reference impedance range, and trigger the display module 603 to output prompt information if there is at least one target impedance in the impedance data, to indicate to a user that the probe is inserted in an incorrect position, and the value of the target impedance is not within the reference impedance range;

and trigger the step of obtaining a physical characteristic field of the subject of the radio-frequency operation, according to the physical characteristic data acquired in real time, if the target impedance does not exist in the impedance data.

Further, the processing module 602 is further configured to scan the subject of the radio-frequency operation by X-ray scanning, to obtain the initial range of the target operating area.

The specific process for the above modules to implement their respective functions can be made reference to the relevant description in the embodiments shown in FIG. 59 to FIG. 61, and will not be repeated here.

In the embodiments of this application, multiple pieces of physical characteristic data of an operating position in a subject of a radio-frequency operation are acquired in real time by multiple probes, a physical characteristic field of the subject of the radio-frequency operation is obtained according to these pieces of data, then the change of range of a to-be-operated area in a target operating area is obtained according to an initial range of the target operating area in the subject of the radio-frequency operation and the change of value of the physical characteristic data in the physical characteristic field, and the range of change is displayed by a three-dimensional model. As a result, the visual prompting of the change of range of the to-be-operated area is realized, the content of the prompt information is much rich, intuitive and vivid, and the accuracy and intelligence of determining the to-be-operated area is increased, thereby improving the effectiveness of information prompts, and thus improving the success rate and effect of the radio-frequency operation.

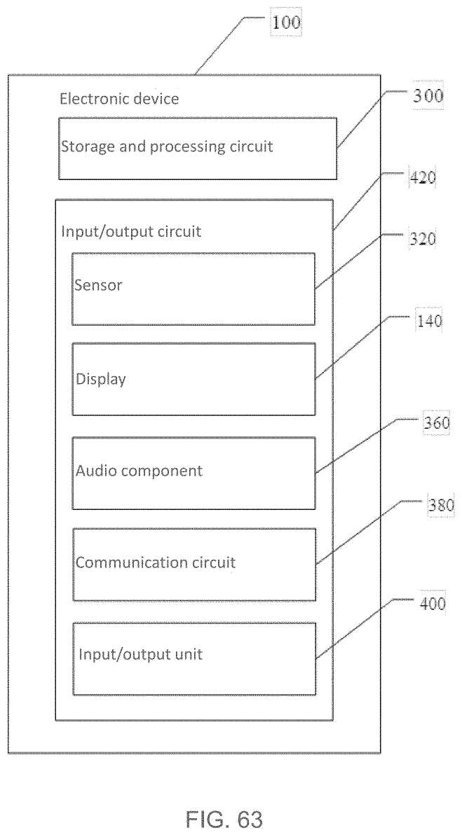

FIG. 63 is a schematic diagram showing a hardware structure of an electronic device provided in an embodiment of the present invention.

Exemplarily, the electronic device may be any of various types of computer devices that are non-movable or movable or portable and capable of wireless or wired communication. Specifically, the electronic device can be a desktop computer, a server, a mobile phone or smart phone (e.g., a phone based on iPhone™, and Android™), a portable game device (e.g. Nintendo DS™, PlayStation Portable™, Gameboy Advance™, iPhone™), a laptop computer, PDA, a portable Internet device, a portable medical device, a smart camera, a music player, a data storage device, and other handheld devices such as watches, earphones, pendants, and headphones, etc. The electronic device may also be other wearable devices (for example, electronic glasses, electronic clothes, electronic bracelets, electronic necklaces and other head-mounted devices (HMD)).

As shown in FIG. 63, the electronic device 100 includes a control circuit, and the control circuit includes a storage and processing circuit 300. The storage and processing circuit 300 includes a storage, for example, hard drive storage, non-volatile storage (such as flash memory or other storages that are used to form solid-state drives and are electronically programmable to confine the deletion, etc.), and volatile storage (such as static or dynamic random access storage) which is not limited in the embodiments of the present invention. The processing circuit in the storage and processing circuit 300 can be used to control the operation of the electronic device 100. The processing circuit can be implemented based on one or more microprocessors, microcontrollers, digital signal processors, baseband processors, power management units, audio codec chips, application specific integrated circuits, and display driver integrated circuits.

The storage and processing circuit 300 can be used to run the software in the electronic device 100, such as Internet browsing applications, Voice over Internet Protocol (VOIP) phone call applications, Email applications, media player applications, and functions of operating system. The software can be used to perform some control operations, For example, camera-based image acquisition, ambient light measurement based on an ambient light sensor, proximity sensor measurement based on a proximity sensor, information display function implemented by a status indicator based on a status indicator lamp such as light emitting diode, touch event detection based on a touch sensor, functions associated with displaying information on multiple (e.g. layered) displays, operations associated with performing wireless communication functions, operations associated with the collection and generation of audio signals, control operations associated with the collection and processing of button press event data, and other functions in electronic device 100, which is not limited in the embodiments of the present invention.

Further, the storage stores an executable program code. The processor coupled to the storage calls the executable program code stored in the storage, and implements the radio-frequency operation prompting method described in various embodiments shown above.

The executable program code includes various modules in the radio-frequency operation prompting device described in the embodiment shown in FIG. 62, for example, the acquisition module 601, the processing module 602, and the display module 603. The specific process for the above modules to implement their functions can be made reference to the relevant description in the embodiments shown in FIG. 62, and will not be repeated here.

The electronic device 100 may also include an input/output circuit 420. The input/output circuit 420 can be used to enable the electronic device 100 to implement the input and output of data, that is, the electronic device 100 is allowed to receive data from an external device and the electronic device 100 is also allowed to output data from the electronic device 100 to the external device. The input/output circuit 420 may further include a sensor 320. The sensor 320 may include one or a combination of an ambient light sensor, a proximity sensor based on light and capacitance, a touch sensor (e.g., light-based touch sensor and/or capacitive touch sensor, wherein, the touch sensor may be part of the touch screen, or it can also be used independently as a touch sensor structure), an accelerometer, and other sensors, etc.

The input/output circuit 420 may also include one or more displays, for example, display 140. The display 140 may include a liquid crystal display, an organic light emitting diode display, an electronic ink display, a plasma display, and a display that uses other display technologies. The display 140 may include a touch sensor array (i.e., the display 140 may be a touch display screen). The touch sensor can be a capacitive touch sensor formed by an array of transparent touch sensor electrodes (such as indium tin oxide (ITO) electrodes), or a touch sensor formed using other touch technologies, for example, sonic touch, pressure sensitive touch, resistive touch, and optical touch, which is not limited in the embodiments of the present invention.

The electronic device 100 can also include an audio component 360. The audio component 360 can be used to provide audio input and output functions for the electronic device 100. The audio component 360 in the electronic device 100 includes a speaker, a microphone, a buzzer, and a tone generator and other components used to generate and detect sound.

A communication circuit 380 can be used to provide the electronic device 100 with an ability to communicate with an external device. The communication circuit 380 may include an analog and digital input/output interface circuit, and a wireless communication circuit based on radio-frequency signals and/or optical signals. The wireless communication circuit in the communication circuit 380 may include a radio-frequency transceiver circuit, a power amplifier circuit, a low-noise amplifier, a switch, a filter, and an antenna. For example, the wireless communication circuit in the communication circuit 380 may include a circuit for supporting near field communication (NFC) by transmitting and receiving near-field coupled electromagnetic signals. For example, the communication circuit 380 may include a near-field communication antenna and a near-field communication transceiver. The communication circuit 380 may also include a cellular phone transceiver and antenna, and a wireless LAN transceiver circuit and antenna, etc.

The electronic device 100 may also further include a battery, a power management circuit and other input/output units 400. The input/output unit 400 may include a button, a joystick, a click wheel, a scroll wheel, a touchpad, a keypad, a keyboard, a camera, a light-emitting diode and other status indicators, etc.

The user can control the operation of the electronic device 100 by inputting a command through the input/output circuit 420, and the output data from the input/output circuit 420 enables the receiving of status information and other outputs from the electronic device 100.

Further, an embodiment of the present invention further provides a non-transitory computer-readable storage medium. The non-transitory computer-readable storage medium can be configured in the server in each of the above embodiments, and a computer program is stored in the non-transitory computer-readable storage medium. When the program is executed by the processor, the radio-frequency operation prompting method described in the embodiments is implemented.

In the embodiments described above, emphasis has been placed on the description of various embodiments. Parts of an embodiment that are not described or illustrated in detail may be found in the description of other embodiments.

It can be recognized by those skilled in the art that the exemplary modules/units and algorithm steps described in connection with embodiments disclosed herein can be implemented by electronic hardware, or a combination of computer software and electronic hardware. Whether these functions are implemented by hardware or software depends on the specific constraints for application and design of the technical solution. For each specific application, different methods can be used by professional and technical personnel to implement the described functions. However, this implementation should not be considered as going beyond the scope of the present invention.

In the embodiments provided in the present invention, it can be understood that the disclosed device/terminal and method can be implemented in other ways. For example, the device/terminal embodiments described above are merely illustrative. For example, a division of the modules or elements is merely division of logical functions, and there may be additional divisions in actual implementation. For example, multiple units or components may be combined or integrated into another system, or some features may be omitted or not performed. Alternatively, the couplings or direct couplings or communicative connections shown or discussed with respect to one another may be indirect couplings or communicative connections via some interfaces, devices or units may be electrical, mechanical or otherwise.

The units described as separate components may or may not be physically separate, and the components shown as units may or may not be physical units, i.e. may be located in one place, or may be distributed over a plurality of network elements. Some or all of the units may be selected to achieve the objectives of the solution of the present embodiment according to practical requirements.

In addition, the functional units in the various embodiments of the present invention may be integrated into one processing unit, may be physically separate from each other or may be integrated in one unit by two or more units. The integrated units described above can be implemented either in the form of hardware, or software functional units.

The integrated unit, if implemented in the form of a software functional unit and sold or used as a stand-alone product, may be stored in a computer readable storage medium. Based on such an understanding, all or part of the processes of the methods in the above-described embodiments implemented in the present invention, may also be implemented by a computer program instructing related hardware. The computer program may be stored in a computer-readable storage medium, and performs the steps of the various method embodiments described above when executed by the processor. The computer program includes a computer program code, which may be in the form of source code, object code, or executable file, or in some intermediate form. The computer readable medium may include: any entity or device capable of carrying the computer program code, recording media, U disks, removable hard disks, magnetic disks, optical disks, computer memory, read-only memory (ROM), random access memory (RAM), electric carrier wave signals, telecommunication signals and software distribution media. It should be noted that the computer readable medium may contain content that may be appropriately augmented or subtracted as required by legislation and patent practice within judicial jurisdictions, e.g., the computer-readable medium does not include electrical carrier wave signals and telecommunications signals in accordance with legislation and patent practices in some jurisdictions.

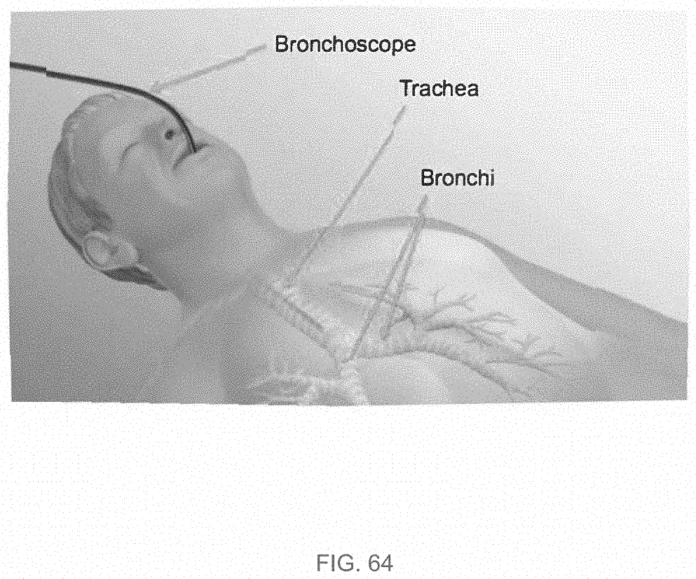

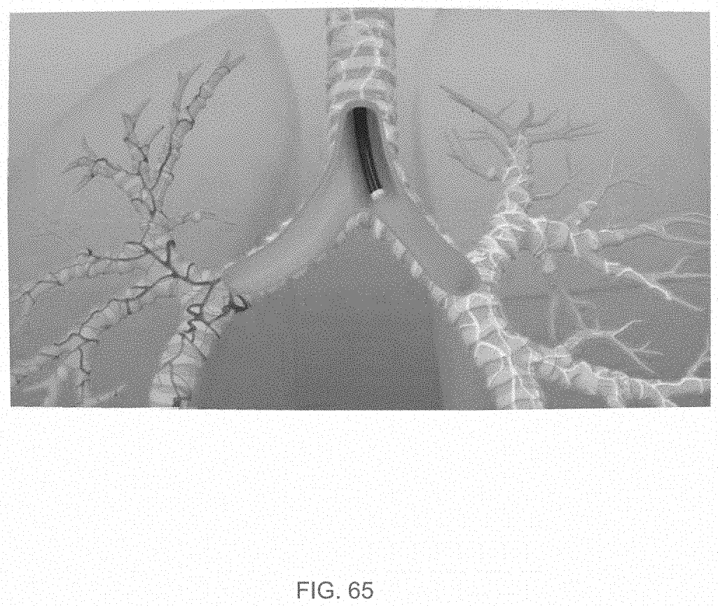

FIGS. 64-67 illustrate a bronchoscopic TLD method in accordance with embodiments of the invention.

With reference to FIG. 64, and typically performed under general anesthesia, a bronchoscope is shown inserted into the mouth of the patient. The trachea and main bronchi are shown in the lungs of the patient through which the bronchoscope is advanced.

With reference to FIG. 65, the distal end of the bronchoscope is shown being advanced along the left main bronchi.

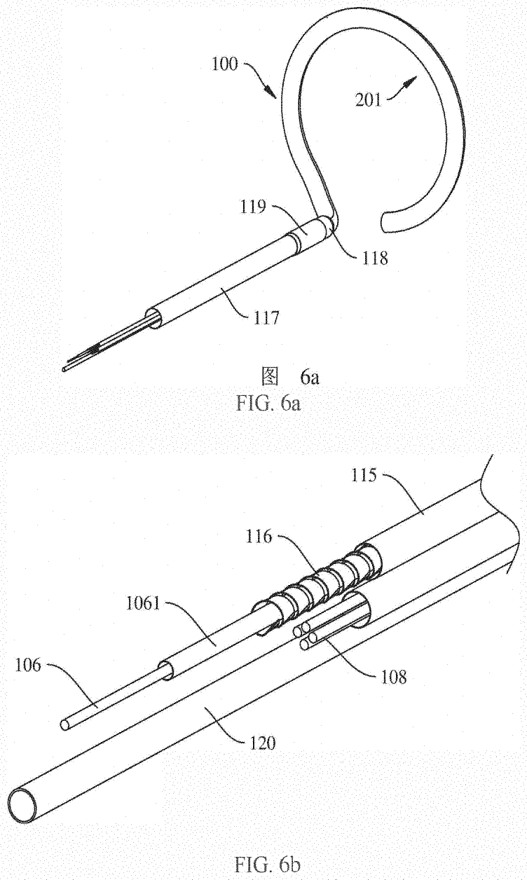

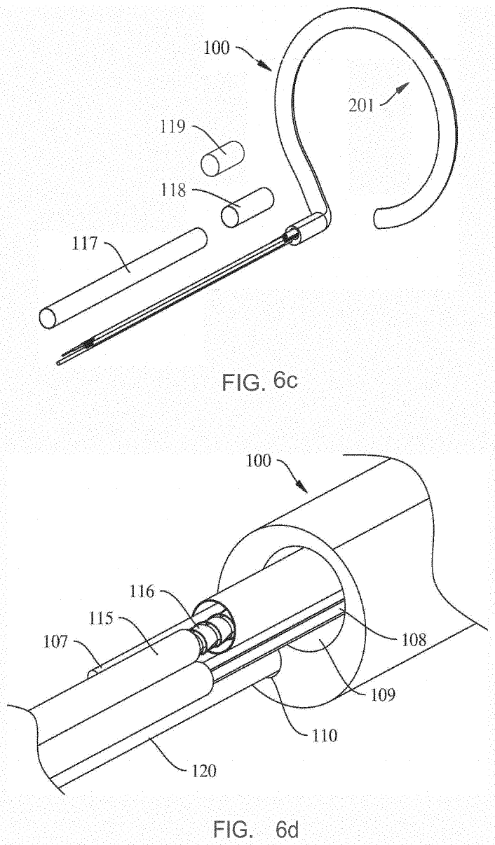

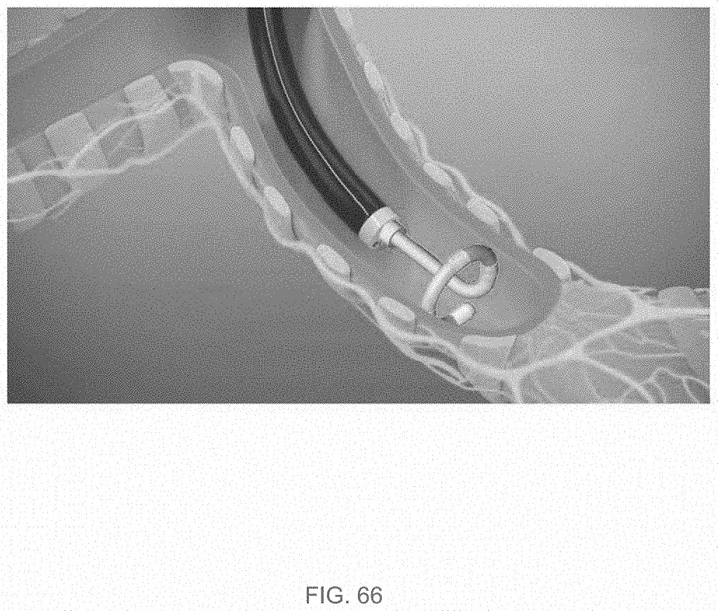

With reference to FIG. 66, a distal treatment section of an ablation catheter is shown protruding from the end of the scope's working channel. The treatment section of the ablation catheter shown in this embodiment is deployed in an open loop configuration. The loop is shown approximately normal to the axis of the catheter shaft. An example of a suitable catheter is described above in connection with FIGS. 1a-7e. Preferably, the deployed treatment section has an open configuration in contrast to inflatable members such as inflatable balloons which occlude the airway.

In preferred embodiments, and as shown in FIG. 66, a plurality of discrete spaced-apart electrodes are arranged along the loop of the treatment section. The electrodes are operable to simultaneously deliver electrosurgical energy (RF energy) and eject a cooling agent (e.g., iced saline). Each electrode is shown contacting a region of the epithelium to which the energy is applied. The controller, described above, is operable to supply power to the electrodes sufficient to heat the peripheral bronchial nerve through the epithelial layer.

In embodiments, a cooling agent is simultaneously ejected from the loop and optionally, from the electrode regions. As described herein, coolant flowrate of each electrode region can be independently controlled. The adjustable infusion of iced saline can form a liquid film at the contact position between the ablation electrode and epithelial tissue, effectively avoiding epithelial injury.

Multi-point ablation with small discrete electrodes has the advantage of providing uniform depth of ablation, improving therapeutic effectiveness while reducing the risk of burning adjacent tissues over an elongate electrode because the cooling agent described herein can be selectively delivered to individual electrode regions along the loop thereby preserving untargeted epithelium.

In preferred embodiments, temperature along the loop is monitored and the infusion flowrate is adjusted based on the local temperature along the loop. In particularly preferred embodiments, temperature at each electrode region is monitored and the coolant flowrate at each electrode region is individually tuned for optimized ablation and to minimize damage to the epithelium.

In embodiments, iced saline is infused at a higher initial flow rate than the ending flow rate, and the ablation parameters are set such that the output energy of any single ablation electrode ranges from 1000-1500 J (power limit of 10-20 W) and more preferably 1080 J˜1360 J (power limit of 12 W˜16 W) to obtain safer and more efficient ablation range. In embodiments, the ablation parameters are selected to sufficiently heat and interrupt the bronchial nerve functionality, or damage the nerve such that the nerve becomes inactive.

In embodiments, unilateral ablation is performed, followed by contralateral bronchial nerve ablation. Preferably, the nerves surrounding the main bronchi on both sides of the pulmonary are treated.

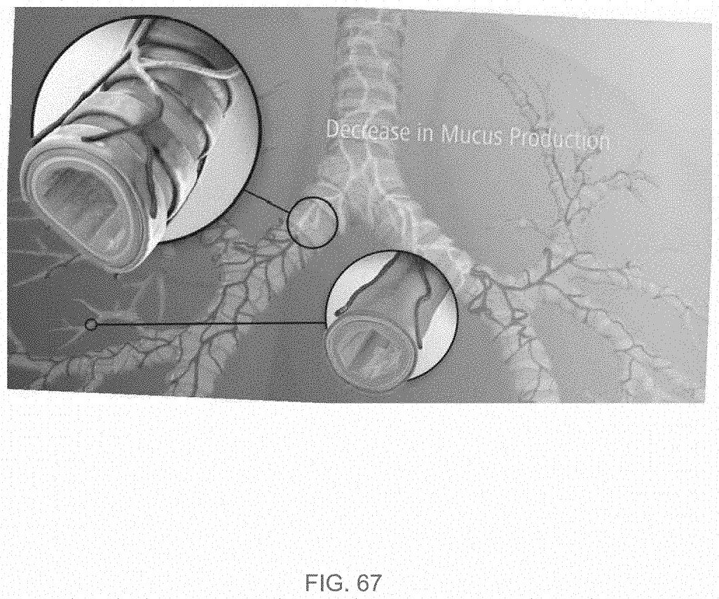

With reference to FIG. 67, prophetic examples of cross sections of treated airways are illustrated following the nerve ablation method in accordance with embodiments of the invention. Without intending to being bound to theory, destroying the motor axons of the peripheral bronchial nerve, blocks parasympathetic transmission in the pulmonary nerve and reduces acetylcholine release, resulting in effects similar to anticholinergics which includes reducing airway smooth muscle tension and mucus production, thereby improving airway obstruction.

The above-described embodiments are merely illustrative of, and not intended to limit the technical solutions of the present invention. Although the present invention has been described in detail with reference to the foregoing embodiments, it should be understood by those of skill in the art that modifications can be made to technical solutions described in the foregoing embodiments, or some of the technical features thereof can be equivalently substituted; and such modifications and substitutions do not cause the nature of the corresponding technical solution to depart from the spirit and scope of the embodiments of the present disclosure and are intended to be included within the scope of this application.

Although a number of embodiments have been disclosed above, it is to be understood that other modifications and variations can be made to the disclosed embodiments without departing from the subject invention. For example, it is intended that the systems, apparatuses, components, and method steps described herein may be combined in any logical way except where the elements are exclusive to one another. For example, it is intended that methods in accordance with embodiments of the invention may be performed using any one or more of the devices described above. However, the invention is not intended to be so limited except as where recited in any appended claims.

1. A method for lung denervation along an airway in the lung comprising:
advancing a catheter along the airway;
deploying an open loop in contact with the epithelium of the airway;
simultaneously delivering radio frequency energy from a plurality of discrete spaced-apart locations along the loop to target regions along the epithelium according to a set of ablation parameters sufficient to heat and interrupt the bronchial nerve functionality; and
forming a liquid film between the loop and the epithelium for protecting damage to the epithelium.

2. The method of claim 1, wherein forming is performed by flowing a cooling agent from the discrete spaced-apart locations onto the epithelium.

3. The method of claim 2, wherein the loop comprises an electrode at each discrete spaced-apart location for delivering radio frequency energy.

4. The method of claim 3, wherein each electrode comprises an array of egress ports, through which the cooling agent is ejected.

5. The method of claim 1, wherein non-targeted regions of the epithelium between the electrodes are protected by the cooling film.

6. The method of claim 1, further comprising retracting the loop, moving the catheter to a new location, deploying the open loop at the new location, and repeating the delivering and forming steps.

7. The method of claim 6, wherein the moving comprises advancing and/or rotating.

8. The method of claim 1, wherein the ablation parameters comprise a single electrode output energy of 1000-1500 J, and a power limit not to exceed 20 W.

9. The method of claim 1, wherein the ablation parameters comprise a single electrode output energy of 1080 J˜1360 J and power limit of 12 W˜16 W.

10. The method of claim 2, wherein the flowing is performed using iced saline.

11. The method of claim 2, wherein the flowing comprises adjusting the flowrate of the cooling agent from high to low.

12. The method of claim 2, further comprising monitoring each location, and independently adjusting the flowrate of the cooling agent to each location based on the monitoring.

13. The method of claim 12, wherein the monitoring comprises monitoring temperature.

14. The method of claim 12, further comprising displaying ablation progress based on the monitoring.

15. The method of claim 2, further comprising independently adjusting the flowrate of the cooling agent to each location such that the coolant is controllably directed to one or more desired areas of the airway and excludes one or more undesired areas.

16. The method of claim 15, wherein each area is monitored for the presence of the coolant, and the controlling is based on the monitoring.

17. An electrosurgical method of treating chronic bronchitis comprising:
destroying motor axons of a peripheral bronchial nerve, blocking parasympathetic transmission in the pulmonary nerve and reducing acetylcholine release, thereby reducing mucus production, thereby improving airway obstruction; and
simultaneously, during the destroying step, ejecting a cooling agent to a plurality of regions along the inner wall of the airway according to a plurality of customized flowrates based on temperature of each region.

18. The method of claim 17, wherein the destroying is performed by applying radiofrequency energy to discrete circumferential locations.

19. The method of claim 18, further comprising displaying ablation progress based on monitoring the temperature of each region.

20. The method of claim 19, wherein the ejecting step is performed at a sufficient rate and geography to protect epithelial tissue yet allow heat penetration to the bronchial nerve.
